(19) United States
(12) Patent Application Publication
(10) Pub. No.: US 2024/0168501 A1
(43) Pub. Date: May 23, 2024

(54) INTELLIGENT POSITIVE AND NEGATIVE PRESSURE SYSTEM AND OPERATION METHOD THEREFOR, AND INTELLIGENT POSITIVE AND NEGATIVE PRESSURE ELECTRIC APPLIANCE

(71) Applicant: Boang Zheng, Beijing (CN)

(72) Inventors: Boang ZHENG, Beijing (CN); Zhong Zheng, Beijing (CN)

(21) Appl. No.: 18/526,731

(22) Filed: Dec. 1, 2023

Related U.S. Application Data
(63) Continuation of application No. PCT/CN2022/095822, filed on May 28, 2022.

(30) Foreign Application Priority Data
Jun. 2, 2021 (CN) 202110616080.3
Jun. 2, 2021 (CN) 202121223466.X

(51) Int. Cl. G05D 16/20; A23N 12/02; A47L 15/00; A47L 15/42; A61L 2/06; A61L 2/14; A61L 2/24; A61L 9/22; D06F 25/00; D06F 33/54; D06F 33/56; D06F 33/60; D06F 33/62; D06F 33/63; D06F 33/68; D06F 34/18; D06F 103/02; D06F 105/02; D06F 105/08; D06F 105/18; D06F 105/24; D06F 105/32; D06F 105/52; D06F 105/54; F25D 11/00; F25D 17/04; F25D 21/14; F25D 23/12; F25D 29/00

(52) U.S. Cl. CPC G05D 16/204; A23N 12/02; A47L 15/0023; A47L 15/0028; A47L 15/0031; A47L 15/0034; A47L 15/0036; A47L 15/0097; A47L 15/4236; A61L 2/06; A61L 2/14; A61L 2/24; A61L 9/22; D06F 25/00; D06F 33/54; D06F 33/56; D06F 33/60; D06F 33/62; D06F 33/63; D06F 33/68; D06F 34/18; F25D 11/003; F25D 17/047; F25D 21/14; F25D 23/126; F25D 29/003; A47L 2501/01; A47L 2501/02; A47L 2501/04; A47L 2501/10; A47L 2501/16; A47L 2501/20; A47L 2501/30; A47L 2501/34; A61L 2202/122; A61L 2202/13; A61L 2202/14; A61L 2202/17; A61L 2209/111; D06F 2103/02; D06F 2105/02; D06F 2105/08; D06F 2105/18; D06F 2105/24; D06F 2105/32; D06F 2105/52; D06F 2105/54; F25D 2317/0415

(57) ABSTRACT

The present disclosure relates to an intelligent positive and negative pressure system and an operation method therefor, and an intelligent positive and negative pressure electric appliance. The system comprises a positive and negative pressure intelligent fresh-keeping refrigerator, a positive and negative pressure intelligent washing machine, a positive and negative pressure dish washing and fruit and vegetable cleaning machine, a positive and negative pressure range hood, a positive and negative pressure baking and frying microwave oven, a positive and negative pressure fresh-keeping compartment container, a positive and negative pressure fresh-keeping warehouse, a positive and negative pressure disinfection machine, and a positive and negative pressure module cabinet. By means of the positive and negative pressure system, a fluid and a carried substance thereof are regulated and controlled by means of positive and negative pressure, so as to affect an object in a specific space in a targeted manner.

### CROSS-REFERENCE TO RELATED APPLICATION

This patent application is a national stage application of International Patent Application No. PCT/CN2022/095822, filed on May 28, 2022, which claims the benefit and priority of Chinese Patent Application No. 202110616080.3 and 202121223466.x, filed with the China National Intellectual Property Administration on Jun. 2, 2021, the disclosure of which is incorporated by reference herein in its entirety as part of the present application.

### TECHNICAL FIELD

The embodiments of the present disclosure belong to the field of electric appliances, and in particular to an intelligent positive and negative pressure system and an operation method therefor, and an intelligent positive and negative pressure electric appliance.

### BACKGROUND

At present, refrigerators, washing machines and other electric appliances are becoming more and more popular. However, in recent years, the renewal of household appliances is not so desirable. For example, for more than 100 years since the advent of refrigerators, there has been no technical breakthrough in the basic storage method of low-temperature bacteriostasis, and techniques such as vacuum, high pressure, super oxygen and air condition have not been really applied in refrigerators and other electric appliances. The techniques of washing machines, dish washing and fruit and vegetable cleaning machines, range hoods and microwave ovens are also facing the problem of renewal. An objective of the embodiments of the present disclosure is to provide a new technique and a new method to solve the above problems, and to update and upgrade some existing production and living appliances, especially household appliances.

### SUMMARY

An intelligent positive and negative pressure provided by an embodiment of the present disclosure is established based on the positive and negative pressure technique. The positive and negative pressure technique is to regulate and control a fluid and a carried substance thereof by means of positive and negative pressure, so as to affect objects in a specific space. The technique principle and basic operation method for the intelligent positive and negative pressure system are as follows: intelligently regulating and controlling fluids (such as air and water) by means of the positive and negative pressure, or carrying effective loads such as super oxygen, catalysts, negative ions, air conditioning gas, disinfectant, cleaner, heat, water to get in, stay in or get out a specific space (e.g., a positive and negative pressure cabin) orderly, and applying the required influence on an object therein. The intelligent positive and negative pressure system, due to the use of the above technique operation methods, has an ultra-large-capacity carrier performance and a cross-border expansion platform advantage, can form targeted influence methods or targeted control modes with different characteristics for various affected objects. Various techniques such as vacuum, high pressure, super oxygen, catalysts, air conditioning fresh-keeping, negative ions, humidification, dehumidification, humidity and temperature control, disinfection and sterilization, air purification, air-to-water production, low-temperature storage, clothes washing and drying and food processing can be digitally regulated, controlled and integrated, and one item of or a combination of more items of the techniques is applied to positive and negative pressure electric appliances, thereby intelligently regulating and controlling and accurately creating the optimal efficiency and optimal working conditions for the operation of various electric appliances; and existing electric appliances and traditional techniques are updated and replaced, and a new field of positive and negative pressure electric appliances is developed. Due to the arrangement of the intelligent positive and negative pressure system and the use of the operation method therefor, the electric appliances are uniformly referred to as intelligent positive and negative pressure electric appliance. The intelligent positive and negative pressure electric appliance in the embodiment of the present disclosure include an intelligent positive and negative pressure fresh-keeping refrigerator, an intelligent positive and negative pressure intelligent washing machine, an intelligent positive and negative pressure dish washing and fruit and vegetable cleaning machine, an intelligent positive and negative pressure range hood, an intelligent positive and negative pressure baking and frying microwave oven, an intelligent positive and negative pressure fresh-keeping compartment container, an intelligent positive and negative pressure fresh-keeping warehouse, an intelligent positive and negative pressure disinfection machine, and an intelligent positive and negative pressure module cabinet.

The embodiments of the present disclosure adopt the following technical solution:

According to a first embodiment of the present disclosure, an intelligent positive and negative pressure system is provided, including a positive and negative pressure cabin (1), an air evacuation pump (2), an air inflation pump (3), a positive and negative pressure intelligent regulation and control apparatus (5), positive and negative pressure fluid carried substance generation processors (6), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water inlet pump (17), and the touch screen and mobile phone monitoring and identification system (12). The positive and negative pressure cabin (1) is classified into a vacuum high pressure cabin (1-1), an atmospheric circulation cabin (1-2), and atmospheric inlet-outlet cabin (1-3) according to a structure and pressure thereof. The positive and negative cabin (1) is classified into a cabinet type positive and negative pressure cabin (1A), a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D), a general refrigerator freezing compartment type positive and negative pressure fresh-keeping cabin (1E), a washing machine type positive and negative pressure cabin (1F), a dish washer type positive and negative pressure cabin (1G), a range hood type positive and negative pressure cabin (1H), a microwave oven type positive and negative pressure cabin (1K), a compartment container type positive and negative pressure cabin (1M), a warehouse type positive and negative pressure cabin (1N), a module type positive and negative pressure cabin (1T), an open type positive and negative pressure cabin (1L), a spherical/semi-spherical positive and negative pressure cabin (1Q), a cylinder type positive and negative pressure cabin (1U), a drawer type positive and negative pressure cabin (1Z), a non-airtight positive and negative pressure cabin (1X), and a shaped positive and negative pressure cabin (1R). An internal structure of the positive and negative pressure cabin (1) includes a cabin body (1.1), a cabin door (1.2), an airtight mechanism (1.3), and an internal and external communicating sealer (1.4). The airtight mechanism (1.3) is arranged between the cabin body (1.1) and the cabin door (1.2); the internal and external communicating sealer (1.4) is fixedly arranged at a rear part or side part of the positive and negative pressure cabin (1), and all pipelines and circuits getting in and out the cabin are connected to and pass through the internal and external communicating sealer (1.4). Each of the positive and negative pressure refrigerating fresh-keeping cabin (1B), a positive and negative pressure freezing fresh-keeping cabin (1C) has a corresponding positive and negative pressure resisting structure, and is provided with an airtight mechanism (1.3), and an internal and external communicating sealer (1.4), which belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the positive and negative pressure cabin. The general refrigerator refrigerating compartment type positive and negative pressure fresh-keeping cabin (1D) and the general refrigerator freezing compartment type positive and negative pressure fresh-keeping cabin (1E) are that a refrigerating compartment and a freezing compartment of the general refrigerator are additionally provided with the positive and negative pressure system, but without the airtight mechanism (1.3) and the internal and external communicating sealer (1.4), thus the general refrigerator refrigerating compartment type positive and negative pressure fresh-keeping cabin (1D) and the general refrigerator freezing compartment type positive and negative pressure fresh-keeping cabin (1E) do not have a vacuum high pressure resistance structure, and belongs to a normal sealed atmospheric circulation cabin (1-2) or atmospheric inlet-outlet cabin (1-3) structure. The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a super oxygen generation water mixer (6.6), a disinfection, sterilization and degradation apparatus (6.9), a filtering and refreshing apparatus (6.10), a stain removal and oil dispelling cleaning apparatus (6.11), a refrigeration, heating and temperature control apparatus (6.13), a carried substance generator item addition and upgrading module (6.16), an airflow carried substance generator (6.01), an airflow carried substance generator integration (6.02), a waterflow carried substance generator (6.03), a waterflow carried substance generator integration (6.04), and an integrated pipeline type positive and negative fluid carried substance generator (6.05). The air conditioning apparatus (6.1) is a membrane-based air separation type air conditioning apparatus. The carried substance generator item addition and upgrading module (6.16) is a combined module capable of providing or installing any individual fluid carried substance generator additional item in the carried substance generator integration and upgrading and updating the same; each of the airflow carried substance generator integration (6.02), the waterflow carried substance generator integration (6.04) and the integrated pipeline type positive and negative pressure fluid carried substance generator (6.05) includes any one or more in the individual fluid carried substance generators (6.1) to (6.16). The integrated pipeline type positive and negative pressure fluid carried substance generator (6.05) is a small, intelligent and integrated fluid carried substance generator and sensor and connected pipelines and circuits; the positive and negative pressure system, according to fluid classification, includes a positive and negative pressure airflow system, and a positive and negative pressure waterflow system. A standard structure of the positive and negative pressure airflow system includes a positive and negative pressure cabin (1), an air evacuation pump (2), an air inflation pump (3), a positive and negative pressure intelligent regulation and control apparatus (5), a touch screen and mobile phone monitoring and identification system (12), and individual fluid carried substance generators (6.1) to (6.16), or a positive and negative pressure airflow carried substance generator integration (6.02), or an integrated pipeline type positive and negative fluid carried substance generator (6.05). The positive and negative pressure airflow system also adopts a nonstandard structure other than the above standard structure, and the nonstandard structure of the positive and negative pressure airflow system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the individual fluid carried substance generators (6.1) to (6.16), the positive and negative pressure airflow carried substance generator integration (6.02), the integrated pipeline type positive and negative fluid carried substance generator (6.05), and the touch screen and mobile phone monitoring and identification system (12). The positive and negative pressure cabin is externally provided with the air evacuation pump (2), the air inflation pump (3), the intelligent positive and negative pressure regulation and control apparatus (5), the touch screen and mobile phone monitoring and identification system (12) and the positive and negative pressure airflow carried substance generator integration (6.02). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the positive and negative pressure cabin 1 to form an air evacuation and air return port (c). An air exhaust pipeline (A2) is installed at an air outlet port (b) of the air evacuation pump (2), a solenoid valve (B2) is installed at a middle part of the air exhaust pipeline (A2), and the other end of the air exhaust pipeline is an exhaust port. A carrying circulation pipeline (A5.1) is further connected to the solenoid valve (B1), the carrying circulation pipeline (A5.1) extends into the positive and negative pressure cabin (1) through the solenoid valve (B1) and the air evacuation pipeline (A1) to form a carrying air inlet port (j1), and the other end of the carrying circulation pipeline (A5.1) is connected to a carrying circulation air inlet port (b2) of the air evacuation pump (2). A carrying waste gas evacuation and exhaust pipeline (A8) is also connected to the solenoid valve (B1), and the other end of the carrying waste gas evacuation and exhaust pipeline (A8) is connected to a carrying waste gas evacuation and exhaust port (s2) of an integrated air conditioning apparatus (6.1) in the positive and negative pressure airflow carried substance generator integration (6.02). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the positive and negative pressure cabin (1) to form an air inflation inlet (f) thereof. An air intake pipeline (A4) is installed at an air inlet port (3) of the air inflation pump (3), a solenoid valve (B4) is installed at a middle part of the air intake pipeline (A4), and the other end of the air intake pipeline (A4) is connected to atmosphere. A carrying circulation pipeline (A5) is further connected to the solenoid valve (B3), the carrying circulation pipeline (A5) extends into the positive and negative pressure cabin 1 through the solenoid valve (B3) and the air inflation pipeline (A3) to form a carrying air return port (j) thereof, and the other end of the carrying circulation pipeline (A5) is connected to a carrying circulation air inlet port (e2). A carrying air introduction pipeline (A6) is installed at an air inlet port (t) of the positive and negative pressure airflow carried substance generator integration (6.02), and a solenoid valve (B6) is installed on the other end of the carrying air introduction pipeline (A6). A carrying air introduction pipeline (A7) is installed at an air outlet port (s) of the positive and negative pressure airflow carried substance generator integration (6.02), a solenoid valve (B7) is installed at a middle part of the carrying air introduction pipeline (A7), and the other end of the carrying air introduction pipeline (A7) extends into the positive and negative pressure cabin (1) to form a carrying air inflation port (i). The solenoid valve (B6) is respectively connected to a carrying air evacuation pipeline (A6.1) and a carrying air intake pipeline (A6.2), the carrying air evacuation pipeline (A6.1) is connected to the air evacuation pipeline (A1) and then is connected to an air evacuation port (a) of the air evacuation pump (2), and the carrying air intake pipeline (A6.2) is connected to the air inflation pipeline (A3) and then communicates with the air outlet port (d) of the air inflation pump (3). A solenoid valve (B6.2) is installed at a middle part of the carrying air intake pipeline (A6.2), and the other end of the carrying air intake pipeline (A6.2) extends into the positive and negative pressure cabin (1) and then is connected to the integrated pipeline type positive and negative pressure fluid carried substance generator (6.05). When a carrying airflow entering the positive and negative pressure cabin (1) requires air inflation circulation, the air inflation inlet (f) serves as the carrying air return port (j) of the positive and negative pressure cabin (1). When the carrying airflow entering the positive and negative pressure cabin (1) requires air evacuation circulation, the air evacuation and air return port (c) serves as the carrying air inlet port (j1) of the positive and negative pressure cabin (1), and the carrying air inflation port (i) serves as a carrying air evacuation port (i1) of the positive and negative pressure cabin (1). A standard structure of the positive and negative pressure waterflow system includes a positive and negative pressure cabin (1), a water suction pump (16), a water input pump (17), a positive and negative pressure intelligent regulation and control apparatus (5), a touch screen and mobile phone monitoring and identification system (12), and a waterflow carried substance generator (6.03), or a waterflow carried substance generator integration (6.04). The positive and negative pressure airflow system also adopts a nonstandard structure other than the above standard structure, and the nonstandard structure of the positive and negative pressure airflow system includes the water suction pump (16) or water input pump (17), and at least one or more of the positive and negative pressure cabin (1), the water suction pump (16), the water input pump (17), the positive and negative pressure intelligent regulation and control apparatus (5), individual fluid carried substance generators (6.1) to (6.16), the waterflow carried substance generator integration (6.04), and the touch screen and mobile phone monitoring and identification system (12). The positive and negative pressure cabin is externally provided with the water suction pump (16), the water input pump (17), the positive and negative pressure intelligent regulation and control apparatus (5), the touch screen and mobile phone monitoring and identification system (12) and the waterflow carried substance generator integration (6.04). A water suction pipeline (A20) is installed at a water suction port (w) of the water suction pump (16), a solenoid valve (B20) is installed at a middle part of the water suction pipeline (A20), and the other end of the water suction pipeline (A20) extends into the positive and negative pressure cabin (1) to form a water suction and water output port (L). A water drainage pipeline (A21) is installed at a water drainage port (p) of the water suction pump (16), a solenoid valve (B21) is installed at a middle part of the water drainage pipeline (A21), and the other end of the water drainage pipeline (A21) is a water drainage port. A carrying circulation pipeline (A19.1) is further connected to the solenoid valve (B20), the carrying circulation pipeline (A19.1) extends into the positive and negative pressure cabin (1) through the solenoid valve (B20) and the water suction pipeline (A20) to form a carrying water inlet port (L1) of the positive and negative pressure cabin (1), and the other end of the carrying circulation pipeline (A19.1) is connected to a carrying circulation water output port (p2) of the water suction pump (16). A water intake pipeline (A15) is installed at a water output port (u) of the water input pump (17), a solenoid valve (B15) is installed at a middle part of the water intake pipeline (A15), and the other end of the water intake pipeline (A15) extends into the positive and negative pressure cabin (1) to form a water source intake (T). A water intake pipeline (A14) is installed at a water inlet port (r) of the water input pump (17), a solenoid valve (B14) is installed at a middle part of the water intake pipeline (A14), and the other end of the water intake pipeline (A14) is connected to a water source. A carrying circulation pipeline (A19) is further connected to the solenoid valve (B15), the carrying circulation pipeline (A19) extends into the positive and negative pressure cabin (1) through the solenoid valve (B15) and the water intake pipeline (A15) to form a carrying water return port (L2) of the positive and negative pressure cabin (1), and the other end of the carrying circulation pipeline (A19) is connected to a carrying circulation water inlet port (r2) of the water input pump (17). A carrying water introduction pipeline (A16) is installed at a water inlet port v of the positive and negative pressure waterflow carried substance generator integration (6.04), and a solenoid valve (B16) is installed at the other end of the carrying water introduction pipeline (A16). A carrying water introduction pipeline (A17) is installed at a water output port (o) of the positive and negative pressure waterflow carried substance generator integration (6.04), a solenoid valve (B17) is installed at a middle part of the carrying water introduction pipeline (A17), and the other end of the carrying water introduction pipeline (A17) extends into the positive and negative pressure cabin (1) to form a carrying water inlet port (T1) of the positive and negative pressure cabin (1). The solenoid valve (b16) is respectively connected to a carrying water suction pipeline (A16.1) and a carrying water intake pipeline (A16.2), the carrying water suction pipeline (A16.1) is connected to the water suction pipeline (A20) and then communicates with a water suction port (w) of the water suction pump (16). The carrying water intake pipeline (A16.2) is connected to the water intake pipeline (A15) and then communicates with the water output port (u) of the water input pump (7). When a carrying waterflow entering the positive and negative pressure cabin (1) requires water intake circulation, the water source intake (t) serves as the carrying water return port (L2) of the positive and negative pressure cabin (1). When a carrying waterflow entering the positive and negative pressure cabin (1) requires water suction circulation, the water suction output port (L) serves as the carrying water inlet port (L1) of the positive and negative pressure cabin (1). The touch screen and mobile phone monitoring and identification system (12) includes a refrigerator door touch screen (12.1), a mobile phone APP (12.2), a high-definition anti-fog camera apparatus (12.3), and a radar scanning recognition apparatus (12.5). The high-definition anti-fog camera apparatus (12.3) and the radar scanning recognition apparatus (12.5) are installed at positions needing to be monitored inside and outside the positive and negative pressure cabin. The positive and negative pressure cabin (1) is internally provided with positive and negative pressure fluid carried substance generators (6), including: a super oxygen generator (6.2), a negative ion generator (6.3), a catalyst release controller (6.4), a humidification, dehumidification and humidity control apparatus (6.5), a disinfection, sterilization and degradation apparatus (6.9), a filtering and refreshing apparatus (6.10), a refrigeration, heating and temperature control apparatus (6.13), a carried substance generator item addition and upgrading module (6.16), or integrated pipeline type positive and negative pressure fluid carried substance generator (6.05). The positive and negative pressure cabin (1) is internally provided with sensors (C), including a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), a carried substance sensor item addition and upgrading module (C12), and a sensor integration (CA). The carried substance sensor item addition and upgrading module (C12) is a combined module capable of providing and installing any individual sensor additional item into the sensor integration and upgrading and updating the same; the sensor integration (CA) includes at least one of more of individual sensors (C1) to (C12). All fluid carried substance generation processors (6), sensors (C) and solenoid valves (B) and the air evacuation pump (2), the air inflation pump (3), the water suction pump (16), the water input pump (17) and the touch screen and mobile phone monitoring and identification system (12) are connected to the positive and negative pressure intelligent regulation and control apparatus (5) by lines (D) or bundled lines (E).

According to a second embodiment of the present disclosure, an operation method for the intelligent positive and negative pressure system is provided. The intelligent positive and negative pressure system is established according to the positive and negative pressure technique, and the positive and negative pressure technique is to regulate and control a fluid and a carried substance thereof by means of positive and negative pressure, so as to affect objects in a specific space. The technical principle and a basic operation method for the intelligent positive and negative pressure system are as follows: intelligently regulating and controlling fluids (for example, air and water) with positive and negative pressure, or carrying multiple effective loads such as super oxygen, a catalyst, a negative ion, an air-conditioned gas, a disinfectant, a cleaning agent, heat and water to orderly get in and out of or stay in a specific space (for example, a positive and negative pressure cabin), and exerting required effects on objects therein. The specific operation method for the intelligent positive and negative pressure system is that according to built-in program, pre-stored data and cloud data of the intelligent positive and negative pressure system, and real-time feedback information of a touch screen and mobile phone monitoring and identification system (12) and various sensors (C11) to (C12), a positive and negative pressure intelligent regulation and control apparatus (5), after calculation processing, forms various targeted influence methods or targeted control modes with different characteristics for various affected objects, and sends specific instructions to an air evacuation pump (2), an air inflation pump (3), a water suction pump (16), a water input pump (17), related positive and negative pressure fluid carried substance generators (6) and related solenoid valves (c) in real time, so as to accurately control and timely adjust open, close and switching of various air evacuation, inflation and circulation pipelines or water suction, intake and circulation pipelines. The level of positive and negative pressure in the positive and negative pressure cabin (1) and flowing-in, staying and flowing-out of an airflow and a carried substance thereof or a waterflow and a carried substance thereof are intelligent regulated and controlled, with specific operation method as follows: operation method (I): an operation method for regulating an interior of the positive and negative pressure cabin (1) into negative pressure, namely, vacuum, is as follows: sending an instruction by the positive and negative pressure intelligent regulation and control apparatus (5) to enable solenoid valves (B1) and (B2) to open air evacuation pipelines (A1) and (A2), air evacuation and air return port c of the positive and negative pressure cabin→air evacuation pipeline (A1)→solenoid valve (B1)→(A1)→air inlet port (a) of the air evacuation pump→air outlet port (b) of the air evacuation pump→(A2)→(B2)→(A2)→exhaust to atmosphere) (when any pipeline is opened, other unrelated solenoid valves are all closed, the following is the same and will not be described again), and turning on the air evacuation pump (2) to vacuumize the interior of the positive and negative pressure cabin (1) to set negative pressure vacuum; operation method (II): an operation method for regulating and controlling the interior of the positive and negative pressure cabin 1 to positive pressure, namely, high pressure, is as follows: sending an instruction by the positive and negative pressure intelligent regulation and control apparatus (5) to enable solenoid valves (B3) and (B4) to open air inflation pipelines (A3) and (A4) as follows: (intake from atmosphere→(A4)→(B4)→(A4)→air inlet port (e) of air inflation pump→air outlet port (d) of air inflation pump→(A3)→(B3)→(A3)→air inflation and intake (f) of the positive and negative pressure cabin, and turning on the air inflation pump (3) to inflate the interior of the positive and negative pressure cabin (1) to the set positive pressure, namely, high pressure; the above operation methods (1) and (2) are only conducted in a vacuum or high-pressure space, such that the positive and negative pressure cabin (1) is classified into a vacuum high pressure cabin (1-1), and a positive and negative pressure cold-preservation fresh-keeping cabin (1B), a positive and negative pressure freezing fresh-keeping cabin (1C), a washing machine type positive and negative pressure cabin (1F), a dish washer type positive and negative pressure cabin (1G), and a microwave oven type positive and negative pressure cabin (1K) all belong to the vacuum high pressure cabin; operation method (III): an operation method for regulating airflow and a carried substance thereof to flow in, stay in or flow out of the positive and negative pressure cabin (1) is as follows: (i) outflow, when the cabin is under the negative pressure or atmospheric, evacuating the airflow and the carried substance thereof out of the positive and negative pressure cabin (1) from air evacuation pipelines using the air evacuation pump (2) according to the above specific operation method (I), and when the cabin is under the high pressure, opening the corresponding solenoid valves for the airflow and the carried substance to flow out from the air evacuation pipelines; (ii) inflow, inflating the airflow and the carried substance thereof into the positive and negative pressure cabin from air inflation pipelines or carrying pipelines using the air inflation pump (3), with specific method as follows: A: inflow of the airflow: when the cabin is under the high pressure or atmospheric, inflating the airflow into the positive and negative pressure cabin from the air inflation pipeline according to the specific operation method (II) using the air inflation pump (3), and when the cabin is under the negative pressure, opening related solenoid valves to enable the airflow to automatically flow into the cabin from the air inflation pipeline; B: inflow of carrying airflow generated by an airflow carried substance generator integration (6.02): sending an instruction by the positive and negative pressure intelligent regulation and control apparatus (5) to enable solenoid valves (B6) and (B7) to open carrying air inflation pipelines (A3), (A6.2), (A6) and (A7), air intake from atmosphere→(A4)→(B4)→(A4)→air inlet port (e) of air inflation pump→air outlet port (d) of air inflation pump→(A3)→(A6.2)→(A6)→(B6)→(A6)→related airflow carried substance generator in airflow carried substance integration (6.02)→(A7)→(B7)→(A7)→carrying air inlet port (i) of positive and negative pressure cabin, and turning on the air inflation pump (3) and the related airflow carried substance generator to make the pressure in the positive and negative pressure cabin (1) and inflow of the airflow carried substance reach a set standard; C: inflow of a carrying airflow generated by an integrated pipeline type fluid carried substance generator (6.05): sending an instruction by the positive and negative pressure intelligent regulation and control apparatus (5) to enable a solenoid valve (B6.2) to open carrying air inflation pipelines (A3) and (A6.2), air intake from atmosphere→(A4)→(B4)→(A4)→air inlet port (e) of air inflation pump→air outlet port (d) of air inflation pump→(A3)→(A6.2)→(B6.2)→(A6.2)→related airflow carried substance generator in integrated pipeline type fluid carried substance generator (6.05)→positive and negative pressure cabin, and turning on the air inflation pump (3) and the related airflow carried substance generator in the integrated pipeline type fluid carried substance generator (6.05) to make the pressure in the positive and negative pressure cabin and inflow of the airflow carried substance reach a set standard; (iii) staying, when the airflow and the carried substance thereof need to stay in the positive and negative pressure cabin, sending an instruction by the positive and negative pressure intelligent regulation and control apparatus (5) to make the airflow and pressure and carried substance in the cabin reach the standard, and then closing the corresponding solenoid valves; (iv) circulation: A: air inflation circulation: when the airflow and the carried substance thereof need to circulate to flow through the positive and negative pressure fluid carried substance generator to make the pressure, concentration and composition of the air flow and the carried substance thereof in the positive and negative pressure cabin reach the standard, sending an instruction by the positive and negative pressure intelligent regulation and control apparatus (5) to open carried substance circulation pipelines (A3), (A5), (A6), (A6.2) and (A7) and close (B4) and corresponding solenoid valves, and turning on the air inflation pump (3) and related airflow carried substance generators to make carried substance gases circulate and flow, during the operation of an air conditioning apparatus (6.1), moderately increasing partial pressure difference between both sides of a membrane to improve air separation efficiency, and evacuating and exhausting oxygen-enriched waste gas or carried waste gas; and opening carrying waste gas evacuation and exhaust pipelines (A8), (A1) and (A2) by the solenoid valves (B1) and (B2), carrying waste gas evacuation and exhaust port (s2) of air conditioning apparatus (6.1)→solenoid valve (B1)→(A1)→air inlet port (a) of air evacuation pump→air outlet port (b) of air evacuation pump→(A2)→(B2)→(A2)→exhaust to the atmosphere, and turning on the air evacuation pump (2), operating circularly until corresponding sensors in the positive and negative pressure cabin feed back that the concentration, composition or pressure of the air-conditioned or carrying gas reach the standard, and sending a stop instruction by the positive and negative pressure intelligent regulation and control apparatus (5); B: air evacuation circulation: sending an instruction by the positive and negative pressure intelligent regulation and control apparatus (5) to open carrying air evacuation pipelines (A1), (A5.1), (A6.1) (A6) and (A7) and close (B2) and related solenoid valves, and turning on the air evacuation pump (2) and related airflow carried substance generators to make carried substance gases circulate and flow until corresponding sensors in the positive and negative pressure cabin feed back that the concentration, composition or pressure of the air-conditioned gas reach the standard, and sending a stop instruction by the positive and negative pressure intelligent regulation and control apparatus (5); as the operation methods for the positive and negative pressure cabin (1), namely, (i) outflow, (ii) inflow, (iii) staying and (iv) circulation, are conducted in an atmospheric space, the positive and negative pressure cabin (1) is classified into an atmospheric circulation cabin (1-2) and an atmospheric inlet-outlet cabin (1-3); the general refrigerator refrigerating compartment type positive and negative pressure fresh-keeping cabin (1D) and the general refrigerator freezing compartment type positive and negative pressure fresh-keeping cabin (1E) belong to the atmospheric circulation cabin or the atmospheric inlet-outlet cabin, positive and negative pressure systems are added and used inside and outside the refrigerating compartment and freezing compartment of the general refrigerator, but an airtight mechanism (1.3) and an internal and external communicating sealer (1.4) are not added; the general refrigerator can use the positive and negative pressure system and any one or more of (i) outflow, (ii) inflow, (iii) staying and (iv) circulation procedures in the operation method therefor under atmospheric, thus significantly enhancing the fresh-keeping capacity of the refrigerator and increase the function and efficacy of the refrigerator in many aspects; operation method (IV): an operation method for regulating a waterflow and a carried substance thereof to flow in, stay in or flow out of the positive and negative pressure cabin (1) is as follows: (i) outflow, pumping the waterflow and the carried substance thereof out of the positive and negative pressure cabin from water suction pipelines using a water suction pump (16), with specific methods as follows: sending an instruction by the positive and negative pressure intelligent regulation and control apparatus (5) to open water suction pipelines (A20) and (A21) by solenoid valves (B20) and (B21), water suction port (L) of positive and negative pressure cabin→water suction pipeline (A20)→solenoid valve (B20)→(A20)→water inlet port (w) of water suction pump→water drainage port (p) of water suction pump→(A21)→(B21)→(A21)→drainage to sewer, and turning on the water suction pump (16) to drain the water in the positive and negative pressure cabin (1) to a set standard; (ii) inflow, feeding the waterflow and the carried substance thereof into the positive and negative pressure cabin from air feeding and intake pipelines or carrying water intake pipelines using the water input pump (17), with specific method as follows: A: water feeding and intake: sending an instruction by the positive and negative pressure intelligent regulation and control apparatus (5) to open water intake pipelines (A14) and (A15) by solenoid valves (B14) and (B15), water intake from water source→(A14)→(B14)→(A14)→water inlet port (r) of water input pump→water output port (u) of water input pump→(A15)→(B15)→(A15)→water feeding and inlet port (T) of the positive and negative pressure cabin, and turning on the water input pump (17) to make the water feeding in the positive and negative pressure cabin (1) reach the standard; B: carrying water intake: sending an instruction by the positive and negative pressure intelligent regulation and control apparatus (5) to open carrying water intake pipelines (A14), (A15), (A16), (A16.2) and (A17) by solenoid valves (B14), (B16) and (B17), water intake from water source→(A14)→(B14)→(A14)→water inlet port (r) of water input pump→water output port (u) of water input pump→(A15)→(A16.2)→(B16)→(A16)→corresponding waterflow carried substance generator→(A17)→(B17)→(A17)-carrying water inlet port (T1) of positive and negative pressure cabin, and turning on the water input pump (17) and the corresponding waterflow carried substance generator to make water feeding in the positive and negative pressure cabin (1) and inflow of the waterflow carried substance to a set standard; (iii) staying, when the waterflow and the carried substance thereof need to stay in the positive and negative pressure cabin, sending an instruction by the positive and negative pressure intelligent regulation and control apparatus (5), firstly making the waterflow pressure and carried substance in the cabin reach the standard, and then closing the corresponding solenoid valves until staying time reaches the standard, and sending a stop instruction by the positive and negative pressure intelligent regulation and control apparatus (5); (iv) circulation: A: water intake circulation: when the waterflow and the carried substance thereof need to circulate to flow through the positive and negative pressure fluid carried substance generator to make the pressure, concentration and composition of the waterflow and the carried substance thereof in the positive and negative pressure cabin reach the standard, sending an instruction by the positive and negative pressure intelligent regulation and control apparatus (5) to open carrying water intake circulation pipelines (A15), (A16), (A16.2), (A17) and (A19) and close (B14) and related solenoid valves, and turning on the water input pump (17) and related waterflow carried substance generators to make substance-carrying waterflow circulate and flow until corresponding sensors in the positive and negative pressure cabin feed back that the concentration, the composition or pressure of the carrying waterflow reach the standard, and sending a stop instruction by the positive and negative pressure intelligent regulation and control apparatus (5); B: water suction circulation: sending an instruction by the positive and negative pressure intelligent regulation and control apparatus (5) to open carrying water suction pipelines (A20), (A16), (A16.1), (A17) and (A19.1) and close (B21) and related solenoid valves, and turning on the water suction pump (16) and related waterflow carried substance generators to make the substance-carrying waterflow circuit and flow until corresponding sensors in the positive and negative pressure cabin feed back that the concentration, the composition or pressure of the waterflow reach the standard, and sending a stop instruction by the positive and negative pressure intelligent regulation and control apparatus (5); one or more of the operation methods, such as, (i) outflow, (ii) inflow, (iii) staying and (iv) circulation, for regulating and controlling the waterflow and carried substance thereof to flow in, stay in or flow out the positive and negative pressure cabin (1) are conducted not only in a vacuum high pressure cabin, such as a washing machine type positive and negative pressure cabin (1F), a dish washer type positive and negative pressure cabin (1G), but also in an atmospheric circulation cabin (1-2) and the atmospheric inlet-outlet cabin (1-3), such as a range hood type positive and negative pressure cabin (1H), an open type positive and negative pressure cabin (1L), and a non-airtight positive and negative pressure cabin (1X); the intelligent positive and negative pressure system, due to the use of above technical operation methods, has an ultra-large-capacity carrier and renewable performance and a platform advantage of upgrading, extending and adding items. Various techniques such as vacuum, high pressure, super oxygen, catalysts, air conditioning, negative ions, disinfection and purification, moisture and temperature control, air-to-water production, low-temperature storage, clothes washing and drying and food processing can be intelligently regulated, controlled and integrated, and one item of or a combination of more items of the techniques is applied to electrical equipment, transport compartments, containers and warehouses. Due to the use of the intelligent positive and negative pressure system and operation method therefor, the electrical equipment, transport carriages, containers and warehouses are uniformly referred to as intelligent positive and negative pressure electric appliances. The intelligent positive and negative pressure electric appliances in embodiments of the present disclosure include an intelligent positive and negative pressure fresh-keeping refrigerator, an intelligent positive and negative pressure washing machine, an intelligent positive and negative pressure dish washing and fruit and vegetable cleaning machine, an intelligent positive and negative pressure range hood, an intelligent positive and negative pressure baking and frying microwave oven, an intelligent positive and negative pressure fresh-keeping compartment container, an intelligent positive and negative pressure fresh-keeping warehouse, an intelligent positive and negative pressure disinfection machine, and an intelligent positive and negative pressure module cabinet.

According to a third embodiment of the present disclosure, an intelligent positive and negative pressure refrigerator is provided, including a refrigerator body (7). The refrigerator body (7) is internally provided with an intelligent positive and negative pressure system, a refrigeration system (8), and an air-to-water production apparatus (11). The intelligent positive and negative pressure system includes a positive and negative pressure cabin (1), an air evacuation pump (2), an air inflation pump (3), a positive and negative pressure intelligent regulation and control apparatus (5), airflow carried substance generator integrations (6.021), (6.022), (6.023), (6.024) and (6.025), and a touch screen and mobile phone monitoring system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), a positive and negative pressure fluid carried substance generation processor (6), and the touch screen and mobile phone monitoring and identification system (12). The positive and negative pressure cabin (1), according to cold preservation and freezing requirements of a fresh-keeping refrigerator, is designed as a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D), and a general refrigerator freezing compartment type positive and negative pressure fresh-keeping cabin (1E). The positive and negative pressure refrigerating fresh-keeping cabin (1B) and the positive and negative pressure freezing fresh-keeping cabin (1C) are both vacuum high pressure cabins (1-1), a positive and negative pressure resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the refrigerator; one or more of positive and negative pressure refrigerating fresh-keeping cabins (1B) and positive and negative pressure freezing fresh-keeping cabins (1C) are respectively arranged in the refrigerator body (7) in up-down or left-right arrangement and asymmetrical irregular arrangement modes. The positive and negative pressure refrigerating fresh-keeping cabin (1B) and the positive and negative pressure freezing fresh-keeping cabin (1C), according to the shape and characteristic classification, include a drawer type positive and negative pressure refrigerating fresh-keeping cabin (1B-1), a drawer type positive and negative pressure freezing fresh-keeping cabin (1C-1), a side door cabinet type positive and negative pressure refrigerating fresh-keeping cabin (1B-2), and a side door cabinet type positive and negative pressure freezing fresh-keeping cabin (1C-2). The drawer type positive and negative pressure refrigerating fresh-keeping cabin (1B-1) and the drawer type positive and negative pressure freezing fresh-keeping cabin (1C-1) include cabin bodies (1.1), a positive and negative pressure refrigerating fresh-keeping drawer (1.81), a positive and negative pressure freezing fresh-keeping drawer (1.82), and internal and external communicating sealers (1.4). A drawer type airtight mechanism (1.9) is arranged between the cabin body of the drawer type positive and negative pressure refrigerating fresh-keeping cabin (1B-1) and the positive and negative pressure refrigerating fresh-keeping drawer (1.81) and between the cabin body of the drawer type positive and negative pressure freezing fresh-keeping cabin (1C-1) and the positive and negative pressure freezing fresh-keeping drawer (1.82). The drawer type airtight mechanism (1.9) includes a locking ring (1.91), a lock bolt (1.92), and an airtight gasket (1.93); the locking ring (1.91) is in movable fit with the lock bolt (1.92), when the fresh-keeping drawer is closed, the locking bolt (1.92) extends into the locking ring (1.92) to lock a cabin door, and the airtight gasket (1.93) is used for keeping sealing. The side door cabinet type positive and negative pressure refrigerating fresh-keeping cabin (1B-2) and the side door cabinet type positive and negative pressure freezing fresh-keeping cabin (1C-2) each include a cabin door (1.1), a cabin door (1.2), an airtight mechanism (3), and an internal and external communicating sealer (1.4). The airtight mechanism (1.3) is arranged between the cabin door (1.2) and the cabin body (1.1). The airtight mechanism (1.3) includes a locking ring (1.31), a lock bolt (1.32), an airtight gasket (1.33), and a door rim sealing gasket; the locking ring (1.31) is in movable fit with the lock bolt (1.32). When the cabin door is closed, the locking bolt (1.32) extends into the locking ring (1.32) to lock the cabin door, and the airtight gasket (1.33) is used for keeping sealing. The internal and external communicating sealer (1.4) is arranged on a side part or a side part of each of the drawer type positive and negative pressure refrigerating fresh-keeping cabin (1B-1), the drawer type positive and negative pressure freezing fresh-keeping cabin (1C-1), the side door cabinet type positive and negative pressure refrigerating fresh-keeping cabin (1B-2), and the side door cabinet type positive and negative pressure freezing fresh-keeping cabin (1C-2), all pipelines and circuits getting in and out the cabin are connected to and pass through the internal and external communicating sealer (1.4), so as to keep the sealing property of the positive and negative pressure cabin (1). The general refrigerator refrigerating compartment type positive and negative pressure fresh-keeping cabin (1D) and the general refrigerator freezing compartment type positive and negative pressure fresh-keeping cabin (1E) are both atmospheric circulation cabins, each of a refrigerating compartment and a freezing compartment of the general refrigerator is provided with a positive and negative pressure system, but without the airtight mechanism and internal and external communicating sealer. The general refrigerator refrigerating compartment type positive and negative pressure fresh-keeping cabin (1D) includes a cabin body (1.1) and a cabin door (1.2), and an airtight mechanism and an internal and external communicating sealer do not need to be provided between the cabin body (1.1) and the cabin door (1.2), and the cabin door (1.2) is a refrigerator door of the fresh-keeping refrigerator. The general refrigerator freezing compartment type positive and negative pressure fresh-keeping cabin (1E) includes a cabin door (1.1), and a non-airtight fresh-keeping drawer (1.83). An airtight mechanism and an internal and external communicating sealer do not need to be provided between the cabin body (1.1) and the non-airtight fresh-keeping drawer (1.83). The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), airflow carried substance generator integrations (6.021), (6.022), (6.023), (6.024) and (6.025). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. Each of the airflow carried substance generator integrations (6.021), (6.022), (6.023), (6.024) and (6.025) includes any one or more of the individual fluid carried substance generators (6.1) to (6.16) as required. Each of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) is externally provided with an air evacuation pump (2), an air inflation pump (3), a positive and negative pressure intelligent regulation and control apparatus (5), a positive and negative pressure airflow carried substance generator integration (6.021), a carried substance decomposition processor (6.7), an air filter (6.8), a refrigeration system (8), an air-to-water production apparatus (11), and a touch screen and mobile phone monitoring system (12). An air evacuation pipeline (A1) is installed at an air inlet port (a) of the air evacuation pump (2), air evacuation branch pipelines (A1.1), (A1.2), (A1.3) and (A1.4) are respectively connected to the air evacuation pipeline (A1), solenoid valves (B1.1), (B1.2), (B1.3) and (B1.4) are respectively installed at middle parts of the air evacuation branch pipelines (A1.1), (A1.2), (A1.3) and (A1.4), and the other ends of the air evacuation branch pipelines (A1.1), (A1.2), (A1.3) and (A1.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form air evacuation and air return ports (c1), (c2), (c3) and (c4) thereof. Carrying waste gas evacuation and exhaust pipelines (A8), (A8.1), (A8.2), (A8.3) and (A8.4) are respectively connected to the air evacuation pipeline (A1), solenoid valves (B8), (B8.1), (B8.2), (B8.3) and (B8.4) are respectively installed at middle parts of the carrying waste gas evacuation and exhaust pipelines (A8), (A8.1), (A8.2), (A8.3) and (A8.4), and the other ends of the carrying waste gas evacuation and exhaust pipelines (A8), (A8.1), (A8.2), (A8.3) and (A8.4) are respectively connected to carrying waste gas evacuation and exhaust ports (s3), (s4), (s5), (s6) and (s7) of the air conditioning apparatus (6.1) integrated in the positive and negative pressure airflow carried substance generator integrations (6.021), (6.022), (6.023), (6.024) and (6.025). An air exhaust pipeline (A2) is installed at an air outlet port (b) of the air evacuation pump (2), a carried substance decomposition processor (6.7) and a solenoid valve (B2) are installed at a middle part of the air exhaust pipeline (A2), and the other end of the air exhaust pipeline (A2) is connected to an air inlet port (h) of an air-to-water production apparatus (11), and then is connected to atmosphere via an air outlet port (n) of the air exhaust pipeline (A2) after passing through the air-to-water production apparatus (11). An air inflation pipeline (A3) is installed at an air outlet port (d) of the air inflation pump (3), a solenoid valve (B3) is installed at a middle part of the air inflation pipeline (A3), air inflation branch pipelines (A3.1), (A3.2), (A3.3) and (A3.4) are respectively connected to the air inflation pipeline (A3), solenoid valves (B3.1), (B3.2), (B3.3) and (B3.4) are respectively installed at middle parts of the air inflation branch pipelines (A3.1), (A3.2), (A3.3) and (A3.4), and the other ends of the air inflation branch pipelines (A3.1), (A3.2), (A3.3) and (A3.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form air inflation and inlet ports (f1), (f2), (f3) and (f4) thereof. A carrying air introduction pipeline (A6) is further connected to the air inflation pipeline (A3), a solenoid valve (B6) is installed at a middle part of the carrying air introduction pipeline (A6), and the other end of the carrying air introduction pipeline (A6) is connected to an air inlet port (t) of the positive and negative pressure airflow carried substance generator integration (6.021). Carrying air introduction branch pipelines (A7.2) and (A7.3) are respectively installed at air outlet ports (s1) and (s2) of the positive and negative pressure airflow carried substance generator integration (6.021), solenoid valves (B7.2) and (B7.3) are respectively installed at middle parts of the carrying air introduction branch pipelines (A7.2) and (A7.3), and the other ends of the carrying air introduction branch pipelines (A7.2) and (A7.3) respectively extend into the positive and negative pressure refrigerating fresh-keeping cabin (1B) and the positive and negative pressure freezing fresh-keeping cabin (1C) to form carrying air inlet ports (12) and (i3) thereof. Carrying air introduction branch pipelines (A7.1) and (A7.4) are respectively connected to the solenoid valves (B7.2) and (B7.3), solenoid valves (B7.1) and (B7.4) are respectively installed at middle parts of the carrying air introduction branch pipelines (A7.1) and (A7.4), and the other ends of the carrying air introduction branch pipelines (A7.1) and (A7.4) respectively extend into the general refrigerator refrigerating compartment type positive and negative pressure fresh-keeping cabin (1D) and the general refrigerator freezing compartment type positive and negative pressure fresh-keeping cabin (1E) to form carrying air inlet ports (i1) and (14) thereof. Carrying branch pipelines (A6.1), (A6.2), (A6.3) and (A6.4) are respectively connected to the air inflation pipeline (A3), solenoid valves (B6.1), (B6.2), (B6.3) and (B6.4) are respectively installed at middle parts of the carrying branch pipelines (A6.1), (A6.2), (A6.3) and (A6.4), and the other ends of the carrying branch pipelines (A6.1), (A6.2), (A6.3) and (A6.4) are respectively connected to air inlet ports (t1), (t2), (t3) and (t4) of the positive and negative pressure airflow carried substance generator integrations (6.023), (6.024) and (6.025). An air intake pipeline (A4) is installed at an air inlet port (e) of the air inflation pump (3), an air filter (6.8) and a solenoid valve (B4) are installed at middle parts of the air intake pipeline (A4), and the other end of the air intake pipeline (A4) is connected to atmosphere. A circulation pipeline (A5) is further connected to the solenoid valve (B4), and circulation branch pipelines (A5.1), (A5.2), (A5.3) and (A5.4) are respectively connected to the circulation pipeline (A5), solenoid valves (B5.1), (B5.2), (B5.3) and (B5.4) are installed at middle parts of the circulation branch pipelines (A5.1), (A5.2), (A5.3) and (A5.4), and the other ends of the circulation branch pipelines (A5.1), (A5.2), (A5.3) and (A5.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form circulation air return ports (j1), (j2), (j3) and (j4) thereof. An air-to-water production inflation pipeline (A9) is further connected to the circulation pipeline (5) through a solenoid valve (B9), the air-to-water production inflation pipeline (A9) is connected to the solenoid valve (B2) and then communicates with an air inlet port (h) of the air-to-water production apparatus (11) through the air exhaust pipeline (A2), the air-to-water production inflation pipeline (A9) is connected to the circulation pipeline (A5) and communicates with the solenoid valve (B3), and then communicates with an air outlet port (d) of the air inflation pump (3) through the air inflation pipeline (A3). The touch screen and mobile phone monitoring and identification system (12) includes a refrigerator door touch screen (12.1), a mobile phone APP (12.2), a high-definition anti-fog camera apparatus (12.3), and a radar scanning recognition apparatus (12.5). The high-definition anti-fog camera apparatus (12.3) and the radar scanning recognition apparatus (12.5) are installed at a position needing to be monitored and recognized inside and outside the refrigerator body (7). The cabins of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) are internally provided with airflow carried substance generator integrations (6.022), (6.023), (6.024) and (6.025) and sensor integrations (CA). Each of the airflow carried substance generator integrations (6.022), (6.023), (6.024) and (6.025) includes one or more of the following individual airflow carried substance generators (6.1) to (6.16): an air conditioning apparatus (6.1), a super oxygen generator (6.2), a negative ion generator (6.3), a catalyst release controller (6.4), a humidification, dehumidification and humidity control apparatus (6.5), a disinfection, sterilization and degradation apparatus (6.9), a carried substance generator item addition and upgrading module (6.16). The sensor includes the sensor integration (CA), the sensor integration (CA) includes one or more of the following individual sensors (C1) to (C12) as required: a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), a carried substance sensor item addition and upgrading module (C12). The carried substance sensor item addition and upgrading module (C12) is a combined module capable of providing and installing any individual sensor additional item into the sensor integration and upgrading and updating the same; the sensor integration (CA) includes at least one of more of individual sensors (C1) to (C12). All airflow carried substance generation processors (6), sensors (C) and solenoid valves (B) and the air evacuation pump (2), the air inflation pump (3), the refrigeration system (8), the air-to-water production apparatus (11) and the mobile phone monitoring and identification system (12) are connected to the positive and negative pressure intelligent regulation and control apparatus (5) by lines (D) or bundled lines (E).

According to a fourth embodiment, an operation method for an intelligent positive and negative pressure fresh-keeping refrigerator is provided. The intelligent positive and negative pressure fresh-keeping refrigerator adopts an intelligent positive and negative pressure system. The intelligent positive and negative pressure system is used to accurately regulate and organically integrate various techniques such as vacuum, high pressure, super oxygen, catalysts, air conditioning fresh-keeping, negative ions, humidification, dehumidification, humidity and temperature control, disinfection and sterilization, degradation treatment, air purification, air-to-water production, and low-temperature storage, one item of or a combination of more items of the techniques is applied to the positive and negative pressure fresh-keeping refrigerator, and digital techniques such as Internet, big data, cloud computing, machine learning, artificial intelligence and expert system are also applied to the refrigerator at the same time, so as to form a regulatable and controllable artificial intelligence microclimate in the positive and negative pressure fresh-keeping refrigerator and to create a high-energy and efficient precise fresh-keeping microenvironment. Various fresh-keeping elements such as air composition, airflow pressure, humidity and temperature, aseptic cleanliness and the like of the positive and negative pressure fresh-keeping refrigerator are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization, quickly degrade harmful residues on the surface of preservation, achieve all-round and multi-angle digital targeted treatment, eliminate putrefying factors, accurately keep freshness and comprehensively preserve nutrition; various fresh goods stored in the positive and negative pressure fresh-keeping refrigerator are in an optimal pressure range, an optimal gas composition and optimal humidity and temperature, and an optimal preservation environment. Fruits and vegetables are hibernated at low temperature in an existing refrigerator, while the positive and negative pressure and digital techniques are used in the positive and negative pressure fresh-keeping refrigerator to make fruits and vegetables in a dormant or suspended animation state, such that the fruits and vegetables have the weakest life, the slowest aging, the lowest breathing and the least consumption of nutrition, and the nutritional components and the original freshness of fruits, vegetables, meat, fish and other stored products are quickly stored for a long time; in the positive and negative pressure system, the digital technique is used to analyze and identify fresh-loss sites and fresh-keeping elements of various stored products one by one to form a complete large database according to different categories, and to design a personalized targeted control comprehensive fresh-keeping method at the cellular and molecular level for the fresh-loss sites and fresh-keeping elements of the stored products, so as to accurately maintain the fresh-keeping elements and remove the fresh-loss factors at a fixed point, without affecting or less affecting the normal tissues and cells of the stored products, and thus a set of digital expert system for preservation and fresh-keeping is formed and gradually improved, which is intelligently applied to the positive and negative pressure fresh-keeping refrigerator to achieve the purpose of keeping freshness and quality of the stored products. In a specific operation method for the intelligent positive and negative pressure fresh-keeping refrigerator, an air evacuation pump or air inflation pump is used by the positive and negative pressure system to intelligently regulate and control airflow pressure, or carry super oxygen, catalysts, negative ions, air conditioning gas, disinfection and degradation substances, heat, water and various effective loads by the airflow to get in, stay in or get out of a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D) and a general refrigerator freezing compartment type positive and negative pressure fresh-keeping cabin (1E) orderly, and the following methods are used to apply various forces helpful for long-term freshness and quality keeping to fresh fruits and vegetables stored in the refrigerator: (1) a positive atmospheric pressure high pressure action method: forming moderate positive atmospheric pressure at proper time in a cabin of the compartment container type positive and negative pressure cabin (1M), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the compartment container type positive and negative pressure cabin (1M), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) an airflow carried substance action method: carrying single or a combination of multiple super oxygen, catalyst, negative ion, air conditioning gas, disinfection and degradation substances, heat, water and various effective loads by positive and negative pressure airflow to get in, stay in or get out of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) orderly, integrating various fresh-keeping techniques to apply various accurate and effective action forces for the long-term freshness and quality keeping of the stored fruits, vegetables and fresh goods. Based on above three action force methods, the positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of the touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping; the targeted control comprehensive fresh-keeping methods for the intelligent positive and negative pressure system and specific use methods and operation procedures of the positive and negative pressure fresh-keeping refrigerator are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1.1) or (B1.2 or (B1.3) or (B1.4) to open corresponding air exhaust pipelines (A2) and (A1) and (A1.1) or (A1.2 or (A1.3) or (A1.4) of the related positive and negative pressure cabin, and turning on the air evacuation pump (2) to exhaust or pump the related positive and negative pressure cabin to vacuum negative pressure between −0.001 KPa and −0.1 MPa (taking the local instant atmospheric pressure as the zero standard, and the vacuum degree can be improved according to the specific demands and refrigerator configuration), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2), pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within the positive and negative pressure cabin, thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) atmospheric or pressurized sterilization, degradation and deodorization procedure for super-oxygen negative ion or catalyst positive and negative pressure carried substance: according to the procedure setting and feedback of the related sensors (C), sending, by the positive and negative pressure intelligent regulation and control apparatus (5), an instruction at a proper time to turn on a super oxygen generator (6.2), a negative ion generator (6.3), a catalyst release controller (6.4), a humidity control apparatus (6.5) or a disinfection and degradation apparatus (6.9) in each related airflow carried substance generator integration, or a carried substance generator installed in a carried substance generation item addition and upgrading module (6.16), thus enabling the super oxygen, catalyst, negative ion, water and related carried substances in the positive and negative pressure cabin reach required standards; meanwhile, enabling solenoid valves (B3) and (B4) or (B3.1) or (B3.2) or (B3.3) or (B3.4) to open corresponding air inflation pipelines (A4) and (A3) or (A3.1) or (A3.2) or (A3.3) or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and refrigerator configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric or pressurized sterilization, degradation and deodorization procedure for air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3), (B6) and (B7.1), (B7.2), (B7.3) or (B7.4) to open related carrying air inflation pipelines (A3), (A6) and (A7.1), (A7.2), (A7.3) or (A7.4) and turn on the air inflation pump (3) to drive the airflow to enter the related positive and negative pressure cabin after passing through an air conditioning apparatus (6.1) and other related positive and negative pressure fluid carried substance generators (6); and enabling solenoid valves (B3), (B4), (B9) and (B5.1), (B5.2), (B5.3) or (B5.4) to open carrying circulation pipelines to circulate and operate; during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B8) or (B8.1), (B8.2), (B8.3) or (B8.4) to open carrying waste gas evacuation and exhaust pipelines (A8) or (A8.1), (A8.2), (A8.3) or (A8.4), carrying waste gas evacuation and exhaust port (s3), (s4), (s5), (s6) or (s7) of air conditioning apparatus (6.1)→(A8) or (A8.1), (A8.2), (A8.3) or (A8.4)→solenoid valves (B8) or (B8.1), (B8.2), (B8.3) or (B8.4)→(A8), (A8.1), (A8.2), (A8.3) or (A8.4)→(A1)→air inlet port (a) of air evacuation pump→air outlet port (b) of air evacuation pump→(A2)→carried substance decomposition processor (6.7)→(B2)→(A2)→air-to-water production apparatus (11)→exhaust to atmosphere; turning on the air evacuation pump (2) to exhaust decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) atmospheric, pressurized, or sterilized preservation procedure for leftovers: placing the leftovers into the positive and negative pressure cabin, turning on the air evacuation pump (2) and related air pipelines by the regulation and control apparatus (5) until the odor already released by the leftovers and the polluted air inside the cabin are pumped out of the cabin; turning on the air inflation pump (3) and related air inflation pipelines for properly pressurized preservation, which not only prevents the odor and water from excessively scattering and losing, but also inhibits the food spoilage; meanwhile, according to feedback information of the humidity sensor, turning on the humidity control apparatus (6.5) at proper time to supplement the water in the air, or turning on related carried substance generation processors at proper time to kill the bacteria and virus in the air in the cabin to prevent food spoilage; (6) low-temperature assisted fresh-keeping procedure: after the positive and negative pressure fresh-keeping refrigerator is turned on, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (7) open-to-exhaust protection function: if the refrigerator door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to a carried substance decomposition processor (6.7) at the rear part of the refrigerator to be exhausted to atmosphere after being decomposed, where, as the air flows from the outside of the refrigerator door to the positive and negative pressure cabin for supplementing air evacuation negative pressure, the super oxygen or high-nitrogen low-oxygen carried substance gas cannot flow out of the door; (8) air-to-water production procedure: enabling moist waste gas pumped from the vacuum high pressure cabin by the air evacuation pump 2 to enter the super oxygen decomposer (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B9) and B2 to open air-to-water production inflation pipelines, thus driving the indoor air to enter the air-to-water production apparatus (11) for water production; (9) remote and short-range control monitoring and identification function: controlling and monitoring, by the touch screen and mobile phone monitoring and identification system (12), the fresh-keeping refrigerator and other positive and negative pressure electric appliances and a positive and negative pressure module cabinet in real time by the WIFI and mobile phone APP at remote and short-range, thus making the fresh-keeping refrigerator and other all positive and negative pressure electric appliances and module cabinets to intelligently achieve various functions at high efficiency and low consumption according to the set procedures and instant instructions, where a high-definition anti-fog camera and identification apparatus (12.3) and a radar scanning and identification apparatus (12.5) are installed at positions needing monitoring and identification inside and outside the refrigerator body (7); shooting and scanning statuses of objects in the refrigerator in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling various fresh-keeping environmental factors such as airflow pressure, gas composition, air cleanliness, environment temperature and humidity in the positive and negative pressure refrigerating-freezing cabin in real time, and automatically performing harmless disinfection and sterilization, and rapidly degrading residual pesticides, fertilizers, hormones and harmful additives to form an adjustable and controllable artificial microclimate in the positive and negative pressure refrigerating-freezing cabin, thus creating a microenvironment favorable for fresh-keeping, which makes various fresh foods stored in the positive and negative pressure refrigerating-freezing cabin in the optimal atmospheric pressure range, the optimal gas composition, the optimal humidity, and the optimal preservation environment for keeping fresh, tender and original flavor; displaying and reminding, by a refrigerator door touch screen (12.1) or a mobile phone APP (12.2), commodity information such as production date, price, expiration date and manufacturers and information of purchase shopping malls and online stores, automatically recording, analyzing and handling the big data such as the time, quantity, frequency and preference of preserving the objects in, and taking the objects out, the refrigerator, and performing intelligent analysis by combining cloud data; timely reminding on the mobile phone APP (12.2) or directly pushing the suggested goods to the mobile phone APP (12.2) or the refrigerator door touch screen (12.1), where, in addition to real-time monitoring and identification at a fixed position, the camera and identification apparatus (12.3) is required to be installed in the refrigerator door (1.2) and to automatically and continuously shoot multiple panoramic photos of the objects in the refrigerator and automatically record small videos with the closing movement of the refrigerator door (1.2) by starting from the position away from the refrigerator body in the process of closing the refrigerator door, thus facilitating a user to view in the mobile phone APP, or view the panoramic photos or videos of the objects in the refrigerator with good light and wide field of vision before and during the last closing of the door in the refrigerator door touch screen at any time without opening the refrigerator door, releasing the vacuum and lowering the temperature during short range; and dynamically identifying the objects and uploading the data; and (10) positive and negative pressure module combined intelligent smart home mode: designing and manufacturing the intelligent positive and negative pressure fresh-keeping refrigerator according to the module functions, where the intelligent positive and negative pressure refrigerator not only is independently produced and used as a separated invention, or but also is organically combined with other positive and negative pressure electric appliances and the module cabinets under the control of the touch screen and mobile phone monitoring and identification system (12) to form a fully functional intelligent positive and negative pressure module combined home; the fully functional intelligent positive and negative pressure module combined home not only can share the positive and negative pressure system, the refrigeration system and the air-to-water production apparatus, but also has basically uniform, neat and harmonious appearance, shape and color which are in line with the popular trend. In conclusion, in accordance with the specific operation methods for the intelligent positive and negative pressure fresh-keeping refrigerator, multiple methods, such as vacuum, high pressure, super oxygen, catalysts, air-conditioning fresh-keeping, negative ions, humidification, humidity and temperature control, disinfection and sterilization, air purification, air-to-water production, and low-temperature storage, are intelligently regulated and integrated under positive pressure or negative pressure, are organically applied to the positive and negative pressure fresh-keeping refrigerator, and are organically integrated and optimally matched. One item of or a combination of more items of the above techniques is used in individually or alternately or circularly, and multiple positive and negative pressure cabins with different types and different functions are arranged in a positive and negative pressure fresh-keeping compartment, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping refrigerator updates the existing method that the traditional refrigerator achieves low-temperature storage only by controlling the temperature of the refrigerator; the positive and negative pressure system accurately regulates and controls various fresh-keeping environment elements such as the airflow pressure, gas composition, air cleanliness, environmental temperature and humidity in the positive and negative pressure refrigerating-freezing cabin in real time, performs harmless disinfection and sterilization in the refrigerator, and rapidly degrades residual pesticides, fertilizers, hormones and harmful additives, achieve accurate positioning and targeted removal of fresh-loss factors, thus making various fresh foods stored in positive and negative pressure refrigerating-freezing cabin keep fresh and moist quality and flavor for a long time. The positive and negative pressure system endows the fresh-keeping refrigerator with breathing function and circulation characteristics, makes the cold fresh-keeping refrigerator come alive and become a life appliance, and upgrades the existing refrigerator and traditional methods. As the positive and negative pressure system has an ultra-large-capacity carrier and renewable performance and a platform advantage of upgrading, extending and adding items, the positive and negative pressure fresh-keeping refrigerator is endowed with the upgrading and expanding function. With the gradual development of science and technique, various newly developed methods of fresh-keeping and preservation are absorbed and integrated into the positive and negative pressure fresh-keeping refrigerator, making the positive and negative pressure fresh-keeping refrigerator innovate and develop as mobile phones and computers; and a chip processor and various software of the positive and negative pressure intelligent regulation and control apparatus are upgraded and updated gradually, making the positive and negative pressure fresh-keeping refrigerator more multifunctional, efficient, environmentally friendly, low-consumption, diverse and suitable for use.

According to a fifth embodiment of the present disclosure, an intelligent positive and negative pressure washing machine is provided, including a machine body (7). The machine body (7) is internally provided with an intelligent positive and negative pressure system, a washing-dehydrating-drying system (15). The intelligent positive and negative pressure system includes a positive and negative pressure cabin (1), an air evacuation pump (2), an air inflation pump (3), a positive and negative pressure intelligent regulation and controlling apparatus (5), a positive and negative pressure waterflow carried substance generator integration (6.04), a carried substance decomposition processor (6.7), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water input pump (17) and the touch screen and mobile phone monitoring and identification system (12). The positive and negative pressure cabin (1) is designed as a washing machine type positive and negative pressure cabin (1F) according to particular requirements for washing and drying. The washing machine type positive and negative pressure cabin (1F) is a vacuum high pressure cabin (1-1) structure. The positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a washing machine configuration. The washing machine type positive and negative pressure cabin (1F) includes a cabin body (1.1), a cabin door (1.2), an airtight mechanism (1.3), and an internal and external communicating sealer (1.4). The airtight mechanism (1.3) is arranged between the cabin door (1.2) and the cabin body (1.1). The airtight mechanism (1.3) includes a lock ring (1.31), a lock bolt (1.32), an airtight gasket (1.33), and a door rim sealing gasket (1.34); the lock ring (1.31) is in movable fit with the lock bolt (1.32), the lock bolt (1.32) extends into the lock ring (1.31) to lock the cabin door during the closing of the cabin door; when the positive and negative atmospheric pressure is formed inside the washing machine type positive and negative pressure cabin (1F), the airtight gasket (1.33) is used for keeping sealing. The internal and external communicating sealer (1.4) is fixedly arranged at a rear part or side part of washing machine type positive and negative pressure cabin (1F), and all pipes and circuits getting in and out the cabin are connected to and pass through the internal and external communicating sealer (1.4) so as to keep the sealing property of the washing machine type positive and negative pressure cabin (1F). The positive and negative pressure intelligent regulation and control apparatus (5) includes an integrated circuit and chip, and a regulation and control system for a fluid and a carried substance thereof, and is connected to the Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), a carried substance generation item addition and upgrading module (6.16). The carried substance generation item addition and upgrading module (6.16) is a combined module capable of providing or installing any individual fluid carried substance generator additional item in the carried substance generator integration and upgrading and updating the same. The waterflow carried substance generator integration (6.04) includes at least one or more of individual fluid carried substance generators (6.1) to (6.16) as required. The washing-dehydrating-drying system (15) includes a washing machine drum mechanism (15.1), a washing-dehydrating-drying control mechanism (15.2), a drum sealing bearing housing (15.3), a drum big belt pulley (15.4), and a motor assembly (15.5). The washing machine type positive and negative pressure cabin (1F) is internally provided with the washing machine drum mechanism (15.1), an anti-shower air evacuation port (2.1), the drum sealing bearing housing (15.3), a pressure sensor (C1), a negative pressure sensor (C2), a super oxygen sensor (C3), and a carried substance sensor item addition and upgrading module (C12). The carried substance sensor item addition and upgrading module (C12) is a combined module capable of providing or installing any individual sensor additional item in the sensor integration and upgrading and updating the same. The washing machine type positive and negative pressure cabin (1F) is externally provided with the air evacuation pump (2), the air inflation pump (3), the intelligent positive and negative pressure regulation and control apparatus (5), the positive and negative pressure waterflow carried substance generator integration (6.04), the carried substance decomposition processor (6.7), the washing-dehydrating-drying control mechanism (15.2), the drum big belt pulley (15.4), the motor assembly (15.5), the water suction pump (16), the water input pump (17), a detergent pull box (18), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the washing machine type positive and negative pressure cabin (1F) and then is connected with the anti-shower air evacuation port (2.1) to form an air evacuation and air return port (c) or a circulation air return port (i), and an air outlet port (b) of the air evacuation pump (2) is connected to the atmosphere. A circulation pipeline (A5) is further connected to the solenoid valve (B1), a solenoid valve (B5) is installed at a middle part of the circulation pipeline (A5), and the other end of the circulation pipeline (A5) is a circulation exhaust port (i2) or a circulation air inlet port (j2). An air inflation pipeline (A3) is installed at an air outlet port (d) of the air inflation pump (3), a solenoid valve (B3) is installed at a middle part of the air inflation pipeline (A3), the other end of the air inflation pipeline (A3) extends into washing machine type positive and negative pressure cabin (1F) to form an air inflatable inlet port (f) or a circulation air inlet port (j), and an air inlet port (e) of the air inflation pump (3) is connected to the atmosphere. A circulation pipeline (A6) is further connected to the solenoid valve (B3), a solenoid valve (B6) is installed at a middle part of the circulation pipeline (A6), and the other end of the circulation pipeline (A6) is connected to a circulation air inlet port (e2) of the air inflation pump (3). A circulation pipeline (A7) is further connected to the solenoid valve (B6), and the other end of the circulation pipeline (A7) is connected to the solenoid valve (B5). A water intake pipeline (A14) is installed at a water inlet port (r) of the water input pump (17), a solenoid valve (B14) is installed at a middle part of the water intake pipeline (A14), and the other end of the water intake pipeline (A14) communicates with a tap water source. A washing machine water intake pipeline (A15) is installed at a water output port (u) of the water input pump (17), a solenoid valve (B15) is installed at a middle part of the water intake pipeline (A15), and the other end of the water intake pipeline (A15) communicates with a water inlet port (v) of the positive and negative pressure waterflow carried substance generator integration (6.04). A washing machine water intake pipeline (A16) is installed at a water output port (o) of the water input pump (17), a solenoid valve (B16) is installed at a middle part of the water intake pipeline (A16), and the other end of the water intake pipeline (A16) communicates with a water inlet port (z) of the detergent pull box (18). A washing machine water intake pipeline (A17) is installed at a water output port (1) of the detergent pull box (18), a solenoid valve (B17) is installed at a middle part of the water intake pipeline (A17), and the other end of the water intake pipeline (A17) extends into the washing machine type positive and negative pressure cabin (1F) to form a water inlet port (T) thereof. A water intake pipeline (A18) is further installed on the solenoid valve (B14), and the solenoid valve (B14) communicates with the solenoid valve (B16) so as to directly feed water when carrying water is not needed and the tap water pressure reaches the standard. A water intake pipeline (A19) is further installed on the solenoid valve (B15), and the solenoid valve (B15) is connected to the water intake pipeline (A18) to communicate with the solenoid valve (B16), such that the water is directly fed by the water input pump (17) when the carrying water is not needed. A water drainage pipeline (A20) is installed at a water output port (w) of the water suction pump (16), a solenoid valve (B20) is installed at a middle part of the water drainage pipeline (A20), and the other end of the water drainage pipeline (A20) extends into the washing machine type positive and negative pressure cabin (1F) to form a water drainage port (L) thereof. A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) is connected to a sewer. A water drainage pipeline (A23) is installed at the solenoid valve (B20), and the solenoid valve (B20) communicates with the solenoid valve (B22), such that the water is directly drained when the carried substance does not need to be decomposed and free drainage is achieved. A water drainage pipeline (A24) is further installed on the solenoid valve (B21), and the solenoid valve (B21) is connected to the water drainage pipeline (A23) and then communicates with the solenoid valve (B22), such that the water is directly drained by the water suction pump (16) when the carried substance does not need to be decomposed; the big belt pulley (15.4) is driven by the motor assembly (15.5), and the big belt pulley (15.4) drives the drum mechanism (15.1) connected thereto to operate. All fluid carried substance generation processors (6), the sensors (C), the solenoid valves (B), and the air evacuation pump (2), the air inflation pump (3), the washing-dehydrating-drying control mechanism (15.2), the water suction pump (16), the water input pump (17), and the touch screen and mobile phone monitoring and identification system (12) are all connected to the positive and negative pressure intelligent regulation and control apparatus (5).

According to a sixth embodiment of the present disclosure, an operation method for an intelligent positive and negative pressure washing machine is provided. The intelligent positive and negative pressure washing machine adopts an intelligent positive and negative pressure system. The intelligent positive and negative pressure system is used to accurately regulate and organically integrate various techniques such as vacuum, high pressure, super oxygen, disinfection and sterilization, degradation treatment, and clothes cleaning and drying, and one item of or a combination of more items of the techniques is applied to the positive and negative pressure washing machine, a vacuum and high pressure airflow-waterflow stain removal drying technique is developed, and digital techniques such as Internet, big data, cloud computing, machine learning, artificial intelligence and expert system are also applied to the washing machine at the same time to analyze and identify stain-causing elements and control targets of various different cleaning materials one by one, so as to form a complete large database according to different categories, and correspondingly use different targeted stain removal techniques. According to built-in program and pre-stored data of the positive and negative pressure washing machine, and real-time feedback information of various sensors, various washing-dehydrating-drying elements, such as waterflow and airflow pressure, gas composition, water temperature and air temperature, are regulated and controlled in real time, so as to disinfect, sterilize and degrade the cleaning materials harmlessly, and create a regulatable and controllable accurate washing-dehydrating-drying intelligent microenvironment with the optimal effect, shortest time, highest efficiency, minimum wear and least environmental pollution. A personalized targeted stain removal comprehensive washing-drying method is designed at the molecular and microscopic levels for stain-causing sites of the cleaning material and a contaminant, so as to accurately removal the stain-causing factors at fixed points, while avoiding or reducing the damage to the structure and color of the cleaning materials, and thus a set of digital expert system for clothes cleaning and drying is formed and gradually improved, which is intelligently applied to the positive and negative pressure washing machine to achieve the purpose of fast, efficient, sanitary and environmental-friendly cleaning. A specific operation method for the intelligent positive and negative pressure washing machine is that the positive and negative pressure system intelligently regulates and controls the airflow pressure by an air evacuation pump or an air inflation pump, and carries the effective loads of super oxygen, a disinfectant, a cleaning builder, a softening and bulking agent and a decomposition treatment agent by the airflow or waterflow to get in, stay in, or get out a positive and negative pressure cabin (1F) orderly, and the following methods are used to apply various action forces helpful for rapid and efficient cleaning and drying of the laundry: (1) positive atmospheric pressure high pressure action method: A: during washing, turning on the air inflation pump (3) at proper time to inflate the positive and negative pressure cabin (1F) by means of an air inflation pipeline (A3), so as to form strong bubbles and rapid waterflow to participate in clothes cleaning, speed up the washing process and improve the washing efficiency, where the high atmospheric pressure formed in the cabin is beneficial for a detergent to penetrate into the clothes to facilitate the cleaning; B: during water drainage, inflating and pressurizing the positive and negative pressure cabin (1F) to speed up the water drainage speed; C: during dehydration, inflating and pressurizing to force water to quickly separate from the clothes, so as to improve dehydration efficiency and shorten water drainage and dehydration time; D: at the beginning of a drying procedure, inflating and pressurizing the positive and negative pressure cabin (1F) of the washing machine, which is beneficial for hot air to enter clothes fibers to vaporize water, and then releasing pressure rapidly to drain the water vapor to accelerate the drying process and effectively shorten the drying time; (2) negative atmospheric pressure vacuum action method: A, during water intake, turning on the air evacuation pump (2) to pump the positive and negative cabin (1F) to negative pressure by means of an air evacuation pipeline (A1), so as to speed up water intake and shorten water intake time; B: at the beginning of washing, evacuating the positive and negative cabin (1F) to a moderate vacuum, where both the clothes fibers and stains swell in the moderate vacuum, the adhesive force of the stains is weaken or the stains fall off, such that the cleaning efficiency is improved, and the washing time is shortened; C: during washing, circulating and repeating air evacuation and air inflation for alternate fiber swelling and turbulent rubbing, thus improving cleaning degree and clothes washing efficiency, and accelerating washing and rinsing progress; D: during dehydration, evacuating the positive and negative pressure cabin to the negative pressure to make the clothes fibers swell, where the water in the clothes escape to the negative pressure space and is pumped out of the cabin, making the dehydrated and hardened clothes fluff, and circulating and repeating the air evacuation and air inflation to force the water out and pump the water out of the cabin alternately, thus greatly improving the clothes dehydration efficiency and shortening the water drainage and dehydration time; E: carrying out air evacuation at proper time during drying, evacuating the water, and making the clothes fibers swell by the negative pressure to facilitate quick drying of water vapor, where, with the repeated circulation of air inflation, heating gasification and water evacuation are alternately conducted, which greatly accelerates the clothes drying progress, and the negative pressure makes the clothes fluff, which improves the clothes washing and drying quality and effectively shortens the clothes drying time; (3) airflow and waterflow carried substance action method: carrying effective loads of super oxygen, a disinfectant, a cleaning builder, a softening and bulking agent and a decomposition treatment agent by the airflow or waterflow, where a single or a combination of the plurality of effective loads gets in, stays in, or gets out the washing machine type positive and negative pressure cabin (1F) orderly, so as to apply various action forces beneficial for fast and efficient cleaning and drying of the laundry; in the intelligent positive and negative pressure system, a positive and negative pressure intelligent regulation and control apparatus (5) is used to analyze and identify stain-causing factors and stain removal targets of various different cleaning scenarios and cleaning clothes one by one according to built-in program, pre-stored data and internet cloud data of the positive and negative pressure intelligent regulation and control apparatus (5), and real-time feedback information of a touch screen and mobile phone monitoring and identification system (12) and various sensors (C1) to (C12), and after computing, different accurate stain removal techniques are correspondingly used to form targeted stain removal comprehensive cleaning methods with different characteristics, and the positive and negative pressure intelligent regulation and control apparatus (5) is used to send specific instructions to the air evacuation pump (2), the air inflation pump (3), related positive and negative pressure fluid carried substance generators (6) and related solenoid valves (B) in real time to accurately control and adjust open, close and switching of various air evacuation, inflation and circulation pipelines and intelligently regulate and control the level of the positive and negative pressure in the positive and negative pressure cabin (1) and the flowing-in, staying and flowing-out of a fluid and a carried substance thereof, so as to remove the stain-causing factors of the cleaning materials at fixed points and accurately maintain fragile elements of the cleaning materials to achieve the efficient and low-consumption cleaning effect. The specific operation methods for the intelligent positive and negative pressure system targeted stain removal comprehensive washing and drying and the positive and negative pressure washing machine are as follows: (1) water intake procedure: placing clothes into a drum (15.1), closing a cabin door (1.2) to seal and lock the washing machine type positive and negative pressure cabin (1F) after the washing machine is turned on; sending an instruction by the positive and negative pressure intelligent regulation and control apparatus (5) to enable solenoid valves (B14), (B15), (B16) and (B17) to open the corresponding water intake pipelines: (i) direct tap water intake line being: (A14)→(B14)→(A18)→(B16)→(A16)→water inlet port (z) of detergent pull box→water output port (1)→(A17)→(B17)→(A17)→water inlet port (T) of positive and negative pressure cabin; (ii) when washing-aid carried substance is required for washing, a water intake line being: (A14)→(B14)→(A14)→water inlet port (r) of water input pump→water output port (u)→(A15)→(B15)→(A15)→water inlet port (v) of waterflow carried substance generator integration→water output port (0)→(A16)→(B16)→(A16)→detergent pull box (z)→water output port (1)→(A17)→(B17)→(A17)→water inlet port (T) of positive and negative pressure cabin; (iii) when washing-aid carried substance is not required for cleaning, a water intake line being: (A14)→(B14)→(A14)→water inlet port (r) of water input pump→water output port (u)→(A15)→(B15)→(A19)→(A18)→(B16)→(A16)→water inlet port (z) of detergent pull box→water output port (1)→(A17)→(B17)→(A17)→water inlet port (T) of positive and negative pressure cabin, during the procedures (ii) and (iii), turning on the water input pump (17), directly feeding the tap water accordingly, or by the waterflow carried substance generator integration (6.04), rapidly mixing the tap water with the washing aid carried substance generated by waterflow carried substance generator integration (6.04), and then enabling the mixed water to enter the washing machine type positive and negative pressure cabin (1F), where the super-oxygen or carried substance mixed water can accelerate the decomposition of organic fouling on the clothes, thus facilitating the rapid cleaning; after the water intake is started, enabling, by the positive and negative pressure intelligent regulation and control apparatus (5), a solenoid valve (B1) to open an air evacuation pipeline (A1), air inlet port (a) of air evacuation pump→(A1)→(B1)→(A1)→air evacuation and air return port (c) of positive and negative pressure cabin, and turning on the air evacuation pump (2) to make the washing machine type positive and negative pressure cabin (1F) in a negative pressure, thus accelerating the water intake and shortening the time for water intake; (2) vacuum washing procedure: after the water intake is finished, turning on, by the positive and negative pressure intelligent regulation and control apparatus (5), a motor assembly (15.5) to drive the drum mechanism (15.1) to rotate for washing, and meanwhile, enabling the solenoid valve (B1) to open the air evacuation pipeline (A1), air inlet port (a) of air evacuation pump→(A1)→(B1)→(A1)→air evacuation and air return port (c) of positive and negative pressure cabin, and turning on the air evacuation pump (2) to pump the washing machine type positive and negative pressure cabin (1F) to a moderate vacuum, where both the clothes fibers and stains swell in the moderate vacuum and the air therein escapes, such that the adhesive force of the stains is weaken or the stains fall off, the cleaning efficiency is improved, and the washing time is shortened, and during the laundry process, the super oxygen or carried substance mixed water directly kills bacteria and viral microorganisms on clothes, the organic matters in the dust and fouling on the clothes are decomposed and then dissolved into the water, the stain removability of the detergent is enhanced, the degree of cleaning is improved, the cleaning process is accelerated, and the function of sterilization and deodorization is achieved at the same time; (3) high-pressure washing procedure: after washing at vacuum for proper time, closing the solenoid valve (B1) and turning off the air evacuation pump (2) by the positive and negative pressure intelligent regulation and control apparatus (5), enabling the solenoid valve (B3) to open an air inflation pipeline (A3): air outlet port (d) of air inflation pump→(A3)→(B3)→(A3)→air inflation port (f) of positive and negative pressure cabin, and opening circulation pipelines (A1), (A5) and (A6) at the same time: (i) atmospheric circulation: circulation air return port (i) of positive and negative pressure cabin→(A1)→(B1)→(A5)→(B5)→(A5)→circulation exhaust port (i2)→exhaust to atmosphere for circulation; or (ii) in-machine circulation: circulation air return port (i) of positive and negative pressure cabin→(A1)→(B1)→(A5)→(B5)→(A7)→(B6)→(A6)→circulation air return port (e2) of air inflation pump, and turning on the air inflation pump (3), enabling high pressure airflow to enter the washing machine type positive and negative pressure cabin (1F) through an air inflation pipeline (A3), so as to form strong bubbles and rapid waterflow in the cabin to participate in clothes cleaning, where the high atmospheric pressure formed in the washing machine type positive and negative pressure cabin (1F) is beneficial for the detergent to penetrate into the clothes to facilitate the cleaning, and by circulating the air evacuation and air inflation repeatedly, and vacuum and high pressure are circulated and repeated, leading to fiber swell and turbulent rubbing, so as to greatly improve the cleaning degree and laundry efficiency and accelerate the washing and rinsing progress; (4) water drainage and dehydration procedure: sending an instruction by the positive and negative pressure intelligent regulation and control apparatus (5) when the water drainage is required, enabling solenoid valves (B20), (B21) and (B22) to open the corresponding water drainage pipelines: (i) when both the water suction pump and the decomposition of super oxygen and carried substance are not required, a direct water drainage line being: water drainage port (L) of washing machine type positive and negative pressure cabin→(A20)→(B20)→(A23)→(B22)→(A22)→sewer; (ii) when both the water suction pump for drainage and the decomposition of super oxygen are required, a water drainage line being: water drainage port (L) of positive and negative pressure cabin→(A20)→(B20)→(A20)→water inlet port (w) of water suction pump→water output port (p)→(A21)→(B21)→(A21)→water inlet port (g) of carried substance decomposition processor→water output port (m)→(A22)→(B22)→(A22)→sewer; (iii) when the water suction pump is required for drainage and the decomposition of super oxygen and carried substance is not required, a water drainage line being: a water drainage port (L) of washing machine type positive and negative pressure cabin→(A20)→(B20)→(A20)→water inlet port (w) of water suction pump→water output port (p)→(A21)→(B21)→(A24)→(A23)→(B22)→(A22)→sewer, during the procedures (ii) and (iii), turning on the water suction pump (16) for water drainage, during water drainage, turning on the air inflation pump (3) to inflate and pressurize the washing machine type positive and negative pressure cabin (1F), thus accelerating the water drainage speed; after the water drainage is finished, sending an instruction by the positive and negative pressure intelligent regulation and control apparatus (5) to start dehydration; during dehydration, inflating and pressurizing to force the water to rapidly separate from the clothes; turning on the air evacuation pump (2) alternately to pump the cabin to a negative pressure to make the clothes fibers in the washing machine type positive and negative pressure cabin (1F) swell, where the water in the clothes escapes to a negative pressure space and is pumped out of the cabin, such that the clothes hardened on an inner wall of the drum during dehydrating and high pressure are loosened to prevent wrinkles, and by circulating the air evacuation and air inflation repeatedly, squeezing out water and evacuating the water out the cabin are carried out alternately, the efficiency of dehydrating the clothes is greatly improved, and time for water drainage and dehydration is shortened; (5) vacuum high pressure drying procedure: when a drying procedure is started, turning on the air inflation pump (3) according to an instruction sent by the positive and negative pressure intelligent regulation and control apparatus (5), and opening an air inflation pipeline (A3) to inflate and pressurize the washing machine type positive and negative pressure cabin (1F), thus facilitating hot air to enter the clothes fibers to gasify the water, turning off the air inflation pump (3) and closing the air inflation pipeline (A3), turning on the air evacuation pump (2) and opening an air evacuation pipeline (A1) to pump the water out, and opening circulation pipelines (A5), (A7), (A6) and (A3): circulation air inlet port (j2)→(A5)→(B5)→(A7)→(B6)→(A6)→(B3)→(A3)→circulation air inlet port (j) of the positive and negative pressure cabin, evacuating out the water and making the clothes fibers swell with the negative pressure to facilitate the water emission and quick drying of the clothes, such that, by circulating the air evacuation and inflation repeatedly, heating gasification and water pump-out are alternately conducted, the drying progress is greatly accelerated, the fluffy clothes improves the drying quality, and the drying time is effectively shortened; (6) vacuum self-cleaning sterile placement procedure: closing the cabin door (1.2) after the laundry is finished, controlling, by the positive and negative pressure intelligent regulation and control apparatus (5), the washing-dehydrating-drying control mechanism to start drying hot air to blow-dry the inside of the washing machine, especially the drum mechanism (15.1), and then turning on the air evacuation pump (2) and opening the air evacuation pipeline for air evacuation, evacuating out the residual super oxygen and carried substance in the washing machine type positive and negative pressure cabin (1F), and enabling the drum mechanism (15.1) and the equipment in the cabin to be in moderate vacuum, such that the drum mechanism and the equipment in the cabin are sterile, dust-free and pollution-free for keeping clean, and oxidation-free, rust-free and undamaged for vacancy, and the service life of the washing machine is prolonged; in conclusion, the positive and negative pressure washing machine greatly accelerates the multiple processes of washing, dehydrating and drying, shortens the operation duration of the various procedures, and improves the cleaning degree of laundry and drying quality, moreover, the super oxygen also provides a thorough sterilization, disinfection and odor removal for the washed clothes; and meanwhile, the concentration and operation duration of the super oxygen and carried substance are intelligently controlled at the moderate region for sterilization, degradation and washing assistance, not reach, or as little as possible to reach, a critical point of possible oxidation fading of dark clothes; and (7) positive and negative pressure module combined intelligent smart home mode: designing and manufacturing the intelligent positive and negative pressure washing machine according to the module functions, where the intelligent positive and negative pressure washing machine not only is independently produced and used as a separated invention, but also is organically combined with other positive and negative pressure electric appliances and the module cabinets under the control of the touch screen and mobile phone monitoring and identification system (12) to form a fully functional intelligent positive and negative pressure module combined home, which not only can share the positive and negative pressure system, but also has basically uniform, neat and harmonious appearance, shape and color which are in line with the popular trend; in conclusion, targeted stain removal comprehensive washing and drying effective methods are used in the intelligent positive and negative pressure system, which make the washing, dehydrating and drying of clothes faster, more efficient and low-consumption, and the washed clothes less in consumption, cleaner, fluffier, and more sanitary.

According to a seventh embodiment of the present disclosure, an intelligent positive and negative pressure dish washing and fruit and vegetable cleaning machine is provided, including a machine body (7). The machine body (7) is internally provided with an intelligent positive and negative pressure system, a dish washing and drying system (19), and a fruit and vegetable cleaning system (20). The intelligent positive and negative pressure system includes a positive and negative pressure cabin (1), an air evacuation pump (2), a positive and negative pressure intelligent regulation and control apparatus (5), waterflow carried substance generator integration (6.04), a carried substance decomposition processor (6.7), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water inlet pump (17), and the touch screen and mobile phone monitoring and identification system (12). The positive and negative pressure cabin (1) is designed as a dish washer type positive and negative pressure cabin (1G) according to intelligent dish washing and drying and fruit and vegetable cleaning requirements, and the dish washer type positive and negative pressure cabin (1G) is a vacuum high pressure cabin (1-1) structure. A positive and negative resistance standard of the vacuum high pressure cabin (1-1) takes the local instantaneous atmospheric pressure as zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the dish washing and fruit and vegetable cleaning machine. The dish washer type positive and negative pressure cabin (1G) includes a cabin body (1.1), a cabin door (1.2), an airtight mechanism (1.3), and an internal and external communicating sealer (1.4), and the airtight mechanism (1.3) is arranged between the cabin door (1.2) and the cabin body (1.1) of the dish washer type positive and negative pressure cabin (1G). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When positive and negative atmospheric pressure is formed inside the dish washer type positive and negative pressure cabin (1G), the airtight gasket (1.33) is used for keeping sealing. The internal and external communicating sealer (1.4) is fixedly arranged at a rear part or side part of the dish washer type positive and negative pressure cabin (1G), and all pipes and circuits getting in and out the cabin are connected to and pass through the internal and external communicating sealer (1.4), so as to keep the sealing property of the dish washer type positive and negative pressure cabin (1G). The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), and a carried substance generator item addition and upgrading module (6.16). The carried substance generator item addition and upgrading module (6.16) is a combined module capable of providing or installing any individual fluid carried substance generator additional item in the carried substance generator integration and upgrading and updating the same. The dish washer type positive and negative pressure cabin (1G) is internally provided with a dish washing and drying system (19), a fruit and vegetable cleaning system (20), an anti-shower air evacuation port (2.1), a negative pressure sensor (C2), a super oxygen sensor (C3), a temperature sensor (C9), and a carried substance sensor item addition and upgrading module (C12). The carried substance sensor item addition and upgrading module (C12) is a combined module capable of providing or installing any individual sensor additional item in the sensor integration and upgrading and updating the same. The dish washer type positive and negative pressure cabin (1G) is externally provided with an air evacuation pump (2), a positive and negative pressure intelligent regulation and control apparatus (5), a waterflow carried substance generator integration (6.04), a carried substance decomposition processor (6.7), a water suction pump (16), a water input pump (17), and a touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), the other end of the air evacuation pipeline (A1) extends into the dish washer type positive and negative pressure cabin (1G) and then is connected with the anti-shower air evacuation port (2.1), so as to form an air evacuation and air return port (c), and an air outlet port (b) of the air evacuation pump (2) is connected to the atmosphere. A water intake pipeline (A14) is installed at a water inlet port (r) of the water input pump (17), a solenoid valve (B14) is installed at a middle part of the water intake pipeline (A14), and the other end of the water intake pipeline (A14) communicates with a tap water source. A water intake pipeline (A15) is installed at a water output port (u) of the water input pump (17), a solenoid valve (B15) is installed at a middle part of the water intake pipeline (A15), and the other end of the water intake pipeline (A15) communicates with a water inlet port (k) of the waterflow carried substance generator integration (6.04). A water intake pipeline (A16) is installed at a water output port (q) of the waterflow carried substance generator integration (6.04), a solenoid valve (B16) is installed at a middle part of the water intake pipeline (A16), and the other end of the water intake pipeline (A16) extends into the dish washer type positive and negative pressure cabin (1G) to be connected to a solenoid valve (B25). The solenoid valve (B25) is respectively connected to a fruit and vegetable cleaning water intake pipeline (A25) and a dish washing and drying water intake pipeline (A27), and the water intake pipeline (A25) is connected to a water inlet port (e) of the fruit and vegetable cleaning system (20). The water intake pipeline (A27) is connected to a water inlet port (G) of the dish washing and drying system (19); a water intake pipeline (A19) is further installed at the solenoid valve (B15), and the solenoid valve (B15) is connected to the solenoid valve (B16), such that the water is directly fed by the water input pump (17) when super-oxygenated water is not required. A water drainage pipeline (A20) is installed at a water inlet port (w) of the water suction pump (16), a solenoid valve (B20) is installed at a middle part of the water drainage pipeline (A20), and the other end of the water drainage pipeline (A20) extends into the dish washer type positive and negative pressure cabin (1G) and is respectively connected to a fruit and vegetable cleaning water drainage pipeline (A26) and a dish washing and drying water drainage pipeline (A28), and the water drainage pipeline (A26) is connected to a water output port (F) of the fruit and vegetable cleaning system (20). The water drainage pipeline (A28) is connected to a water drainage port (H) of the dish washing and drying system (19). A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) communicates with a sewer. A water drainage pipeline (A24) is further installed at the solenoid valve (B21), and the solenoid valve (B21) is connected to (A23) and then communicates with the solenoid valve (B22), such that the water is directly drained by the water suction pump when the super oxygen and the carried substance do not need to be decomposed. All fluid carried substance generation processors (6), sensors (C) and solenoid valves (B) and the air evacuation pump (2), the air inflation pump (3), the water suction pump (16), the water input pump (17), the dish washing and drying system (19), the fruit and vegetable cleaning system (20), and the touch screen and mobile phone monitoring and identification system (12) are all connected to the positive and negative pressure intelligent regulation and control apparatus (5).

According to an eighth embodiment of the present disclosure, an operation method for an intelligent positive and negative pressure dish washing and fruit and vegetable cleaning machine is provided. The intelligent positive and negative pressure dish washing and fruit and vegetable cleaning machine adopts an intelligent positive and negative pressure system. The intelligent positive and negative pressure system is used to accurately regulate and organically integrate various techniques such as vacuum, super oxygen, disinfection and sterilization, degradation treatment, dish washing, and fruit and vegetable cleaning, and one item of or a combination of more items of the techniques is applied to the intelligent positive and negative pressure dish washing and fruit and vegetable cleaning machine, a vacuum and high pressure airflow-waterflow dish washing and fruit and vegetable cleaning and drying technique is developed, and digital techniques such as Internet, big data, cloud computing, machine learning, artificial intelligence and expert system are also applied to the dish washing and fruit and vegetable cleaning machine at the same time to analyze and identify stain-causing elements and control targets of various different cleaning materials one by one, so as to form a complete large database according to different categories, and correspondingly use different targeted stain removal techniques. According to built-in program and pre-stored data of the positive and negative pressure dish washing and fruit and vegetable cleaning machine, and real-time feedback information of various sensors, various dish washing and fruit and vegetable cleaning and drying elements, such as waterflow and airflow pressure, gas composition, water temperature and air temperature, are regulated and controlled in real time, so as to disinfect, sterilize and degrade the cleaning materials harmlessly, and create a regulatable and controllable accurate dish washing and fruit and vegetable cleaning and drying intelligent microenvironment with the optimal effect, shortest time, highest efficiency, minimum wear and least environmental pollution. For stain-causing sites of both cleaning materials and contaminant, stain-causing factors and stain removal targets of various different cleaning scenarios and cleaning materials are analyzed one by one on the molecular and microscopic level, and after computing, different accurate stain removal techniques are correspondingly used to form targeted stain removal comprehensive cleaning methods with different characteristics for removing the stain-causing factors at fixed points, so as to form a set of digital expert system for dish washing and fruit and vegetable cleaning and drying, which is intelligently applied to achieve the purpose of fast, efficient, sanitary and environmental-friendly dish washing and fruit and vegetable cleaning. A specific operation method for the intelligent positive and negative pressure dish washing and fruit and vegetable cleaning machine includes the following steps: (1) dish washing water intake and cleaning procedure: sending an instruction by the positive and negative pressure intelligent regulation and control apparatus (5) after the machine door is closed, enabling solenoid valves (B14), (B15), (B16) and (B25) to open corresponding water intake pipelines: (i) when the super oxygen or substance-carrying water is required for dish washing, water intake line of water input pump being: (A14)→(B14)→(A14)→water inlet port (r) of water input pump→water output port (u)→(A15)→(B15)→(A15)→water inlet port (k) of waterflow carried substance generator integration→water output port (q)→(A16)→(B16)→(A16)→(B25)→(A27)→water inlet port (G) of dish washing and drying system; (ii) when the super oxygen or substance-carrying water is not required for dish washing, water intake line of water input pump being: (A14)→(B14)→(A14)→water inlet port (r) of water input pump→water output port (u)→(A15)→(B15)→(A19)→(B16)→(A16)→(B25)→(A27)→water inlet port (G) of dish washing and drying system, turning on the water input pump (17), rapidly mixing, by the waterflow carried substance generator integration, the tap water with mixed super-oxygenated or substance-carrying water generated by the waterflow carried substance generator integration, and feeding the mixture into a dish washer type positive and negative pressure cabin (1G) to accelerate the decomposition of organic fouling on the tableware, thus facilitating the rapid cleaning; after the water intake is finished, turning on the dish washing and drying system (19) by the positive and negative pressure intelligent regulation and control apparatus (5) to spray water for dish washing, and enabling a solenoid valve (B1) to open an air evacuation pipeline (A1): air inlet port (a) of air evacuation pump→(A1)→(B1)→(A1)→air evacuation and air return port (c) of positive and negative pressure cabin, and turning on the air evacuation pump (2) for vacuumizing, where, during the vacuumizing, the stain stained on the tableware expands in the negative vacuum pressure to make air escape from the stain, thus the adhesive force of the stain is weakened, or the stain fall off from the tableware, the cleaning efficiency is improved, and the washing process is shortened, moreover, the mixed super-oxygenated or substance-carrying water directly kills the bacteria and viral microorganism on the tableware, the organic matters in the stain on the tableware are decomposed and then dissolved into the water, the stain removability of the detergent is enhanced, the degree of cleaning is improved, the cleaning process is accelerated, and the function of sterilization and deodorization is achieved at the same time; (2) dish washing drainage and drying disinfection procedure: when the water drainage is required at proper time of cleaning, sending an instruction by the positive and negative pressure intelligent regulation and control apparatus (5), enabling solenoid valves (B20), (B21) and (B22) to open the corresponding water drainage pipelines: (i) when both the water suction pump and the decomposition of super oxygen and carried substance are not required, a direct water drainage line being: water output port (H) of dish washing and drying system→(A28)→(A20)→(B20)→(A23)→(B22)→(A22)→sewer; (ii) when both the water suction pump for water drainage and the decomposition of super oxygen and carried substance are required, a water drainage line being: water output port (H) of dish washing and drying system→(A28)→(A20)→(B20)→(A20)→water inlet port (w) of water drainage pump→water output port (p)→(A21)→(B21)→(A21)→water inlet port (g) of carried substance decomposition processor→water output port (m)→(A22)→(B22)→(A22)→sewer; and (iii) when the water suction pump is required for water drainage and the super oxygen does not need to be decomposed, a water drainage line being: water output port (H) of dish washing and drying system→(A28)→(A20)→(B20)→(A23)→(B22)→(A22)→sewer, draining water directly or turning on the water suction pump 16, or turning on the carried substance decomposition decomposer (6.7) to decompose waste water and then drain the decomposed waste water into the sewer; meanwhile, spraying the tableware with clear water for cleaning completely; during the drying procedure, turning on, by the positive and negative pressure regulation and control apparatus (5), the air evacuation pump (2) again to rapidly pump out the water vapor so as to accelerate the drying progress and accelerate the dish washing time, where the super oxygen or carried substance also thoroughly sterilize, disinfect and removal order of the washed tableware; (3) fruit and vegetable cleaning water intake and cleaning procedure: sending an instruction by the positive and negative pressure intelligent regulation and control apparatus (5) after the machine door is closed, enabling solenoid valves (B14), (B15), (B16) and (B25) to open corresponding water intake pipelines: (i) when the super-oxygenated water is required for fruit and vegetable cleaning, a water intake line for the water input pump being: (A14)→(B14)→(A14)→water inlet port (r) of water input pump→water output port (u)→(A15)→(B15)→(A15)→water inlet port (k) of super oxygen water mixer→water output port generation (q)→(A16)→(B16)→(A16)→(B25)→(A25)→water inlet port (e) of fruit and vegetable cleaning system; (ii) when the super-oxygenated water is not required for fruit and vegetable cleaning, a water intake line for the water input pump being: (A14)→(B14)→(A14)→water inlet port (r) of water input pump→water output port (u)→(A15)→(B15)→(A19)→(B16)→(A16)→(B25)→(A25)→water inlet port (e) of fruit and vegetable cleaning system, and turning on the water input pump (17), rapidly mixing, by the waterflow carried substance generator integration (6.04), the tap water with mixed super-oxygenated or substance-carrying water generated by the waterflow carried substance generator integration, and feeding the mixture into the dish washer type positive and negative pressure cabin (1G); after the water intake is finished, turning on the fruit and vegetable cleaning system (19) by the positive and negative pressure intelligent regulation and control apparatus (5) to spray water for fruit and vegetable cleaning, and enabling the solenoid valve (B1) to open the air evacuation pipeline (A1): air inlet port (a) of air evacuation pump→(A1)→(B1)→(A1)→air evacuation and air return port (c) of positive and negative pressure cabin, and turning on the air evacuation pump (2) for vacuumizing, where, during the vacuumizing, the stain stained on the fruit and vegetable expands in the negative vacuum pressure to make air escape from the stain, thus the adhesive force of the stain is weakened, or the stain fall off from the tableware, the cleaning efficiency is improved, and the washing process is shortened, moreover, the mixed super-oxygenated or substance-carrying water directly kills the bacteria and viral microorganism on the skin of the fruits and vegetables, the organic matters in stain on skin of the fruits and vegetables are decomposed and then dissolved into the water, the cleaning degree is improved, and the cleaning process is accelerated; and meanwhile, the negative pressure promotes the escape of pesticide and heavy metal residues in the tissues of the fruits and vegetables and the harmful gases such as the volatile metabolites ethylene acetaldehyde ethanol, thus a favorable environment is created for the super-oxygenated water to rapidly clean and deeply degrade the pesticide residues and decompose the harmful gases such as ethylene; sending an instruction by the positive and negative pressure intelligent regulation and control apparatus (5) after cleaning by the mixed super-oxygenated or substance-carrying water, and spraying and cleaning the fruits and vegetables with clear water; (4) water drainage procedure after fruit and vegetable cleaning: when the water drainage is required after cleaning for proper time, sending an instruction by the positive and negative pressure intelligent regulation and control apparatus (5), enabling solenoid valves (B20), (B21) and (B22) to open corresponding water drainage pipelines: (i) when both the water suction pump and the decomposition of super oxygen or carried substance are not required, a direct water drainage line being: water output port (F) of fruit and vegetable cleaning system→(A26)→(A20)→(B20)→(A23)→(B22)→(A22)→sewer; (ii) when both the water suction pump for water drainage and the decomposition of super oxygen and carried substance are required, a water drainage line being: water output port (F) of fruit and vegetable cleaning system→(A26)→(A20)→(B20)→(A20)→water inlet port (w) of water suction pump→water output port (p)→(A21)→(B21)→(A21)→water output port (g) of carried substance decomposition processor→water output port (m)→(A22)→(B22)→(A22)→sewer; (iii) when the water suction pump is required for water drainage and the super oxygen or carried substance does not need to be decomposed, a water drainage line being: water output port (F) of fruit and vegetable cleaning system→(A26)→(A20)→(B20)→(A23)→(B22)→(A22)→sewer, directly draining water or turning on the water suction pump (16) or turning on the carried substance decomposition processor (6.7) again to decompose waste water, and then draining the decomposed waste water into the sewer; (5) vacuum self-cleaning sterile placement sterile: closing the machine door (1.2) after use, starting drying hot air by the positive and negative pressure intelligent regulation and control apparatus (5) to blow-dry the inside of the dish washer type positive and negative pressure cabin (1G), especially the dish washing and drying system (19) and the fruit and vegetable cleaning system (20), and then turning on the air evacuation pump (2) and opening the air evacuation pipeline for air evacuation, evacuating out the residual super oxygen or carried substance in the dish washer type positive and negative pressure cabin (1G), and enabling the dish washing and drying system (19), the fruit and vegetable cleaning system (20) and the equipment in the cabin to be in a moderate vacuum, such that the dish washing-drying system (19) and the fruit and vegetable cleaning system (20) and the equipment in the cabin are sterile, dust-free, pollution-free, and kept clean for health, and are oxidation-free, rust-free and undamaged for vacancy, and the service life of the electric appliance is prolonged; and (6) positive and negative pressure module combined intelligent smart home mode: designing and manufacturing the intelligent positive and negative pressure dish washing and fruit and vegetable cleaning machine according to the module functions, where the intelligent positive and negative pressure dish washing and fruit and vegetable cleaning machine not only is independently produced and used as a separated invention, but also is organically combined with other positive and negative pressure electric appliances and the module cabinets under the control of the touch screen and mobile phone monitoring and identification system (12) to form a fully functional intelligent positive and negative pressure module combined home, which not only shares the water input pump (17), the water suction pump (16), the waterflow carried substance generator integration (6.04) and the carried substance decomposition processor (6.7), but also has basically uniform, neat and harmonious appearance, shape and color which are in line with the popular trend, such that the dish washing and fruit and vegetable cleaning machine is more efficient, low-consumption, and suitable for use.

According to a ninth embodiment of the present disclosure, an intelligent positive and negative pressure range hood is provided, including a machine body (7). A lower half part of the machine body (7) is a range hood type positive and negative pressure cabin (1H), and an upper half part of the machine body (7) is an equipment fume tube cabin (21). The equipment fume tube cabin (21) is internally provided with a positive and negative pressure system, a fume evacuation control mechanism (26), and a water heating apparatus (27). The positive and negative pressure system includes: a positive and negative pressure intelligent regulation and control apparatus (5), a water input pump (17), a water suction pump (16), a waterflow carried substance generator integration (6.04), and a carried substance decomposition processor (6.7). The range hood type positive and negative pressure cabin (1H) is an atmospheric circulation cabin (1-2) structure, including a triangular cabin body (1.1) and a cabin door (1.2). The cabin door (1.2) is a machine door of the positive and negative pressure range hood, and a waterproof mechanism (1.7) is arranged between the cabin door (1.2) and the cabin body (1.1). The waterproof mechanism (1.7) includes a lock ring (1.71), a lock bolt (1.72), an airtight gasket (1.73), and a cabin door hinge (1.74). The lock ring (1.71) is in movable fit with the lock bolt (1.72), the lock bolt (1.72) extends into the lock ring (1.71) to lock the cabin door (1.2) during the closing of the cabin door. When spraying and cleaning are conducted in the range hood type positive and negative pressure cabin (1H), the waterproof gasket (1.73) is used for keeping sealing. The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), a carried substance generator item addition and upgrading module (6.16). The carried substance generator item addition and upgrading module (6.16) is a combined module capable of providing or installing any individual fluid carried substance generator additional item in the carried substance generator integration and upgrading and updating the same. The range hood type positive and negative pressure cabin (1H) is internally provided with a motor-turbine assembly (22), an automatic rotary spraying ball (23), an oil collecting and water drainage groove (24), a super oxygen sensor (C3), and carried substance sensor item addition and upgrading module (C12), and is provided with a touch screen and mobile phone monitoring and identification system (12) on a front surface. The carried substance sensor item addition and upgrading module (6.16) is a combined module capable of providing or installing any individual sensor additional item in the sensor integration and upgrading and updating the same. A water intake pipeline (A14) is installed at a water inlet port (r) of the water input pump (17), and the water inlet port (r) communicates with a tap water source; a pipeline (A15) is installed at a water output port (u) of the water input pump (17), a solenoid valve (B15) is installed at a middle part of the pipeline (A15), and the other end of the pipeline (A15) is connected to a water inlet port (z) of the water heating apparatus (27). A pipeline (A16) is installed at a water output port (q) of the water heating apparatus (27), a solenoid valve (B16) is installed at a middle part of the pipeline (A16), and the other end of the pipeline (A16) communicates with a water inlet port (v) of the waterflow carried substance generator integration (6.04). A pipeline (A18) is installed at a water output port (o) of the waterflow carried substance generator integration (6.04), a solenoid valve (B18) is installed at a middle part of the pipeline (A18), and the other end of the pipeline (A18) extends into the range hood type positive and negative pressure cabin (1H) to be connected to a spraying pipeline (A30). A pipeline (A17) is installed at a water output port (k) of the water heating apparatus (27), a solenoid valve (B17) is installed at a middle part of the pipeline (A17), and the other end of the pipeline (A17) communicates with the solenoid valve (B18), and extends into the range hood type positive and negative pressure cabin (1H) to be connected to the spraying pipeline (A30). A plurality of automatic rotary spraying balls (23) are installed on the spraying pipeline A30, and an included angle between the axis of each spraying ball and the axis of a circle of spraying holes in the middle is 45 degrees, and the spraying ball 23 rotates automatically when waterflow is sprayed. A water drainage pipeline (A20) is installed at a water inlet port (w) of the water suction pump (16), a solenoid valve (B20) is installed at a middle part of the water drainage pipeline (A20), and the other end of the water drainage pipeline (A20) extends into the oil collecting and water drainage groove (24) at the bottom of the range hood type positive and negative pressure cabin (1H) to form a water drainage port (L). A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) communicates with the sewer. A water drainage pipeline (A24) is further installed on the solenoid valve (B21), and the solenoid valve (B21) is connected to the solenoid valve (B22). All fluid carried substance generation processors (6), sensors (C) and solenoid valves (B) and the water suction pump (16), the water input pump (17), the fume evacuation control mechanism (26), the water heating apparatus (17), and the touch screen and mobile phone monitoring and identification system (12) are all connected to the positive and negative pressure intelligent regulation and control apparatus (5).

According to a tenth embodiment of the present disclosure, an operation method for an intelligent positive and negative pressure range hood is provided. The intelligent positive and negative pressure range hood adopts an intelligent positive and negative pressure system; the intelligent positive and negative pressure system is used to accurately regulate and organically integrate various techniques such as super oxygen, disinfection and sterilization, degradation treatment, and high pressure cleaning, and one item of or a combination of more items of the techniques is applied to the intelligent positive and negative pressure range hood. For stain-causing sites of both cleaning materials and contaminant in the range hood, stain-causing factors and stain removal targets of various different cleaning scenarios and cleaning materials are analyzed one by one on the molecular and microscopic level, and after computing, different accurate stain removal techniques are correspondingly used to form targeted stain removal comprehensive cleaning methods with different characteristics for removing the stain-causing factors at fixed points, so as to achieve the purpose of fast, efficient, sanitary and environmental-friendly range hood cleaning. The method includes the following steps: (1) fume extraction procedure: when the range hood is started, opening the sealed waterproof cabin door (1.2) for air intake and fume extraction, and closing the sealed cabin door (1.2) after the range hood stops operating; (2) self-cleaning procedure: sending an instruction by the positive and negative pressure intelligent regulation and control apparatus (5), enabling solenoid valves (B15), (B18), (B16) or (B17) to open corresponding water intake pipelines: (I) when the super oxygen or related carrying water is required for cleaning, a water intake line being: (A14)→water inlet port (r) of water input pump→water output port (u)→(A15)→(B15)→(A15)→water inlet port (z) of water heating apparatus→water output port (q)→(A16)→(B16)→(A16)→water inlet port (v) of related carried substance generator of waterflow carried generator substance integration→water output port (o)→(A18)→(B18)→(A18)→(A30)→automatic rotary spraying ball (23); (II) when super oxygen is not required to carry hot water for hydrolysis for cleaning, a water intake line being: (A14)→water inlet port (r) of water input pump→water output port (u)→(A15)→(B15)→(A15)-water inlet port (z) of water heating apparatus→water output port (k)→(A17)→(B17)→(A17)→(A15)→(B16)→(A16)→(A30)→automatic rotary spraying ball (23), turning on the water input pump (A17), enabling tap water to form super-oxygenated or carrying water through the water heating apparatus (27) and the waterflow carried substance generator integration (6.04) to enter the range hood type positive and negative pressure cabin (1H), comprehensively spraying and cleaning various parts, including a motor turbine assembly (22), in a machine case using the automatic rotary spraying ball (23); firstly spraying the supe-oxygenated or substance-carrying hot water to efficiently decompose and remove the oil stains, and then spraying with clean hot water until the oil stains are completely cleaned; enabling the sewage obtained after cleaning to flow downwards to be collected into an oil collecting and water drainage groove (24) at the lowest part of the machine case; when the water drainage is needed, sending an instruction by the positive and negative pressure intelligent regulation and control apparatus (5), enabling solenoid valves (B20), (B21) and (B22) to open corresponding water drainage pipelines: (I) when the super oxygen or carried substance needs to be decomposed, a water drainage line being: water drainage port (L) of oil collecting and water drainage groove→(A20)→(B20)→(A20)→water inlet port (w) of water suction pump→water output port (p)→(A21)→(B21)→(A21)→water inlet port (g) of carried substance decomposition processor→water output port (m)→(A22)→(B22)→(A22)→sewer; (II) when the super oxygen or carried substance does not need to be decomposed, a water drainage line being: water drainage port (L)w of oil collecting and water drainage groove→(A20)→(B20)→(A20)→water inlet port (w) of water suction pump→water output port (p)→(A21)→(B21)→(A24)→(B22)→(A22)→sewer, and turning on the water suction pump (16) to drain the water to the sewer; (3) drying placement procedure: after cleaning, turning on the range hood according to an instruction sent by the positive and negative pressure intelligent regulation and control apparatus (5) to completely blow-dry the inside of the machine case, and then turning off the range hood to prevent corrosion; (4) turn-on protection function: when the range hood is turned on, enabling the air to immediately flow into the positive and negative pressure water washing range hood type positive and negative pressure cabin (1H) from the outside of the range hood cabin and to be exhausted from a fume exhaust pipe (29), such that the range hood type positive and negative pressure cabin (1H) is not exposed to any super oxygen carried substance; and (5) module combined intelligent smart home mode: designing and manufacturing the intelligent positive and negative pressure range hood according to the module functions, where the intelligent positive and negative pressure range hood not only is independently produced and used as a separated invention, but also is organically combined with other positive and negative pressure electric appliances and the module cabinet under the control of the touch screen and mobile phone monitoring and identification system (12) to form a fully functional intelligent positive and negative pressure module combined home; the fully functional intelligent positive and negative pressure module combined home not only shares the water input pump (17), the water suction pump (16), the waterflow carried substance generator integration (6.04) and the carried substance decomposition processor (6.7) with the dish washing and fruit and vegetable cleaning machine, but also has basically uniform, neat and harmonious appearance, shape and color which are in line with the popular trend, such that the intelligent range hood is more efficient, low-consumption, and suitable for use.

According to an eleventh embodiment of the present disclosure, an intelligent positive and negative pressure baking and frying microwave oven is provided, including a machine body (7). The machine body (7) is internally provided with an intelligent positive and negative pressure system, a baking system (30), an air frying system (31), and a microwave system (32). The intelligent positive and negative pressure system includes a positive and negative pressure cabin (1), an air evacuation pump (2), an air inflation pump (3), a positive and negative pressure intelligent regulation and control apparatus (5), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), and the touch screen and mobile phone monitoring and identification system (12). The positive and negative pressure cabin (1) is designed as a microwave oven type positive and negative pressure cabin (1K) according to the requirements of an oven, an air fryer and the microwave oven, and the microwave oven type positive and negative pressure cabin (1K) is a vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the baking and frying microwave oven. The microwave oven type positive and negative pressure cabin (1K) includes a cabin body (1.1), a cabin door (1.2), an airtight mechanism (1.3), and an internal and external communicating sealer (1.4). The cabin door (1.2) is a machine door of the positive and negative pressure baking and frying microwave oven. The airtight mechanism (1.3) is arranged between the cabin door (1.2) and the cabin body (1.1) of the microwave oven type positive and negative pressure cabin (1K). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When the positive and negative pressure is formed inside microwave oven type positive and negative pressure cabin (1K), the airtight gasket (1.33) is used for keeping sealing. The internal and external communicating sealer (1.4) is fixedly arranged at a rear side part of the microwave oven type positive and negative pressure cabin (1K), and all pipes and circuits getting in and out the cabin are connected to and pass through the internal and external communicating sealer (1.4), so as to keep the sealing property of the microwave oven type positive and negative pressure cabin (1K). The positive and negative pressure intelligent regulation and control apparatus (5) includes an integrated circuit, a chip, and a regulation and control system for a fluid and a carried substance thereof, and is connected to an Internet in a wired or wireless mode. The microwave oven type positive and negative pressure cabin (1K) is internally provided with the baking system (30), an air frying system (31), a microwave system (32), a positive and negative pressure sensor (C15), and a sensor item addition and upgrading module (C12). A non-standard structure other than above standard structure is also used inside the microwave oven type positive and negative pressure cabin (1K), which includes at least one of the microwave system (32), the baking system (30), and the air frying system (31). The microwave oven type positive and negative pressure cabin (1K) is externally provided with the air evacuation pump (2), the air inflation pump (3), the positive and negative pressure intelligent regulation and control apparatus (5), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the microwave oven type positive and negative pressure cabin (1K) to form an air evacuation and air return port (c) thereof. An air air exhaust pipeline (A2) is installed at an air outlet port (b) of the air evacuation pump (2), and is connected to the atmosphere; an air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the microwave oven type positive and negative pressure cabin (1K) to form an air inflation and inlet port (f). An air intake pipeline (A4) is installed at an air inlet port (e) of the air inflation pump (3) and is connected to the atmosphere. All sensors (C) and solenoid valves (B), and the air evacuation pump (2), the air inflation pump (3), the baking system (30), the air frying system (31), the microwave system (32), and the touch screen and mobile phone monitoring and identification system (12) are all connected to the positive and negative pressure intelligent regulation and control apparatus (5).

According to a twelfth embodiment of the present disclosure, an intelligent positive and negative pressure fresh-keeping transport compartment container is provided, including a compartment body (7). The compartment body (7) is internally provided with an intelligent positive and negative pressure system, and a refrigeration system (8). The intelligent positive and negative pressure system includes a positive and negative pressure cabin (1), an air evacuation pump (2), an air inflation pump (3), a positive and negative pressure intelligent regulation and control apparatus (5), a positive and negative pressure fluid carried substance generation processor (6), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a compartment container type positive and negative pressure cabin (1M) or an atmospheric compartment container type positive and negative pressure cabin (1M-2) according to the requirements of the intelligent positive and negative pressure fresh-keeping transport compartment container. An internal structure of the compartment container type positive and negative pressure cabin (1M) includes a cabin body (1.1), a cabin door (1.2), an airtight mechanism (1.3), and an internal and external communicating sealer (1.4). The cabin door (1.2) is a compartment door of the positive and negative pressure fresh-keeping transport compartment container; the airtight mechanism (1.3) is arranged between the cabin door (1.2) and the cabin body (1.1) of the compartment container type positive and negative pressure cabin (1M). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When the positive and negative pressure is formed inside the compartment container type positive and negative pressure cabin (1M), the airtight gasket (1.33) is used for keeping sealing. The internal and external communicating sealer 1.4 is fixedly arranged at a rear part of the compartment container type positive and negative pressure cabin (1M), and all pipes and circuits getting in and out the cabin are connected to and pass through the internal and external communicating sealer (1.4). The compartment container type positive and negative pressure cabin (1M), due to the installation of the airtight mechanism (1.3) and the internal and external communicating sealer (1.4) and the possess of a corresponding vacuum high pressure resistance structure, belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the compartment container. The atmospheric compartment container type positive and negative pressure cabin (1M-2) is a general compartment container with the positive and negative pressure system and without the airtight mechanism (1.3) and the internal and external communicating sealer (1.4). A general compartment container space is an atmospheric positive and negative pressure cabin without a vacuum high pressure resistance structure, belonging to a general sealed atmospheric circulation cabin (1-2) or atmospheric inlet-outlet cabin (3) structure. The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), an airflow carried substance generator integration (6.02), and an integrated pipeline type positive and negative fluid carried substance generator (6.05). The air conditioning apparatus (6.1) is a membrane-based air separation type air conditioning apparatus. The carried substance generator item addition and upgrading module (6.16) is a combined module capable of providing or installing any individual fluid carried substance generator additional item in the carried substance generator integration and upgrading and updating the same. Each of the airflow carried substance generator integration (6.02) and the integrated pipeline type positive and negative pressure fluid carried substance generator (6.05) includes any one or more in the individual fluid carried substance generators (6.1) to (6.16). The compartment container type positive and negative pressure cabin (1M) or the atmospheric compartment container type positive and negative pressure cabin (1M-2) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration system (8), an air-to-water production apparatus (11), and a touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the compartment container type positive and negative pressure cabin (1M) or the atmospheric compartment container type positive and negative pressure cabin (1M-2) to form an air evacuation and air return port (c) thereof. Exhaust and evacuation pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), and passes through the air-to-water production apparatus (11) and then is connected to the atmosphere through an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the compartment container type positive and negative pressure cabin (1M) or the atmospheric compartment container type positive and negative pressure cabin (1M-2) to form an air inflation and inlet port (f) thereof. An air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to atmosphere; a carrying circulation pipeline (A5) is installed at an air inlet port (e2) of the air inflation pump (3), the other end of the carrying circulation pipeline (A5) communicates with a solenoid valve (B3), and then extends into the compartment container type positive and negative pressure cabin (1M) or the atmospheric compartment container type positive and negative pressure cabin (1M-2) through the air inflation pipeline (A3) to form a circulating air return port (j) thereof. Carrying air introduction pipelines (A6), (A6.1) and (A6.2) are further connected to the air inflation pipeline (A3), a solenoid valve (B6) is installed at a middle part of the carrying air introduction pipeline (A6), and the other end of the carrying air introduction pipeline (A6) is connected to an air inlet port (t) of the airflow carried substance generator integration (6.02). A carrying air introduction pipeline (A7) is installed at an air outlet port (s) of the airflow carried substance generator integration (6.02), a solenoid valve (B7) is installed at a middle part of the carrying air introduction pipeline (A7), and the other end of the carrying air introduction pipeline (A7) extends into the compartment container type positive and negative pressure cabin (1M) or the atmospheric compartment container type positive and negative pressure cabin (1M-2) to form a carrying air inlet port (i) thereof. Solenoid valves (B6.1) and (B6.2) are respectively installed at middle parts of the air introduction pipelines (A6.1) and (A6.2), and the other ends of the air introduction pipelines (A6.1) and (A6.2) extend into the compartment container type positive and negative pressure cabin (1M) or the atmospheric compartment container type positive and negative pressure cabin (1M-2) to be connected to air inlet ports of the airflow carried substance generator integration (6.021) and the integrated pipeline type positive and negative pressure fluid carried substance generator (6.05). An air-to-water production air inflation pipeline (A9) is further connected to the air inflation pipeline (A3), the air-to-water production air inflation pipeline (A9) is connected to the solenoid valve (B2) and then is connected to the air exhaust pipeline (A2); the middle part of the air exhaust pipeline (A2) communicates with the air-to-water production apparatus (11), and the other end of the air exhaust pipeline (A2) is connected to the atmosphere. The touch screen and mobile phone monitoring and identification system (12) includes a door touch screen (12.1), a mobile phone APP (12.2), a wireless anti-fog camera identification apparatus (12.4), and a wireless radar scanning and identification apparatus (12.6). The wireless anti-fog camera identification apparatus (12.4) and the wireless radar scanning and identification apparatus (12.6) are installed at positions inside/outside the compartment body (7) needing to be monitored and identified. The compartment container type positive and negative pressure cabin (1M) or the atmospheric compartment container type positive and negative pressure cabin (1M-2) is internally provided with an airflow carried substance generator integration (6.021), an integrated pipeline type positive and negative pressure fluid carried substance generator (6.05), and corresponding sensors thereof. The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

According to a thirteenth embodiment of the present disclosure, an operation method for an intelligent positive and negative pressure fresh-keeping compartment container is provided. The intelligent positive and negative pressure fresh-keeping compartment container adopts an intelligent positive and negative pressure system. The intelligent positive and negative pressure system is used to accurately regulate and organically integrate various techniques such as super oxygen, catalysts, air conditioning fresh-keeping, negative ion, humidification, dehumidification, disinfection and degradation, air purification, air-to-water production and low-temperature storage, and the one item of or a combination of more items of the techniques is applied to the positive and negative pressure fresh-keeping compartment container, and digital techniques such as Internet, big data, cloud computing, machine learning, artificial intelligence and expert system are also applied to the compartment container at the same time, so as to form a regulatable and controllable artificial intelligence microclimate in the positive and negative pressure fresh-keeping compartment container and to create a high-energy and efficient precise fresh-keeping microenvironment. Various fresh-keeping elements such as air composition, airflow pressure, humidity and temperature, aseptic cleanliness and the like of the positive and negative pressure fresh-keeping compartment container are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization and disinfection, quickly degrade harmful residues on the surface of preservation, achieve all-round and multi-angle digital targeted treatment, eliminate putrefying factors, accurately keep freshness and comprehensively preserve nutrition. Various fresh goods stored in and transported by the positive and negative pressure fresh-keeping compartment container are in an optimal pressure range, an optimal gas composition and optimal humidity and temperature, and an optimal preservation environment; fruits and vegetables are hibernated at low temperature in an existing refrigerating compartment container, while the positive and negative pressure and digital techniques are used in the positive and negative pressure fresh-keeping compartment container to make fruits and vegetables in a dormant or suspended animation state, such that the fruits and vegetables have the weakest life, the slowest aging, the lowest breathing and the least consumption of nutrition, and the nutritional components and the original freshness of fruits, vegetables, meat, fish and other stored and transported products are quickly stored for a long time. In the positive and negative pressure system, the digital technique is used to analyze and identify fresh-loss sites and fresh-keeping elements of various stored and transported products one by one to form a complete large database according to different categories, and to design a personalized targeted control comprehensive fresh-keeping method at the cellular and molecular level for the fresh-loss sites and fresh-keeping elements of the stored and transported products, so as to accurately maintain the fresh-keeping elements and remove the fresh-loss factors at a fixed point, without affecting or less affecting the normal tissues and cells of the stored and transported products, and thus a set of digital expert system for fresh-keeping of stored products is formed and gradually improved, which is intelligently applied to the positive and negative pressure fresh-keeping compartment container to achieve the purpose of keeping freshness and quality of the stored and transported products. In a specific operation method for the intelligent positive and negative pressure fresh-keeping compartment container, an air evacuation pump or air inflation pump is used by the positive and negative pressure system to intelligently regulate and control airflow pressure, or carry super oxygen, catalyst, negative ion, air conditioning gas, disinfectant, detergent, heat, water and various effective loads by the airflow to get in, stay in or get out of a compartment container type positive and negative pressure cabin (1M) or an atmospheric compartment container type positive and negative pressure cabin (1M-2) orderly, and the following methods are used to apply various forces helpful for long-term freshness and quality keeping to fresh fruits and vegetables stored in the compartment container: (1): positive atmospheric pressure high pressure action method: forming moderate positive atmospheric pressure at proper time in a cabin of the compartment container type positive and negative pressure cabin (1M), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the compartment container type positive and negative pressure cabin (1M), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) airflow carried substance action method: carrying single or a combination of super oxygen, catalyst, negative ion, air conditioning gas, disinfection and degradation substances, heat, water and various effective loads by positive and negative pressure airflow to get in, stay in or get out of the compartment container type positive and negative pressure cabin (1M), or the atmospheric compartment container type positive and negative pressure cabin (1M-2) orderly, integrating various fresh-keeping techniques to apply various accurate and effective action forces for the long-term freshness and quality keeping of the stored and transported fruits, vegetables and fresh goods. Based on above three action force methods, a positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of a touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping; the targeted control comprehensive fresh-keeping methods for the intelligent positive and negative pressure system and specific use methods and operation procedures of the positive and negative pressure fresh-keeping compartment container are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1) to open corresponding air exhaust pipelines (A2) and (A1), and turning on the air evacuation pump (2) to exhaust or pump the atmospheric compartment container type positive and negative pressure cabin (1M-2) or the compartment container type positive and negative pressure cabin (1M) to vacuum or negative pressure between −0.001 KPa and −0.1 MPa (taking the local instant atmospheric pressure as the zero standard), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within compartment container type positive and negative pressure cabin (1M), thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) pressurized or atmospheric circulating sterilization, degradation and deodorization procedure for super oxygen, negative ion or catalyst positive and negative pressure carried substance: according to the procedure setting and feedback of sensors (C), sending, by the positive and negative pressure intelligent regulation and control apparatus (5), an instruction to turn on a super oxygen generator (6.2), a negative ion generator (6.3), a catalyst release controller (6.4), a humidity control apparatus (6.5) or a disinfection and degradation apparatus (6.9) in an airflow carried substance generator integration (6.02) or (6.021) or an integrated pipeline type fluid carried substance generator (6.05), or a carried substance generator installed in a carried substance generator module (6.16), thus enabling the super oxygen, catalyst, negative ion, water and related carried substances in the positive and negative pressure cabin reach required standards; meanwhile, enabling solenoid valves (B3), (B4) and (B3.1), (B3.2), (B3.3) or (B3.4) to open corresponding air inflation pipelines (A4) and (A3) and (A3.1), (A3.2), (A3.3), or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and compartment container configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric, pressurized or humidified preservation procedure of air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3) and (B4), (B6), (B6.1) or (B6.2) to open corresponding related carried substance air inflation pipelines (A4) and (A3), (A6), (A6.1) or (A6.2), or enabling solenoid valves (B3), (B4), (B6) and (B7) to open corresponding related carried substance circulation pipelines (A3), (A5), (A6) and (A7), and turning on the air inflation pump (3) for the inflation and pressurization or atmospheric circulation of the compartment container type positive and negative pressure cabin (1M) or the atmospheric compartment container type positive and negative pressure cabin (1M-2); during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B1) and (B2) or (B8.1) to open carrying waste gas evacuation and exhaust pipelines (A8) or (A8.1), (A1) or (A2), carrying waste gas evacuation and exhaust port (s1) or (s2) of air conditioning apparatus (6.1)→(A8) or (A8.1)→solenoid valves (B1)→(A1)→air inlet port (a) of air evacuation pump→air outlet port (b) of air evacuation pump→(A2)→carried substance decomposition processor (6.7)→(A2)→(B2)→(A2)→air-to-water production apparatus (11)→(A2)→exhaust to atmosphere; turning on the air evacuation pump (2) to exhaust and pump decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) low-temperature assisted fresh-keeping procedure: after the positive and negative pressure fresh-keeping compartment container is turned on and the door is closed, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (6) open-to-exhaust protection function: if the compartment door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to the carried substance decomposition processor (6.7) to be exhausted to atmosphere after being decomposed, where, as the air flows from the outside of the fresh-keeping compartment container door to the positive and negative pressure cabin for supplementing air evacuation negative pressure, the super oxygen or high-nitrogen low-oxygen carried substance gas cannot flow out of the door; (7) air-to-water production procedure: enabling moist waste gas pumped from the vacuum high pressure cabin by the air evacuation pump 2 to enter the carried substance decomposition processor (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B2) to open air-to-water production inflation pipelines, thus driving the indoor air to enter the air-to-water production apparatus (11) for water production; (8) remote and short-range control monitoring function: installing a high-definition anti-fog camera and identification apparatus (12.3) and a radar scanning and identification apparatus (12.5) at positions needing monitoring and identification inside and outside the compartment body (7); shooting and scanning statuses of objects in the fresh-keeping compartment container in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling, by the positive and negative pressure system, various fresh-keeping environmental factors such as airflow pressure, gas composition, air cleanliness, environment temperature and humidity in the positive and negative pressure cabin in real time, accurately positioning and removing the fresh-loss factors in a targeted manner, and automatically performing harmless disinfection and sterilization, and rapidly degrading residual pesticides, fertilizers, hormones and harmful additives. In conclusion, in the specific operation method for the intelligent positive and negative pressure fresh-keeping compartment container, various techniques such as vacuum, high pressure, super oxygen, catalyst, air-conditioning fresh-keeping, negative ion, humidification, humidity removal, humidity control, disinfection and sterilization, air purification, air-to-water production and low-temperature storage are intelligently regulated, controlled and integrated under the positive pressure and negative pressure, which are organically applied to the intelligent positive and negative pressure fresh-keeping compartment container and are organically integrated and optimally matched, one item of or a combination of more items of the techniques is used individually or alternately or circularly, and multiple positive and negative pressure cabins with different types and different functions are arranged in a positive and negative pressure fresh-keeping compartment, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping compartment container updates the existing method that the traditional refrigerating compartment container achieves low-temperature storage only by controlling the temperature of the fresh-keeping compartment container. A regulatable and controllable artificial intelligence microclimate is formed in the positive and negative pressure cabin to create a precise fresh-keeping microenvironment, in which various fresh food stored can keep fresh, moist and original quality and flavor for a long time and to the maximum extent.

According to a fourteenth embodiment of the present disclosure, an intelligent positive and negative pressure fresh-keeping warehouse is provided, including a warehouse body (7). The warehouse body (7) is internally provided with an intelligent positive and negative pressure system, and a refrigeration system (8). The intelligent positive and negative pressure system includes a positive and negative pressure cabin (1), an air evacuation pump (2), an air inflation pump (3), a positive and negative pressure intelligent regulation and control apparatus (5), a positive and negative pressure fluid carried substance generation processor (6), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a warehouse type positive and negative pressure cabin (1N) or an atmospheric warehouse type positive and negative pressure cabin (1N-2) according to the requirements of the intelligent positive and negative pressure fresh-keeping warehouse. An internal structure of the warehouse type positive and negative pressure cabin (1N) includes a cabin body (1.1), a cabin door (1.2), an airtight mechanism (1.3), and an internal and external communicating sealer (1.4). The cabin door (1.2) is a warehouse door of the positive and negative pressure fresh-keeping warehouse. The airtight mechanism (1.3) is arranged between the cabin door (1.2) and the cabin body (1.1) of the warehouse type positive and negative pressure cabin (1N). The airtight mechanism (1.3) includes a lock ring (1.31), a lock bolt (1.32), and an airtight gasket (1.33). The lock ring (1.31) is in movable fit with the lock bolt (1.32), the lock bolt (1.32) extends into the lock ring (1.31) to lock the cabin door during the closing of the cabin door, and when the positive and negative pressure is formed inside the warehouse type positive and negative pressure cabin (1N), the airtight gasket (1.33) is used for keeping sealing. The internal and external communicating sealer (1.4) is fixedly arranged at a rear part of the warehouse type positive and negative pressure cabin (1N), and all pipes and circuits getting in and out the cabin are connected to and pass through the internal and external communicating sealer (1.4). The warehouse type positive and negative pressure cabin (1N), due to the installation of the airtight mechanism (1.3) and the internal and external communicating sealer (1.4) and the possess of a corresponding vacuum high pressure resistance structure, belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the fresh-keeping warehouse. The atmospheric warehouse type positive and negative pressure cabin (1N-2) is a general warehouse with the positive and negative pressure system and without the airtight mechanism (1.3) and the internal and external communicating sealer (1.4). A general warehouse space is an atmospheric positive and negative pressure cabin without a vacuum high pressure resistance structure, belonging to a general sealed atmospheric circulation cabin (1-2) or atmospheric inlet-outlet cabin (3) structure; the intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), and an airflow carried substance generator integration (6.02) or an integrated pipeline type positive and negative fluid carried substance generator (6.05). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. Each of the airflow carried substance generator integration (6.02) and the integrated pipeline type positive and negative pressure fluid carried substance generator (6.05) includes any one or more in the individual fluid carried substance generators (6.1) to (6.16). The warehouse type positive and negative pressure cabin (1N) or the atmospheric warehouse type positive and negative pressure cabin (1N-2) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration system (8), an air-to-water production apparatus (11), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the warehouse type positive and negative pressure cabin (1N) or the atmospheric warehouse type positive and negative pressure cabin (1N-2) to form an air evacuation and air return port (c) thereof. Exhaust and evacuation pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), and passes through the air-to-water production apparatus (11) and then is connected to the atmosphere through an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the warehouse type positive and negative pressure cabin (1N) or the atmospheric warehouse type positive and negative pressure cabin (1N-2) to form an air inflation and inlet port (f) thereof; an air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to the atmosphere. A carrying circulation pipeline (A5) is installed at an air inlet port (e2) of the air inflation pump (3), the other end of the carrying circulation pipeline (A5) communicates with a solenoid valve (B3), and then extends into the warehouse type positive and negative pressure cabin (1N) or the atmospheric warehouse type positive and negative pressure cabin (1N-2) through the air inflation pipeline (A3) to form a circulating air return port (j) thereof. Carrying air introduction pipelines (A6), (A6.1) and (A6.2) are further connected to the air inflation pipeline (A3), a solenoid valve (B6) is installed at a middle part of the carrying air introduction pipeline (A6), and the other end of the carrying air introduction pipeline (A6) is connected to an air inlet port (t) of the airflow carried substance generator integration (6.02). A carrying air introduction pipeline (A7) is installed at an air outlet port (s) of the airflow carried substance generator integration (6.02), a solenoid valve (B7) is installed at a middle part of the carrying air introduction pipeline (A7), and the other end of the carrying air introduction pipeline (A7) extends into the warehouse type positive and negative pressure cabin (1N) or the atmospheric warehouse type positive and negative pressure cabin (1N-2) to form a carrying air inlet port (i) thereof. Solenoid valves (B6.1) and (B6.2) are respectively installed at middle parts of the air introduction pipelines (A6.1) and (A6.2), and the other ends of the air introduction pipelines (A6.1) and (A6.2) extend into the warehouse type positive and negative pressure cabin (1N) or the atmospheric warehouse type positive and negative pressure cabin (1N-2) to be connected to air inlet ports of the airflow carried substance generator integration (6.021) and the integrated pipeline type positive and negative pressure fluid carried substance generator (6.05). An air-to-water production air inflation pipeline (A9) is further connected to the air inflation pipeline (A3), the air-to-water production air inflation pipeline (A9) is connected to the solenoid valve (B2) and then is connected to the air exhaust pipeline (A2). The middle part of the air exhaust pipeline (A2) communicates with the air-to-water production apparatus (11), and the other end of the air exhaust pipeline (A2) is connected to the atmosphere. The touch screen and mobile phone monitoring and identification system (12) includes a door touch screen (12.1), a mobile phone APP (12.2), a wireless anti-fog camera identification apparatus (12.4), and a wireless radar scanning and identification apparatus (12.6). The wireless anti-fog camera identification apparatus (12.4) and the wireless radar scanning and identification apparatus (12.6) are installed at positions inside/outside the warehouse body (7) needing to be monitored and identified. The warehouse type positive and negative pressure cabin (1N) or the atmospheric warehouse type positive and negative pressure cabin (1N-2) is internally provided with an airflow carried substance generator integration (6.02), or an integrated pipeline type positive and negative pressure fluid carried substance generator (6.05), and corresponding sensors thereof. The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

According to a fifteenth embodiment of the present disclosure, an operation method for an intelligent positive and negative pressure fresh-keeping warehouse is provided. The intelligent positive and negative pressure fresh-keeping warehouse adopts an intelligent positive and negative pressure system. The intelligent positive and negative pressure system is used to accurately regulate and organically integrate various techniques such as super oxygen, catalysts, air conditioning fresh-keeping, negative ions, humidification, dehumidification, disinfection and degradation, air purification, air-to-water production and low-temperature storage, one item of or a combination of more items of the techniques is applied to the positive and negative pressure fresh-keeping warehouse, and digital techniques such as Internet, big data, cloud computing, machine learning, artificial intelligence and expert system are also applied to the warehouse at the same time, so as to form a regulatable and controllable artificial intelligence microclimate in the positive and negative pressure fresh-keeping warehouse and to create a high-energy and efficient precise fresh-keeping microenvironment. Various fresh-keeping elements such as air composition, airflow pressure, humidity and temperature, aseptic cleanliness and the like of the positive and negative pressure fresh-keeping warehouse are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization and disinfection, quickly degrade harmful residues on the surface of preservation, achieve all-round and multi-angle digital targeted treatment, eliminate putrefying factors, accurately keep freshness and comprehensively preserve nutrition. Various fresh goods stored in the positive and negative pressure fresh-keeping warehouse are in an optimal pressure range, an optimal gas composition and optimal humidity and temperature, and an optimal preservation environment. Fruits and vegetables are hibernated at low temperature in an existing refrigerating warehouse, while the positive and negative pressure and digital techniques are used in the positive and negative pressure fresh-keeping warehouse to make fruits and vegetables in a dormant or suspended animation state, such that the fruits and vegetables have the weakest life, the slowest aging, the lowest breathing and the least consumption of nutrition. The nutritional components, the original freshness and original flavor of fruits, vegetables, meat, fish and other stored products are quickly stored for a long time; in the positive and negative pressure system, the digital technique is used to analyze and identify fresh-loss sites and fresh-keeping elements of various stored products one by one to form a complete large database according to different categories, and to design a personalized targeted control comprehensive fresh-keeping method at the cellular and molecular level for the fresh-loss sites and fresh-keeping elements of the stored products, so as to accurately maintain the fresh-keeping elements and remove the fresh-loss factors at a fixed point, without affecting or less affecting the normal tissues and cells of the stored products, and thus a set of digital expert system for fresh-keeping of stored products is formed and gradually improved, which is intelligently applied to the positive and negative pressure fresh-keeping warehouse to achieve the purpose of keeping freshness and quality of the stored and transported products. In a specific operation method for the intelligent positive and negative pressure fresh-keeping warehouse, an air evacuation pump or air inflation pump is used by the positive and negative pressure system to intelligently regulate and control airflow pressure, or carry super oxygen, catalyst, negative ions, air conditioning gas, disinfectant, detergent, heat, water and various effective loads by the airflow to get in, stay in or get out of a warehouse type positive and negative pressure cabin (1N) or an atmospheric warehouse type positive and negative pressure cabin (1N-2) orderly, and the following methods are used to apply various forces helpful for long-term freshness and quality keeping to fresh fruits and vegetables stored in the warehouse: (1): positive atmospheric pressure high pressure action method: forming moderate positive atmospheric pressure at proper time in a cabin of the warehouse type positive and negative pressure cabin (1N), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the warehouse type positive and negative pressure cabin (1N), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) airflow carried substance action method: carrying single or a combination of super oxygen, catalyst, negative ion, air conditioning gas, disinfection and degradation substances, heat, water and various effective loads by positive and negative pressure airflow to get in, stay in or get out of the warehouse type positive and negative pressure cabin (1N), or the atmospheric warehouse type positive and negative pressure cabin (1N-2) orderly, integrating various fresh-keeping techniques to apply various accurate and effective action forces for the long-term freshness and quality keeping of the stored fruits, vegetables and fresh goods. Based on above three action force methods, a positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of a touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping. The targeted control comprehensive fresh-keeping methods for the intelligent positive and negative pressure system and specific use methods and operation procedures of the positive and negative pressure fresh-keeping warehouse are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1) to open corresponding air exhaust pipelines (A2) and (A1), and turning on the air evacuation pump (2) to exhaust or pump the atmospheric warehouse type positive and negative pressure cabin (1N-2) or the warehouse type positive and negative pressure cabin (1N) to vacuum or negative pressure between −0.001 KPa and −0.1 MPa (taking the local instant atmospheric pressure as the zero standard), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within warehouse type positive and negative pressure cabin (1N), thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) pressurized or atmospheric circulating sterilization, degradation and deodorization procedure for super oxygen, negative ion or catalyst positive and negative pressure carried substance: according to the procedure setting and feedback of sensors (C), sending, by the positive and negative pressure intelligent regulation and control apparatus (5), an instruction to turn on a super oxygen generator (6.2), a negative ion generator (6.3), a catalyst release controller (6.4), a humidity control apparatus (6.5) or a disinfection and degradation apparatus (6.9) in an airflow carried substance generator integration (6.02) or (6.021) or an integrated pipeline type fluid carried substance generator (6.05), or a carried substance generator installed in a carried substance generator module (6.16), thus enabling the super oxygen, catalyst, negative ion, water and related carried substances in the positive and negative pressure cabin reach required standards; meanwhile, enabling solenoid valves (B3), (B4) and (B3.1), (B3.2), (B3.3) or (B3.4) to open corresponding air inflation pipelines (A4), (A3) and (A3.1), (A3.2), (A3.3), or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and warehouse configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric, pressurized or humidified preservation procedure of air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3) and (B4), (B6), (B6.1) or (B6.2) to open corresponding related carried substance air inflation pipelines (A4) and (A3), (A6), (A6.1) or (A6.2), or enabling solenoid valves (B3), (B4), (B6) and (B7) to open corresponding related carried substance circulation pipelines (A3), (A5), (A6) and (A7), and turning on the air inflation pump (3) for the inflation and pressurization or atmospheric circulation of the warehouse type positive and negative pressure cabin (1N) or the atmospheric warehouse type positive and negative pressure cabin (1N-2); during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B1) and (B2) or (B8.1) to open carrying waste gas evacuation and exhaust pipelines (A8) or (A8.1), (A1) or (A2), carrying waste gas evacuation and exhaust port (s1) or (s2) of air conditioning apparatus (6.1)→(A8) or (A8.1)→solenoid valves (B1)→(A1)→air inlet port (a) of air evacuation pump→air outlet port (b) of air evacuation pump→(A2)→carried substance decomposition processor (6.7)→(A2)→(B2)→(A2)→air-to-water production apparatus (11)→(A2)→exhaust to atmosphere; turning on the air evacuation pump (2) to exhaust and pump decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) low-temperature assisted fresh-keeping procedure: after the positive and negative pressure fresh-keeping warehouse is turned on and the door is closed, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (6) open-to-exhaust protection function: if the warehouse door is opened during super-oxygen sterilization and disinfection or air-condition preservation, providing feedback immediately by a warehouse door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to the carried substance decomposition processor (6.7) to be exhausted to atmosphere after being decomposed, wherein, as the air flows from the outside of the warehouse door to the positive and negative pressure cabin for supplementing air evacuation negative pressure, the super oxygen or high-nitrogen low-oxygen carried substance gas cannot flow out of the door; (7) air-to-water production procedure: enabling moist waste gas pumped from the vacuum high pressure cabin by the air evacuation pump (2) to enter the carried substance decomposition processor (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B2) to open air-to-water production inflation pipelines, thus driving the indoor air to enter the air-to-water production apparatus (11) for water production; (8) remote and short-range control monitoring and identification function: installing a high-definition anti-fog camera and identification apparatus (12.3) and a radar scanning and identification apparatus (12.5) at positions needing monitoring and identification inside and outside the warehouse body (7); shooting and scanning statuses of objects in the warehouse in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling, by the positive and negative pressure system, various fresh-keeping environmental factors such as airflow pressure, gas composition, air cleanliness, environment temperature and humidity in the positive and negative pressure cabin in real time, accurately positioning and removing the fresh-loss factors in a targeted manner, and automatically performing harmless disinfection and sterilization, and rapidly degrading residual pesticides, fertilizers, hormones and harmful additives. In conclusion, in the specific operation method for the intelligent positive and negative pressure fresh-keeping warehouse, various techniques such as vacuum, high pressure, super oxygen, catalyst, air-conditioning fresh-keeping, negative ion, humidification, humidity removal, humidity control, disinfection and sterilization, air purification, air-to-water production and low-temperature storage are intelligently regulated, controlled and integrated under the positive pressure and negative pressure, which are organically applied to the intelligent positive and negative pressure fresh-keeping warehouse and are organically integrated and optimally matched, one item of or a combination of more items of the techniques is used individually or alternately or circularly, and a plurality of positive and negative pressure cabins with different types and different functions are arranged in a positive and negative pressure fresh-keeping warehouse, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping warehouse updates the existing method that the traditional refrigerating warehouse achieves low-temperature storage only by controlling the temperature of the warehouse. A regulatable and controllable artificial intelligence microclimate is formed in the positive and negative pressure cabin to create a precise fresh-keeping microenvironment, in which various fresh food stored can keep fresh, moist and original quality and flavor for a long time and to the maximum extent.

According to a sixteenth embodiment of the present disclosure, an intelligent positive and negative pressure disinfection machine is provided, including a positive and negative pressure full-functional disinfection machine, a positive and negative pressure multifunctional disinfection machine, a positive and negative pressure automatic humidification and disinfection machine, a positive and negative pressure humidification and disinfection machine, and a positive and negative pressure disinfection machine, where the positive and negative pressure disinfection machine includes a machine body (7). The machine body (7) is internally provided with an intelligent positive and negative pressure system, an air-to-water production apparatus (11), and a power supply and rechargeable battery (33). The intelligent positive and negative pressure system includes an air evacuation pump (2), an air inflation pump (3), a positive and negative pressure intelligent regulation and control apparatus (5), a positive and negative pressure fluid carried substance generation processor (6), a touch screen and mobile phone monitoring system (12), and sensors (C). The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes a super oxygen generator air disinfection apparatus (6.2), a negative ion generator air improvement apparatus (6.3), a humidification and humidity controller air regulation apparatus (6.5), and an instantaneous disinfector filtering and disinfection apparatus (6.11). The touch screen and mobile phone monitoring system (12) includes a touch screen (12.1), a mobile phone monitoring system (12.2), a location positioning system (12.5). The sensors include a super oxygen sensor (C3), a negative ion sensor (C4), a humidity sensor (C6), a disinfection and degradation sensor (C9), and a water level sensor (13). An air inflation pipeline (A3) is installed at an air outlet port (d) of the air inflation pump (3), and air inflation branch pipelines (A3.1), (A3.2), (A3.3), (A3.4), (A3.5) are respectively connected to the other end of the air inflation pipeline (A3), a solenoid valve (B3.1) is installed at a middle part of the air inflation branch pipeline (A3.1), and the other end of the air inflation branch pipeline (A3.1) is connected to an air inflation and outlet port of the machine body (7). A solenoid valve (B3.2) and the negative ion generator air improvement apparatus (6.3) are installed at a middle part of the air inflation branch pipeline (A3.2), and the other end of the air inflation branch pipeline (A3.2) is connected to a negative ion outlet port of the machine body (7). A solenoid valve (B3.3) and the super oxygen generator air infection apparatus (6.2) are installed at a middle part of the air inflation branch pipeline (A3.3), and the other end of the air inflation branch pipeline (A3.3) is connected to a super oxygen outlet port of the machine body (7). A solenoid valve (B3.4) and the humidification and humidity controller air regulation apparatus (6.5) are installed at a middle part of an air inflation branch pipeline (A3.4), and the other end of the air inflation branch pipeline (A3.4) is connected to a humidification and air outlet port of the machine body (7). A solenoid valve (B3.5) and the air-to-water production apparatus (11) are installed at a middle part of an air inflation branch pipeline (A3.5), and the other end of the air inflation branch pipeline (A3.5) is connected to a water production and air exhaust port of the machine body (7). An air intake pipeline (A4) is installed at an air inlet port (e) of the air inflation pump (3), a solenoid valve (B4) is installed at a middle part of the air intake pipeline (A4), and the other end of the air intake pipeline (A4) is connected to a direct air inlet port of the machine body (7). A filtering and air intake pipeline (A4.1) is further connected to the solenoid valve (B4), the air filter (6.8) and a solenoid valve (B4.1) are installed at a middle part of the filtering and air intake pipeline (A4.1), and the other end of the filtering and air intake pipeline (A4.1) is connected to a filtering and air inlet port of the machine body (7). A circulating air return pipeline (A5) is further connected to the solenoid valve (B4.1), the other end of the circulating air return pipeline (A5) is connected to a circulating air return port of the machine body (7). An air-to-water production pipeline (A10) is installed at a water output port (x) of the air-to-water production apparatus (11), the air-to-water production pipeline (A10) communicates with a water inlet port (y) of a filtering water tank (11.1). A filtering water pipeline (A12) is installed at a water output port (v) of the filtering water tank (11.1), the filtering water pipeline (A12) communicates with a water inlet port of the humidification and humidity controller air regulation apparatus (6.5), a water adding pipeline (A11) is installed at a spare water adding port (u) of the filtering water tank (11.1), a solenoid valve (B11) is installed at a middle part of the water adding pipeline (A11), and the other end of the water adding pipeline (A11) is connected to a spare water adding port of the machine body (7). An air exhaust pipeline (A1) is installed at an air inlet port (a) of the air evacuation pump (2), the instantaneous disinfector filtering and disinfection apparatus (6.11) is installed at a middle part of the air exhaust pipeline (A1), and the other end of the air exhaust pipeline (A1) is connected to an air evacuation and air return port of the machine body (7). An air exhaust pipeline (A2) is installed at an air outlet port (b) of the air evacuation pump (2), a solenoid valve (B2) is installed at a middle part of the air exhaust pipeline (A2), and the other end of the air exhaust pipeline (A2) is connected to the atmosphere. The solenoid valve (B2) is connected to a solenoid valve (A3.5) through the pipeline (A2.1), communicates with the air-to-water production pipeline (A3.5) and an air inlet port (h) of the air-to-water production apparatus (11) in sequence, and then communicates with a water production and air exhaust port via the air outlet port (n) of the air-to-water production apparatus (11) after passing through the air-to-water production apparatus (11). The positive and negative pressure multifunctional disinfection machine does not include the negative ion generator air improvement apparatus (6.3), other structures of which are the same as those of the positive and negative pressure full-functional disinfection machine. The positive and negative pressure automatic humidification and disinfection machine does not include the instantaneous disinfector filtering and disinfection apparatus (6.11), other structures of which are the same as those of the positive and negative pressure full-functional disinfection machine. The positive and negative pressure humidification and disinfection machine does not include the air-to-water production apparatus (11), other structures of which are the same as those of the positive and negative pressure automatic humidification and disinfection machine. The positive and negative pressure disinfection machine does not include a humidification and humidity control air regulation apparatus (6.5), other structures of which are the same as those of the positive and negative pressure humidification and disinfection machine. The fluid carried substance generation processor (6), the sensor (C), the solenoid valve (B), the air evacuation pump (2), the air inflation pump (3), the air-to-water production apparatus (11) and the touch screen and mobile phone monitoring and identification system (12) of each machine model are connected to the positive and negative pressure regulation and control apparatus (5).

According to a seventeenth embodiment of the present disclosure, an operation method for an intelligent positive and negative pressure disinfection machine is provided. An intelligent positive and negative pressure is used in the intelligent positive and negative pressure disinfection machine, the intelligent positive and negative pressure system is used to regulate, control and integrate various techniques, such as a super oxygen and air disinfection method, a negative ion and air regulation method, a humidification and humidity control and humidity control method, an instantaneous disinfection, and filtering and disinfection, one item of or a combination of more items of the techniques is applied to the positive and negative pressure disinfection machine. An air evacuation pump or air inflation pump is used to intelligently regulate and control airflow, and the airflow is used to carry super oxygen, air disinfectant, water, negative ion and a plurality of effective loads, and the following methods are used for killing viruses and bacteria in the inhabited environment, disinfect the air and the surface of objects, and effectively regulate the air humidity and air composition: (1) the intelligent positive and negative pressure disinfection machine integrates super oxygen and air disinfection methods to inactivate viruses and bacteria including novel coronavirus, kill viruses and bacteria within a certain range, and disinfect the air and the surfaces of the objects, Fujita Health University of Japan has studied that many viruses, including novel coronavirus, are eliminated in the inhabited environment with a low concentration of about 0.05-0.10 ppm of super oxygen, which is acceptable to the human body; the first-class standard of ozone concentration limit in ambient air quality standard (GB3095-1996) in China is 0.12 mg/m3, so the positive and negative pressure disinfection machine is harmless to the human body when used to disinfect the environment to eliminate viruses and bacteria, including novel coronavirus; (2) on the basis of the positive and negative pressure disinfection machine, the positive and negative pressure humidification and disinfection machine is provided with a humidification and humidity control air regulation apparatus, when the super oxygen is used for inactivating the novel coronavirus, the increase of air humidity can enhance the inactivation efficiency; Fujita Health University of Japan has studied that, when the humidity is 80%, the environment is disinfected with about 0.10 ppm of super oxygen, and the virus infectivity drops to 5.7% after 10 hours; (3) on the basis of the positive and negative pressure humidification and disinfection machine, the positive and negative pressure automatic humidification and disinfection machine is additionally provided with an air-to-water production apparatus to provide uninterrupted self-made water for the disinfection machine, thus reducing the disadvantages of frequent manual water addition to a humidifier; (4) on the basis of the positive and negative pressure automatic humidification and disinfection machine, the positive and negative pressure multifunctional disinfection machine is additionally provided with an instantaneous disinfector or a filtering disinfection apparatus, thus providing an instantaneous disinfection function for the disinfection machine other than super oxygen disinfection to make up for the shortage that super oxygen cannot achieve instantaneous disinfection; and (5) on the basis of the positive and negative pressure multifunctional disinfection machine, the positive and negative pressure full-functional disinfection machine is additionally provided with a negative ion generator air improvement apparatus, so as to additionally provide an air improvement function for the disinfection machine.

According to an eighteenth embodiment of the present disclosure, an intelligent positive and negative pressure module cabinet is provided, including a cabinet body (7). The cabinet body (7) is internally provided with an intelligent positive and negative pressure system, and a refrigeration and heating system (8.3). The positive and negative pressure system includes a positive and negative pressure cabin (1), an air evacuation pump (2), an air inflation pump (3), a positive and negative pressure intelligent regulation and control apparatus (5), a positive and negative pressure airflow carried substance generation processor (6), and a touch screen and mobile phone monitoring and identification system (12). The intelligent positive and negative pressure system also adopts a nonstandard structure other than the above standard structures in specific applications; the nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a module type positive and negative pressure cabin (1T) according to the requirements of the intelligent positive and negative pressure module cabinet, and the module type positive and negative pressure cabin (1T) is a vacuum high-pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the module cabinet; the module type positive and negative pressure cabin (1T) includes a cabin body (1.1), a cabin door (1.2), a mechanical airtight mechanism (1.3), and an internal and external communicating sealer (1.4). The cabin door (1.2) is a cabinet door of the positive and negative pressure module cabinet; the airtight mechanism (1.3) is arranged between the cabin door (1.2) and the cabin body (1.1) of the module type positive and negative pressure cabin (1T). The internal and external communicating sealer (1.4) is fixedly arranged at a rear part of the module type positive and negative pressure cabin (1T), all pipelines and circuits getting in and out the cabin are connected to and pass through the internal and external communicating sealer (1.4), so as to keep the sealing property of the module type positive and negative pressure cabin (1T). The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), an airflow carried substance generator integration (6.02) or (6.021), or an integrated pipeline type positive and negative fluid carried substance generator (6.05). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. The airflow carried substance generator integration (6.02) or (6.021) or the integrated pipeline type positive and negative pressure fluid carried substance generator (6.05) includes any one or more in the individual fluid carried substance generators (6.1) to (6.16) as required. The module type positive and negative pressure cabin (1T) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration and heating system (8.3), an air-to-water production apparatus (11), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the module type positive and negative pressure cabin (1T) to form an air evacuation and air return port (c) thereof. Carrying waste gas carrying waste gas evacuation and exhaust pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to a carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), passes through the air-to-water production apparatus (11), and then is connected to the atmosphere from an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the module type positive and negative pressure cabin (1T) to form an air inflation and inlet port (f) thereof. An air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to the atmosphere. A circulation pipeline (A5) is further connected to the solenoid valve (B4), and the other end of the circulation pipeline (A5) communicates with the solenoid valve (B3), and extends into the module type positive and negative pressure cabin (1T) through the air inflation pipeline (A3) to form a circulating air return port (j) thereof; carrying air introduction pipelines (A6), (A6.1) and (A6.2) are further connected to the air inflation pipeline (A3), a solenoid valve (B6) is installed at a middle part of the carrying air introduction pipeline (A6), the other end of the carrying air introduction pipeline (A6) is connected to an air inlet port (t) of the positive and negative pressure airflow carried substance generator integration (6.02). A carrying air introduction pipeline (A7) is installed at an air outlet port (s) of the positive and negative pressure airflow carried substance generator integration (6.02), a solenoid valve (B7) is installed at a middle part of the carrying air introduction pipeline (A7), and the other end of the carrying air introduction pipeline (A7) extends into the positive and negative pressure cabin (1T) to form a carrying air inlet port (i) thereof. Solenoid valves (B6.1) and (B6.2) are respectively installed at middle parts of the carrying air introduction pipelines (A6.1) and (A6.2), the other ends of the carrying air introduction pipelines (A6.1) and (A6.2) extend into the positive and negative pressure cabin (1) to be connected to air inlet ports of the positive and negative pressure airflow carried substance generator integration (6.021) and the integrated pipeline type positive and negative pressure fluid carried substance generator (6.05). An air-to-water production inflation pipeline (A9) is further connected to the air inflation pipeline (A3), the air-to-water production inflation pipeline (A9) is connected to the solenoid valve (B2) and is then connected to the air exhaust pipeline (A2). The air-to-water production apparatus (11) communicates with the middle part of the air exhaust pipeline (A2), and the other end of the air exhaust pipeline (A2) is connected to the atmosphere; the touch screen and mobile phone monitoring and identification system (12) includes a cabinet door touch screen (12.1), a mobile phone APP (12.2), a high-definition anti-fog camera device (12.3), and a radar scanning and identification apparatus (12.5). The high-definition anti-fog camera device (12.3) and the radar scanning and identification apparatus (12.5) are installed at positions inside/outside the cabinet body (7) needing to be monitored. The module type positive and negative pressure cabin (1T) is internally provided with the airflow carried substance generator integration (6.021) or the integrated pipeline type fluid carried substance generator (6.05) and sensors (C). The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

It must be emphasized that: the above described intelligent positive and negative pressure system and the operation method therefor and multiple intelligent positive and negative pressure electric appliances using the system and the operation method therefor are several preferred embodiments of the solutions of the embodiments of the present disclosure, and are not intended to limit the embodiments of the present disclosure. Any modification, equivalent replacement, improvement and the like made within the spirit and principles of the embodiments of the present disclosure shall be included in the scope of protection of the embodiments of the present disclosure.

Compared with the prior art, the intelligent positive and negative pressure system and the operation method therefor and multiple intelligent positive and negative pressure electric appliances using the system and the operation method therefor according to the embodiments of the present disclosure have the advantages that:
Firstly. The intelligent positive and negative pressure is established based on the positive and negative pressure technique. The positive and negative pressure technique is to regulate and control a fluid and a carried substance thereof by means of positive and negative pressure, so as to affect objects in a specific space. The technique principle and basic operation method for the intelligent positive and negative pressure system are as follows: intelligently regulating and controlling fluids (such as air and water) by means of the positive and negative pressure, or carrying effective loads such as super oxygen, catalysts, negative ions, air conditioning gas, disinfectant, cleaner, heat, water to get in, stay in or get out a specific space (e.g., a positive and negative pressure cabin) orderly, and applying the required influence on an object therein. The intelligent positive and negative pressure system, due to the use of the above technique operation methods, has an ultra-large-capacity carrier performance and a cross-border expansion platform advantage, can form targeted influence methods or targeted control modes with different characteristics for various affected objects. Various techniques such as vacuum, high pressure, super oxygen, catalysts, air conditioning fresh-keeping, negative ions, humidification, dehumidification, humidity and temperature control, disinfection and sterilization, air purification, air-to-water production, low-temperature storage, clothes washing and drying and food processing can be digitally regulated, controlled and integrated, and one item of or a combination of more items of the techniques is applied to positive and negative pressure electric appliances, thereby intelligently regulating and controlling and accurately creating the optimal efficiency and optimal working conditions for the operation of various electric appliances; and existing electric appliances and traditional techniques are updated and replaced, and a new field of positive and negative pressure electric appliances is developed.
Secondly. There is no technical breakthrough in the method that the existing refrigerator and refrigerating type compartment container only relies on low-temperature bacteriostatic storage. According to the intelligent positive and negative pressure system and the operation method therefor, the intelligent positive and negative pressure technique and the operation method therefor are used to intelligently regulate, control and integrate multiple techniques such as vacuum, high pressure, super oxygen, catalysts, air conditioning fresh-keeping, negative ions, humidification, dehumidification, disinfection, air purification, air-to-water production, and low-temperature storage, the techniques are applied to the refrigerator, the refrigerating compartment container and other electric appliances to intelligently regulate and control the airflow, or carry super oxygen, air catalysts, negative ions, air conditioning gas, water and various effective loads to get in, stay in or get out the positive and negative pressure cabin orderly, just like human breath, the vitality can be maintained by breathing out waste gas and breathing in fresh air. Digital techniques such as Internet, big data, cloud computing, machine learning, artificial intelligence and expert system are also applied to the refrigerator and refrigerating compartment container by the positive and negative pressure system, so as to form a regulatable and controllable artificial intelligence microclimate in the positive and negative pressure fresh-keeping refrigerator and to create a high-energy and efficient precise fresh-keeping microenvironment. Various fresh-keeping elements such as air composition, airflow pressure, humidity and temperature, aseptic cleanliness and the like of the positive and negative pressure fresh-keeping refrigerator or refrigerating compartment container are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization, quickly degrade harmful residues on the surface of preservation, achieve all-round and multi-angle digital targeted treatment, eliminate putrefying factors, accurately keep freshness and comprehensively preserve nutrition. Various fresh goods stored in the positive and negative pressure cabin are in an optimal pressure range, an optimal gas composition and optimal humidity and temperature, and an optimal preservation environment. Fruits and vegetables are hibernated at low temperature in an existing refrigerator and refrigerating compartment container, while the positive and negative pressure and digital techniques are used in the positive and negative pressure fresh-keeping refrigerator and refrigerating compartment container to make fruits and vegetables in a dormant or suspended animation state, such that the fruits and vegetables have the weakest life, the slowest aging, the lowest breathing and the least consumption of nutrition, and the nutritional components and the original freshness of fruits, vegetables, meat, fish and other stored products are quickly stored for a long time. In the positive and negative pressure system, the digital technique is used to analyze and identify fresh-loss sites and fresh-keeping elements of various stored products one by one to form a complete large database according to different categories, and to design a personalized targeted control comprehensive fresh-keeping method at the cellular and molecular level for the fresh-loss sites and fresh-keeping elements of the stored products, so as to accurately maintain the fresh-keeping elements and remove the fresh-loss factors at a fixed point, without affecting or less affecting the normal tissues and cells of the stored products, and thus a set of digital expert system for preservation and fresh-keeping is formed and gradually improved, which is intelligently applied to the positive and negative pressure fresh-keeping refrigerator to achieve the purpose of keeping freshness and quality of the stored products. With rapid development of the scientific technology, the positive and negative pressure involved in the embodiments of the present disclosure may be gradually developed to the deeper vacuum and ultra-high pressure, the electric appliances including the positive and negative pressure cabins, air evacuation and air inflation pumps and other hardware equipment, may be more economic and suitable for use, and the electric appliances including chip processors and other software and hardware are gradually upgraded and updated, the positive and negative pressure technology will be perfected day by day, and the positive and negative pressure electric appliances can bring people a better life.
Thirdly. The existing washing machine is configured to wash clothes by the mechanical movement of pulsator or drum assisted with detergent, which is large in abrasion and low in laundry efficiency. The intelligent positive and negative pressure washing machine provided by the embodiment of the present disclosure intelligently regulates, controls and integrates techniques such as high pressure, vacuum, and super oxygen. The techniques are organically combined and applied to clothes washing, dehydrating and drying processes. A vacuum and high pressure waterflow and airflow stain removal drying technique is developed. According to built-in program and pre-stored data of the positive and negative pressure washing machine, and real-time feedback information of various sensors and through the application of the digital techniques such as Internet, big data, cloud computing, and machine learning, stain-causing factors and control targets of various different cleaning substances are analyzed one by one, different targeted stain removal techniques are correspondingly used, a personalized targeted stain removal comprehensive washing-drying method is designed at the molecular and microscopic levels to accurately removal the stain-causing factors at fixed points, while avoiding or reducing the damage to the structure and color of the cleaning materials. Various washing-dehydrating-drying elements such as waterflow and airflow pressure, gas composition, water temperature and air temperature are regulated and controlled in real time, and disinfection, sterilization and harmless degradation are achieved at the same time. A regulatable and controllable accurate washing-dehydrating-drying intelligent microenvironment with the optimal effect, shortest time, highest efficiency, minimum wear and least environmental pollution is created to achieve the purpose of rapid, efficient, sanitary and environment-friendly cleaning, thus the abrasion of the clothes is reduced, the degree of cleaning and bulkiness of the clothes are improved, the super oxygen disinfection procedure is increased, and the clothes washing time is effectively reduced.
Fourthly. There is no ideal solution to the problem of kitchen stuff cleaning of existing range hoods. In accordance with the embodiment of the present disclosure, the entire machine body of the positive and negative pressure range hood is designed as a fully sealed open type positive and negative pressure circulation cabin except for the upper oil fume exhaust port, and a cover plate of the fume extraction port is designed as a sealed cabin door at the same time. When the automatic cleaning is started, the cabin door is tightly locked and sealed, and multiple automatic rotary spraying heads are arranged at the top part of the cabinet and volute impellers, etc., firstly, super-oxygenated water is sprayed to efficiently decompose and remove kitchen stuff, then all parts in the machine case are sprayed with degreaser and clean hot water until they are completely cleaned, and finally, the interior of the machine case is automatically dried by hot air and the waste gas is exhausted to the gas duct. When the range hood is turned on by a user, the negative pressure is formed inside the machine case, the air flows into the case of the range hood from the human side, such that the human body is free of contact with any super oxygen. The sewage after cleaning the whole range hood flows downwards to be collected into the oil collecting groove at the lowest part of the triangular machine case and then is drained to the sewer through the oil and water drainage port, and the automatic cleaning problem is thoroughly solved.
Fifthly. In accordance with the intelligent positive and negative pressure baking and air frying microwave oven provided by the embodiment of the present disclosure, the baking and air frying microwave oven system is arranged in the vacuum high-pressure chamber to actually achieve vacuum cooking and high-pressure cooking. Food cooked with vacuum has special flavor because of the expansion of ingredients, especially the French fries and cakes baked by the air fryer in a moderate vacuum are fluffy and tasty, and food cooked with high pressure is particularly tasty because seasonings are prone to penetrating into ingredients. When not in use, the positive and negative pressure chamber is pumped to a moderate vacuum, the oven microwave system and the circuit system are in a vacuum state, which are sterile, dust-free and pollution-free so as to keep clean for health, and are oxidation-free, rusting-free and lossless for preservation, and thus the service life of the electric appliance is prolonged.
Sixthly. The inactivation of viruses such as novel coronavirus in a room basically depends on the spraying of disinfectant at present, which is harmful to the human body. According to the positive and negative pressure disinfection machine provided by the present disclosure, the positive and negative pressure carries super oxygen to disinfect the air and the surfaces of objects in the room with human, which is harmless to the human body. A powerful weapon is discovered for the inactivation of the viruses such as novel coronavirus, and the air quality can be effectively improved.

The intelligent positive and negative pressure module cabinet provided by the embodiment of the present disclosure has a vacuum, high-pressure, constant-temperature, constant-humidity, constant-pressure, sterile and dust-free environment due to the providing of the positive and negative pressure system, the functional system of various electric appliances and equipment can be designed and arranged in the positive and negative pressure module cabinet or directly placed therein, including a positive and negative pressure disinfection cabinet, a positive and negative pressure kitchenware/tableware cabinet, a positive and negative pressure wine/water/fruits display refrigerating cabinet, a positive and negative pressure air conditioner indoor unit, a positive and negative pressure humidifier, a positive and negative pressure dehumidifier, a positive and negative pressure air purifier, a positive and negative pressure coffee/juice small electric appliance cabinet, a positive and negative pressure closet/bookcase, a positive and negative painting/antique cabinet, a positive and negative pressure safe box, and a positive and negative pressure display cabinet with various functions. In addition, the intelligent positive and negative pressure system and an operation method therefor can be applied to the functional use, disinfection, storage and placement of various electrical equipment and objects and utensils, as well as the lossless storage during the suspension of operation.
Seventhly. The existing electric appliances are basically designed independently, used independently, and placed independently. The positive and negative pressure intelligent electric appliances and positive and negative pressure module cabinets are designed according to modules, which are basically uniform, neat and harmonious in appearance, shape and color which are in line with the popular trend. The positive and negative pressure electric appliance not only can be independently used, but also can be organically combined with other positive and negative pressure electric appliances and the module cabinets under the control of the touch screen and mobile phone APP control and identification to form a fully functional intelligent positive and negative pressure module combination home. In accordance with the various positive and negative pressure module cabinets, various related electrical equipment and objects as well as utensils are organically combined in different categories, centrally placed neatly and visual-pleasantly, and can share resources such as a set of intelligent positive and negative pressure system and an air inflation pump and an air evacuation pump, save energy consumption and reduce noise, and integrate various related electrical equipment and articles and utensils into the same intelligent home system conveniently and naturally.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a schematic diagram of the technical principle of a standard structure of an intelligent positive and negative pressure system in accordance with an embodiment of the present disclosure;

FIG. 2 is an enlarged view of the technical principle of a positive and negative pressure airflow carried substance generator integration 6.02 in accordance with an embodiment of the present disclosure;

FIG. 3 is an enlarged view of the technical principle of a positive and negative pressure waterflow carried substance generator integration 6.04 in accordance with an embodiment of the present disclosure;

FIG. 4 is a schematic diagram of the technical principle of an intelligent positive and negative pressure fresh-keeping refrigerator and a drawer type positive and negative pressure fresh-keeping cabin in accordance with an embodiment of the present disclosure;

FIG. 5 is a schematic diagram of the technical principle of an intelligent positive and negative pressure fresh-keeping refrigerator and side-open cabinet type and drawer type positive and negative pressure fresh-keeping cabins in accordance with an embodiment of the present disclosure;

FIG. 6 is an enlarged view of the technical principle of a positive and negative pressure airflow carried substance generator integration 6.021 in accordance with an embodiment of the present disclosure;

FIG. 7 is an enlarged view of the technical principle of a positive and negative pressure airflow carried substance generator integration 6.022 in accordance with an embodiment of the present disclosure;

FIG. 8 is an enlarged view of the technical principle of a positive and negative pressure airflow carried substance generator integration 6.025 in accordance with an embodiment of the present disclosure;

FIGS. 9A-B are schematic diagrams of the technical principle of an intelligent positive and negative pressure washing machine and an enlarged view of an airtight mechanism in accordance with an embodiment of the present disclosure;

FIGS. 10A-B are schematic diagrams of the technical principle of an intelligent positive and negative pressure dish washing and fruit and vegetable cleaning machine and an enlarged view of an airtight mechanism in accordance with an embodiment of the present disclosure;

FIGS. 11A-B are schematic diagrams of the technical principle of an intelligent positive and negative pressure range hood, a side view and a bottom section of an automatic rotary spraying ball, and an enlarged view of an airtight mechanism in accordance with an embodiment of the present disclosure;

FIGS. 12A-B are schematic diagrams of the technical principle of an intelligent positive and negative pressure baking and air frying microwave oven, and an enlarged view of an airtight mechanism in accordance with an embodiment of the present disclosure;

FIG. 13 is a schematic diagram of the technical principle of an intelligent positive and negative pressure fresh-keeping compartment container in accordance with an embodiment of the present disclosure;

FIG. 14 is a schematic diagram of the technical principle of an intelligent positive and negative pressure fresh-keeping warehouse in accordance with an embodiment of the present disclosure;

FIG. 15 is a schematic diagram of the technical principle of an intelligent positive and negative pressure disinfection machine in accordance with an embodiment of the present disclosure;

FIG. 16 is a schematic diagram of the technical principle of an intelligent positive and negative pressure humidification and disinfection machine in accordance with an embodiment of the present disclosure;

FIG. 17 is a schematic diagram of the technical principle of an intelligent positive and negative pressure automatic humidification and disinfection machine in accordance with an embodiment of the present disclosure;

FIG. 18 is a schematic diagram of the technical principle of an intelligent positive and negative pressure multifunctional humidification and disinfection machine in accordance with an embodiment of the present disclosure;

FIG. 19 is a schematic diagram of the technical principle of an intelligent positive and negative pressure full-functional humidification and disinfection machine in accordance with an embodiment of the present disclosure;

FIG. 20 is a schematic diagram of the technical principle of an intelligent positive and negative pressure module cabinet in accordance with an embodiment of the present disclosure.

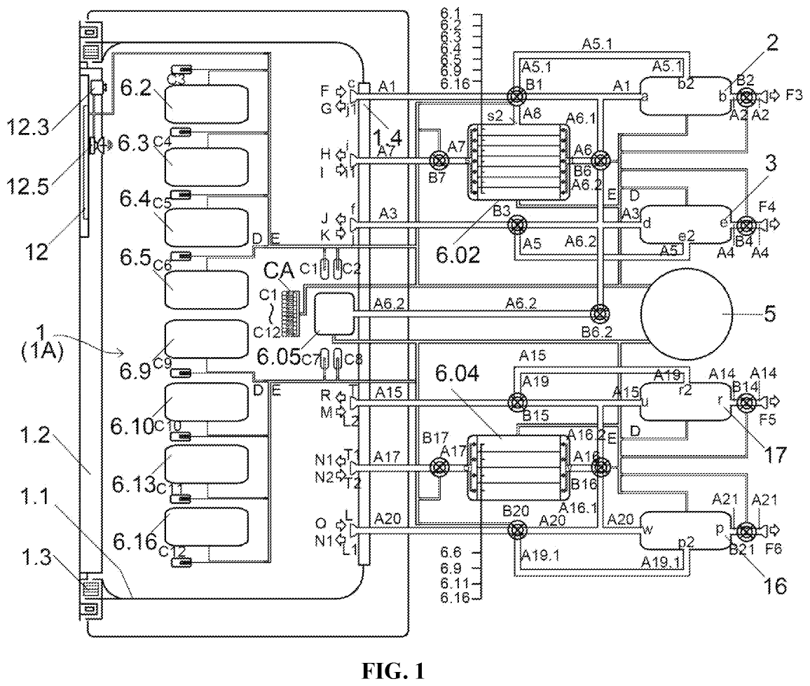

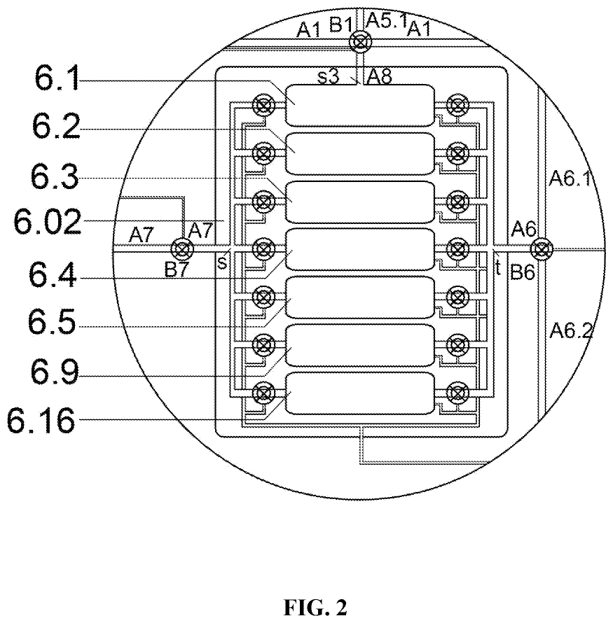

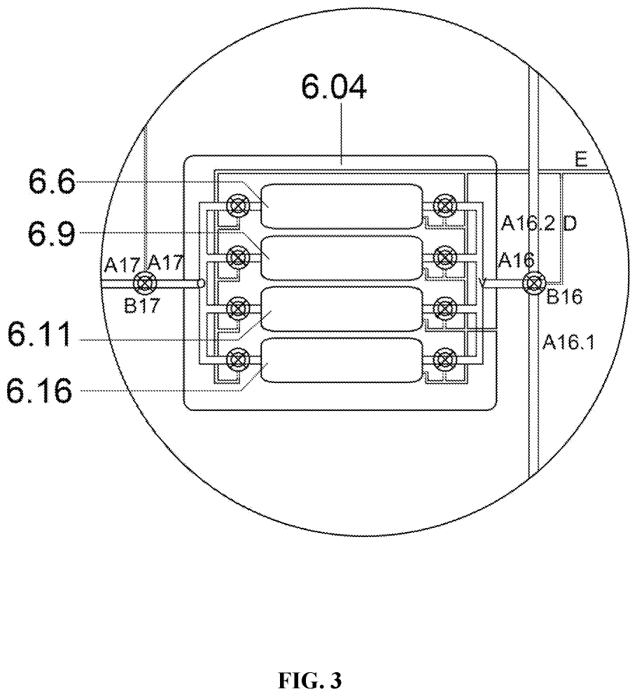

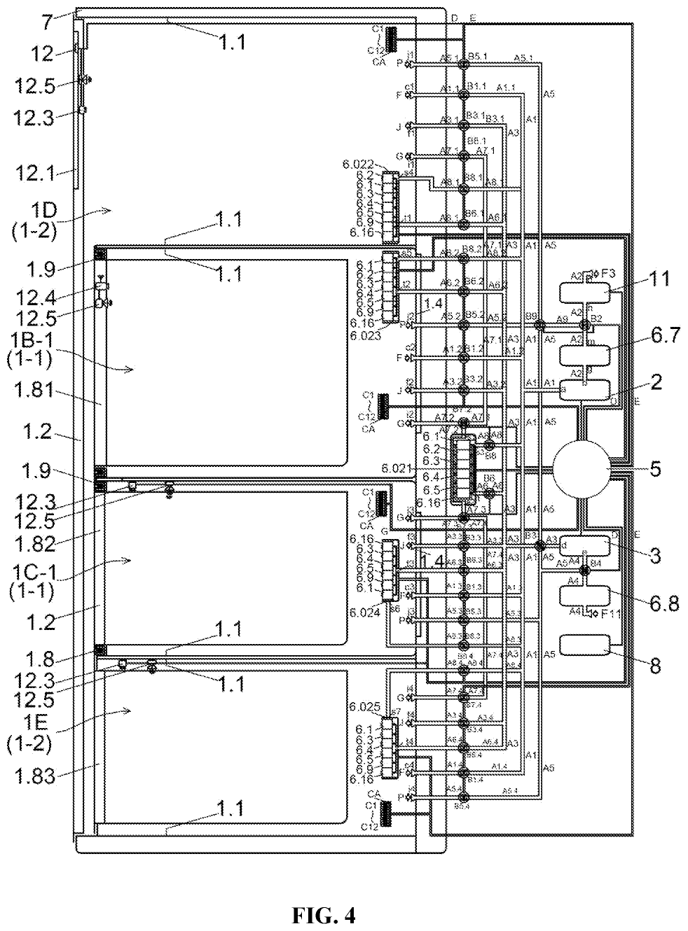

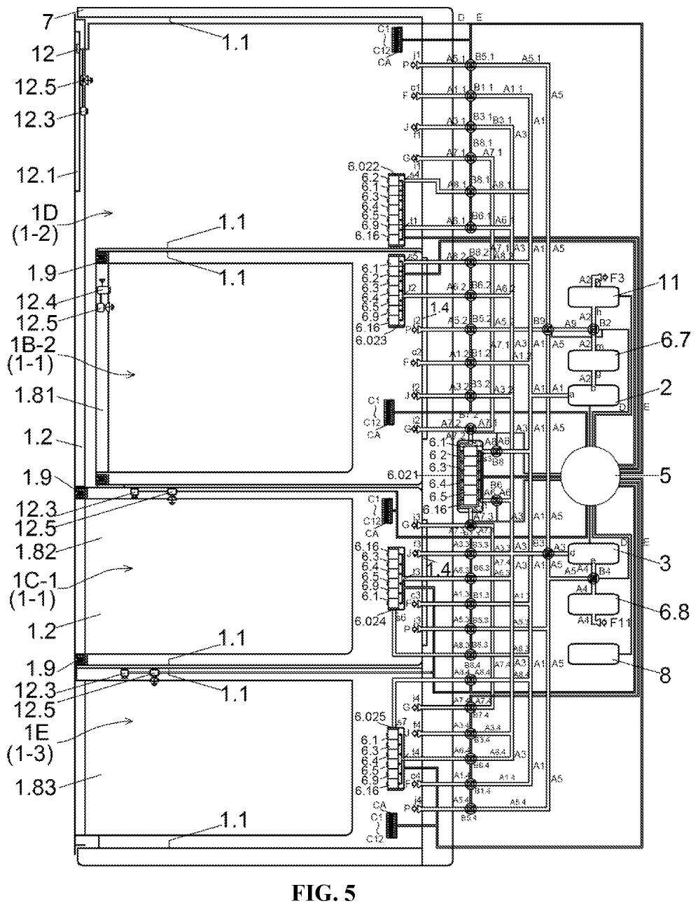

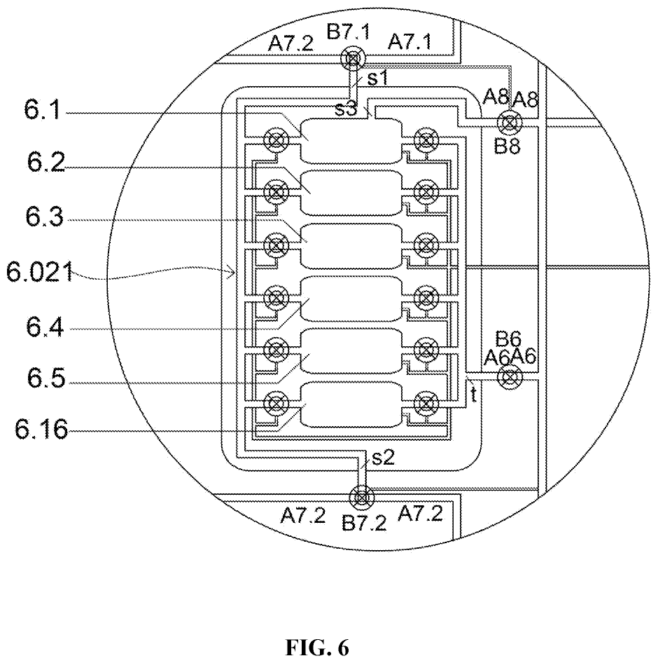

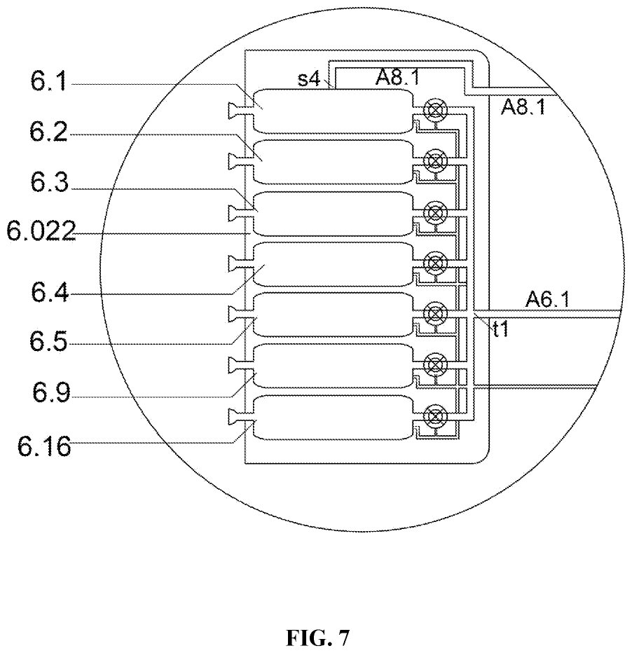

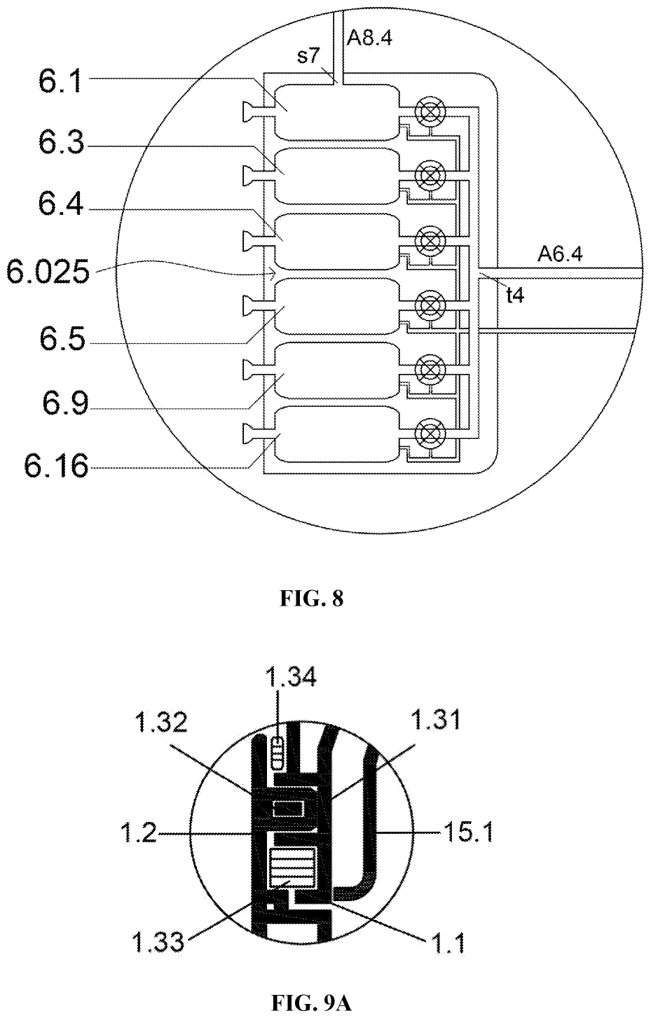

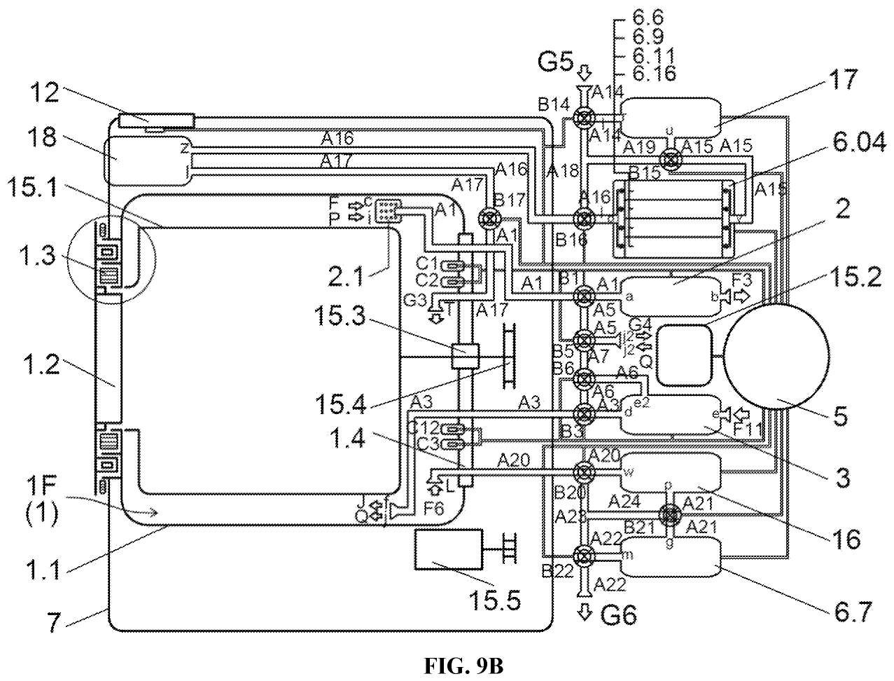

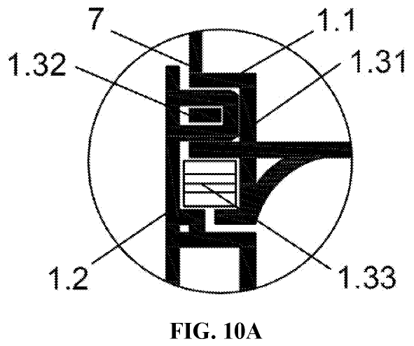

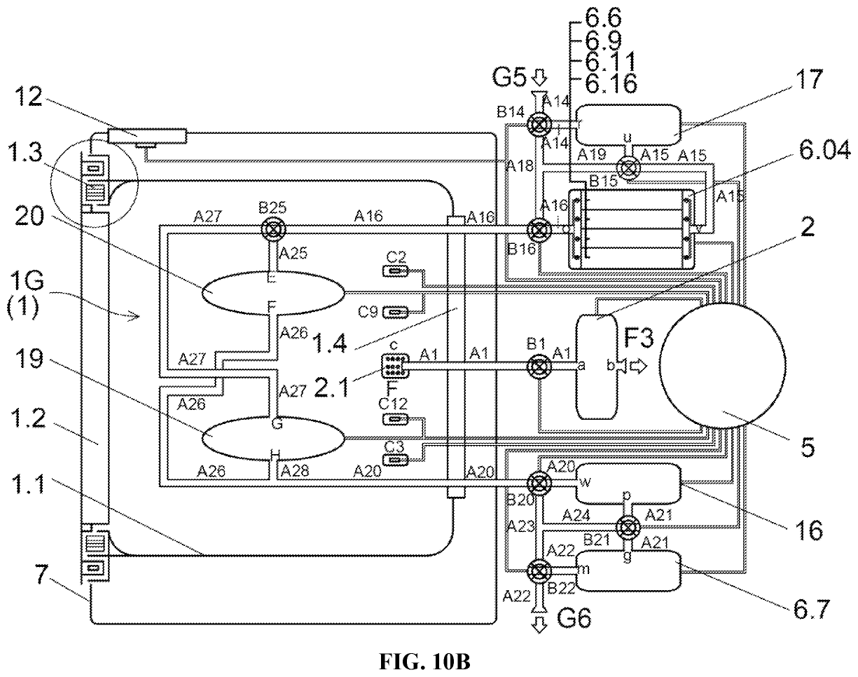

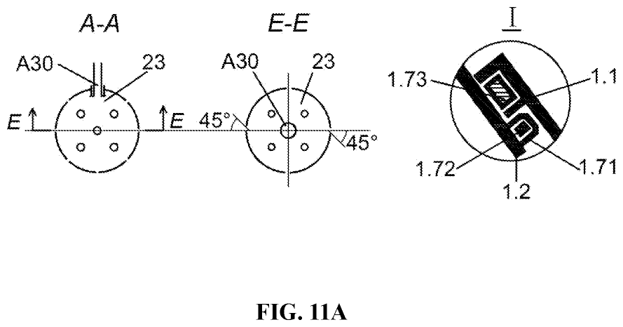

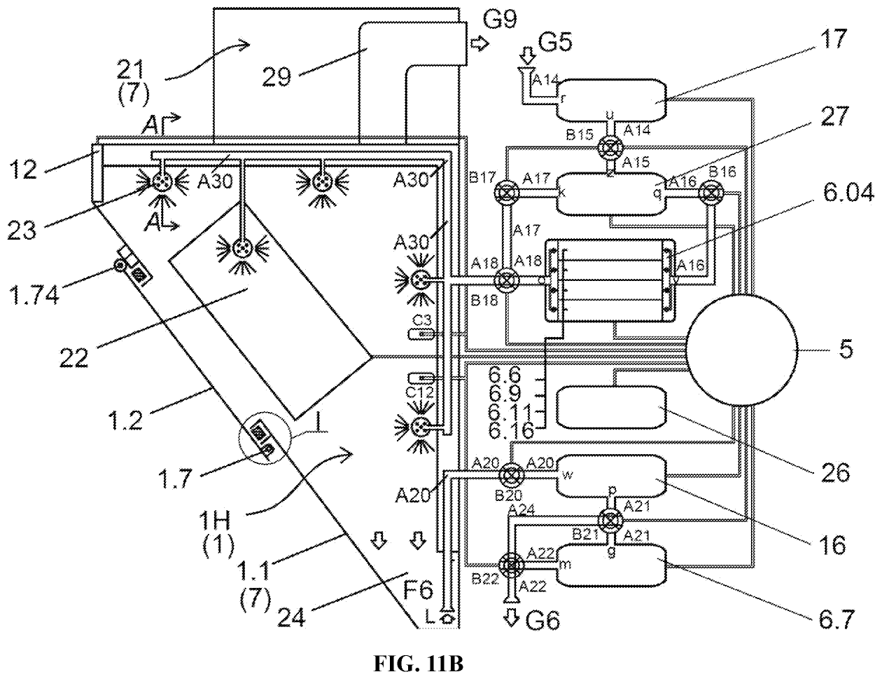

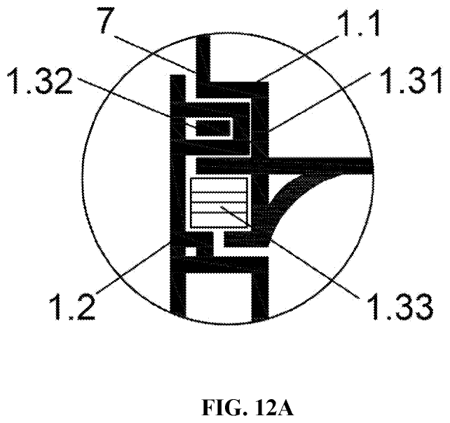

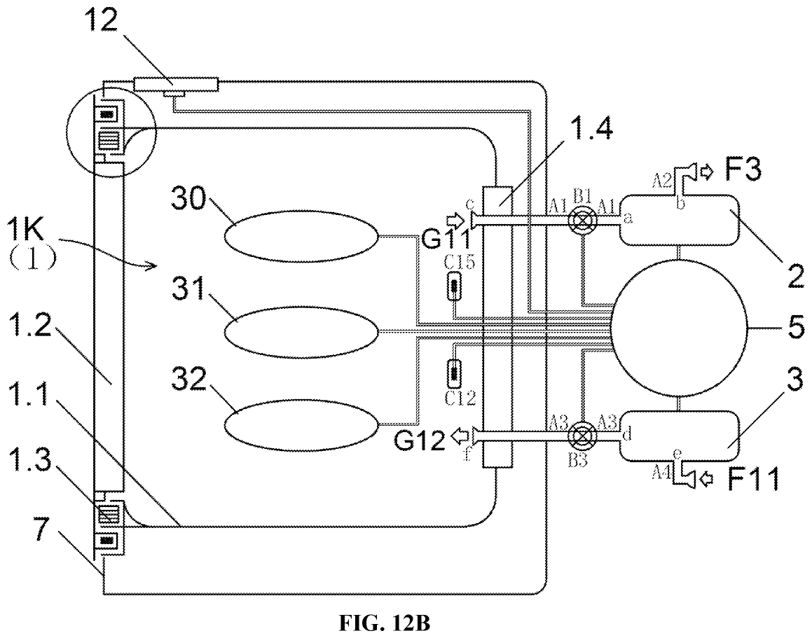

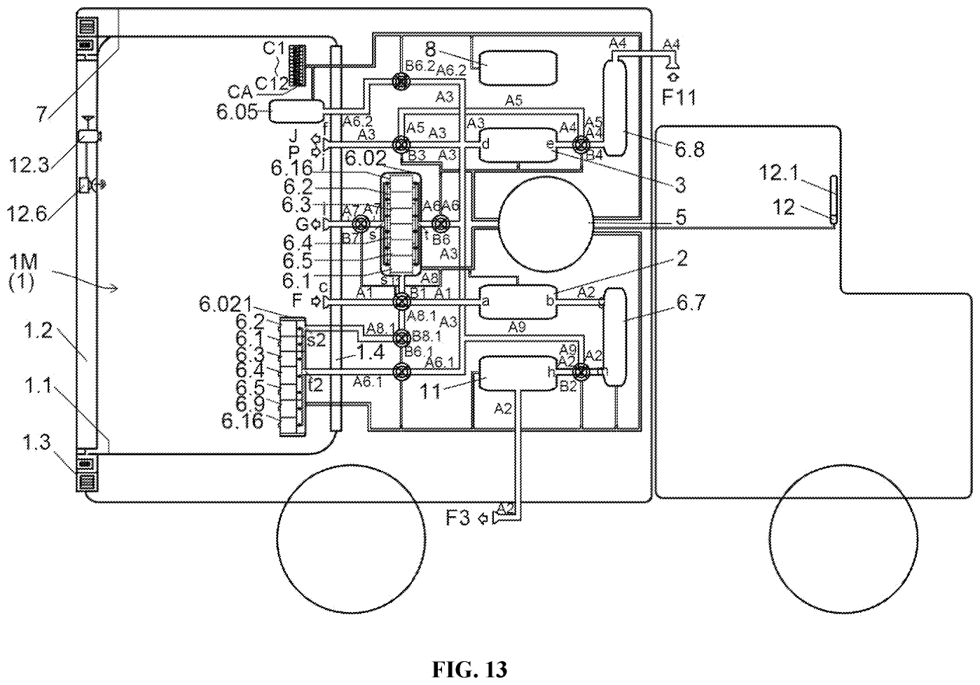

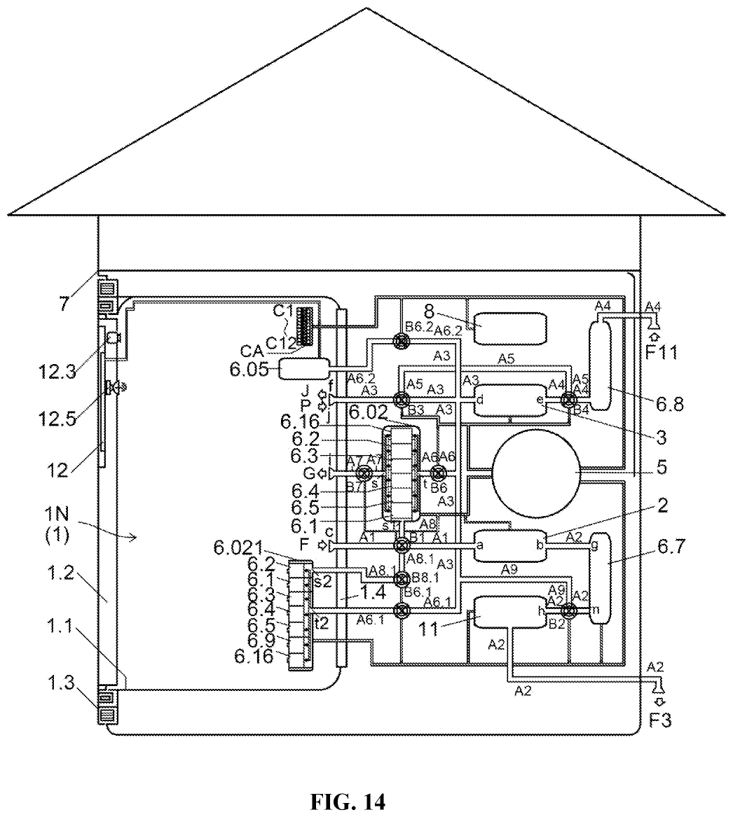

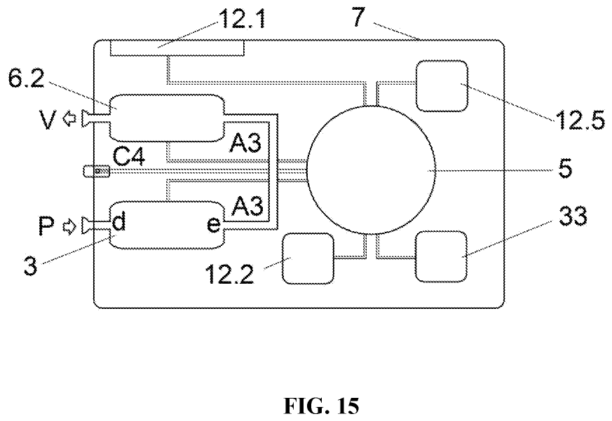

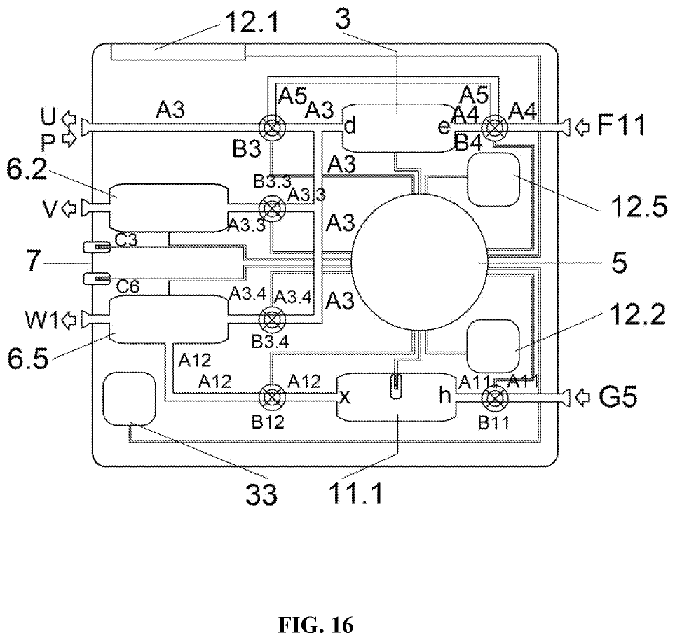

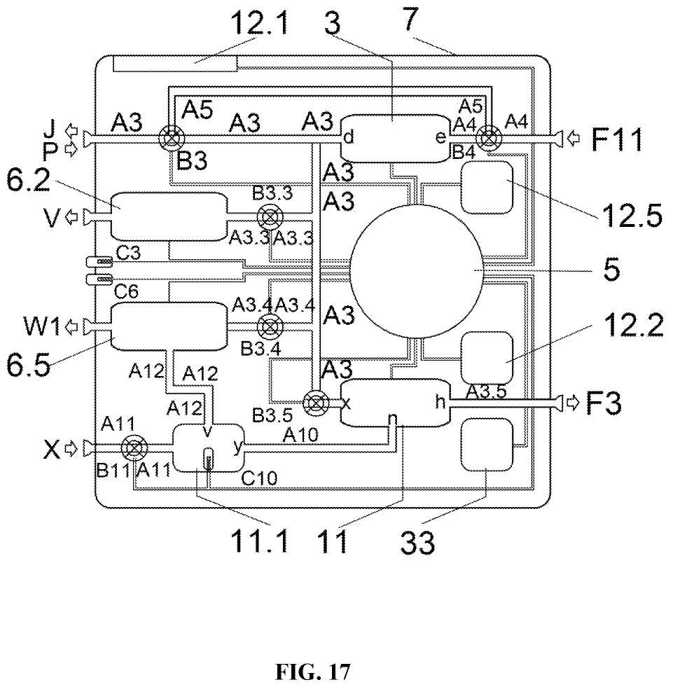

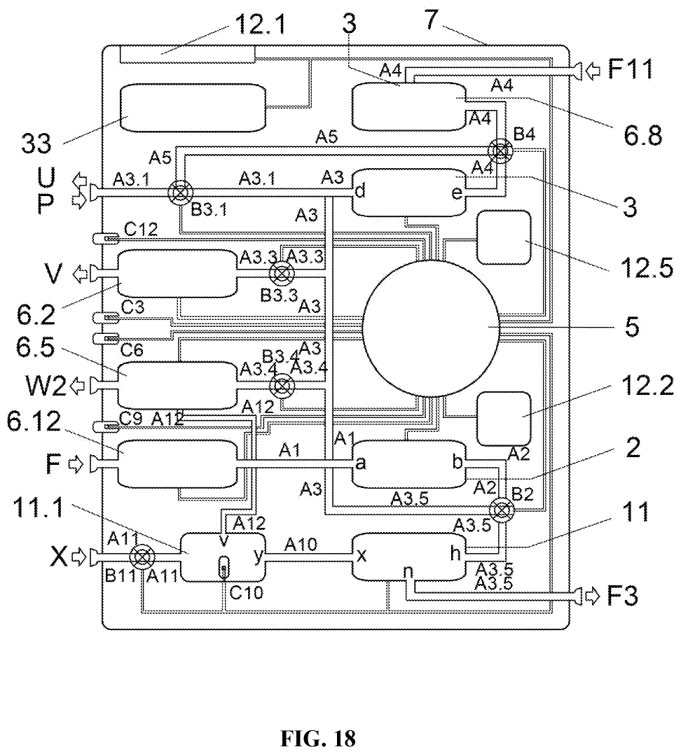

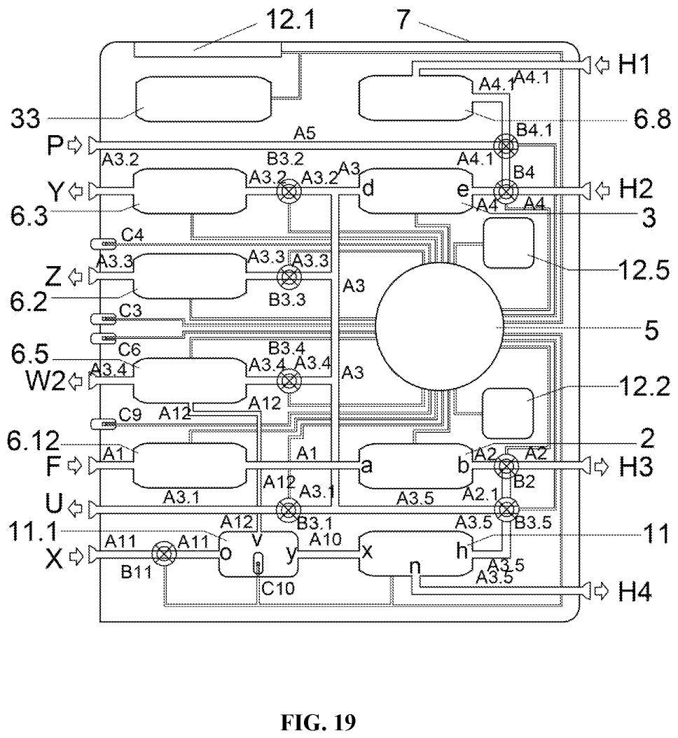

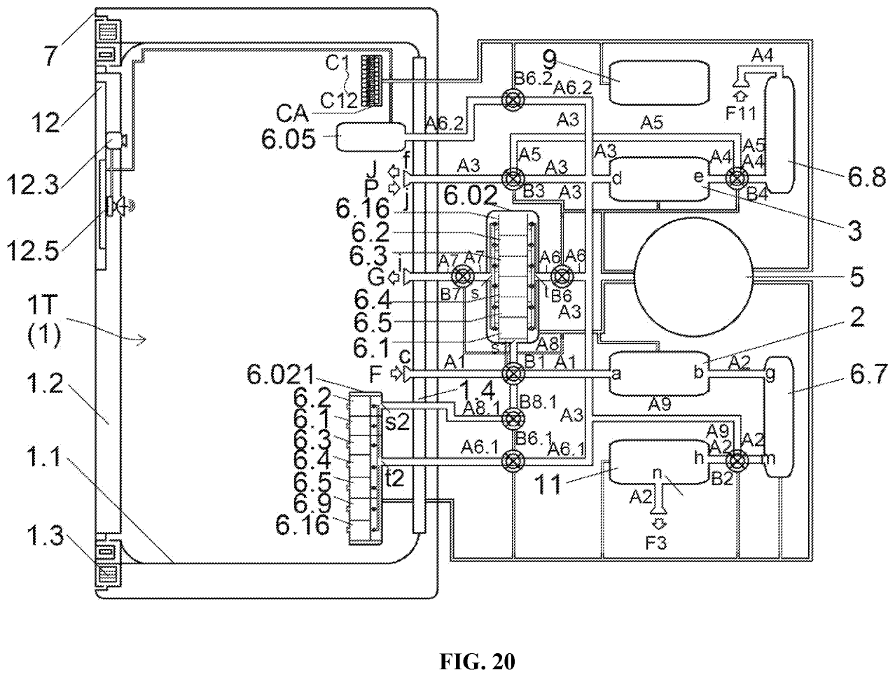

In the drawings: 1—positive and negative pressure cabin (1.1—cabin body, 1.2—cabin door, 1.3—airtight mechanism, 1.31—lock ring, 1.32—lock bolt, 1.33—airtight gasket, 1.34—door rim sealing gasket, 1.4—internal and external communicating sealer, 1.7—wareproof mechanism, 1.71—lock ring, 1.72—lock bolt, 1.73—waterproof gasket, 1.74—cabin door hinge, 1.81—positive and negative pressure refrigerating fresh-keeping drawer, 1.82—positive and negative pressure freezing fresh-keeping drawer, 1.83—non-airtight positive and negative pressure fresh-keeping drawer, 1.9—drawer type airtight mechanism, 1.91—lock ring, 1.92—lock bolt, 1.93—airtight gasket) (1-1—vacuum high pressure cabin, 1-2—atmospheric circulation cabin, 1-3—atmospheric inlet-outlet cabin) (1A—cabinet type positive and negative pressure cabin, 1B—positive and negative pressure refrigerating fresh-keeping cabin, 1B—1-drawer type positive and negative pressure refrigerating fresh-keeping cabin, 1B—2-side-door cabinet type positive and negative pressure refrigerating fresh-keeping cabin, 1C—positive and negative pressure freezing fresh-keeping cabin, 1C—1-drawer type positive and negative pressure freezing fresh-keeping cabin, 1C—2-side-door cabinet type positive and negative pressure freezing fresh-keeping cabin, 1D—general refrigerator refrigerating compartment type positive and negative pressure fresh-keeping cabin, 1E—general refrigerator freezing compartment type positive and negative pressure fresh-keeping cabin, 1F—washing machine type positive and negative pressure cabin, 1G—dish washer type positive and negative pressure cabin, 1H—range hook type positive and negative pressure cabin, 1K—microwave oven type positive and negative pressure cabin, 1M—compartment container type positive and negative pressure cabin, 1N—warehouse type positive and negative pressure cabin, 1T—module type positive and negative pressure cabin), 2—air evacuation pump (2.1—anti-shower air evacuation port), 3—air inflation pump, 5—positive and negative pressure intelligent regulation and control apparatus, 6—positive and negative pressure fluid carried substance generator (6.1—air conditioning apparatus, 6.2—super oxygen generator, 6.3—negative ion generator, 6.4—catalyst release controller, 6.5—humidification, humidification and humidity control apparatus, 6.6—super oxygen generation water mixer, 6.7—carried substance decomposition processor, 6.8—air filter, 6.9—disinfection, sterilization and degradation apparatus, 6.10—filtering and refreshing apparatus, 6.11—stain removal and oil dispelling cleaning apparatus, 6.12—instantaneous disinfector filtering and disinfection apparatus, 6.13—refrigeration and heating and temperature control apparatus, 6.16—carried substance generator item addition and upgrading module) (6.02—air carried substance generator integration (6.021, 6.022, 6.023, 6.024, 6.025), 6.04—waterflow carried substance generator integration, 6.05—integrated pipeline type fluid carried substance generator), 7—machine body, 8—refrigeration system, 9—refrigeration and heating system, 11—air-to-water production apparatus, 12—touch screen and mobile phone monitoring and identification system (12.1—refrigerator door touch screen, 12.3—high-definition anti-fog camera and identification apparatus, 12.4—wireless anti-fog camera and identification apparatus, 12.5—radar scanning and identification apparatus, 12.6—wireless radar scanning and identification apparatus), 15—washing-dehydrating-drying system (15.1—drum mechanism, 15.2—washing-dehydrating-drying control mechanism, 15.3—drum sealing bearing housing, 15.4—big belt pulley, 15.5—motor assembly), 16—water suction pump, 17—water input pump, 18—detergent pull box, 19—dish washing and drying system, 20—fruit and vegetable cleaning system, 21—equipment fume tube cabin, 22—motor turbine assembly, 23—automatic rotary spraying ball, 24—oil collecting and drainage groove, 26—fume evacuation control mechanism, 27—water heating apparatus, 29—fume exhaust pipe, 30—baking system, 31—air frying system; 32—microwave system, A1-A30—pipeline, B1-B25—solenoid valve, C1-C12—sensor (C1—pressure sensor, C2—negative pressure sensor, C3—super oxygen sensor, C4—negative ion sensor, C5—catalyst sensor, C6—humidity sensor, C7—oxygen sensor, C8—nitrogen sensor, C9—disinfection and degradation sensor, C10—air particle sensor, C11—temperature sensing controller, C12—carried substance sensor item addition and upgrading module, C15—positive and negative sensor), CA—sensor integration, D—normal lines, E—bundled line, F—air evacuation and air return, G—carrying air intake, H—carrying air inflation, I—carrying air evacuation, J—air inflation and air intake, U—air inflation and air outlet, K—carrying air return, R—water source intake, M—carrying water return, N1—carrying water intake, N2—carrying water output, O—water suction and water output, P—circulating air return, Q—circulating air intake, V—disinfection air outlet, W1—humidification air intake, W2—humidification air outlet, X—standby water addition, Y—negative ion air outlet, Z—super oxygen air outlet, F3—air exhaust, F11—air intake, F4—atmosphere, F5—water source, F6—water drainage, G3—water intake, G4—circulating air exhaust, G5—tap water, G6—sewer, G9—oily fume, G11—air evacuation, G12—air inflation, H1—filtering air intake, H2—direct air intake, H3—direct air exhaust, H4—water production and air exhaust.

### DETAILED DESCRIPTION OF THE EMBODIMENTS

The following are specific implementations of the embodiments of the present disclosure, and the technical solution of the embodiments of the present disclosure is further described, but the embodiments of the present invention are not limited these embodiments and implementations.

Specific embodiment 1: an intelligent positive and negative pressure system includes a positive and negative pressure cabin (1), an air evacuation pump (2), an air inflation pump (3), a positive and negative pressure intelligent regulation and control apparatus (5), positive and negative pressure fluid carried substance generation processors (6), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water inlet pump (17), and the touch screen and mobile phone monitoring and identification system (12). The positive and negative pressure cabin (1) is classified into a vacuum high pressure cabin (1-1), an atmospheric circulation cabin (1-2), and atmospheric inlet-outlet cabin (1-3) according to a structure and pressure thereof. The positive and negative cabin (1) is classified into a cabinet type positive and negative pressure cabin (1A), a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D), a general refrigerator freezing compartment type positive and negative pressure fresh-keeping cabin (1E), a washing machine type positive and negative pressure cabin (1F), a dish washer type positive and negative pressure cabin (1G), a range hood type positive and negative pressure cabin (1H), a microwave oven type positive and negative pressure cabin (1K), a compartment container type positive and negative pressure cabin (1M), a warehouse type positive and negative pressure cabin (1N), a module type positive and negative pressure cabin (1T), an open type positive and negative pressure cabin (1L), a spherical/semi-spherical positive and negative pressure cabin (1Q), a cylinder type positive and negative pressure cabin (1U), a drawer type positive and negative pressure cabin (1Z), a non-airtight positive and negative pressure cabin (1X), and a shaped positive and negative pressure cabin (1R). An internal structure of the positive and negative pressure cabin (1) includes a cabin body (1.1), a cabin door (1.2), an airtight mechanism (1.3), and an internal and external communicating sealer (1.4). The airtight mechanism (1.3) is arranged between the cabin body (1.1) and the cabin door (1.2); the internal and external communicating sealer (1.4) is fixedly arranged at a rear part or side part of the positive and negative pressure cabin (1), and all pipelines and circuits getting in and out the cabin are connected to and pass through the internal and external communicating sealer (1.4). Each of the positive and negative pressure refrigerating fresh-keeping cabin (1B), a positive and negative pressure freezing fresh-keeping cabin (1C) has a corresponding positive and negative pressure resisting structure, and is provided with an airtight mechanism (1.3), and an internal and external communicating sealer (1.4), which belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the positive and negative pressure cabin. The general refrigerator refrigerating compartment type positive and negative pressure fresh-keeping cabin (1D) and the general refrigerator freezing compartment type positive and negative pressure fresh-keeping cabin (1E) are that a refrigerating compartment and a freezing compartment of the general refrigerator are additionally provided with the positive and negative pressure system, but without the airtight mechanism (1.3) and the internal and external communicating sealer (1.4), thus the general refrigerator refrigerating compartment type positive and negative pressure fresh-keeping cabin (1D) and the general refrigerator freezing compartment type positive and negative pressure fresh-keeping cabin (1E) do not have a vacuum high pressure resistance structure, and belongs to a normal sealed atmospheric circulation cabin (1-2) or atmospheric inlet-outlet cabin (1-3) structure. The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a super oxygen generation water mixer (6.6), a disinfection, sterilization and degradation apparatus (6.9), a filtering and refreshing apparatus (6.10), a stain removal and oil dispelling cleaning apparatus (6.11), a refrigeration, heating and temperature control apparatus (6.13), a carried substance generator item addition and upgrading module (6.16), an airflow carried substance generator (6.01), an airflow carried substance generator integration (6.02), a waterflow carried substance generator (6.03), a waterflow carried substance generator integration (6.04), and an integrated pipeline type positive and negative fluid carried substance generator (6.05). The air conditioning apparatus (6.1) is a membrane-based air separation type air conditioning apparatus. The carried substance generator item addition and upgrading module (6.16) is a combined module capable of providing or installing any individual fluid carried substance generator additional item in the carried substance generator integration and upgrading and updating the same; each of the airflow carried substance generator integration (6.02), the waterflow carried substance generator integration (6.04) and the integrated pipeline type positive and negative pressure fluid carried substance generator (6.05) includes any one or more in the individual fluid carried substance generators (6.1) to (6.16). The integrated pipeline type positive and negative pressure fluid carried substance generator (6.05) is a small, intelligent and integrated fluid carried substance generator and sensor and connected pipelines and circuits; the positive and negative pressure system, according to fluid classification, includes a positive and negative pressure airflow system, and a positive and negative pressure waterflow system. A standard structure of the positive and negative pressure airflow system includes a positive and negative pressure cabin (1), an air evacuation pump (2), an air inflation pump (3), a positive and negative pressure intelligent regulation and control apparatus (5), a touch screen and mobile phone monitoring and identification system (12), and individual fluid carried substance generators (6.1) to (6.16), or a positive and negative pressure airflow carried substance generator integration (6.02), or an integrated pipeline type positive and negative fluid carried substance generator (6.05). The positive and negative pressure airflow system also adopts a nonstandard structure other than the above standard structure, and the nonstandard structure of the positive and negative pressure airflow system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the individual fluid carried substance generators (6.1) to (6.16), the positive and negative pressure airflow carried substance generator integration (6.02), the integrated pipeline type positive and negative fluid carried substance generator (6.05), and the touch screen and mobile phone monitoring and identification system (12). The positive and negative pressure cabin is externally provided with the air evacuation pump (2), the air inflation pump (3), the intelligent positive and negative pressure regulation and control apparatus (5), the touch screen and mobile phone monitoring and identification system (12) and the positive and negative pressure airflow carried substance generator integration (6.02). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the positive and negative pressure cabin 1 to form an air evacuation and air return port (c). An air exhaust pipeline (A2) is installed at an air outlet port (b) of the air evacuation pump (2), a solenoid valve (B2) is installed at a middle part of the air exhaust pipeline (A2), and the other end of the air exhaust pipeline is an exhaust port. A carrying circulation pipeline (A5.1) is further connected to the solenoid valve (B1), the carrying circulation pipeline (A5.1) extends into the positive and negative pressure cabin (1) through the solenoid valve (B1) and the air evacuation pipeline (A1) to form a carrying air inlet port (j1), and the other end of the carrying circulation pipeline (A5.1) is connected to a carrying circulation air inlet port (b2) of the air evacuation pump (2). A carrying waste gas evacuation and exhaust pipeline (A8) is also connected to the solenoid valve (B1), and the other end of the carrying waste gas evacuation and exhaust pipeline (A8) is connected to a carrying waste gas evacuation and exhaust port (s2) of an integrated air conditioning apparatus (6.1) in the positive and negative pressure airflow carried substance generator integration (6.02). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the positive and negative pressure cabin (1) to form an air inflation inlet (f) thereof. An air intake pipeline (A4) is installed at an air inlet port (3) of the air inflation pump (3), a solenoid valve (B4) is installed at a middle part of the air intake pipeline (A4), and the other end of the air intake pipeline (A4) is connected to atmosphere. A carrying circulation pipeline (A5) is further connected to the solenoid valve (B3), the carrying circulation pipeline (A5) extends into the positive and negative pressure cabin 1 through the solenoid valve (B3) and the air inflation pipeline (A3) to form a carrying air return port (j) thereof, and the other end of the carrying circulation pipeline (A5) is connected to a carrying circulation air inlet port (e2). A carrying air introduction pipeline (A6) is installed at an air inlet port (t) of the positive and negative pressure airflow carried substance generator integration (6.02), and a solenoid valve (B6) is installed on the other end of the carrying air introduction pipeline (A6). A carrying air introduction pipeline (A7) is installed at an air outlet port (s) of the positive and negative pressure airflow carried substance generator integration (6.02), a solenoid valve (B7) is installed at a middle part of the carrying air introduction pipeline (A7), and the other end of the carrying air introduction pipeline (A7) extends into the positive and negative pressure cabin (1) to form a carrying air inflation port (i). The solenoid valve (B6) is respectively connected to a carrying air evacuation pipeline (A6.1) and a carrying air intake pipeline (A6.2), the carrying air evacuation pipeline (A6.1) is connected to the air evacuation pipeline (A1) and then is connected to an air evacuation port (a) of the air evacuation pump (2), and the carrying air intake pipeline (A6.2) is connected to the air inflation pipeline (A3) and then communicates with the air outlet port (d) of the air inflation pump (3). A solenoid valve (B6.2) is installed at a middle part of the carrying air intake pipeline (A6.2), and the other end of the carrying air intake pipeline (A6.2) extends into the positive and negative pressure cabin (1) and then is connected to the integrated pipeline type positive and negative pressure fluid carried substance generator (6.05). When a carrying airflow entering the positive and negative pressure cabin (1) requires air inflation circulation, the air inflation inlet (f) serves as the carrying air return port (j) of the positive and negative pressure cabin (1). When the carrying airflow entering the positive and negative pressure cabin (1) requires air evacuation circulation, the air evacuation and air return port (c) serves as the carrying air inlet port (j1) of the positive and negative pressure cabin (1), and the carrying air inflation port (i) serves as a carrying air evacuation port (i1) of the positive and negative pressure cabin (1). A standard structure of the positive and negative pressure waterflow system includes a positive and negative pressure cabin (1), a water suction pump (16), a water input pump (17), a positive and negative pressure intelligent regulation and control apparatus (5), a touch screen and mobile phone monitoring and identification system (12), and a waterflow carried substance generator (6.03), or a waterflow carried substance generator integration (6.04). The positive and negative pressure airflow system also adopts a nonstandard structure other than the above standard structure, and the nonstandard structure of the positive and negative pressure airflow system includes the water suction pump (16) or water input pump (17), and at least one or more of the positive and negative pressure cabin (1), the water suction pump (16), the water input pump (17), the positive and negative pressure intelligent regulation and control apparatus (5), individual fluid carried substance generators (6.1) to (6.16), the waterflow carried substance generator integration (6.04), and the touch screen and mobile phone monitoring and identification system (12). The positive and negative pressure cabin is externally provided with the water suction pump (16), the water input pump (17), the positive and negative pressure intelligent regulation and control apparatus (5), the touch screen and mobile phone monitoring and identification system (12) and the waterflow carried substance generator integration (6.04). A water suction pipeline (A20) is installed at a water suction port (w) of the water suction pump (16), a solenoid valve (B20) is installed at a middle part of the water suction pipeline (A20), and the other end of the water suction pipeline (A20) extends into the positive and negative pressure cabin (1) to form a water suction and water output port (L). A water drainage pipeline (A21) is installed at a water drainage port (p) of the water suction pump (16), a solenoid valve (B21) is installed at a middle part of the water drainage pipeline (A21), and the other end of the water drainage pipeline (A21) is a water drainage port. A carrying circulation pipeline (A19.1) is further connected to the solenoid valve (B20), the carrying circulation pipeline (A19.1) extends into the positive and negative pressure cabin (1) through the solenoid valve (B20) and the water suction pipeline (A20) to form a carrying water inlet port (L1) of the positive and negative pressure cabin (1), and the other end of the carrying circulation pipeline (A19.1) is connected to a carrying circulation water output port (p2) of the water suction pump (16). A water intake pipeline (A15) is installed at a water output port (u) of the water input pump (17), a solenoid valve (B15) is installed at a middle part of the water intake pipeline (A15), and the other end of the water intake pipeline (A15) extends into the positive and negative pressure cabin (1) to form a water source intake (T). A water intake pipeline (A14) is installed at a water inlet port (r) of the water input pump (17), a solenoid valve (B14) is installed at a middle part of the water intake pipeline (A14), and the other end of the water intake pipeline (A14) is connected to a water source. A carrying circulation pipeline (A19) is further connected to the solenoid valve (B15), the carrying circulation pipeline (A19) extends into the positive and negative pressure cabin (1) through the solenoid valve (B15) and the water intake pipeline (A15) to form a carrying water return port (L2) of the positive and negative pressure cabin (1), and the other end of the carrying circulation pipeline (A19) is connected to a carrying circulation water inlet port (r2) of the water input pump (17). A carrying water introduction pipeline (A16) is installed at a water inlet port v of the positive and negative pressure waterflow carried substance generator integration (6.04), and a solenoid valve (B16) is installed at the other end of the carrying water introduction pipeline (A16). A carrying water introduction pipeline (A17) is installed at a water output port (o) of the positive and negative pressure waterflow carried substance generator integration (6.04), a solenoid valve (B17) is installed at a middle part of the carrying water introduction pipeline (A17), and the other end of the carrying water introduction pipeline (A17) extends into the positive and negative pressure cabin (1) to form a carrying water inlet port (T1) of the positive and negative pressure cabin (1). The solenoid valve (b16) is respectively connected to a carrying water suction pipeline (A16.1) and a carrying water intake pipeline (A16.2), the carrying water suction pipeline (A16.1) is connected to the water suction pipeline (A20) and then communicates with a water suction port (w) of the water suction pump (16). The carrying water intake pipeline (A16.2) is connected to the water intake pipeline (A15) and then communicates with the water output port (u) of the water input pump (7). When a carrying waterflow entering the positive and negative pressure cabin (1) requires water intake circulation, the water source intake (t) serves as the carrying water return port (L2) of the positive and negative pressure cabin (1). When a carrying waterflow entering the positive and negative pressure cabin (1) requires water suction circulation, the water suction output port (L) serves as the carrying water inlet port (L1) of the positive and negative pressure cabin (1). The touch screen and mobile phone monitoring and identification system (12) includes a refrigerator door touch screen (12.1), a mobile phone APP (12.2), a high-definition anti-fog camera apparatus (12.3), and a radar scanning recognition apparatus (12.5). The high-definition anti-fog camera apparatus (12.3) and the radar scanning recognition apparatus (12.5) are installed at positions needing to be monitored inside and outside the positive and negative pressure cabin. The positive and negative pressure cabin (1) is internally provided with positive and negative pressure fluid carried substance generators (6), including: a super oxygen generator (6.2), a negative ion generator (6.3), a catalyst release controller (6.4), a humidification, dehumidification and humidity control apparatus (6.5), a disinfection, sterilization and degradation apparatus (6.9), a filtering and refreshing apparatus (6.10), a refrigeration, heating and temperature control apparatus (6.13), a carried substance generator item addition and upgrading module (6.16), or integrated pipeline type positive and negative pressure fluid carried substance generator (6.05). The positive and negative pressure cabin (1) is internally provided with sensors (C), including a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), a carried substance sensor item addition and upgrading module (C12), and a sensor integration (CA). The carried substance sensor item addition and upgrading module (C12) is a combined module capable of providing and installing any individual sensor additional item into the sensor integration and upgrading and updating the same; the sensor integration (CA) includes at least one of more of individual sensors (C1) to (C12). All fluid carried substance generation processors (6), sensors (C) and solenoid valves (B) and the air evacuation pump (2), the air inflation pump (3), the water suction pump (16), the water input pump (17) and the touch screen and mobile phone monitoring and identification system (12) are connected to the positive and negative pressure intelligent regulation and control apparatus (5) by lines (D) or bundled lines (E).

Specific embodiment 2: an operation method for an intelligent positive and negative pressure system is provided. The intelligent positive and negative pressure system is established according to the positive and negative pressure technique, and the positive and negative pressure technique is to regulate and control a fluid and a carried substance thereof by means of positive and negative pressure, so as to affect objects in a specific space. The technical principle and a basic operation method for the intelligent positive and negative pressure system are as follows: intelligently regulating and controlling fluids (for example, air and water) with positive and negative pressure, or carrying multiple effective loads such as super oxygen, a catalyst, a negative ion, an air-conditioned gas, a disinfectant, a cleaning agent, heat and water to orderly get in and out of or stay in a specific space (for example, a positive and negative pressure cabin), and exerting required effects on objects therein. The specific operation method for the intelligent positive and negative pressure system is that according to built-in program, pre-stored data and cloud data of the intelligent positive and negative pressure system, and real-time feedback information of a touch screen and mobile phone monitoring and identification system (12) and various sensors (C11) to (C12), a positive and negative pressure intelligent regulation and control apparatus (5), after calculation processing, forms various targeted influence methods or targeted control modes with different characteristics for various affected objects, and sends specific instructions to an air evacuation pump (2), an air inflation pump (3), a water suction pump (16), a water input pump (17), related positive and negative pressure fluid carried substance generators (6) and related solenoid valves (c) in real time, so as to accurately control and timely adjust open, close and switching of various air evacuation, inflation and circulation pipelines or water suction, intake and circulation pipelines. The level of positive and negative pressure in the positive and negative pressure cabin (1) and flowing-in, staying and flowing-out of an airflow and a carried substance thereof or a waterflow and a carried substance thereof are intelligent regulated and controlled, with specific operation method as follows: operation method (I): an operation method for regulating an interior of the positive and negative pressure cabin (1) into negative pressure, namely, vacuum, is as follows: sending an instruction by the positive and negative pressure intelligent regulation and control apparatus (5) to enable solenoid valves (B1) and (B2) to open air evacuation pipelines (A1) and (A2), air evacuation and air return port c of the positive and negative pressure cabin→air evacuation pipeline (A1)→solenoid valve (B1)→(A1)→air inlet port (a) of the air evacuation pump→air outlet port (b) of the air evacuation pump→(A2)→(B2)→(A2)→exhaust to atmosphere) (when any pipeline is opened, other unrelated solenoid valves are all closed, the following is the same and will not be described again), and turning on the air evacuation pump (2) to vacuumize the interior of the positive and negative pressure cabin (1) to set negative pressure vacuum; operation method (II): an operation method for regulating and controlling the interior of the positive and negative pressure cabin 1 to positive pressure, namely, high pressure, is as follows: sending an instruction by the positive and negative pressure intelligent regulation and control apparatus (5) to enable solenoid valves (B3) and (B4) to open air inflation pipelines (A3) and (A4) as follows: (intake from atmosphere→(A4)→(B4)→(A4)→air inlet port (e) of air inflation pump→air outlet port (d) of air inflation pump→(A3)→(B3)→(A3)→air inflation and intake (f) of the positive and negative pressure cabin, and turning on the air inflation pump (3) to inflate the interior of the positive and negative pressure cabin (1) to the set positive pressure, namely, high pressure; the above operation methods (1) and (2) are only conducted in a vacuum or high-pressure space, such that the positive and negative pressure cabin (1) is classified into a vacuum high pressure cabin (1-1), and a positive and negative pressure cold-preservation fresh-keeping cabin (1B), a positive and negative pressure freezing fresh-keeping cabin (1C), a washing machine type positive and negative pressure cabin (1F), a dish washer type positive and negative pressure cabin (1G), and a microwave oven type positive and negative pressure cabin (1K) all belong to the vacuum high pressure cabin; operation method (III): an operation method for regulating airflow and a carried substance thereof to flow in, stay in or flow out of the positive and negative pressure cabin (1) is as follows: (i) outflow, when the cabin is under the negative pressure or atmospheric, evacuating the airflow and the carried substance thereof out of the positive and negative pressure cabin (1) from air evacuation pipelines using the air evacuation pump (2) according to the above specific operation method (I), and when the cabin is under the high pressure, opening the corresponding solenoid valves for the airflow and the carried substance to flow out from the air evacuation pipelines; (ii) inflow, inflating the airflow and the carried substance thereof into the positive and negative pressure cabin from air inflation pipelines or carrying pipelines using the air inflation pump (3), with specific method as follows: A: inflow of the airflow: when the cabin is under the high pressure or atmospheric, inflating the airflow into the positive and negative pressure cabin from the air inflation pipeline according to the specific operation method (II) using the air inflation pump (3), and when the cabin is under the negative pressure, opening related solenoid valves to enable the airflow to automatically flow into the cabin from the air inflation pipeline; B: inflow of carrying airflow generated by an airflow carried substance generator integration (6.02): sending an instruction by the positive and negative pressure intelligent regulation and control apparatus (5) to enable solenoid valves (B6) and (B7) to open carrying air inflation pipelines (A3), (A6.2), (A6) and (A7), air intake from atmosphere→(A4)→(B4)→(A4)→air inlet port (e) of air inflation pump→air outlet port (d) of air inflation pump→(A3)→(A6.2)→(A6)→(B6)→(A6)→related airflow carried substance generator in airflow carried substance generator integration (6.02)→(A7)→(B7)→(A7)→carrying air inlet port (i) of positive and negative pressure cabin, and turning on the air inflation pump (3) and the related airflow carried substance generator to make the pressure in the positive and negative pressure cabin (1) and inflow of the airflow carried substance reach a set standard; C: inflow of a carrying airflow generated by an integrated pipeline type fluid carried substance generator (6.05): sending an instruction by the positive and negative pressure intelligent regulation and control apparatus (5) to enable a solenoid valve (B6.2) to open carrying air inflation pipelines (A3) and (A6.2), air intake from atmosphere→(A4)→(B4)→(A4)→air inlet port (e) of air inflation pump→air outlet port (d) of air inflation pump→(A3)→(A6.2)→(B6.2)→(A6.2)→related airflow carried substance generator in integrated pipeline type fluid carried substance generator (6.05)→positive and negative pressure cabin, and turning on the air inflation pump (3) and the related airflow carried substance generator in the integrated pipeline type fluid carried substance generator (6.05) to make the pressure in the positive and negative pressure cabin and inflow of the airflow carried substance reach a set standard; (iii) staying, when the airflow and the carried substance thereof need to stay in the positive and negative pressure cabin, sending an instruction by the positive and negative pressure intelligent regulation and control apparatus (5) to make the airflow and pressure and carried substance in the cabin reach the standard, and then closing the corresponding solenoid valves; (iv) circulation: A: air inflation circulation: when the airflow and the carried substance thereof need to circulate to flow through the positive and negative pressure fluid carried substance generator to make the pressure, concentration and composition of the air flow and the carried substance thereof in the positive and negative pressure cabin reach the standard, sending an instruction by the positive and negative pressure intelligent regulation and control apparatus (5) to open carried substance circulation pipelines (A3), (A5), (A6), (A6.2) and (A7) and close (B4) and corresponding solenoid valves, and turning on the air inflation pump (3) and related airflow carried substance generators to make carried substance gases circulate and flow, during the operation of an air conditioning apparatus (6.1), moderately increasing partial pressure difference between both sides of a membrane to improve air separation efficiency, and evacuating and exhausting oxygen-enriched waste gas or carried waste gas; and opening carrying waste gas evacuation and exhaust pipelines (A8), (A1) and (A2) by the solenoid valves (B1) and (B2), carrying waste gas evacuation and exhaust port (s2) of air conditioning apparatus (6.1)→solenoid valve (B1)→(A1)→air inlet port (a) of air evacuation pump→air outlet port (b) of air evacuation pump→(A2)→(B2)→(A2)→exhaust to the atmosphere, and turning on the air evacuation pump (2), operating circularly until corresponding sensors in the positive and negative pressure cabin feed back that the concentration, composition or pressure of the air-conditioned or carrying gas reach the standard, and sending a stop instruction by the positive and negative pressure intelligent regulation and control apparatus (5); B: air evacuation circulation: sending an instruction by the positive and negative pressure intelligent regulation and control apparatus (5) to open carrying air evacuation pipelines (A1), (A5.1), (A6.1) (A6) and (A7) and close (B2) and related solenoid valves, and turning on the air evacuation pump (2) and related airflow carried substance generators to make carried substance gases circulate and flow until corresponding sensors in the positive and negative pressure cabin feed back that the concentration, composition or pressure of the air-conditioned gas reach the standard, and sending a stop instruction by the positive and negative pressure intelligent regulation and control apparatus (5); as the operation methods for the positive and negative pressure cabin (1), namely, (i) outflow, (ii) inflow, (iii) staying and (iv) circulation, are conducted in an atmospheric space, the positive and negative pressure cabin (1) is classified into an atmospheric circulation cabin (1-2) and an atmospheric inlet-outlet cabin (1-3); the general refrigerator refrigerating compartment type positive and negative pressure fresh-keeping cabin (1D) and the general refrigerator freezing compartment type positive and negative pressure fresh-keeping cabin (1E) belong to the atmospheric circulation cabin or the atmospheric inlet-outlet cabin, positive and negative pressure systems are added and used inside and outside the refrigerating compartment and freezing compartment of the general refrigerator, but an airtight mechanism (1.3) and an internal and external communicating sealer (1.4) are not added; the general refrigerator can use the positive and negative pressure system and any one or more of (i) outflow, (ii) inflow, (iii) staying and (iv) circulation procedures in the operation method therefor under atmospheric, thus significantly enhancing the fresh-keeping capacity of the refrigerator and increase the function and efficacy of the refrigerator in many aspects; operation method (IV): an operation method for regulating a waterflow and a carried substance thereof to flow in, stay in or flow out of the positive and negative pressure cabin (1) is as follows: (i) outflow, pumping the waterflow and the carried substance thereof out of the positive and negative pressure cabin from water suction pipelines using a water suction pump (16), with specific methods as follows: sending an instruction by the positive and negative pressure intelligent regulation and control apparatus (5) to open water suction pipelines (A20) and (A21) by solenoid valves (B20) and (B21), water suction port (L) of positive and negative pressure cabin→water suction pipeline (A20)→solenoid valve (B20)→(A20)→water inlet port (w) of water suction pump→water drainage port (p) of water suction pump→(A21)→(B21)→(A21)→drainage to sewer, and turning on the water suction pump (16) to drain the water in the positive and negative pressure cabin (1) to a set standard; (ii) inflow, feeding the waterflow and the carried substance thereof into the positive and negative pressure cabin from air feeding and intake pipelines or carrying water intake pipelines using the water input pump (17), with specific method as follows: A: water feeding and intake: sending an instruction by the positive and negative pressure intelligent regulation and control apparatus (5) to open water intake pipelines (A14) and (A15) by solenoid valves (B14) and (B15), water intake from water source→(A14)→(B14)→(A14)→water inlet port (r) of water input pump→water output port (u) of water input pump→(A15)→(B15)→(A15)→water feeding and inlet port (T) of the positive and negative pressure cabin, and turning on the water input pump (17) to make the water feeding in the positive and negative pressure cabin (1) reach the standard; B: carrying water intake: sending an instruction by the positive and negative pressure intelligent regulation and control apparatus (5) to open carrying water intake pipelines (A14), (A15), (A16), (A16.2) and (A17) by solenoid valves (B14), (B16) and (B17), water intake from water source→(A14)→(B14)→(A14)→water inlet port (r) of water input pump→water output port (u) of water input pump→(A15)→(A16.2)→(B16)→(A16)→corresponding waterflow carried substance generator→(A17)→(B17)→(A17)-carrying water inlet port (T1) of positive and negative pressure cabin, and turning on the water input pump (17) and the corresponding waterflow carried substance generator to make water feeding in the positive and negative pressure cabin (1) and inflow of the waterflow carried substance to a set standard; (iii) staying, when the waterflow and the carried substance thereof need to stay in the positive and negative pressure cabin, sending an instruction by the positive and negative pressure intelligent regulation and control apparatus (5), firstly making the waterflow pressure and carried substance in the cabin reach the standard, and then closing the corresponding solenoid valves until staying time reaches the standard, and sending a stop instruction by the positive and negative pressure intelligent regulation and control apparatus (5); (iv) circulation: A: water intake circulation: when the waterflow and the carried substance thereof need to circulate to flow through the positive and negative pressure fluid carried substance generator to make the pressure, concentration and composition of the waterflow and the carried substance thereof in the positive and negative pressure cabin reach the standard, sending an instruction by the positive and negative pressure intelligent regulation and control apparatus (5) to open carrying water intake circulation pipelines (A15), (A16), (A16.2), (A17) and (A19) and close (B14) and related solenoid valves, and turning on the water input pump (17) and related waterflow carried substance generators to make substance-carrying waterflow circulate and flow until corresponding sensors in the positive and negative pressure cabin feed back that the concentration, the composition or pressure of the carrying waterflow reach the standard, and sending a stop instruction by the positive and negative pressure intelligent regulation and control apparatus (5); B: water suction circulation: sending an instruction by the positive and negative pressure intelligent regulation and control apparatus (5) to open carrying water suction pipelines (A20), (A16), (A16.1), (A17) and (A19.1) and close (B21) and related solenoid valves, and turning on the water suction pump (16) and related waterflow carried substance generators to make the substance-carrying waterflow circuit and flow until corresponding sensors in the positive and negative pressure cabin feed back that the concentration, the composition or pressure of the waterflow reach the standard, and sending a stop instruction by the positive and negative pressure intelligent regulation and control apparatus (5); one or more of the operation methods, such as, (i) outflow, (ii) inflow, (iii) staying and (iv) circulation, for regulating and controlling the waterflow and carried substance thereof to flow in, stay in or flow out the positive and negative pressure cabin (1) are conducted not only in a vacuum high pressure cabin, such as a washing machine type positive and negative pressure cabin (1F), a dish washer type positive and negative pressure cabin (1G), but also in an atmospheric circulation cabin (1-2) and the atmospheric inlet-outlet cabin (1-3), such as a range hood type positive and negative pressure cabin (1H), an open type positive and negative pressure cabin (1L), and a non-airtight positive and negative pressure cabin (1X); the intelligent positive and negative pressure system, due to the use of above technical operation methods, has an ultra-large-capacity carrier and renewable performance and a platform advantage of upgrading, extending and adding items. Various techniques such as vacuum, high pressure, super oxygen, catalysts, air conditioning, negative ions, disinfection and purification, moisture and temperature control, air-to-water production, low-temperature storage, clothes washing and drying and food processing can be intelligently regulated, controlled and integrated, and one item of or a combination of more items of the techniques is applied to electrical equipment, transport compartments, containers and warehouses. Due to the use of the intelligent positive and negative pressure system and operation method therefor, the electrical equipment, transport carriages, containers and warehouses are uniformly referred to as intelligent positive and negative pressure electric appliances. The intelligent positive and negative pressure electric appliances in embodiments of the present disclosure include an intelligent positive and negative pressure fresh-keeping refrigerator, an intelligent positive and negative pressure washing machine, an intelligent positive and negative pressure dish washing and fruit and vegetable cleaning machine, an intelligent positive and negative pressure range hood, an intelligent positive and negative pressure baking and frying microwave oven, an intelligent positive and negative pressure fresh-keeping compartment container, an intelligent positive and negative pressure fresh-keeping warehouse, an intelligent positive and negative pressure disinfection machine, and an intelligent positive and negative pressure module cabinet.

Specific embodiment 3: an intelligent positive and negative pressure refrigerator includes a refrigerator body (7). The refrigerator body (7) is internally provided with an intelligent positive and negative pressure system, a refrigeration system (8), and an air-to-water production apparatus (11). The intelligent positive and negative pressure system includes a positive and negative pressure cabin (1), an air evacuation pump (2), an air inflation pump (3), a positive and negative pressure intelligent regulation and control apparatus (5), airflow carried substance generator integrations (6.021), (6.022), (6.023), (6.024) and (6.025), and a touch screen and mobile phone monitoring system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), a positive and negative pressure fluid carried substance generation processor (6), and the touch screen and mobile phone monitoring and identification system (12). The positive and negative pressure cabin (1), according to cold preservation and freezing requirements of a fresh-keeping refrigerator, is designed as a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D), and a general refrigerator freezing compartment type positive and negative pressure fresh-keeping cabin (1E). The positive and negative pressure refrigerating fresh-keeping cabin (1B) and the positive and negative pressure freezing fresh-keeping cabin (1C) are both vacuum high pressure cabins (1-1), a positive and negative pressure resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the refrigerator; one or more of positive and negative pressure refrigerating fresh-keeping cabins (1B) and positive and negative pressure freezing fresh-keeping cabins (1C) are respectively arranged in the refrigerator body (7) in up-down or left-right arrangement and asymmetrical irregular arrangement modes. The positive and negative pressure refrigerating fresh-keeping cabin (1B) and the positive and negative pressure freezing fresh-keeping cabin (1C), according to the shape and characteristic classification, include a drawer type positive and negative pressure refrigerating fresh-keeping cabin (1B-1), a drawer type positive and negative pressure freezing fresh-keeping cabin (1C-1), a side door cabinet type positive and negative pressure refrigerating fresh-keeping cabin (1B-2), and a side door cabinet type positive and negative pressure freezing fresh-keeping cabin (1C-2). The drawer type positive and negative pressure refrigerating fresh-keeping cabin (1B-1) and the drawer type positive and negative pressure freezing fresh-keeping cabin (1C-1) include cabin bodies (1.1), a positive and negative pressure refrigerating fresh-keeping drawer (1.81), a positive and negative pressure freezing fresh-keeping drawer (1.82), and internal and external communicating sealers (1.4). A drawer type airtight mechanism (1.9) is arranged between the cabin body of the drawer type positive and negative pressure refrigerating fresh-keeping cabin (1B-1) and the positive and negative pressure refrigerating fresh-keeping drawer (1.81) and between the cabin body of the drawer type positive and negative pressure freezing fresh-keeping cabin (1C-1) and the positive and negative pressure freezing fresh-keeping drawer (1.82). The drawer type airtight mechanism (1.9) includes a locking ring (1.91), a lock bolt (1.92), and an airtight gasket (1.93); the locking ring (1.91) is in movable fit with the lock bolt (1.92), when the fresh-keeping drawer is closed, the locking bolt (1.92) extends into the locking ring (1.92) to lock a cabin door, and the airtight gasket (1.93) is used for keeping sealing. The side door cabinet type positive and negative pressure refrigerating fresh-keeping cabin (1B-2) and the side door cabinet type positive and negative pressure freezing fresh-keeping cabin (1C-2) each include a cabin door (1.1), a cabin door (1.2), an airtight mechanism (3), and an internal and external communicating sealer (1.4). The airtight mechanism (1.3) is arranged between the cabin door (1.2) and the cabin body (1.1). The airtight mechanism (1.3) includes a locking ring (1.31), a lock bolt (1.32), an airtight gasket (1.33), and a door rim sealing gasket; the locking ring (1.31) is in movable fit with the lock bolt (1.32). When the cabin door is closed, the locking bolt (1.32) extends into the locking ring (1.32) to lock the cabin door, and the airtight gasket (1.33) is used for keeping sealing. The internal and external communicating sealer (1.4) is arranged on a side part or a side part of each of the drawer type positive and negative pressure refrigerating fresh-keeping cabin (1B-1), the drawer type positive and negative pressure freezing fresh-keeping cabin (1C-1), the side door cabinet type positive and negative pressure refrigerating fresh-keeping cabin (1B-2), and the side door cabinet type positive and negative pressure freezing fresh-keeping cabin (1C-2), all pipelines and circuits getting in and out the cabin are connected to and pass through the internal and external communicating sealer (1.4), so as to keep the sealing property of the positive and negative pressure cabin (1). The general refrigerator refrigerating compartment type positive and negative pressure fresh-keeping cabin (1D) and the general refrigerator freezing compartment type positive and negative pressure fresh-keeping cabin (1E) are both atmospheric circulation cabins, each of a refrigerating compartment and a freezing compartment of the general refrigerator is provided with a positive and negative pressure system, but without the airtight mechanism and internal and external communicating sealer. The general refrigerator refrigerating compartment type positive and negative pressure fresh-keeping cabin (1D) includes a cabin body (1.1) and a cabin door (1.2), and an airtight mechanism and an internal and external communicating sealer do not need to be provided between the cabin body (1.1) and the cabin door (1.2), and the cabin door (1.2) is a refrigerator door of the fresh-keeping refrigerator. The general refrigerator freezing compartment type positive and negative pressure fresh-keeping cabin (1E) includes a cabin door (1.1), and a non-airtight fresh-keeping drawer (1.83). An airtight mechanism and an internal and external communicating sealer do not need to be provided between the cabin body (1.1) and the non-airtight fresh-keeping drawer (1.83). The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), airflow carried substance generator integrations (6.021), (6.022), (6.023), (6.024) and (6.025). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. Each of the airflow carried substance generator integrations (6.021), (6.022), (6.023), (6.024) and (6.025) includes any one or more of the individual fluid carried substance generators (6.1) to (6.16) as required. Each of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) is externally provided with an air evacuation pump (2), an air inflation pump (3), a positive and negative pressure intelligent regulation and control apparatus (5), a positive and negative pressure airflow carried substance generator integration (6.021), a carried substance decomposition processor (6.7), an air filter (6.8), a refrigeration system (8), an air-to-water production apparatus (11), and a touch screen and mobile phone monitoring system (12). An air evacuation pipeline (A1) is installed at an air inlet port (a) of the air evacuation pump (2), air evacuation branch pipelines (A1.1), (A1.2), (A1.3) and (A1.4) are respectively connected to the air evacuation pipeline (A1), solenoid valves (B1.1), (B1.2), (B1.3) and (B1.4) are respectively installed at middle parts of the air evacuation branch pipelines (A1.1), (A1.2), (A1.3) and (A1.4), and the other ends of the air evacuation branch pipelines (A1.1), (A1.2), (A1.3) and (A1.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form air evacuation and air return ports (c1), (c2), (c3) and (c4) thereof. Carrying waste gas evacuation and exhaust pipelines (A8), (A8.1), (A8.2), (A8.3) and (A8.4) are respectively connected to the air evacuation pipeline (A1), solenoid valves (B8), (B8.1), (B8.2), (B8.3) and (B8.4) are respectively installed at middle parts of the carrying waste gas evacuation and exhaust pipelines (A8), (A8.1), (A8.2), (A8.3) and (A8.4), and the other ends of the carrying waste gas evacuation and exhaust pipelines (A8), (A8.1), (A8.2), (A8.3) and (A8.4) are respectively connected to carrying waste gas evacuation and exhaust ports (s3), (s4), (s5), (s6) and (s7) of the air conditioning apparatus (6.1) integrated in the positive and negative pressure airflow carried substance generator integrations (6.021), (6.022), (6.023), (6.024) and (6.025). An air exhaust pipeline (A2) is installed at an air outlet port (b) of the air evacuation pump (2), a carried substance decomposition processor (6.7) and a solenoid valve (B2) are installed at a middle part of the air exhaust pipeline (A2), and the other end of the air exhaust pipeline (A2) is connected to an air inlet port (h) of an air-to-water production apparatus (11), and then is connected to atmosphere via an air outlet port (n) of the air exhaust pipeline (A2) after passing through the air-to-water production apparatus (11). An air inflation pipeline (A3) is installed at an air outlet port (d) of the air inflation pump (3), a solenoid valve (B3) is installed at a middle part of the air inflation pipeline (A3), air inflation branch pipelines (A3.1), (A3.2), (A3.3) and (A3.4) are respectively connected to the air inflation pipeline (A3), solenoid valves (B3.1), (B3.2), (B3.3) and (B3.4) are respectively installed at middle parts of the air inflation branch pipelines (A3.1), (A3.2), (A3.3) and (A3.4), and the other ends of the air inflation branch pipelines (A3.1), (A3.2), (A3.3) and (A3.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form air inflation and inlet ports (f1), (f2), (f3) and (f4) thereof. A carrying air introduction pipeline (A6) is further connected to the air inflation pipeline (A3), a solenoid valve (B6) is installed at a middle part of the carrying air introduction pipeline (A6), and the other end of the carrying air introduction pipeline (A6) is connected to an air inlet port (t) of the positive and negative pressure airflow carried substance generator integration (6.021). Carrying air introduction branch pipelines (A7.2) and (A7.3) are respectively installed at air outlet ports (s1) and (s2) of the positive and negative pressure airflow carried substance generator integration (6.021), solenoid valves (B7.2) and (B7.3) are respectively installed at middle parts of the carrying air introduction branch pipelines (A7.2) and (A7.3), and the other ends of the carrying air introduction branch pipelines (A7.2) and (A7.3) respectively extend into the positive and negative pressure refrigerating fresh-keeping cabin (1B) and the positive and negative pressure freezing fresh-keeping cabin (1C) to form carrying air inlet ports (12) and (i3) thereof. Carrying air introduction branch pipelines (A7.1) and (A7.4) are respectively connected to the solenoid valves (B7.2) and (B7.3), solenoid valves (B7.1) and (B7.4) are respectively installed at middle parts of the carrying air introduction branch pipelines (A7.1) and (A7.4), and the other ends of the carrying air introduction branch pipelines (A7.1) and (A7.4) respectively extend into the general refrigerator refrigerating compartment type positive and negative pressure fresh-keeping cabin (1D) and the general refrigerator freezing compartment type positive and negative pressure fresh-keeping cabin (1E) to form carrying air inlet ports (i1) and (i4) thereof. Carrying branch pipelines (A6.1), (A6.2), (A6.3) and (A6.4) are respectively connected to the air inflation pipeline (A3), solenoid valves (B6.1), (B6.2), (B6.3) and (B6.4) are respectively installed at middle parts of the carrying branch pipelines (A6.1), (A6.2), (A6.3) and (A6.4), and the other ends of the carrying branch pipelines (A6.1), (A6.2), (A6.3) and (A6.4) are respectively connected to air inlet ports (t1), (t2), (t3) and (t4) of the positive and negative pressure airflow carried substance generator integrations (6.023), (6.024) and (6.025). An air intake pipeline (A4) is installed at an air inlet port (e) of the air inflation pump (3), an air filter (6.8) and a solenoid valve (B4) are installed at middle parts of the air intake pipeline (A4), and the other end of the air intake pipeline (A4) is connected to atmosphere. A circulation pipeline (A5) is further connected to the solenoid valve (B4), and circulation branch pipelines (A5.1), (A5.2), (A5.3) and (A5.4) are respectively connected to the circulation pipeline (A5), solenoid valves (B5.1), (B5.2), (B5.3) and (B5.4) are installed at middle parts of the circulation branch pipelines (A5.1), (A5.2), (A5.3) and (A5.4), and the other ends of the circulation branch pipelines (A5.1), (A5.2), (A5.3) and (A5.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form circulation air return ports (j1), (j2), (j3) and (j4) thereof. An air-to-water production inflation pipeline (A9) is further connected to the circulation pipeline (5) through a solenoid valve (B9), the air-to-water production inflation pipeline (A9) is connected to the solenoid valve (B2) and then communicates with an air inlet port (h) of the air-to-water production apparatus (11) through the air exhaust pipeline (A2), the air-to-water production inflation pipeline (A9) is connected to the circulation pipeline (A5) and communicates with the solenoid valve (B3), and then communicates with an air outlet port (d) of the air inflation pump (3) through the air inflation pipeline (A3). The touch screen and mobile phone monitoring and identification system (12) includes a refrigerator door touch screen (12.1), a mobile phone APP (12.2), a high-definition anti-fog camera apparatus (12.3), and a radar scanning recognition apparatus (12.5). The high-definition anti-fog camera apparatus (12.3) and the radar scanning recognition apparatus (12.5) are installed at a position needing to be monitored and recognized inside and outside the refrigerator body (7). The cabins of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) are internally provided with airflow carried substance generator integrations (6.022), (6.023), (6.024) and (6.025) and sensor integrations (CA). Each of the airflow carried substance generator integrations (6.022), (6.023), (6.024) and (6.025) includes one or more of the following individual airflow carried substance generators (6.1) to (6.16): an air conditioning apparatus (6.1), a super oxygen generator (6.2), a negative ion generator (6.3), a catalyst release controller (6.4), a humidification, dehumidification and humidity control apparatus (6.5), a disinfection, sterilization and degradation apparatus (6.9), a carried substance generator item addition and upgrading module (6.16). The sensor includes the sensor integration (CA), the sensor integration (CA) includes one or more of the following individual sensors (C1) to (C12) as required: a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), a carried substance sensor item addition and upgrading module (C12). The carried substance sensor item addition and upgrading module (C12) is a combined module capable of providing and installing any individual sensor additional item into the sensor integration and upgrading and updating the same; the sensor integration (CA) includes at least one of more of individual sensors (C1) to (C12). All airflow carried substance generation processors (6), sensors (C) and solenoid valves (B) and the air evacuation pump (2), the air inflation pump (3), the refrigeration system (8), the air-to-water production apparatus (11) and the mobile phone monitoring and identification system (12) are connected to the positive and negative pressure intelligent regulation and control apparatus (5) by lines (D) or bundled lines (E).

Specific embodiment 4: an operation method for an intelligent positive and negative pressure fresh-keeping refrigerator is provided. The intelligent positive and negative pressure fresh-keeping refrigerator adopts an intelligent positive and negative pressure system. The intelligent positive and negative pressure system is used to accurately regulate and organically integrate various techniques such as vacuum, high pressure, super oxygen, catalysts, air conditioning fresh-keeping, negative ions, humidification, dehumidification, humidity and temperature control, disinfection and sterilization, degradation treatment, air purification, air-to-water production, and low-temperature storage, one item of or a combination of more items of the techniques is applied to the positive and negative pressure fresh-keeping refrigerator, and digital techniques such as Internet, big data, cloud computing, machine learning, artificial intelligence and expert system are also applied to the refrigerator at the same time, so as to form a regulatable and controllable artificial intelligence microclimate in the positive and negative pressure fresh-keeping refrigerator and to create a high-energy and efficient precise fresh-keeping microenvironment. Various fresh-keeping elements such as air composition, airflow pressure, humidity and temperature, aseptic cleanliness and the like of the positive and negative pressure fresh-keeping refrigerator are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization, quickly degrade harmful residues on the surface of preservation, achieve all-round and multi-angle digital targeted treatment, eliminate putrefying factors, accurately keep freshness and comprehensively preserve nutrition; various fresh goods stored in the positive and negative pressure fresh-keeping refrigerator are in an optimal pressure range, an optimal gas composition and optimal humidity and temperature, and an optimal preservation environment. Fruits and vegetables are hibernated at low temperature in an existing refrigerator, while the positive and negative pressure and digital techniques are used in the positive and negative pressure fresh-keeping refrigerator to make fruits and vegetables in a dormant or suspended animation state, such that the fruits and vegetables have the weakest life, the slowest aging, the lowest breathing and the least consumption of nutrition, and the nutritional components and the original freshness of fruits, vegetables, meat, fish and other stored products are quickly stored for a long time; in the positive and negative pressure system, the digital technique is used to analyze and identify fresh-loss sites and fresh-keeping elements of various stored products one by one to form a complete large database according to different categories, and to design a personalized targeted control comprehensive fresh-keeping method at the cellular and molecular level for the fresh-loss sites and fresh-keeping elements of the stored products, so as to accurately maintain the fresh-keeping elements and remove the fresh-loss factors at a fixed point, without affecting or less affecting the normal tissues and cells of the stored products, and thus a set of digital expert system for preservation and fresh-keeping is formed and gradually improved, which is intelligently applied to the positive and negative pressure fresh-keeping refrigerator to achieve the purpose of keeping freshness and quality of the stored products. In a specific operation method for the intelligent positive and negative pressure fresh-keeping refrigerator, an air evacuation pump or air inflation pump is used by the positive and negative pressure system to intelligently regulate and control airflow pressure, or carry super oxygen, catalysts, negative ions, air conditioning gas, disinfection and degradation substances, heat, water and various effective loads by the airflow to get in, stay in or get out of a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D) and a general refrigerator freezing compartment type positive and negative pressure fresh-keeping cabin (1E) orderly, and the following methods are used to apply various forces helpful for long-term freshness and quality keeping to fresh fruits and vegetables stored in the refrigerator: (1) a positive atmospheric pressure high pressure action method: forming moderate positive atmospheric pressure at proper time in a cabin of the compartment container type positive and negative pressure cabin (1M), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the compartment container type positive and negative pressure cabin (1M), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) an airflow carried substance action method: carrying single or a combination of multiple super oxygen, catalyst, negative ion, air conditioning gas, disinfection and degradation substances, heat, water and various effective loads by positive and negative pressure airflow to get in, stay in or get out of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) orderly, integrating various fresh-keeping techniques to apply various accurate and effective action forces for the long-term freshness and quality keeping of the stored fruits, vegetables and fresh goods. Based on above three action force methods, the positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of the touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping; the targeted control comprehensive fresh-keeping methods for the intelligent positive and negative pressure system and specific use methods and operation procedures of the positive and negative pressure fresh-keeping refrigerator are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1.1) or (B1.2 or (B1.3) or (B1.4) to open corresponding air exhaust pipelines (A2) and (A1) and (A1.1) or (A1.2 or (A1.3) or (A1.4) of the related positive and negative pressure cabin, and turning on the air evacuation pump (2) to exhaust or pump the related positive and negative pressure cabin to vacuum negative pressure between −0.001 KPa and −0.1 MPa (taking the local instant atmospheric pressure as the zero standard, and the vacuum degree can be improved according to the specific demands and refrigerator configuration), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2), pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within the positive and negative pressure cabin, thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) atmospheric or pressurized sterilization, degradation and deodorization procedure for super-oxygen negative ion or catalyst positive and negative pressure carried substance: according to the procedure setting and feedback of the related sensors (C), sending, by the positive and negative pressure intelligent regulation and control apparatus (5), an instruction at a proper time to turn on a super oxygen generator (6.2), a negative ion generator (6.3), a catalyst release controller (6.4), a humidity control apparatus (6.5) or a disinfection and degradation apparatus (6.9) in each related airflow carried substance generator integration, or a carried substance generator installed in a carried substance generation item addition and upgrading module (6.16), thus enabling the super oxygen, catalyst, negative ion, water and related carried substances in the positive and negative pressure cabin reach required standards; meanwhile, enabling solenoid valves (B3) and (B4) or (B3.1) or (B3.2) or (B3.3) or (B3.4) to open corresponding air inflation pipelines (A4) and (A3) or (A3.1) or (A3.2) or (A3.3) or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and refrigerator configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric or pressurized sterilization, degradation and deodorization procedure for air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3), (B6) and (B7.1), (B7.2), (B7.3) or (B7.4) to open related carrying air inflation pipelines (A3), (A6) and (A7.1), (A7.2), (A7.3) or (A7.4) and turn on the air inflation pump (3) to drive the airflow to enter the related positive and negative pressure cabin after passing through an air conditioning apparatus (6.1) and other related positive and negative pressure fluid carried substance generators (6); and enabling solenoid valves (B3), (B4), (B9) and (B5.1), (B5.2), (B5.3) or (B5.4) to open carrying circulation pipelines to circulate and operate; during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B8) or (B8.1), (B8.2), (B8.3) or (B8.4) to open carrying waste gas evacuation and exhaust pipelines (A8) or (A8.1), (A8.2), (A8.3) or (A8.4), carrying waste gas evacuation and exhaust port (s3), (s4), (s5), (s6) or (s7) of air conditioning apparatus (6.1)→(A8) or (A8.1), (A8.2), (A8.3) or (A8.4)→solenoid valves (B8) or (B8.1), (B8.2), (B8.3) or (B8.4)→(A8), (A8.1), (A8.2), (A8.3) or (A8.4)→(A1)→air inlet port (a) of air evacuation pump→air outlet port (b) of air evacuation pump→(A2)→carried substance decomposition processor (6.7)→(B2)→(A2)→air-to-water production apparatus (11)→exhaust to atmosphere; turning on the air evacuation pump (2) to exhaust decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) atmospheric, pressurized, or sterilized preservation procedure for leftovers: placing the leftovers into the positive and negative pressure cabin, turning on the air evacuation pump (2) and related air pipelines by the regulation and control apparatus (5) until the odor already released by the leftovers and the polluted air inside the cabin are pumped out of the cabin; turning on the air inflation pump (3) and related air inflation pipelines for properly pressurized preservation, which not only prevents the odor and water from excessively scattering and losing, but also inhibits the food spoilage; meanwhile, according to feedback information of the humidity sensor, turning on the humidity control apparatus (6.5) at proper time to supplement the water in the air, or turning on related carried substance generation processors at proper time to kill the bacteria and virus in the air in the cabin to prevent food spoilage; (6) low-temperature assisted fresh-keeping procedure: after the positive and negative pressure fresh-keeping refrigerator is turned on, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (7) open-to-exhaust protection function: if the refrigerator door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to a carried substance decomposition processor (6.7) at the rear part of the refrigerator to be exhausted to atmosphere after being decomposed, where, as the air flows from the outside of the refrigerator door to the positive and negative pressure cabin for supplementing air evacuation negative pressure, the super oxygen or high-nitrogen low-oxygen carried substance gas cannot flow out of the door; (8) air-to-water production procedure: enabling moist waste gas pumped from the vacuum high pressure cabin by the air evacuation pump 2 to enter the super oxygen decomposer (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B9) and B2 to open air-to-water production inflation pipelines, thus driving the indoor air to enter the air-to-water production apparatus (11) for water production; (9) remote and short-range control monitoring and identification function: controlling and monitoring, by the touch screen and mobile phone monitoring and identification system (12), the fresh-keeping refrigerator and other positive and negative pressure electric appliances and a positive and negative pressure module cabinet in real time by the WIFI and mobile phone APP at remote and short-range, thus making the fresh-keeping refrigerator and other all positive and negative pressure electric appliances and module cabinets to intelligently achieve various functions at high efficiency and low consumption according to the set procedures and instant instructions, where a high-definition anti-fog camera and identification apparatus (12.3) and a radar scanning and identification apparatus (12.5) are installed at positions needing monitoring and identification inside and outside the refrigerator body (7); shooting and scanning statuses of objects in the refrigerator in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling various fresh-keeping environmental factors such as airflow pressure, gas composition, air cleanliness, environment temperature and humidity in the positive and negative pressure refrigerating-freezing cabin in real time, and automatically performing harmless disinfection and sterilization, and rapidly degrading residual pesticides, fertilizers, hormones and harmful additives to form an adjustable and controllable artificial microclimate in the positive and negative pressure refrigerating-freezing cabin, thus creating a microenvironment favorable for fresh-keeping, which makes various fresh foods stored in the positive and negative pressure refrigerating-freezing cabin in the optimal atmospheric pressure range, the optimal gas composition, the optimal humidity, and the optimal preservation environment for keeping fresh, tender and original flavor; displaying and reminding, by a refrigerator door touch screen (12.1) or a mobile phone APP (12.2), commodity information such as production date, price, expiration date and manufacturers and information of purchase shopping malls and online stores, automatically recording, analyzing and handling the big data such as the time, quantity, frequency and preference of preserving the objects in, and taking the objects out, the refrigerator, and performing intelligent analysis by combining cloud data; timely reminding on the mobile phone APP (12.2) or directly pushing the suggested goods to the mobile phone APP (12.2) or the refrigerator door touch screen (12.1), where, in addition to real-time monitoring and identification at a fixed position, the camera and identification apparatus (12.3) is required to be installed in the refrigerator door (1.2) and to automatically and continuously shoot multiple panoramic photos of the objects in the refrigerator and automatically record small videos with the closing movement of the refrigerator door (1.2) by starting from the position away from the refrigerator body in the process of closing the refrigerator door, thus facilitating a user to view in the mobile phone APP, or view the panoramic photos or videos of the objects in the refrigerator with good light and wide field of vision before and during the last closing of the door in the refrigerator door touch screen at any time without opening the refrigerator door, releasing the vacuum and lowering the temperature during short range; and dynamically identifying the objects and uploading the data; and (10) positive and negative pressure module combined intelligent smart home mode: designing and manufacturing the intelligent positive and negative pressure fresh-keeping refrigerator according to the module functions, where the intelligent positive and negative pressure refrigerator not only is independently produced and used as a separated invention, or but also is organically combined with other positive and negative pressure electric appliances and the module cabinets under the control of the touch screen and mobile phone monitoring and identification system (12) to form a fully functional intelligent positive and negative pressure module combined home; the fully functional intelligent positive and negative pressure module combined home not only can share the positive and negative pressure system, the refrigeration system and the air-to-water production apparatus, but also has basically uniform, neat and harmonious appearance, shape and color which are in line with the popular trend. In conclusion, in accordance with the specific operation methods for the intelligent positive and negative pressure fresh-keeping refrigerator, multiple methods, such as vacuum, high pressure, super oxygen, catalysts, air-conditioning fresh-keeping, negative ions, humidification, humidity and temperature control, disinfection and sterilization, air purification, air-to-water production, and low-temperature storage, are intelligently regulated and integrated under positive pressure or negative pressure, are organically applied to the positive and negative pressure fresh-keeping refrigerator, and are organically integrated and optimally matched. One item of or a combination of more items of the above techniques is used in individually or alternately or circularly, and multiple positive and negative pressure cabins with different types and different functions are arranged in a positive and negative pressure fresh-keeping compartment, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping refrigerator updates the existing method that the traditional refrigerator achieves low-temperature storage only by controlling the temperature of the refrigerator; the positive and negative pressure system accurately regulates and controls various fresh-keeping environment elements such as the airflow pressure, gas composition, air cleanliness, environmental temperature and humidity in the positive and negative pressure refrigerating-freezing cabin in real time, performs harmless disinfection and sterilization in the refrigerator, and rapidly degrades residual pesticides, fertilizers, hormones and harmful additives, achieve accurate positioning and targeted removal of fresh-loss factors, thus making various fresh foods stored in positive and negative pressure refrigerating-freezing cabin keep fresh and moist quality and flavor for a long time. The positive and negative pressure system endows the fresh-keeping refrigerator with breathing function and circulation characteristics, makes the cold fresh-keeping refrigerator come alive and become a life appliance, and upgrades the existing refrigerator and traditional methods. As the positive and negative pressure system has an ultra-large-capacity carrier and renewable performance and a platform advantage of upgrading, extending and adding items, the positive and negative pressure fresh-keeping refrigerator is endowed with the upgrading and expanding function. With the gradual development of science and technique, various newly developed methods of fresh-keeping and preservation are absorbed and integrated into the positive and negative pressure fresh-keeping refrigerator, making the positive and negative pressure fresh-keeping refrigerator innovate and develop as mobile phones and computers; and a chip processor and various software of the positive and negative pressure intelligent regulation and control apparatus are upgraded and updated gradually, making the positive and negative pressure fresh-keeping refrigerator more multifunctional, efficient, environmentally friendly, low-consumption, diverse and suitable for use.

Specific embodiment 5: an intelligent positive and negative pressure washing machine includes a machine body (7). The machine body (7) is internally provided with an intelligent positive and negative pressure system, a washing-dehydrating-drying system (15). The intelligent positive and negative pressure system includes a positive and negative pressure cabin (1), an air evacuation pump (2), an air inflation pump (3), a positive and negative pressure intelligent regulation and controlling apparatus (5), a positive and negative pressure waterflow carried substance generator integration (6.04), a carried substance decomposition processor (6.7), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water input pump (17) and the touch screen and mobile phone monitoring and identification system (12). The positive and negative pressure cabin (1) is designed as a washing machine type positive and negative pressure cabin (1F) according to particular requirements for washing and drying. The washing machine type positive and negative pressure cabin (1F) is a vacuum high pressure cabin (1-1) structure. The positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a washing machine configuration. The washing machine type positive and negative pressure cabin (1F) includes a cabin body (1.1), a cabin door (1.2), an airtight mechanism (1.3), and an internal and external communicating sealer (1.4). The airtight mechanism (1.3) is arranged between the cabin door (1.2) and the cabin body (1.1). The airtight mechanism (1.3) includes a lock ring (1.31), a lock bolt (1.32), an airtight gasket (1.33), and a door rim sealing gasket (1.34); the lock ring (1.31) is in movable fit with the lock bolt (1.32), the lock bolt (1.32) extends into the lock ring (1.31) to lock the cabin door during the closing of the cabin door; when the positive and negative atmospheric pressure is formed inside the washing machine type positive and negative pressure cabin (1F), the airtight gasket (1.33) is used for keeping sealing. The internal and external communicating sealer (1.4) is fixedly arranged at a rear part or side part of washing machine type positive and negative pressure cabin (1F), and all pipes and circuits getting in and out the cabin are connected to and pass through the internal and external communicating sealer (1.4) so as to keep the sealing property of the washing machine type positive and negative pressure cabin (1F). The positive and negative pressure intelligent regulation and control apparatus (5) includes an integrated circuit and chip, and a regulation and control system for a fluid and a carried substance thereof, and is connected to the Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), a carried substance generation item addition and upgrading module (6.16). The carried substance generation item addition and upgrading module (6.16) is a combined module capable of providing or installing any individual fluid carried substance generator additional item in the carried substance generator integration and upgrading and updating the same. The waterflow carried substance generator integration (6.04) includes at least one or more of individual fluid carried substance generators (6.1) to (6.16) as required. The washing-dehydrating-drying system (15) includes a washing machine drum mechanism (15.1), a washing-dehydrating-drying control mechanism (15.2), a drum sealing bearing housing (15.3), a drum big belt pulley (15.4), and a motor assembly (15.5). The washing machine type positive and negative pressure cabin (1F) is internally provided with the washing machine drum mechanism (15.1), an anti-shower air evacuation port (2.1), the drum sealing bearing housing (15.3), a pressure sensor (C1), a negative pressure sensor (C2), a super oxygen sensor (C3), and a carried substance sensor item addition and upgrading module (C12). The carried substance sensor item addition and upgrading module (C12) is a combined module capable of providing or installing any individual sensor additional item in the sensor integration and upgrading and updating the same. The washing machine type positive and negative pressure cabin (1F) is externally provided with the air evacuation pump (2), the air inflation pump (3), the intelligent positive and negative pressure regulation and control apparatus (5), the positive and negative pressure waterflow carried substance generator integration (6.04), the carried substance decomposition processor (6.7), the washing-dehydrating-drying control mechanism (15.2), the drum big belt pulley (15.4), the motor assembly (15.5), the water suction pump (16), the water input pump (17), a detergent pull box (18), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the washing machine type positive and negative pressure cabin (1F) and then is connected with the anti-shower air evacuation port (2.1) to form an air evacuation and air return port (c) or a circulation air return port (i), and an air outlet port (b) of the air evacuation pump (2) is connected to the atmosphere. A circulation pipeline (A5) is further connected to the solenoid valve (B1), a solenoid valve (B5) is installed at a middle part of the circulation pipeline (A5), and the other end of the circulation pipeline (A5) is a circulation exhaust port (i2) or a circulation air inlet port (j2). An air inflation pipeline (A3) is installed at an air outlet port (d) of the air inflation pump (3), a solenoid valve (B3) is installed at a middle part of the air inflation pipeline (A3), the other end of the air inflation pipeline (A3) extends into washing machine type positive and negative pressure cabin (1F) to form an air inflatable inlet port (f) or a circulation air inlet port (j), and an air inlet port (e) of the air inflation pump (3) is connected to the atmosphere. A circulation pipeline (A6) is further connected to the solenoid valve (B3), a solenoid valve (B6) is installed at a middle part of the circulation pipeline (A6), and the other end of the circulation pipeline (A6) is connected to a circulation air inlet port (e2) of the air inflation pump (3). A circulation pipeline (A7) is further connected to the solenoid valve (B6), and the other end of the circulation pipeline (A7) is connected to the solenoid valve (B5). A water intake pipeline (A14) is installed at a water inlet port (r) of the water input pump (17), a solenoid valve (B14) is installed at a middle part of the water intake pipeline (A14), and the other end of the water intake pipeline (A14) communicates with a tap water source. A washing machine water intake pipeline (A15) is installed at a water output port (u) of the water input pump (17), a solenoid valve (B15) is installed at a middle part of the water intake pipeline (A15), and the other end of the water intake pipeline (A15) communicates with a water inlet port (v) of the positive and negative pressure waterflow carried substance generator integration (6.04). A washing machine water intake pipeline (A16) is installed at a water output port (o) of the water input pump (17), a solenoid valve (B16) is installed at a middle part of the water intake pipeline (A16), and the other end of the water intake pipeline (A16) communicates with a water inlet port (z) of the detergent pull box (18). A washing machine water intake pipeline (A17) is installed at a water output port (1) of the detergent pull box (18), a solenoid valve (B17) is installed at a middle part of the water intake pipeline (A17), and the other end of the water intake pipeline (A17) extends into the washing machine type positive and negative pressure cabin (1F) to form a water inlet port (T) thereof. A water intake pipeline (A18) is further installed on the solenoid valve (B14), and the solenoid valve (B14) communicates with the solenoid valve (B16) so as to directly feed water when carrying water is not needed and the tap water pressure reaches the standard. A water intake pipeline (A19) is further installed on the solenoid valve (B15), and the solenoid valve (B15) is connected to the water intake pipeline (A18) to communicate with the solenoid valve (B16), such that the water is directly fed by the water input pump (17) when the carrying water is not needed. A water drainage pipeline (A20) is installed at a water output port (w) of the water suction pump (16), a solenoid valve (B20) is installed at a middle part of the water drainage pipeline (A20), and the other end of the water drainage pipeline (A20) extends into the washing machine type positive and negative pressure cabin (1F) to form a water drainage port (L) thereof. A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) is connected to a sewer. A water drainage pipeline (A23) is installed at the solenoid valve (B20), and the solenoid valve (B20) communicates with the solenoid valve (B22), such that the water is directly drained when the carried substance does not need to be decomposed and free drainage is achieved. A water drainage pipeline (A24) is further installed on the solenoid valve (B21), and the solenoid valve (B21) is connected to the water drainage pipeline (A23) and then communicates with the solenoid valve (B22), such that the water is directly drained by the water suction pump (16) when the carried substance does not need to be decomposed; the big belt pulley (15.4) is driven by the motor assembly (15.5), and the big belt pulley (15.4) drives the drum mechanism (15.1) connected thereto to operate. All fluid carried substance generation processors (6), the sensors (C), the solenoid valves (B), and the air evacuation pump (2), the air inflation pump (3), the washing-dehydrating-drying control mechanism (15.2), the water suction pump (16), the water input pump (17), and the touch screen and mobile phone monitoring and identification system (12) are all connected to the positive and negative pressure intelligent regulation and control apparatus (5).

Specific embodiment 6: an operation method for an intelligent positive and negative pressure washing machine is provided. The intelligent positive and negative pressure washing machine adopts an intelligent positive and negative pressure system. The intelligent positive and negative pressure system is used to accurately regulate and organically integrate various techniques such as vacuum, high pressure, super oxygen, disinfection and sterilization, degradation treatment, and clothes cleaning and drying, and one item of or a combination of more items of the techniques is applied to the positive and negative pressure washing machine, a vacuum and high pressure airflow-waterflow stain removal drying technique is developed, and digital techniques such as Internet, big data, cloud computing, machine learning, artificial intelligence and expert system are also applied to the washing machine at the same time to analyze and identify stain-causing elements and control targets of various different cleaning materials one by one, so as to form a complete large database according to different categories, and correspondingly use different targeted stain removal techniques. According to built-in program and pre-stored data of the positive and negative pressure washing machine, and real-time feedback information of various sensors, various washing-dehydrating-drying elements, such as waterflow and airflow pressure, gas composition, water temperature and air temperature, are regulated and controlled in real time, so as to disinfect, sterilize and degrade the cleaning materials harmlessly, and create a regulatable and controllable accurate washing-dehydrating-drying intelligent microenvironment with the optimal effect, shortest time, highest efficiency, minimum wear and least environmental pollution. A personalized targeted stain removal comprehensive washing-drying method is designed at the molecular and microscopic levels for stain-causing sites of the cleaning material and a contaminant, so as to accurately removal the stain-causing factors at fixed points, while avoiding or reducing the damage to the structure and color of the cleaning materials, and thus a set of digital expert system for clothes cleaning and drying is formed and gradually improved, which is intelligently applied to the positive and negative pressure washing machine to achieve the purpose of fast, efficient, sanitary and environmental-friendly cleaning. A specific operation method for the intelligent positive and negative pressure washing machine is that the positive and negative pressure system intelligently regulates and controls the airflow pressure by an air evacuation pump or an air inflation pump, and carries the effective loads of super oxygen, a disinfectant, a cleaning builder, a softening and bulking agent and a decomposition treatment agent by the airflow or waterflow to get in, stay in, or get out a positive and negative pressure cabin (1F) orderly, and the following methods are used to apply various action forces helpful for rapid and efficient cleaning and drying of the laundry: (1) positive atmospheric pressure high pressure action method: A: during washing, turning on the air inflation pump (3) at proper time to inflate the positive and negative pressure cabin (1F) by means of an air inflation pipeline (A3), so as to form strong bubbles and rapid waterflow to participate in clothes cleaning, speed up the washing process and improve the washing efficiency, where the high atmospheric pressure formed in the cabin is beneficial for a detergent to penetrate into the clothes to facilitate the cleaning; B: during water drainage, inflating and pressurizing the positive and negative pressure cabin (1F) to speed up the water drainage speed; C: during dehydration, inflating and pressurizing to force water to quickly separate from the clothes, so as to improve dehydration efficiency and shorten water drainage and dehydration time; D: at the beginning of a drying procedure, inflating and pressurizing the positive and negative pressure cabin (1F) of the washing machine, which is beneficial for hot air to enter clothes fibers to vaporize water, and then releasing pressure rapidly to drain the water vapor to accelerate the drying process and effectively shorten the drying time; (2) negative atmospheric pressure vacuum action method: A, during water intake, turning on the air evacuation pump (2) to pump the positive and negative cabin (1F) to negative pressure by means of an air evacuation pipeline (A1), so as to speed up water intake and shorten water intake time; B: at the beginning of washing, evacuating the positive and negative cabin (1F) to a moderate vacuum, where both the clothes fibers and stains swell in the moderate vacuum, the adhesive force of the stains is weaken or the stains fall off, such that the cleaning efficiency is improved, and the washing time is shortened; C: during washing, circulating and repeating air evacuation and air inflation for alternate fiber swelling and turbulent rubbing, thus improving cleaning degree and clothes washing efficiency, and accelerating washing and rinsing progress; D: during dehydration, evacuating the positive and negative pressure cabin to the negative pressure to make the clothes fibers swell, where the water in the clothes escape to the negative pressure space and is pumped out of the cabin, making the dehydrated and hardened clothes fluff, and circulating and repeating the air evacuation and air inflation to force the water out and pump the water out of the cabin alternately, thus greatly improving the clothes dehydration efficiency and shortening the water drainage and dehydration time; E: carrying out air evacuation at proper time during drying, evacuating the water, and making the clothes fibers swell by the negative pressure to facilitate quick drying of water vapor, where, with the repeated circulation of air inflation, heating gasification and water evacuation are alternately conducted, which greatly accelerates the clothes drying progress, and the negative pressure makes the clothes fluff, which improves the clothes washing and drying quality and effectively shortens the clothes drying time; (3) airflow and waterflow carried substance action method: carrying effective loads of super oxygen, a disinfectant, a cleaning builder, a softening and bulking agent and a decomposition treatment agent by the airflow or waterflow, where a single or a combination of the plurality of effective loads gets in, stays in, or gets out the washing machine type positive and negative pressure cabin (1F) orderly, so as to apply various action forces beneficial for fast and efficient cleaning and drying of the laundry; in the intelligent positive and negative pressure system, a positive and negative pressure intelligent regulation and control apparatus (5) is used to analyze and identify stain-causing factors and stain removal targets of various different cleaning scenarios and cleaning clothes one by one according to built-in program, pre-stored data and internet cloud data of the positive and negative pressure intelligent regulation and control apparatus (5), and real-time feedback information of a touch screen and mobile phone monitoring and identification system (12) and various sensors (C1) to (C12), and after computing, different accurate stain removal techniques are correspondingly used to form targeted stain removal comprehensive cleaning methods with different characteristics, and the positive and negative pressure intelligent regulation and control apparatus (5) is used to send specific instructions to the air evacuation pump (2), the air inflation pump (3), related positive and negative pressure fluid carried substance generators (6) and related solenoid valves (B) in real time to accurately control and adjust open, close and switching of various air evacuation, inflation and circulation pipelines and intelligently regulate and control the level of the positive and negative pressure in the positive and negative pressure cabin (1) and the flowing-in, staying and flowing-out of a fluid and a carried substance thereof, so as to remove the stain-causing factors of the cleaning materials at fixed points and accurately maintain fragile elements of the cleaning materials to achieve the efficient and low-consumption cleaning effect. The specific operation methods for the intelligent positive and negative pressure system targeted stain removal comprehensive washing and drying and the positive and negative pressure washing machine are as follows: (1) water intake procedure: placing clothes into a drum (15.1), closing a cabin door (1.2) to seal and lock the washing machine type positive and negative pressure cabin (1F) after the washing machine is turned on; sending an instruction by the positive and negative pressure intelligent regulation and control apparatus (5) to enable solenoid valves (B14), (B15), (B16) and (B17) to open the corresponding water intake pipelines: (i) direct tap water intake line being: (A14)→(B14)→(A18)→(B16)→(A16)→water inlet port (z) of detergent pull box→water output port (1)→(A17)→(B17)→(A17)→water inlet port (T) of positive and negative pressure cabin; (ii) when washing-aid carried substance is required for washing, a water intake line being: (A14)→(B14)→(A14)→water inlet port (r) of water input pump→water output port (u)→(A15)→(B15)→(A15)→water inlet port (v) of waterflow carried substance generator integration→water output port (o)→(A16)→(B16)→(A16)→detergent pull box (z)→water output port (1)→(A17)→(B17)→(A17)→water inlet port (T) of positive and negative pressure cabin; (iii) when washing-aid carried substance is not required for cleaning, a water intake line being: (A14)→(B14)→(A14)→water inlet port (r) of water input pump→water output port (u)→(A15)→(B15)→(A19)→(A18)→(B16)→(A16)→water inlet port (z) of detergent pull box→water output port (1)→(A17)→(B17)→(A17)→water inlet port (T) of positive and negative pressure cabin, during the procedures (ii) and (iii), turning on the water input pump (17), directly feeding the tap water accordingly, or by the waterflow carried substance generator integration (6.04), rapidly mixing the tap water with the washing aid carried substance generated by waterflow carried substance generator integration (6.04), and then enabling the mixed water to enter the washing machine type positive and negative pressure cabin (1F), where the super-oxygen or carried substance mixed water can accelerate the decomposition of organic fouling on the clothes, thus facilitating the rapid cleaning; after the water intake is started, enabling, by the positive and negative pressure intelligent regulation and control apparatus (5), a solenoid valve (B1) to open an air evacuation pipeline (A1), air inlet port (a) of air evacuation pump→(A1)→(B1)→(A1)→air evacuation and air return port (c) of positive and negative pressure cabin, and turning on the air evacuation pump (2) to make the washing machine type positive and negative pressure cabin (1F) in a negative pressure, thus accelerating the water intake and shortening the time for water intake; (2) vacuum washing procedure: after the water intake is finished, turning on, by the positive and negative pressure intelligent regulation and control apparatus (5), a motor assembly (15.5) to drive the drum mechanism (15.1) to rotate for washing, and meanwhile, enabling the solenoid valve (B1) to open the air evacuation pipeline (A1), air inlet port (a) of air evacuation pump→(A1)→(B1)→(A1)→air evacuation and air return port (c) of positive and negative pressure cabin, and turning on the air evacuation pump (2) to pump the washing machine type positive and negative pressure cabin (1F) to a moderate vacuum, where both the clothes fibers and stains swell in the moderate vacuum and the air therein escapes, such that the adhesive force of the stains is weaken or the stains fall off, the cleaning efficiency is improved, and the washing time is shortened, and during the laundry process, the super oxygen or carried substance mixed water directly kills bacteria and viral microorganisms on clothes, the organic matters in the dust and fouling on the clothes are decomposed and then dissolved into the water, the stain removability of the detergent is enhanced, the degree of cleaning is improved, the cleaning process is accelerated, and the function of sterilization and deodorization is achieved at the same time; (3) high-pressure washing procedure: after washing at vacuum for proper time, closing the solenoid valve (B1) and turning off the air evacuation pump (2) by the positive and negative pressure intelligent regulation and control apparatus (5), enabling the solenoid valve (B3) to open an air inflation pipeline (A3): air outlet port (d) of air inflation pump→(A3)→(B3)→(A3)→air inflation port (f) of positive and negative pressure cabin, and opening circulation pipelines (A1), (A5) and (A6) at the same time: (i) atmospheric circulation: circulation air return port (i) of positive and negative pressure cabin→(A1)→(B1)→(A5)→(B5)→(A5)→circulation exhaust port (i2)→exhaust to atmosphere for circulation; or (ii) in-machine circulation: circulation air return port (i) of positive and negative pressure cabin→(A1)→(B1)→(A5)→(B5)→(A7)→(B6)→(A6)→circulation air return port (e2) of air inflation pump, and turning on the air inflation pump (3), enabling high pressure airflow to enter the washing machine type positive and negative pressure cabin (1F) through an air inflation pipeline (A3), so as to form strong bubbles and rapid waterflow in the cabin to participate in clothes cleaning, where the high atmospheric pressure formed in the washing machine type positive and negative pressure cabin (1F) is beneficial for the detergent to penetrate into the clothes to facilitate the cleaning, and by circulating the air evacuation and air inflation repeatedly, and vacuum and high pressure are circulated and repeated, leading to fiber swell and turbulent rubbing, so as to greatly improve the cleaning degree and laundry efficiency and accelerate the washing and rinsing progress; (4) water drainage and dehydration procedure: sending an instruction by the positive and negative pressure intelligent regulation and control apparatus (5) when the water drainage is required, enabling solenoid valves (B20), (B21) and (B22) to open the corresponding water drainage pipelines: (i) when both the water suction pump and the decomposition of super oxygen and carried substance are not required, a direct water drainage line being: water drainage port (L) of washing machine type positive and negative pressure cabin→(A20)→(B20)→(A23)→(B22)→(A22)→sewer; (ii) when both the water suction pump for drainage and the decomposition of super oxygen are required, a water drainage line being: water drainage port (L) of positive and negative pressure cabin→(A20)→(B20)→(A20)→water inlet port (w) of water suction pump→water output port (p)→(A21)→(B21)→(A21)→water inlet port (g) carried substance decomposition processor→water output port (m)+(A22)→(B22)→(A22)→sewer; (iii) when the water suction pump is required for drainage and the decomposition of super oxygen and carried substance is not required, a water drainage line being: a water drainage port (L) of washing machine type positive and negative pressure cabin→(A20)→(B20)→(A20)→water inlet port (w) of water suction pump→water output port (p)→(A21)→(B21)→(A24)→(A23)→(B22)→(A22)→sewer, during the procedures (ii) and (iii), turning on the water suction pump (16) for water drainage, during water drainage, turning on the air inflation pump (3) to inflate and pressurize the washing machine type positive and negative pressure cabin (1F), thus accelerating the water drainage speed; after the water drainage is finished, sending an instruction by the positive and negative pressure intelligent regulation and control apparatus (5) to start dehydration; during dehydration, inflating and pressurizing to force the water to rapidly separate from the clothes; turning on the air evacuation pump (2) alternately to pump the cabin to a negative pressure to make the clothes fibers in the washing machine type positive and negative pressure cabin (1F) swell, where the water in the clothes escapes to a negative pressure space and is pumped out of the cabin, such that the clothes hardened on an inner wall of the drum during dehydrating and high pressure are loosened to prevent wrinkles, and by circulating the air evacuation and air inflation repeatedly, squeezing out water and evacuating the water out the cabin are carried out alternately, the efficiency of dehydrating the clothes is greatly improved, and time for water drainage and dehydration is shortened; (5) vacuum high pressure drying procedure: when a drying procedure is started, turning on the air inflation pump (3) according to an instruction sent by the positive and negative pressure intelligent regulation and control apparatus (5), and opening an air inflation pipeline (A3) to inflate and pressurize the washing machine type positive and negative pressure cabin (1F), thus facilitating hot air to enter the clothes fibers to gasify the water, turning off the air inflation pump (3) and closing the air inflation pipeline (A3), turning on the air evacuation pump (2) and opening an air evacuation pipeline (A1) to pump the water out, and opening circulation pipelines (A5), (A7), (A6) and (A3): circulation air inlet port (j2)→(A5)→(B5)→(A7)→(B6)→(A6)→(B3)→(A3)→circulation air inlet port (j) of the positive and negative pressure cabin, evacuating out the water and making the clothes fibers swell with the negative pressure to facilitate the water emission and quick drying of the clothes, such that, by circulating the air evacuation and inflation repeatedly, heating gasification and water pump-out are alternately conducted, the drying progress is greatly accelerated, the fluffy clothes improves the drying quality, and the drying time is effectively shortened; (6) vacuum self-cleaning sterile placement procedure: closing the cabin door (1.2) after the laundry is finished, controlling, by the positive and negative pressure intelligent regulation and control apparatus (5), the washing-dehydrating-drying control mechanism to start drying hot air to blow-dry the inside of the washing machine, especially the drum mechanism (15.1), and then turning on the air evacuation pump (2) and opening the air evacuation pipeline for air evacuation, evacuating out the residual super oxygen and carried substance in the washing machine type positive and negative pressure cabin (1F), and enabling the drum mechanism (15.1) and the equipment in the cabin to be in moderate vacuum, such that the drum mechanism and the equipment in the cabin are sterile, dust-free and pollution-free for keeping clean, and oxidation-free, rust-free and undamaged for vacancy, and the service life of the washing machine is prolonged; in conclusion, the positive and negative pressure washing machine greatly accelerates the multiple processes of washing, dehydrating and drying, shortens the operation duration of the various procedures, and improves the cleaning degree of laundry and drying quality, moreover, the super oxygen also provides a thorough sterilization, disinfection and odor removal for the washed clothes; and meanwhile, the concentration and operation duration of the super oxygen and carried substance are intelligently controlled at the moderate region for sterilization, degradation and washing assistance, not reach, or as little as possible to reach, a critical point of possible oxidation fading of dark clothes; and (7) positive and negative pressure module combined intelligent smart home mode: designing and manufacturing the intelligent positive and negative pressure washing machine according to the module functions, where the intelligent positive and negative pressure washing machine not only is independently produced and used as a separated invention, but also is organically combined with other positive and negative pressure electric appliances and the module cabinets under the control of the touch screen and mobile phone monitoring and identification system (12) to form a fully functional intelligent positive and negative pressure module combined home, which not only can share the positive and negative pressure system, but also has basically uniform, neat and harmonious appearance, shape and color which are in line with the popular trend; in conclusion, targeted stain removal comprehensive washing and drying effective methods are used in the intelligent positive and negative pressure system, which make the washing, dehydrating and drying of clothes faster, more efficient and low-consumption, and the washed clothes less in consumption, cleaner, fluffier, and more sanitary.

Specific embodiment 7: an intelligent positive and negative pressure dish washing and fruit and vegetable cleaning machine includes a machine body (7). The machine body (7) is internally provided with an intelligent positive and negative pressure system, a dish washing and drying system (19), and a fruit and vegetable cleaning system (20). The intelligent positive and negative pressure system includes a positive and negative pressure cabin (1), an air evacuation pump (2), a positive and negative pressure intelligent regulation and control apparatus (5), waterflow carried substance generator integration (6.04), a carried substance decomposition processor (6.7), a water suction pump (16), a water input pump (17), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), the water suction pump (16), the water inlet pump (17), and the touch screen and mobile phone monitoring and identification system (12). The positive and negative pressure cabin (1) is designed as a dish washer type positive and negative pressure cabin (1G) according to intelligent dish washing and drying and fruit and vegetable cleaning requirements, and the dish washer type positive and negative pressure cabin (1G) is a vacuum high pressure cabin (1-1) structure. A positive and negative resistance standard of the vacuum high pressure cabin (1-1) takes the local instantaneous atmospheric pressure as zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the dish washing and fruit and vegetable cleaning machine. The dish washer type positive and negative pressure cabin (1G) includes a cabin body (1.1), a cabin door (1.2), an airtight mechanism (1.3), and an internal and external communicating sealer (1.4), and the airtight mechanism (1.3) is arranged between the cabin door (1.2) and the cabin body (1.1) of the dish washer type positive and negative pressure cabin (1G). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When positive and negative atmospheric pressure is formed inside the dish washer type positive and negative pressure cabin (1G), the airtight gasket (1.33) is used for keeping sealing. The internal and external communicating sealer (1.4) is fixedly arranged at a rear part or side part of the dish washer type positive and negative pressure cabin (1G), and all pipes and circuits getting in and out the cabin are connected to and pass through the internal and external communicating sealer (1.4), so as to keep the sealing property of the dish washer type positive and negative pressure cabin (1G). The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), and a carried substance generator item addition and upgrading module (6.16). The carried substance generator item addition and upgrading module (6.16) is a combined module capable of providing or installing any individual fluid carried substance generator additional item in the carried substance generator integration and upgrading and updating the same. The dish washer type positive and negative pressure cabin (1G) is internally provided with a dish washing and drying system (19), a fruit and vegetable cleaning system (20), an anti-shower air evacuation port (2.1), a negative pressure sensor (C2), a super oxygen sensor (C3), a temperature sensor (C9), and a carried substance sensor item addition and upgrading module (C12). The carried substance sensor item addition and upgrading module (C12) is a combined module capable of providing or installing any individual sensor additional item in the sensor integration and upgrading and updating the same. The dish washer type positive and negative pressure cabin (1G) is externally provided with an air evacuation pump (2), a positive and negative pressure intelligent regulation and control apparatus (5), a waterflow carried substance generator integration (6.04), a carried substance decomposition processor (6.7), a water suction pump (16), a water input pump (17), and a touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), the other end of the air evacuation pipeline (A1) extends into the dish washer type positive and negative pressure cabin (1G) and then is connected with the anti-shower air evacuation port (2.1), so as to form an air evacuation and air return port (c), and an air outlet port (b) of the air evacuation pump (2) is connected to the atmosphere. A water intake pipeline (A14) is installed at a water inlet port (r) of the water input pump (17), a solenoid valve (B14) is installed at a middle part of the water intake pipeline (A14), and the other end of the water intake pipeline (A14) communicates with a tap water source. A water intake pipeline (A15) is installed at a water output port (u) of the water input pump (17), a solenoid valve (B15) is installed at a middle part of the water intake pipeline (A15), and the other end of the water intake pipeline (A15) communicates with a water inlet port (k) of the waterflow carried substance generator integration (6.04). A water intake pipeline (A16) is installed at a water output port (q) of the waterflow carried substance generator integration (6.04), a solenoid valve (B16) is installed at a middle part of the water intake pipeline (A16), and the other end of the water intake pipeline (A16) extends into the dish washer type positive and negative pressure cabin (1G) to be connected to a solenoid valve (B25). The solenoid valve (B25) is respectively connected to a fruit and vegetable cleaning water intake pipeline (A25) and a dish washing and drying water intake pipeline (A27), and the water intake pipeline (A25) is connected to a water inlet port (e) of the fruit and vegetable cleaning system (20). The water intake pipeline (A27) is connected to a water inlet port (G) of the dish washing and drying system (19); a water intake pipeline (A19) is further installed at the solenoid valve (B15), and the solenoid valve (B15) is connected to the solenoid valve (B16), such that the water is directly fed by the water input pump (17) when super-oxygenated water is not required. A water drainage pipeline (A20) is installed at a water inlet port (w) of the water suction pump (16), a solenoid valve (B20) is installed at a middle part of the water drainage pipeline (A20), and the other end of the water drainage pipeline (A20) extends into the dish washer type positive and negative pressure cabin (1G) and is respectively connected to a fruit and vegetable cleaning water drainage pipeline (A26) and a dish washing and drying water drainage pipeline (A28), and the water drainage pipeline (A26) is connected to a water output port (F) of the fruit and vegetable cleaning system (20). The water drainage pipeline (A28) is connected to a water drainage port (H) of the dish washing and drying system (19). A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) communicates with a sewer. A water drainage pipeline (A24) is further installed at the solenoid valve (B21), and the solenoid valve (B21) is connected to (A23) and then communicates with the solenoid valve (B22), such that the water is directly drained by the water suction pump when the super oxygen and the carried substance do not need to be decomposed. All fluid carried substance generation processors (6), sensors (C) and solenoid valves (B) and the air evacuation pump (2), the air inflation pump (3), the water suction pump (16), the water input pump (17), the dish washing and drying system (19), the fruit and vegetable cleaning system (20), and the touch screen and mobile phone monitoring and identification system (12) are all connected to the positive and negative pressure intelligent regulation and control apparatus (5).

Specific embodiment 8: an operation method for an intelligent positive and negative pressure dish washing and fruit and vegetable cleaning machine is provided. The intelligent positive and negative pressure dish washing and fruit and vegetable cleaning machine adopts an intelligent positive and negative pressure system. The intelligent positive and negative pressure system is used to accurately regulate and organically integrate various techniques such as vacuum, super oxygen, disinfection and sterilization, degradation treatment, dish washing, and fruit and vegetable cleaning, and one item of or a combination of more items of the techniques is applied to the intelligent positive and negative pressure dish washing and fruit and vegetable cleaning machine, a vacuum and high pressure airflow-waterflow dish washing and fruit and vegetable cleaning and drying technique is developed, and digital techniques such as Internet, big data, cloud computing, machine learning, artificial intelligence and expert system are also applied to the dish washing and fruit and vegetable cleaning machine at the same time to analyze and identify stain-causing elements and control targets of various different cleaning materials one by one, so as to form a complete large database according to different categories, and correspondingly use different targeted stain removal techniques. According to built-in program and pre-stored data of the positive and negative pressure dish washing and fruit and vegetable cleaning machine, and real-time feedback information of various sensors, various dish washing and fruit and vegetable cleaning and drying elements, such as waterflow and airflow pressure, gas composition, water temperature and air temperature, are regulated and controlled in real time, so as to disinfect, sterilize and degrade the cleaning materials harmlessly, and create a regulatable and controllable accurate dish washing and fruit and vegetable cleaning and drying intelligent microenvironment with the optimal effect, shortest time, highest efficiency, minimum wear and least environmental pollution. For stain-causing sites of both cleaning materials and contaminant, stain-causing factors and stain removal targets of various different cleaning scenarios and cleaning materials are analyzed one by one on the molecular and microscopic level, and after computing, different accurate stain removal techniques are correspondingly used to form targeted stain removal comprehensive cleaning methods with different characteristics for removing the stain-causing factors at fixed points, so as to form a set of digital expert system for dish washing and fruit and vegetable cleaning and drying, which is intelligently applied to achieve the purpose of fast, efficient, sanitary and environmental-friendly dish washing and fruit and vegetable cleaning. A specific operation method for the intelligent positive and negative pressure dish washing and fruit and vegetable cleaning machine includes the following steps: (1) dish washing water intake and cleaning procedure: sending an instruction by the positive and negative pressure intelligent regulation and control apparatus (5) after the machine door is closed, enabling solenoid valves (B14), (B15), (B16) and (B25) to open corresponding water intake pipelines: (i) when the super oxygen or substance-carrying water is required for dish washing, water intake line of water input pump being: (A14)→(B14)→(A14)→water inlet port (r) of water input pump→water output port (u)→(A15)→(B15)→(A15)→water inlet port (k) of waterflow carried substance generator integration→water output port (q)→(A16)→(B16)→(A16)→(B25)→(A27)→water inlet port (G) of dish washing and drying system; (ii) when the super oxygen or substance-carrying water is not required for dish washing, water intake line of water input pump being: (A14)→(B14)→(A14)→water inlet port (r) of water input pump→water output port (u)→(A15)→(B15)→(A19)→(B16)→(A16)→(B25)→(A27)→water inlet port (G) of dish washing and drying system, turning on the water input pump (17), rapidly mixing, by the waterflow carried substance generator integration, the tap water with mixed super-oxygenated or substance-carrying water generated by the waterflow carried substance generator integration, and feeding the mixture into a dish washer type positive and negative pressure cabin (1G) to accelerate the decomposition of organic fouling on the tableware, thus facilitating the rapid cleaning; after the water intake is finished, turning on the dish washing and drying system (19) by the positive and negative pressure intelligent regulation and control apparatus (5) to spray water for dish washing, and enabling a solenoid valve (B1) to open an air evacuation pipeline (A1): air inlet port (a) of air evacuation pump→(A1)→(B1)→(A1)→air evacuation and air return port (c) of positive and negative pressure cabin, and turning on the air evacuation pump (2) for vacuumizing, where, during the vacuumizing, the stain stained on the tableware expands in the negative vacuum pressure to make air escape from the stain, thus the adhesive force of the stain is weakened, or the stain fall off from the tableware, the cleaning efficiency is improved, and the washing process is shortened, moreover, the mixed super-oxygenated or substance-carrying water directly kills the bacteria and viral microorganism on the tableware, the organic matters in the stain on the tableware are decomposed and then dissolved into the water, the stain removability of the detergent is enhanced, the degree of cleaning is improved, the cleaning process is accelerated, and the function of sterilization and deodorization is achieved at the same time; (2) dish washing drainage and drying disinfection procedure: when the water drainage is required at proper time of cleaning, sending an instruction by the positive and negative pressure intelligent regulation and control apparatus (5), enabling solenoid valves (B20), (B21) and (B22) to open the corresponding water drainage pipelines: (i) when both the water suction pump and the decomposition of super oxygen and carried substance are not required, a direct water drainage line being: water output port (H) of dish washing and drying system→(A28)→(A20)→(B20)→(A23)→(B22)→(A22)→sewer; (ii) when both the water suction pump for water drainage and the decomposition of super oxygen and carried substance are required, a water drainage line being: water output port (H) of dish washing and drying system→(A28)→(A20)→(B20)→(A20)→water inlet port (w) of water drainage pump→water output port (p)→(A21)→(B21)→(A21)→water inlet port (g) of carried substance decomposition processor→water output port (m)→(A22)→(B22)→(A22)→sewer; and (iii) when the water suction pump is required for water drainage and the super oxygen does not need to be decomposed, a water drainage line being: water output port (H) of dish washing and drying system→(A28)→(A20)→(B20)→(A23)→(B22)→(A22)→sewer, draining water directly or turning on the water suction pump 16, or turning on the carried substance decomposition decomposer (6.7) to decompose waste water and then drain the decomposed waste water into the sewer; meanwhile, spraying the tableware with clear water for cleaning completely; during the drying procedure, turning on, by the positive and negative pressure regulation and control apparatus (5), the air evacuation pump (2) again to rapidly pump out the water vapor so as to accelerate the drying progress and accelerate the dish washing time, where the super oxygen or carried substance also thoroughly sterilize, disinfect and removal order of the washed tableware; (3) fruit and vegetable cleaning water intake and cleaning procedure: sending an instruction by the positive and negative pressure intelligent regulation and control apparatus (5) after the machine door is closed, enabling solenoid valves (B14), (B15), (B16) and (B25) to open corresponding water intake pipelines: (i) when the super-oxygenated water is required for fruit and vegetable cleaning, a water intake line for the water input pump being: (A14)→(B14)→(A14)→water inlet port (r) of water input pump→water output port (u)→(A15)→(B15)→(A15)→water inlet port (k) of super oxygen generation water mixer→water output port (q)→(A16)→(B16)→(A16)→(B25)→(A25)→water inlet port (e) of fruit and vegetable cleaning system; (ii) when the super-oxygenated water is not required for fruit and vegetable cleaning, a water intake line for the water input pump being: (A14)→(B14)→(A14)→water inlet port (r) of water input pump→water output port (u)→(A15)→(B15)→(A19)→(B16)→(A16)→(B25)→(A25)→water inlet port (e) of fruit and vegetable cleaning system, and turning on the water input pump (17), rapidly mixing, by the waterflow carried substance generator integration (6.04), the tap water with mixed super-oxygenated or substance-carrying water generated by the waterflow carried substance generator integration, and feeding the mixture into the dish washer type positive and negative pressure cabin (1G); after the water intake is finished, turning on the fruit and vegetable cleaning system (19) by the positive and negative pressure intelligent regulation and control apparatus (5) to spray water for fruit and vegetable cleaning, and enabling the solenoid valve (B1) to open the air evacuation pipeline (A1): air inlet port (a) of air evacuation pump→(A1)→(B1)→(A1)→air evacuation and air return port (c) of positive and negative pressure cabin, and turning on the air evacuation pump (2) for vacuumizing, where, during the vacuumizing, the stain stained on the fruit and vegetable expands in the negative vacuum pressure to make air escape from the stain, thus the adhesive force of the stain is weakened, or the stain fall off from the tableware, the cleaning efficiency is improved, and the washing process is shortened, moreover, the mixed super-oxygenated or substance-carrying water directly kills the bacteria and viral microorganism on the skin of the fruits and vegetables, the organic matters in stain on skin of the fruits and vegetables are decomposed and then dissolved into the water, the cleaning degree is improved, and the cleaning process is accelerated; and meanwhile, the negative pressure promotes the escape of pesticide and heavy metal residues in the tissues of the fruits and vegetables and the harmful gases such as the volatile metabolites ethylene acetaldehyde ethanol, thus a favorable environment is created for the super-oxygenated water to rapidly clean and deeply degrade the pesticide residues and decompose the harmful gases such as ethylene; sending an instruction by the positive and negative pressure intelligent regulation and control apparatus (5) after cleaning by the mixed super-oxygenated or substance-carrying water, and spraying and cleaning the fruits and vegetables with clear water; (4) water drainage procedure after fruit and vegetable cleaning: when the water drainage is required after cleaning for proper time, sending an instruction by the positive and negative pressure intelligent regulation and control apparatus (5), enabling solenoid valves (B20), (B21) and (B22) to open corresponding water drainage pipelines: (i) when both the water suction pump and the decomposition of super oxygen or carried substance are not required, a direct water drainage line being: water output port (F) of fruit and vegetable cleaning system→(A26)→(A20)→(B20)→(A23)→(B22)→(A22)→sewer; (ii) when both the water suction pump for water drainage and the decomposition of super oxygen and carried substance are required, a water drainage line being: water output port (F) of fruit and vegetable cleaning system→(A26)→(A20)→(B20)→(A20)→water inlet port (w) of water suction pump→water output port (p)→(A21)→(B21)→(A21)→water output port (g) of carried substance decomposition processor→water output port (m)→(A22)→(B22)→(A22)→sewer; (iii) when the water suction pump is required for water drainage and the super oxygen or carried substance does not need to be decomposed, a water drainage line being: water output port (F) of fruit and vegetable cleaning system→(A26)→(A20)→(B20)→(A23)→(B22)→(A22)→sewer, directly draining water or turning on the water suction pump (16) or turning on the carried substance decomposition processor (6.7) again to decompose waste water, and then draining the decomposed waste water into the sewer; (5) vacuum self-cleaning sterile placement sterile: closing the machine door (1.2) after use, starting drying hot air by the positive and negative pressure intelligent regulation and control apparatus (5) to blow-dry the inside of the dish washer type positive and negative pressure cabin (1G), especially the dish washing and drying system (19) and the fruit and vegetable cleaning system (20), and then turning on the air evacuation pump (2) and opening the air evacuation pipeline for air evacuation, evacuating out the residual super oxygen or carried substance in the dish washer type positive and negative pressure cabin (1G), and enabling the dish washing and drying system (19), the fruit and vegetable cleaning system (20) and the equipment in the cabin to be in a moderate vacuum, such that the dish washing-drying system (19) and the fruit and vegetable cleaning system (20) and the equipment in the cabin are sterile, dust-free, pollution-free, and kept clean for health, and are oxidation-free, rust-free and undamaged for vacancy, and the service life of the electric appliance is prolonged; and (6) positive and negative pressure module combined intelligent smart home mode: designing and manufacturing the intelligent positive and negative pressure dish washing and fruit and vegetable cleaning machine according to the module functions, where the intelligent positive and negative pressure dish washing and fruit and vegetable cleaning machine not only is independently produced and used as a separated invention, but also is organically combined with other positive and negative pressure electric appliances and the module cabinets under the control of the touch screen and mobile phone monitoring and identification system (12) to form a fully functional intelligent positive and negative pressure module combined home, which not only shares the water input pump (17), the water suction pump (16), the waterflow carried substance generator integration (6.04) and the carried substance decomposition processor (6.7), but also has basically uniform, neat and harmonious appearance, shape and color which are in line with the popular trend, such that the dish washing and fruit and vegetable cleaning machine is more efficient, low-consumption, and suitable for use.

Specific embodiment 9: an intelligent positive and negative pressure range hood includes a machine body (7). A lower half part of the machine body (7) is a range hood type positive and negative pressure cabin (1H), and an upper half part of the machine body (7) is an equipment fume tube cabin (21). The equipment fume tube cabin (21) is internally provided with a positive and negative pressure system, a fume evacuation control mechanism (26), and a water heating apparatus (27). The positive and negative pressure system includes: a positive and negative pressure intelligent regulation and control apparatus (5), a water input pump (17), a water suction pump (16), a waterflow carried substance generator integration (6.04), and a carried substance decomposition processor (6.7). The range hood type positive and negative pressure cabin (1H) is an atmospheric circulation cabin (1-2) structure, including a triangular cabin body (1.1) and a cabin door (1.2). The cabin door (1.2) is a machine door of the positive and negative pressure range hood, and a waterproof mechanism (1.7) is arranged between the cabin door (1.2) and the cabin body (1.1). The waterproof mechanism (1.7) includes a lock ring (1.71), a lock bolt (1.72), an airtight gasket (1.73), and a cabin door hinge (1.74). The lock ring (1.71) is in movable fit with the lock bolt (1.72), the lock bolt (1.72) extends into the lock ring (1.71) to lock the cabin door (1.2) during the closing of the cabin door. When spraying and cleaning are conducted in the range hood type positive and negative pressure cabin (1H), the waterproof gasket (1.73) is used for keeping sealing. The positive and negative pressure intelligent regulation and control apparatus (5) includes an integrated circuit, a chip, and a regulation and control system for a fluid and a carried substance thereof, and is connected to an Internet in a wired or wireless mode. The positive and negative pressure waterflow carried substance generator integration (6.04) includes a super oxygen generation water mixer (6.6), a disinfection, sterilization and degradation apparatus (6.9), a stain removal and oil dispelling cleaning apparatus (6.11), a carried substance generator item addition and upgrading module (6.16). The carried substance generator item addition and upgrading module (6.16) is a combined module capable of providing or installing any individual fluid carried substance generator additional item in the carried substance generator integration and upgrading and updating the same. The range hood type positive and negative pressure cabin (1H) is internally provided with a motor-turbine assembly (22), an automatic rotary spraying ball (23), an oil collecting and water drainage groove (24), a super oxygen sensor (C3), and carried substance sensor item addition and upgrading module (C12), and is provided with a touch screen and mobile phone monitoring and identification system (12) on a front surface. The carried substance sensor item addition and upgrading module (6.16) is a combined module capable of providing or installing any individual sensor additional item in the sensor integration and upgrading and updating the same. A water intake pipeline (A14) is installed at a water inlet port (r) of the water input pump (17), and the water inlet port (r) communicates with a tap water source; a pipeline (A15) is installed at a water output port (u) of the water input pump (17), a solenoid valve (B15) is installed at a middle part of the pipeline (A15), and the other end of the pipeline (A15) is connected to a water inlet port (z) of the water heating apparatus (27). A pipeline (A16) is installed at a water output port (q) of the water heating apparatus (27), a solenoid valve (B16) is installed at a middle part of the pipeline (A16), and the other end of the pipeline (A16) communicates with a water inlet port (v) of the waterflow carried substance generator integration (6.04). A pipeline (A18) is installed at a water output port (o) of the waterflow carried substance generator integration (6.04), a solenoid valve (B18) is installed at a middle part of the pipeline (A18), and the other end of the pipeline (A18) extends into the range hood type positive and negative pressure cabin (1H) to be connected to a spraying pipeline (A30). A pipeline (A17) is installed at a water output port (k) of the water heating apparatus (27), a solenoid valve (B17) is installed at a middle part of the pipeline (A17), and the other end of the pipeline (A17) communicates with the solenoid valve (B18), and extends into the range hood type positive and negative pressure cabin (1H) to be connected to the spraying pipeline (A30). A plurality of automatic rotary spraying balls (23) are installed on the spraying pipeline A30, and an included angle between the axis of each spraying ball and the axis of a circle of spraying holes in the middle is 45 degrees, and the spraying ball 23 rotates automatically when waterflow is sprayed. A water drainage pipeline (A20) is installed at a water inlet port (w) of the water suction pump (16), a solenoid valve (B20) is installed at a middle part of the water drainage pipeline (A20), and the other end of the water drainage pipeline (A20) extends into the oil collecting and water drainage groove (24) at the bottom of the range hood type positive and negative pressure cabin (1H) to form a water drainage port (L). A water drainage pipeline (A21) is installed at a water output port (p) of the water suction pump (16), a solenoid valve (B21) is installed at a middle part of the water drainage pipeline (A21), and the other end of the water drainage pipeline (A21) communicates with a water inlet port (g) of the carried substance decomposition processor (6.7). A water drainage pipeline (A22) is installed at a water output port (m) of the carried substance decomposition processor (6.7), a solenoid valve (B22) is installed at a middle part of the water drainage pipeline (A22), and the other end of the water drainage pipeline (A22) communicates with the sewer. A water drainage pipeline (A24) is further installed on the solenoid valve (B21), and the solenoid valve (B21) is connected to the solenoid valve (B22). All fluid carried substance generation processors (6), sensors (C) and solenoid valves (B) and the water suction pump (16), the water input pump (17), the fume evacuation control mechanism (26), the water heating apparatus (17), and the touch screen and mobile phone monitoring and identification system (12) are all connected to the positive and negative pressure intelligent regulation and control apparatus (5).

Specific embodiment 10: an operation method for an intelligent positive and negative pressure range hood is provided. The intelligent positive and negative pressure range hood adopts an intelligent positive and negative pressure system; the intelligent positive and negative pressure system is used to accurately regulate and organically integrate various techniques such as super oxygen, disinfection and sterilization, degradation treatment, and high pressure cleaning, and one item of or a combination of more items of the techniques is applied to the intelligent positive and negative pressure range hood. For stain-causing sites of both cleaning materials and contaminant in the range hood, stain-causing factors and stain removal targets of various different cleaning scenarios and cleaning materials are analyzed one by one on the molecular and microscopic level, and after computing, different accurate stain removal techniques are correspondingly used to form targeted stain removal comprehensive cleaning methods with different characteristics for removing the stain-causing factors at fixed points, so as to achieve the purpose of fast, efficient, sanitary and environmental-friendly range hood cleaning. The method includes the following steps: (1) fume extraction procedure: when the range hood is started, opening the sealed waterproof cabin door (1.2) for air intake and fume extraction, and closing the sealed cabin door (1.2) after the range hood stops operating; (2) self-cleaning procedure: sending an instruction by the positive and negative pressure intelligent regulation and control apparatus (5), enabling solenoid valves (B15), (B18), (B16) or (B17) to open corresponding water intake pipelines: (I) when the super oxygen or related carrying water is required for cleaning, a water intake line being: (A14)→water inlet port (r) of water input pump→water output port (u)→(A15)→(B15)→(A15)→water inlet port (z) of water heating apparatus→water output port (q)→(A16)→(B16)→(A16)→water inlet port (v) of related carried substance generator of waterflow carried substance generator integration→water output port (o)→(A18)→(B18)→(A18)→(A30)→automatic rotary spraying ball (23); (II) when super oxygen is not required to carry hot water for hydrolysis for cleaning, a water intake line being: (A14)→water inlet port (r) of water input pump→water output port (u)→(A15)→(B15)→(A15)-water inlet port (z) of water heating apparatus→water output port (k)→(A17)→(B17)→(A17)→(A15)→(B16)→(A16)→(A30)→automatic rotary spraying ball (23), turning on the water input pump (A17), enabling tap water to form super-oxygenated or carrying water through the water heating apparatus (27) and the waterflow carried substance generator integration (6.04) to enter the range hood type positive and negative pressure cabin (1H), comprehensively spraying and cleaning various parts, including a motor turbine assembly (22), in a machine case using the automatic rotary spraying ball (23); firstly spraying the supe-oxygenated or substance-carrying hot water to efficiently decompose and remove the oil stains, and then spraying with clean hot water until the oil stains are completely cleaned; enabling the sewage obtained after cleaning to flow downwards to be collected into an oil collecting and water drainage groove (24) at the lowest part of the machine case; when the water drainage is needed, sending an instruction by the positive and negative pressure intelligent regulation and control apparatus (5), enabling solenoid valves (B20), (B21) and (B22) to open corresponding water drainage pipelines: (I) when the super oxygen or carried substance needs to be decomposed, a water drainage line being: water drainage port (L) of oil collecting and water drainage groove→(A20)→(B20)→(A20)→water inlet port (w) of water suction pump→water output port (p)→(A21)→(B21)→(A21)→water inlet port (g) of carried substance decomposition processor→water output port (m)→(A22)→(B22)→(A22)→sewer; (II) when the super oxygen or carried substance does not need to be decomposed, a water drainage line being: water drainage port (L)w of oil collecting and water drainage groove→(A20)→(B20)→(A20)→water inlet port (w) of water suction pump→water output port (p)→(A21)→(B21)→(A24)→(B22)→(A22)→sewer, and turning on the water suction pump (16) to drain the water to the sewer; (3) drying placement procedure: after cleaning, turning on the range hood according to an instruction sent by the positive and negative pressure intelligent regulation and control apparatus (5) to completely blow-dry the inside of the machine case, and then turning off the range hood to prevent corrosion; (4) turn-on protection function: when the range hood is turned on, enabling the air to immediately flow into the positive and negative pressure water washing range hood type positive and negative pressure cabin (1H) from the outside of the range hood cabin and to be exhausted from a fume exhaust pipe (29), such that the range hood type positive and negative pressure cabin (1H) is not exposed to any super oxygen carried substance; and (5) module combined intelligent smart home mode: designing and manufacturing the intelligent positive and negative pressure range hood according to the module functions, where the intelligent positive and negative pressure range hood not only is independently produced and used as a separated invention, but also is organically combined with other positive and negative pressure electric appliances and the module cabinet under the control of the touch screen and mobile phone monitoring and identification system (12) to form a fully functional intelligent positive and negative pressure module combined home; the fully functional intelligent positive and negative pressure module combined home not only shares the water input pump (17), the water suction pump (16), the waterflow carried substance generator integration (6.04) and the carried substance decomposition processor (6.7) with the dish washing and fruit and vegetable cleaning machine, but also has basically uniform, neat and harmonious appearance, shape and color which are in line with the popular trend, such that the intelligent range hood is more efficient, low-consumption, and suitable for use.

Specific embodiment 11: an intelligent positive and negative pressure baking and frying microwave oven includes a machine body (7). The machine body (7) is internally provided with an intelligent positive and negative pressure system, a baking system (30), an air frying system (31), and a microwave system (32). The intelligent positive and negative pressure system includes a positive and negative pressure cabin (1), an air evacuation pump (2), an air inflation pump (3), a positive and negative pressure intelligent regulation and control apparatus (5), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), and the touch screen and mobile phone monitoring and identification system (12). The positive and negative pressure cabin (1) is designed as a microwave oven type positive and negative pressure cabin (1K) according to the requirements of an oven, an air fryer and the microwave oven, and the microwave oven type positive and negative pressure cabin (1K) is a vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the baking and frying microwave oven. The microwave oven type positive and negative pressure cabin (1K) includes a cabin body (1.1), a cabin door (1.2), an airtight mechanism (1.3), and an internal and external communicating sealer (1.4). The cabin door (1.2) is a machine door of the positive and negative pressure baking and frying microwave oven. The airtight mechanism (1.3) is arranged between the cabin door (1.2) and the cabin body (1.1) of the microwave oven type positive and negative pressure cabin (1K). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When the positive and negative pressure is formed inside microwave oven type positive and negative pressure cabin (1K), the airtight gasket (1.33) is used for keeping sealing. The internal and external communicating sealer (1.4) is fixedly arranged at a rear side part of the microwave oven type positive and negative pressure cabin (1K), and all pipes and circuits getting in and out the cabin are connected to and pass through the internal and external communicating sealer (1.4), so as to keep the sealing property of the microwave oven type positive and negative pressure cabin (1K). The positive and negative pressure intelligent regulation and control apparatus (5) includes an integrated circuit, a chip, and a regulation and control system for a fluid and a carried substance thereof, and is connected to an Internet in a wired or wireless mode. The microwave oven type positive and negative pressure cabin (1K) is internally provided with the baking system (30), an air frying system (31), a microwave system (32), a positive and negative pressure sensor (C15), and a sensor item addition and upgrading module (C12). A non-standard structure other than above standard structure is also used inside the microwave oven type positive and negative pressure cabin (1K), which includes at least one of the microwave system (32), the baking system (30), and the air frying system (31). The microwave oven type positive and negative pressure cabin (1K) is externally provided with the air evacuation pump (2), the air inflation pump (3), the positive and negative pressure intelligent regulation and control apparatus (5), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the microwave oven type positive and negative pressure cabin (1K) to form an air evacuation and air return port (c) thereof. An air air exhaust pipeline (A2) is installed at an air outlet port (b) of the air evacuation pump (2), and is connected to the atmosphere; an air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the microwave oven type positive and negative pressure cabin (1K) to form an air inflation and inlet port (f). An air intake pipeline (A4) is installed at an air inlet port (e) of the air inflation pump (3) and is connected to the atmosphere. All sensors (C) and solenoid valves (B), and the air evacuation pump (2), the air inflation pump (3), the baking system (30), the air frying system (31), the microwave system (32), and the touch screen and mobile phone monitoring and identification system (12) are all connected to the positive and negative pressure intelligent regulation and control apparatus (5).

Specific embodiment 12, an intelligent positive and negative pressure fresh-keeping transport compartment container includes a compartment body (7). The compartment body (7) is internally provided with an intelligent positive and negative pressure system, and a refrigeration system (8). The intelligent positive and negative pressure system includes a positive and negative pressure cabin (1), an air evacuation pump (2), an air inflation pump (3), a positive and negative pressure intelligent regulation and control apparatus (5), a positive and negative pressure fluid carried substance generation processor (6), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a compartment container type positive and negative pressure cabin (1M) or an atmospheric compartment container type positive and negative pressure cabin (1M-2) according to the requirements of the intelligent positive and negative pressure fresh-keeping transport compartment container. An internal structure of the compartment container type positive and negative pressure cabin (1M) includes a cabin body (1.1), a cabin door (1.2), an airtight mechanism (1.3), and an internal and external communicating sealer (1.4). The cabin door (1.2) is a compartment door of the positive and negative pressure fresh-keeping transport compartment container; the airtight mechanism (1.3) is arranged between the cabin door (1.2) and the cabin body (1.1) of the compartment container type positive and negative pressure cabin (1M). The airtight mechanism (1.3) includes a lock ring (1.31), a lock bolt (1.32), and an airtight gasket (1.33). The lock ring (1.31) is in movable fit with the lock bolt (1.32), and the lock bolt (1.32) extends into the lock ring (1.31) to lock the cabin door during the closing of the cabin door. When the positive and negative pressure is formed inside the compartment container type positive and negative pressure cabin (1M), the airtight gasket (1.33) is used for keeping sealing. The internal and external communicating sealer 1.4 is fixedly arranged at a rear part of the compartment container type positive and negative pressure cabin (1M), and all pipes and circuits getting in and out the cabin are connected to and pass through the internal and external communicating sealer (1.4). The compartment container type positive and negative pressure cabin (1M), due to the installation of the airtight mechanism (1.3) and the internal and external communicating sealer (1.4) and the possess of a corresponding vacuum high pressure resistance structure, belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the compartment container. The atmospheric compartment container type positive and negative pressure cabin (1M-2) is a general compartment container with the positive and negative pressure system and without the airtight mechanism (1.3) and the internal and external communicating sealer (1.4). A general compartment container space is an atmospheric positive and negative pressure cabin without a vacuum high pressure resistance structure, belonging to a general sealed atmospheric circulation cabin (1-2) or atmospheric inlet-outlet cabin (3) structure. The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), an airflow carried substance generator integration (6.02), and an integrated pipeline type positive and negative fluid carried substance generator (6.05). The air conditioning apparatus (6.1) is a membrane-based air separation type air conditioning apparatus. The carried substance generator item addition and upgrading module (6.16) is a combined module capable of providing or installing any individual fluid carried substance generator additional item in the carried substance generator integration and upgrading and updating the same. Each of the airflow carried substance generator integration (6.02) and the integrated pipeline type positive and negative pressure fluid carried substance generator (6.05) includes any one or more in the individual fluid carried substance generators (6.1) to (6.16). The compartment container type positive and negative pressure cabin (1M) or the atmospheric compartment container type positive and negative pressure cabin (1M-2) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration system (8), an air-to-water production apparatus (11), and a touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the compartment container type positive and negative pressure cabin (1M) or the atmospheric compartment container type positive and negative pressure cabin (1M-2) to form an air evacuation and air return port (c) thereof. Exhaust and evacuation pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), and passes through the air-to-water production apparatus (11) and then is connected to the atmosphere through an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the compartment container type positive and negative pressure cabin (1M) or the atmospheric compartment container type positive and negative pressure cabin (1M-2) to form an air inflation and inlet port (f) thereof. An air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to atmosphere; a carrying circulation pipeline (A5) is installed at an air inlet port (e2) of the air inflation pump (3), the other end of the carrying circulation pipeline (A5) Communicates with a solenoid valve (B3), and then extends into the compartment container type positive and negative pressure cabin (1M) or the atmospheric compartment container type positive and negative pressure cabin (1M-2) through the air inflation pipeline (A3) to form a circulating air return port (j) thereof. Carrying air introduction pipelines (A6), (A6.1) and (A6.2) are further connected to the air inflation pipeline (A3), a solenoid valve (B6) is installed at a middle part of the carrying air introduction pipeline (A6), and the other end of the carrying air introduction pipeline (A6) is connected to an air inlet port (t) of the airflow carried substance generator integration (6.02). A carrying air introduction pipeline (A7) is installed at an air outlet port (s) of the airflow carried substance generator integration (6.02), a solenoid valve (B7) is installed at a middle part of the carrying air introduction pipeline (A7), and the other end of the carrying air introduction pipeline (A7) extends into the compartment container type positive and negative pressure cabin (1M) or the atmospheric compartment container type positive and negative pressure cabin (1M-2) to form a carrying air inlet port (i) thereof. Solenoid valves (B6.1) and (B6.2) are respectively installed at middle parts of the air introduction pipelines (A6.1) and (A6.2), and the other ends of the air introduction pipelines (A6.1) and (A6.2) extend into the compartment container type positive and negative pressure cabin (1M) or the atmospheric compartment container type positive and negative pressure cabin (1M-2) to be connected to air inlet ports of the airflow carried substance generator integration (6.021) and the integrated pipeline type positive and negative pressure fluid carried substance generator (6.05). An air-to-water production air inflation pipeline (A9) is further connected to the air inflation pipeline (A3), the air-to-water production air inflation pipeline (A9) is connected to the solenoid valve (B2) and then is connected to the air exhaust pipeline (A2); the middle part of the air exhaust pipeline (A2) communicates with the air-to-water production apparatus (11), and the other end of the air exhaust pipeline (A2) is connected to the atmosphere. The touch screen and mobile phone monitoring and identification system (12) includes a door touch screen (12.1), a mobile phone APP (12.2), a wireless anti-fog camera identification apparatus (12.4), and a wireless radar scanning and identification apparatus (12.6). The wireless anti-fog camera identification apparatus (12.4) and the wireless radar scanning and identification apparatus (12.6) are installed at positions inside/outside the compartment body (7) needing to be monitored and identified. The compartment container type positive and negative pressure cabin (1M) or the atmospheric compartment container type positive and negative pressure cabin (1M-2) is internally provided with an airflow carried substance generator integration (6.021), an integrated pipeline type positive and negative pressure fluid carried substance generator (6.05), and corresponding sensors thereof. The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

Specific embodiment 13: an operation method for an intelligent positive and negative pressure fresh-keeping compartment container is provided. The intelligent positive and negative pressure fresh-keeping compartment container adopts an intelligent positive and negative pressure system. The intelligent positive and negative pressure system is used to accurately regulate and organically integrate various techniques such as super oxygen, catalysts, air conditioning fresh-keeping, negative ion, humidification, dehumidification, disinfection and degradation, air purification, air-to-water production and low-temperature storage, and the one item of or a combination of more items of the techniques is applied to the positive and negative pressure fresh-keeping compartment container, and digital techniques such as Internet, big data, cloud computing, machine learning, artificial intelligence and expert system are also applied to the compartment container at the same time, so as to form a regulatable and controllable artificial intelligence microclimate in the positive and negative pressure fresh-keeping compartment container and to create a high-energy and efficient precise fresh-keeping microenvironment. Various fresh-keeping elements such as air composition, airflow pressure, humidity and temperature, aseptic cleanliness and the like of the positive and negative pressure fresh-keeping compartment container are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization and disinfection, quickly degrade harmful residues on the surface of preservation, achieve all-round and multi-angle digital targeted treatment, eliminate putrefying factors, accurately keep freshness and comprehensively preserve nutrition. Various fresh goods stored in and transported by the positive and negative pressure fresh-keeping compartment container are in an optimal pressure range, an optimal gas composition and optimal humidity and temperature, and an optimal preservation environment; fruits and vegetables are hibernated at low temperature in an existing refrigerating compartment container, while the positive and negative pressure and digital techniques are used in the positive and negative pressure fresh-keeping compartment container to make fruits and vegetables in a dormant or suspended animation state, such that the fruits and vegetables have the weakest life, the slowest aging, the lowest breathing and the least consumption of nutrition, and the nutritional components and the original freshness of fruits, vegetables, meat, fish and other stored and transported products are quickly stored for a long time. In the positive and negative pressure system, the digital technique is used to analyze and identify fresh-loss sites and fresh-keeping elements of various stored and transported products one by one to form a complete large database according to different categories, and to design a personalized targeted control comprehensive fresh-keeping method at the cellular and molecular level for the fresh-loss sites and fresh-keeping elements of the stored and transported products, so as to accurately maintain the fresh-keeping elements and remove the fresh-loss factors at a fixed point, without affecting or less affecting the normal tissues and cells of the stored and transported products, and thus a set of digital expert system for fresh-keeping of stored products is formed and gradually improved, which is intelligently applied to the positive and negative pressure fresh-keeping compartment container to achieve the purpose of keeping freshness and quality of the stored and transported products. In a specific operation method for the intelligent positive and negative pressure fresh-keeping compartment container, an air evacuation pump or air inflation pump is used by the positive and negative pressure system to intelligently regulate and control airflow pressure, or carry super oxygen, catalyst, negative ion, air conditioning gas, disinfectant, detergent, heat, water and various effective loads by the airflow to get in, stay in or get out of a compartment container type positive and negative pressure cabin (1M) or an atmospheric compartment container type positive and negative pressure cabin (1M-2) orderly, and the following methods are used to apply various forces helpful for long-term freshness and quality keeping to fresh fruits and vegetables stored in the compartment container: (1): positive atmospheric pressure high pressure action method: forming moderate positive atmospheric pressure at proper time in a cabin of the compartment container type positive and negative pressure cabin (1M), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the compartment container type positive and negative pressure cabin (1M), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) airflow carried substance action method: carrying single or a combination of super oxygen, catalyst, negative ion, air conditioning gas, disinfection and degradation substances, heat, water and various effective loads by positive and negative pressure airflow to get in, stay in or get out of the compartment container type positive and negative pressure cabin (1M), or the atmospheric compartment container type positive and negative pressure cabin (1M-2) orderly, integrating various fresh-keeping techniques to apply various accurate and effective action forces for the long-term freshness and quality keeping of the stored and transported fruits, vegetables and fresh goods. Based on above three action force methods, a positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of a touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping; the targeted control comprehensive fresh-keeping methods for the intelligent positive and negative pressure system and specific use methods and operation procedures of the positive and negative pressure fresh-keeping compartment container are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1) to open corresponding air exhaust pipelines (A2) and (A1), and turning on the air evacuation pump (2) to exhaust or pump the atmospheric compartment container type positive and negative pressure cabin (1M-2) or the compartment container type positive and negative pressure cabin (1M) to vacuum or negative pressure between −0.001 KPa and −0.1 MPa (taking the local instant atmospheric pressure as the zero standard), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within compartment container type positive and negative pressure cabin (1M), thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) pressurized or atmospheric circulating sterilization, degradation and deodorization procedure for super oxygen, negative ion or catalyst positive and negative pressure carried substance: according to the procedure setting and feedback of sensors (C), sending, by the positive and negative pressure intelligent regulation and control apparatus (5), an instruction to turn on a super oxygen generator (6.2), a negative ion generator (6.3), a catalyst release controller (6.4), a humidity control apparatus (6.5) or a disinfection and degradation apparatus (6.9) in an airflow carried substance generator integration (6.02) or (6.021) or an integrated pipeline type fluid carried substance generator (6.05), or a carried substance generator installed in a carried substance generator module (6.16), thus enabling the super oxygen, catalyst, negative ion, water and related carried substances in the positive and negative pressure cabin reach required standards; meanwhile, enabling solenoid valves (B3), (B4) and (B3.1), (B3.2), (B3.3) or (B3.4) to open corresponding air inflation pipelines (A4) and (A3) and (A3.1), (A3.2), (A3.3), or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and compartment container configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric, pressurized or humidified preservation procedure of air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3) and (B4), (B6), (B6.1) or (B6.2) to open corresponding related carried substance air inflation pipelines (A4) and (A3), (A6), (A6.1) or (A6.2), or enabling solenoid valves (B3), (B4), (B6) and (B7) to open corresponding related carried substance circulation pipelines (A3), (A5), (A6) and (A7), and turning on the air inflation pump (3) for the inflation and pressurization or atmospheric circulation of the compartment container type positive and negative pressure cabin (1M) or the atmospheric compartment container type positive and negative pressure cabin (1M-2); during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B1) and (B2) or (B8.1) to open carrying waste gas evacuation and exhaust pipelines (A8) or (A8.1), (A1) or (A2), carrying waste gas evacuation and exhaust port (s1) or (s2) of air conditioning apparatus (6.1)→(A8) or (A8.1)→solenoid valves (B1)→(A1)→air inlet port (a) of air evacuation pump→air outlet port (b) of air evacuation pump→(A2)→carried substance decomposition processor (6.7)→(A2)→(B2)→(A2)→air-to-water production apparatus (11)→(A2)→exhaust to atmosphere; turning on the air evacuation pump (2) to exhaust and pump decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) low-temperature assisted fresh-keeping procedure: after the positive and negative pressure fresh-keeping compartment container is turned on and the door is closed, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (6) open-to-exhaust protection function: if the compartment door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to the carried substance decomposition processor (6.7) to be exhausted to atmosphere after being decomposed, where, as the air flows from the outside of the fresh-keeping compartment container door to the positive and negative pressure cabin for supplementing air evacuation negative pressure, the super oxygen or high-nitrogen low-oxygen carried substance gas cannot flow out of the door; (7) air-to-water production procedure: enabling moist waste gas pumped from the vacuum high pressure cabin by the air evacuation pump 2 to enter the carried substance decomposition processor (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B2) to open air-to-water production inflation pipelines, thus driving the indoor air to enter the air-to-water production apparatus (11) for water production; (8) remote and short-range control monitoring function: installing a high-definition anti-fog camera and identification apparatus (12.3) and a radar scanning and identification apparatus (12.5) at positions needing monitoring and identification inside and outside the compartment body (7); shooting and scanning statuses of objects in the fresh-keeping compartment container in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling, by the positive and negative pressure system, various fresh-keeping environmental factors such as airflow pressure, gas composition, air cleanliness, environment temperature and humidity in the positive and negative pressure cabin in real time, accurately positioning and removing the fresh-loss factors in a targeted manner, and automatically performing harmless disinfection and sterilization, and rapidly degrading residual pesticides, fertilizers, hormones and harmful additives. In conclusion, in the specific operation method for the intelligent positive and negative pressure fresh-keeping compartment container, various techniques such as vacuum, high pressure, super oxygen, catalyst, air-conditioning fresh-keeping, negative ion, humidification, humidity removal, humidity control, disinfection and sterilization, air purification, air-to-water production and low-temperature storage are intelligently regulated, controlled and integrated under the positive pressure and negative pressure, which are organically applied to the intelligent positive and negative pressure fresh-keeping compartment container and are organically integrated and optimally matched, one item of or a combination of more items of the techniques is used individually or alternately or circularly, and multiple positive and negative pressure cabins with different types and different functions are arranged in a positive and negative pressure fresh-keeping compartment, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping compartment container updates the existing method that the traditional refrigerating compartment container achieves low-temperature storage only by controlling the temperature of the fresh-keeping compartment container. A regulatable and controllable artificial intelligence microclimate is formed in the positive and negative pressure cabin to create a precise fresh-keeping microenvironment, in which various fresh food stored can keep fresh, moist and original quality and flavor for a long time and to the maximum extent.

Specific embodiment 14: an intelligent positive and negative pressure fresh-keeping warehouse includes a warehouse body (7). The warehouse body (7) is internally provided with an intelligent positive and negative pressure system, and a refrigeration system (8). The intelligent positive and negative pressure system includes a positive and negative pressure cabin (1), an air evacuation pump (2), an air inflation pump (3), a positive and negative pressure intelligent regulation and control apparatus (5), a positive and negative pressure fluid carried substance generation processor (6), and a touch screen and mobile phone monitoring and identification system (12). The intelligent positive and negative pressure system also adopts a nonstandard structure other than the above standard structures in specific applications. The nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a warehouse type positive and negative pressure cabin (1N) or an atmospheric warehouse type positive and negative pressure cabin (1N-2) according to the requirements of the intelligent positive and negative pressure fresh-keeping warehouse. An internal structure of the warehouse type positive and negative pressure cabin (1N) includes a cabin body (1.1), a cabin door (1.2), an airtight mechanism (1.3), and an internal and external communicating sealer (1.4). The cabin door (1.2) is a warehouse door of the positive and negative pressure fresh-keeping warehouse. The airtight mechanism (1.3) is arranged between the cabin door (1.2) and the cabin body (1.1) of the warehouse type positive and negative pressure cabin (1N). The airtight mechanism (1.3) includes a lock ring (1.31), a lock bolt (1.32), and an airtight gasket (1.33). The lock ring (1.31) is in movable fit with the lock bolt (1.32), the lock bolt (1.32) extends into the lock ring (1.31) to lock the cabin door during the closing of the cabin door, and when the positive and negative pressure is formed inside the warehouse type positive and negative pressure cabin (1N), the airtight gasket (1.33) is used for keeping sealing. The internal and external communicating sealer (1.4) is fixedly arranged at a rear part of the warehouse type positive and negative pressure cabin (1N), and all pipes and circuits getting in and out the cabin are connected to and pass through the internal and external communicating sealer (1.4). The warehouse type positive and negative pressure cabin (1N), due to the installation of the airtight mechanism (1.3) and the internal and external communicating sealer (1.4) and the possess of a corresponding vacuum high pressure resistance structure, belongs to an airtight vacuum high pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the fresh-keeping warehouse. The atmospheric warehouse type positive and negative pressure cabin (1N-2) is a general warehouse with the positive and negative pressure system and without the airtight mechanism (1.3) and the internal and external communicating sealer (1.4). A general warehouse space is an atmospheric positive and negative pressure cabin without a vacuum high pressure resistance structure, belonging to a general sealed atmospheric circulation cabin (1-2) or atmospheric inlet-outlet cabin (3) structure; the intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), and an airflow carried substance generator integration (6.02) or an integrated pipeline type positive and negative fluid carried substance generator (6.05). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. Each of the airflow carried substance generator integration (6.02) and the integrated pipeline type positive and negative pressure fluid carried substance generator (6.05) includes any one or more in the individual fluid carried substance generators (6.1) to (6.16). The warehouse type positive and negative pressure cabin (1N) or the atmospheric warehouse type positive and negative pressure cabin (1N-2) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration system (8), an air-to-water production apparatus (11), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the warehouse type positive and negative pressure cabin (1N) or the atmospheric warehouse type positive and negative pressure cabin (1N-2) to form an air evacuation and air return port (c) thereof. Exhaust and evacuation pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), and passes through the air-to-water production apparatus (11) and then is connected to the atmosphere through an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the warehouse type positive and negative pressure cabin (1N) or the atmospheric warehouse type positive and negative pressure cabin (1N-2) to form an air inflation and inlet port (f) thereof; an air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to the atmosphere. A carrying circulation pipeline (A5) is installed at an air inlet port (e2) of the air inflation pump (3), the other end of the carrying circulation pipeline (A5) communicates with a solenoid valve (B3), and then extends into the warehouse type positive and negative pressure cabin (1N) or the atmospheric warehouse type positive and negative pressure cabin (1N-2) through the air inflation pipeline (A3) to form a circulating air return port (j) thereof. Carrying air introduction pipelines (A6), (A6.1) and (A6.2) are further connected to the air inflation pipeline (A3), a solenoid valve (B6) is installed at a middle part of the carrying air introduction pipeline (A6), and the other end of the carrying air introduction pipeline (A6) is connected to an air inlet port (t) of the airflow carried substance generator integration (6.02). A carrying air introduction pipeline (A7) is installed at an air outlet port (s) of the airflow carried substance generator integration (6.02), a solenoid valve (B7) is installed at a middle part of the carrying air introduction pipeline (A7), and the other end of the carrying air introduction pipeline (A7) extends into the warehouse type positive and negative pressure cabin (1N) or the atmospheric warehouse type positive and negative pressure cabin (1N-2) to form a carrying air inlet port (i) thereof. Solenoid valves (B6.1) and (B6.2) are respectively installed at middle parts of the air introduction pipelines (A6.1) and (A6.2), and the other ends of the air introduction pipelines (A6.1) and (A6.2) extend into the warehouse type positive and negative pressure cabin (1N) or the atmospheric warehouse type positive and negative pressure cabin (1N-2) to be connected to air inlet ports of the airflow carried substance generator integration (6.021) and the integrated pipeline type positive and negative pressure fluid carried substance generator (6.05). An air-to-water production air inflation pipeline (A9) is further connected to the air inflation pipeline (A3), the air-to-water production air inflation pipeline (A9) is connected to the solenoid valve (B2) and then is connected to the air exhaust pipeline (A2). The middle part of the air exhaust pipeline (A2) communicates with the air-to-water production apparatus (11), and the other end of the air exhaust pipeline (A2) is connected to the atmosphere. The touch screen and mobile phone monitoring and identification system (12) includes a door touch screen (12.1), a mobile phone APP (12.2), a wireless anti-fog camera identification apparatus (12.4), and a wireless radar scanning and identification apparatus (12.6). The wireless anti-fog camera identification apparatus (12.4) and the wireless radar scanning and identification apparatus (12.6) are installed at positions inside/outside the warehouse body (7) needing to be monitored and identified. The warehouse type positive and negative pressure cabin (1N) or the atmospheric warehouse type positive and negative pressure cabin (1N-2) is internally provided with an airflow carried substance generator integration (6.02), or an integrated pipeline type positive and negative pressure fluid carried substance generator (6.05), and corresponding sensors thereof. The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

Specific embodiment 15: an operation method for an intelligent positive and negative pressure fresh-keeping warehouse is provided. The intelligent positive and negative pressure fresh-keeping warehouse adopts an intelligent positive and negative pressure system. The intelligent positive and negative pressure system is used to accurately regulate and organically integrate various techniques such as super oxygen, catalysts, air conditioning fresh-keeping, negative ions, humidification, dehumidification, disinfection and degradation, air purification, air-to-water production and low-temperature storage, one item of or a combination of more items of the techniques is applied to the positive and negative pressure fresh-keeping warehouse, and digital techniques such as Internet, big data, cloud computing, machine learning, artificial intelligence and expert system are also applied to the warehouse at the same time, so as to form a regulatable and controllable artificial intelligence microclimate in the positive and negative pressure fresh-keeping warehouse and to create a high-energy and efficient precise fresh-keeping microenvironment. Various fresh-keeping elements such as air composition, airflow pressure, humidity and temperature, aseptic cleanliness and the like of the positive and negative pressure fresh-keeping warehouse are intelligently and accurately regulated and controlled in real time according to built-in program and pre-stored data of the positive and negative pressure system, and real-time feedback information of various sensors by using a breathing and circulation mechanism, so as to carry out harmless and dead-end sterilization and disinfection, quickly degrade harmful residues on the surface of preservation, achieve all-round and multi-angle digital targeted treatment, eliminate putrefying factors, accurately keep freshness and comprehensively preserve nutrition. Various fresh goods stored in the positive and negative pressure fresh-keeping warehouse are in an optimal pressure range, an optimal gas composition and optimal humidity and temperature, and an optimal preservation environment. Fruits and vegetables are hibernated at low temperature in an existing refrigerating warehouse, while the positive and negative pressure and digital techniques are used in the positive and negative pressure fresh-keeping warehouse to make fruits and vegetables in a dormant or suspended animation state, such that the fruits and vegetables have the weakest life, the slowest aging, the lowest breathing and the least consumption of nutrition. The nutritional components, the original freshness and original flavor of fruits, vegetables, meat, fish and other stored products are quickly stored for a long time; in the positive and negative pressure system, the digital technique is used to analyze and identify fresh-loss sites and fresh-keeping elements of various stored products one by one to form a complete large database according to different categories, and to design a personalized targeted control comprehensive fresh-keeping method at the cellular and molecular level for the fresh-loss sites and fresh-keeping elements of the stored products, so as to accurately maintain the fresh-keeping elements and remove the fresh-loss factors at a fixed point, without affecting or less affecting the normal tissues and cells of the stored products, and thus a set of digital expert system for fresh-keeping of stored products is formed and gradually improved, which is intelligently applied to the positive and negative pressure fresh-keeping warehouse to achieve the purpose of keeping freshness and quality of the stored and transported products. In a specific operation method for the intelligent positive and negative pressure fresh-keeping warehouse, an air evacuation pump or air inflation pump is used by the positive and negative pressure system to intelligently regulate and control airflow pressure, or carry super oxygen, catalyst, negative ions, air conditioning gas, disinfectant, detergent, heat, water and various effective loads by the airflow to get in, stay in or get out of a warehouse type positive and negative pressure cabin (1N) or an atmospheric warehouse type positive and negative pressure cabin (1N-2) orderly, and the following methods are used to apply various forces helpful for long-term freshness and quality keeping to fresh fruits and vegetables stored in the warehouse: (1): positive atmospheric pressure high pressure action method: forming moderate positive atmospheric pressure at proper time in a cabin of the warehouse type positive and negative pressure cabin (1N), which, after fruits and vegetables in the cabin are compressed, makes water evaporation channels on the surfaces of the fruits and vegetables contract or close, thus inhibiting anaerobic respiration, growth and aging and water loss of the fruits and vegetables, and changing ways, paths, degrees and progress time of food damage caused by various external factors such as bacterial viruses, food disease sources and polluted particles at the micro level; (2) negative atmospheric pressure vacuum action method: forming moderate negative pressure vacuum at proper in the cabin the warehouse type positive and negative pressure cabin (1N), which inhibits anaerobic respiration, growth and aging of fruits and vegetables and makes bacteria and microorganisms unable to survive in vacuum, thus enhancing he fresh-keeping effect of the stored fruits, vegetables, meat and fish; (3) airflow carried substance action method: carrying single or a combination of super oxygen, catalyst, negative ion, air conditioning gas, disinfection and degradation substances, heat, water and various effective loads by positive and negative pressure airflow to get in, stay in or get out of the warehouse type positive and negative pressure cabin (1N), or the atmospheric warehouse type positive and negative pressure cabin (1N-2) orderly, integrating various fresh-keeping techniques to apply various accurate and effective action forces for the long-term freshness and quality keeping of the stored fruits, vegetables and fresh goods. Based on above three action force methods, a positive and negative pressure intelligent regulation and control apparatus (5) is used to analyze and identify fresh-loss sites and fresh-keeping elements of various stored products one by one according to built-in program, pre-stored data, and internet cloud data of the positive and negative pressure intelligent regulation and control apparatus (5), camera identification, radar scanning and identification of a touch screen and mobile phone monitoring and identification system (12), and real-time feedback information of various sensors (C1) to (C12), and after computing, different targeted fresh-keeping techniques are correspondingly used to form targeted control comprehensive fresh-keeping methods with different characteristics, and the positive and negative pressure intelligent regulation and control apparatus (5) is used to send specific instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof, so as to precisely maintain the fresh-keeping elements of the stored products and remove fresh-loss factors of the stored products at fixed points to achieve the long-term effect of fresh and quality keeping. The targeted control comprehensive fresh-keeping methods for the intelligent positive and negative pressure system and specific use methods and operation procedures of the positive and negative pressure fresh-keeping warehouse are as follows: (1) exhaust and vacuum abatement heat-removal cleaning procedure: closing a cabin door (1.2), sending an instruction by the positive and negative pressure intelligent regulation and control apparatus (5) to enable the solenoid valves (B2) and (B1) to open corresponding air exhaust pipelines (A2) and (A1), and turning on the air evacuation pump (2) to exhaust or pump the atmospheric warehouse type positive and negative pressure cabin (1N-2) or the warehouse type positive and negative pressure cabin (1N) to vacuum or negative pressure between −0.001 KPa and −0.1 MPa (taking the local instant atmospheric pressure as the zero standard), evacuating away the self-contained heat of fruits, vegetables, meat and fish, part of bacteria and microorganisms and polluted air in the cabin, vacuumizing to make volatile metabolites ethylene acetaldehyde ethanol and other harmful gases in the fruit and vegetable tissues to escape and be pumped away, while inhibiting the reproduction of residual bacteria with the negative pressure to reduce the potential risks of disease aging of fruits and vegetables; (2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within warehouse type positive and negative pressure cabin (1N), thus reducing respiration intensity of fruits and vegetables, inhibiting the biosynthesis of ethylene, delaying decomposition of chlorophyll, inhibiting the synthesis of carotenoids and lycopene, and slowing down the processes such as hydrolysis of starch, increase of sugar and consumption of acid to delay mature senescence of the fruits and vegetables, and meanwhile, turning on the a humidification and humidity control apparatus (6.5) to increase and control humidity in the cabin and prevent the water contained in the fruits and vegetables from escaping, and controlling the pressure and humidity under the positive and negative pressure in a targeted manner to accurately regulate and control classified preservation, thus maintaining the fresh state of the fruits and vegetables for a longer time; (3) pressurized or atmospheric circulating sterilization, degradation and deodorization procedure for super oxygen, negative ion or catalyst positive and negative pressure carried substance: according to the procedure setting and feedback of sensors (C), sending, by the positive and negative pressure intelligent regulation and control apparatus (5), an instruction to turn on a super oxygen generator (6.2), a negative ion generator (6.3), a catalyst release controller (6.4), a humidity control apparatus (6.5) or a disinfection and degradation apparatus (6.9) in an airflow carried substance generator integration (6.02) or (6.021) or an integrated pipeline type fluid carried substance generator (6.05), or a carried substance generator installed in a carried substance generator module (6.16), thus enabling the super oxygen, catalyst, negative ion, water and related carried substances in the positive and negative pressure cabin reach required standards; meanwhile, enabling solenoid valves (B3), (B4) and (B3.1), (B3.2), (B3.3) or (B3.4) to open corresponding air inflation pipelines (A4), (A3) and (A3.1), (A3.2), (A3.3), or (A3.4) of the related positive and negative pressure cabin, turning on the air inflation pump (3) to properly pressurize the related positive and negative pressure cabin to 0.001 KPa to 10 MPa (taking the local instant atmospheric pressure as the zero standard, and the vacuum degree can be improved according to the specific demands and warehouse configuration), where the super oxygen, negative ion and catalyst at the appropriate positive pressure can perform sterilization, disinfection and mildew resistance on the fruits, vegetables, meat and fish stored in the positive and negative pressure cabin and degrade pesticide residues and ethylene and other gases exhaled by the metabolism of the fruits and vegetables, thus affecting enzyme activity in the fruits and vegetables, preventing the fruits and vegetables from browning and softening, and delaying the aging of the fruits and vegetables; and the appropriate high pressure further prevents the water contained in the fruits, vegetables, meat and fish from escaping, moreover, the water at the positive pressure is easier to permeate into the fruits, vegetables, meat and fish to supplement the water loss thereof caused by refrigeration and negative pressure, while the negative oxygen ions make the water cluster of water smaller so as to be absorbed by the fruits, vegetables, meat and fish easier and have the functions of inhibiting biological tissue metabolism, reducing respiration intensity, slowing down enzyme activity, and sterilizing and purifying to a certain extent; the super oxygen at the positive pressure can permeate into the deeper interior of the fruits, vegetables, meat and fish to play a better role in sterilization and disinfection; (4) atmospheric, pressurized or humidified preservation procedure of air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling solenoid valves (B3) and (B4), (B6), (B6.1) or (B6.2) to open corresponding related carried substance air inflation pipelines (A4) and (A3), (A6), (A6.1) or (A6.2), or enabling solenoid valves (B3), (B4), (B6) and (B7) to open corresponding related carried substance circulation pipelines (A3), (A5), (A6) and (A7), and turning on the air inflation pump (3) for the inflation and pressurization or atmospheric circulation of the warehouse type positive and negative pressure cabin (1N) or the atmospheric warehouse type positive and negative pressure cabin (1N-2); during the operation of an air conditioning apparatus (6.1), moderately increasing partial pressure difference between both sides of a membrane to improve air separation efficiency, and evacuating and exhausting oxygen-enriched waste gas or carried waste gas; enabling solenoid valves (B1) and (B2) or (B8.1) to open carrying waste gas evacuation and exhaust pipelines (A8) or (A8.1), (A1) or (A2), carrying waste gas evacuation and exhaust port (s1) or (s2) of air conditioning apparatus (6.1)→(A8) or (A8.1)→solenoid valves (B1)→(A1)→air inlet port (a) of air evacuation pump→air outlet port (b) of air evacuation pump→(A2)→carried substance decomposition processor (6.7)→(A2)→(B2)→(A2)→air-to-water production apparatus (11)→(A2)→exhaust to atmosphere; turning on the air evacuation pump (2) to exhaust and pump decomposed waste gas; according to the above method and the optimal standard and fresh-loss sites for fresh-keeping preservation of air-conditioning or carrying gas, accurately regulating and controlling the circulation and operation with the target control of the positive and negative pressure system, thus keeping the gas composition, gas concentration, gas pressure, air dryness and humidity and cleanliness in the positive and negative pressure cabin within an optimal range required for fresh-keeping of fruits and vegetables, effectively controlling the respiration rate of the stored fruits and vegetables, preventing anaerobic respiration and carbon dioxide poisoning, slowing down the aging process, preventing the quality decrease and flavor loss of the stored fruits and vegetables, and maintaining the water and supplementing the water of the fruits and vegetables with the positive pressure so as to maintain the freshness of the fruits and vegetables to the maximum extent; (5) low-temperature assisted fresh-keeping procedure: after the positive and negative pressure fresh-keeping warehouse is turned on and the door is closed, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects; (6) open-to-exhaust protection function: if the warehouse door is opened during super-oxygen sterilization and disinfection or air-condition preservation, providing feedback immediately by a warehouse door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to the carried substance decomposition processor (6.7) to be exhausted to atmosphere after being decomposed, wherein, as the air flows from the outside of the warehouse door to the positive and negative pressure cabin for supplementing air evacuation negative pressure, the super oxygen or high-nitrogen low-oxygen carried substance gas cannot flow out of the door; (7) air-to-water production procedure: enabling moist waste gas pumped from the vacuum high pressure cabin by the air evacuation pump (2) to enter the carried substance decomposition processor (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling solenoid valves (B3), (B2) to open air-to-water production inflation pipelines, thus driving the indoor air to enter the air-to-water production apparatus (11) for water production; (8) remote and short-range control monitoring and identification function: installing a high-definition anti-fog camera and identification apparatus (12.3) and a radar scanning and identification apparatus (12.5) at positions needing monitoring and identification inside and outside the warehouse body (7); shooting and scanning statuses of objects in the warehouse in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling, by the positive and negative pressure system, various fresh-keeping environmental factors such as airflow pressure, gas composition, air cleanliness, environment temperature and humidity in the positive and negative pressure cabin in real time, accurately positioning and removing the fresh-loss factors in a targeted manner, and automatically performing harmless disinfection and sterilization, and rapidly degrading residual pesticides, fertilizers, hormones and harmful additives. In conclusion, in the specific operation method for the intelligent positive and negative pressure fresh-keeping warehouse, various techniques such as vacuum, high pressure, super oxygen, catalyst, air-conditioning fresh-keeping, negative ion, humidification, humidity removal, humidity control, disinfection and sterilization, air purification, air-to-water production and low-temperature storage are intelligently regulated, controlled and integrated under the positive pressure and negative pressure, which are organically applied to the intelligent positive and negative pressure fresh-keeping warehouse and are organically integrated and optimally matched, one item of or a combination of more items of the techniques is used individually or alternately or circularly, and a plurality of positive and negative pressure cabins with different types and different functions are arranged in a positive and negative pressure fresh-keeping warehouse, which are uniformly arranged and allocated according to different demands and convenient use, and are combined arbitrarily combined, adjusted to the cabins, classified and controlled and flexibly applied according to the above procedures, functions and modes. The positive and negative pressure intelligent fresh-keeping warehouse updates the existing method that the traditional refrigerating warehouse achieves low-temperature storage only by controlling the temperature of the warehouse. A regulatable and controllable artificial intelligence microclimate is formed in the positive and negative pressure cabin to create a precise fresh-keeping microenvironment, in which various fresh food stored can keep fresh, moist and original quality and flavor for a long time and to the maximum extent.

Specific embodiment 16: an intelligent positive and negative pressure disinfection machine includes a positive and negative pressure full-functional disinfection machine, a positive and negative pressure multifunctional disinfection machine, a positive and negative pressure automatic humidification and disinfection machine, a positive and negative pressure humidification and disinfection machine, and a positive and negative pressure disinfection machine, where the positive and negative pressure disinfection machine includes a machine body (7). The machine body (7) is internally provided with an intelligent positive and negative pressure system, an air-to-water production apparatus (11), and a power supply and rechargeable battery (33). The intelligent positive and negative pressure system includes an air evacuation pump (2), an air inflation pump (3), a positive and negative pressure intelligent regulation and control apparatus (5), a positive and negative pressure fluid carried substance generation processor (6), a touch screen and mobile phone monitoring system (12), and sensors (C). The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes a super oxygen generator air disinfection apparatus (6.2), a negative ion generator air improvement apparatus (6.3), a humidification and humidity controller air regulation apparatus (6.5), and an instantaneous disinfector filtering and disinfection apparatus (6.11). The touch screen and mobile phone monitoring system (12) includes a touch screen (12.1), a mobile phone monitoring system (12.2), a location positioning system (12.5). The sensors include a super oxygen sensor (C3), a negative ion sensor (C4), a humidity sensor (C6), a disinfection and degradation sensor (C9), and a water level sensor (13). An air inflation pipeline (A3) is installed at an air outlet port (d) of the air inflation pump (3), and air inflation branch pipelines (A3.1), (A3.2), (A3.3), (A3.4), (A3.5) are respectively connected to the other end of the air inflation pipeline (A3), a solenoid valve (B3.1) is installed at a middle part of the air inflation branch pipeline (A3.1), and the other end of the air inflation branch pipeline (A3.1) is connected to an air inflation and outlet port of the machine body (7). A solenoid valve (B3.2) and the negative ion generator air improvement apparatus (6.3) are installed at a middle part of the air inflation branch pipeline (A3.2), and the other end of the air inflation branch pipeline (A3.2) is connected to a negative ion outlet port of the machine body (7). A solenoid valve (B3.3) and the super oxygen generator air infection apparatus (6.2) are installed at a middle part of the air inflation branch pipeline (A3.3), and the other end of the air inflation branch pipeline (A3.3) is connected to a super oxygen outlet port of the machine body (7). A solenoid valve (B3.4) and the humidification and humidity controller air regulation apparatus (6.5) are installed at a middle part of an air inflation branch pipeline (A3.4), and the other end of the air inflation branch pipeline (A3.4) is connected to a humidification and air outlet port of the machine body (7). A solenoid valve (B3.5) and the air-to-water production apparatus (11) are installed at a middle part of an air inflation branch pipeline (A3.5), and the other end of the air inflation branch pipeline (A3.5) is connected to a water production and air exhaust port of the machine body (7). An air intake pipeline (A4) is installed at an air inlet port (e) of the air inflation pump (3), a solenoid valve (B4) is installed at a middle part of the air intake pipeline (A4), and the other end of the air intake pipeline (A4) is connected to a direct air inlet port of the machine body (7). A filtering and air intake pipeline (A4.1) is further connected to the solenoid valve (B4), the air filter (6.8) and a solenoid valve (B4.1) are installed at a middle part of the filtering and air intake pipeline (A4.1), and the other end of the filtering and air intake pipeline (A4.1) is connected to a filtering and air inlet port of the machine body (7). A circulating air return pipeline (A5) is further connected to the solenoid valve (B4.1), the other end of the circulating air return pipeline (A5) is connected to a circulating air return port of the machine body (7). An air-to-water production pipeline (A10) is installed at a water output port (x) of the air-to-water production apparatus (11), the air-to-water production pipeline (A10) communicates with a water inlet port (y) of a filtering water tank (11.1). A filtering water pipeline (A12) is installed at a water output port (v) of the filtering water tank (11.1), the filtering water pipeline (A12) communicates with a water inlet port of the humidification and humidity controller air regulation apparatus (6.5), a water adding pipeline (A11) is installed at a spare water adding port (u) of the filtering water tank (11.1), a solenoid valve (B11) is installed at a middle part of the water adding pipeline (A11), and the other end of the water adding pipeline (A11) is connected to a spare water adding port of the machine body (7). An air exhaust pipeline (A1) is installed at an air inlet port (a) of the air evacuation pump (2), the instantaneous disinfector filtering and disinfection apparatus (6.11) is installed at a middle part of the air exhaust pipeline (A1), and the other end of the air exhaust pipeline (A1) is connected to an air evacuation and air return port of the machine body (7). An air exhaust pipeline (A2) is installed at an air outlet port (b) of the air evacuation pump (2), a solenoid valve (B2) is installed at a middle part of the air exhaust pipeline (A2), and the other end of the air exhaust pipeline (A2) is connected to the atmosphere. The solenoid valve (B2) is connected to a solenoid valve (A3.5) through the pipeline (A2.1), communicates with the air-to-water production pipeline (A3.5) and an air inlet port (h) of the air-to-water production apparatus (11) in sequence, and then communicates with a water production and air exhaust port via the air outlet port (n) of the air-to-water production apparatus (11) after passing through the air-to-water production apparatus (11). The positive and negative pressure multifunctional disinfection machine does not include the negative ion generator air improvement apparatus (6.3), other structures of which are the same as those of the positive and negative pressure full-functional disinfection machine. The positive and negative pressure automatic humidification and disinfection machine does not include the instantaneous disinfector filtering and disinfection apparatus (6.11), other structures of which are the same as those of the positive and negative pressure full-functional disinfection machine. The positive and negative pressure humidification and disinfection machine does not include the air-to-water production apparatus (11), other structures of which are the same as those of the positive and negative pressure automatic humidification and disinfection machine. The positive and negative pressure disinfection machine does not include a humidification and humidity control air regulation apparatus (6.5), other structures of which are the same as those of the positive and negative pressure humidification and disinfection machine. The fluid carried substance generation processor (6), the sensor (C), the solenoid valve (B), the air evacuation pump (2), the air inflation pump (3), the air-to-water production apparatus (11) and the touch screen and mobile phone monitoring and identification system (12) of each machine model are connected to the positive and negative pressure regulation and control apparatus (5).

Specific embodiment 17: an operation method for an intelligent positive and negative pressure disinfection machine is provided. An intelligent positive and negative pressure is used in the intelligent positive and negative pressure disinfection machine, the intelligent positive and negative pressure system is used to regulate, control and integrate various techniques, such as a super oxygen and air disinfection method, a negative ion and air regulation method, a humidification and humidity control and humidity control method, an instantaneous disinfection, and filtering and disinfection, one item of or a combination of more items of the techniques is applied to the positive and negative pressure disinfection machine. An air evacuation pump or air inflation pump is used to intelligently regulate and control airflow, and the airflow is used to carry super oxygen, air disinfectant, water, negative ion and a plurality of effective loads, and the following methods are used for killing viruses and bacteria in the inhabited environment, disinfect the air and the surface of objects, and effectively regulate the air humidity and air composition: (1) the intelligent positive and negative pressure disinfection machine integrates super oxygen and air disinfection methods to inactivate viruses and bacteria including novel coronavirus, kill viruses and bacteria within a certain range, and disinfect the air and the surfaces of the objects, Fujita Health University of Japan has studied that many viruses, including novel coronavirus, are eliminated in the inhabited environment with a low concentration of about 0.05-0.10 ppm of super oxygen, which is acceptable to the human body; the first-class standard of ozone concentration limit in ambient air quality standard (GB3095-1996) in China is 0.12 mg/m3, so the positive and negative pressure disinfection machine is harmless to the human body when used to disinfect the environment to eliminate viruses and bacteria, including novel coronavirus; (2) on the basis of the positive and negative pressure disinfection machine, the positive and negative pressure humidification and disinfection machine is provided with a humidification and humidity control air regulation apparatus, when the super oxygen is used for inactivating the novel coronavirus, the increase of air humidity can enhance the inactivation efficiency; Fujita Health University of Japan has studied that, when the humidity is 80%, the environment is disinfected with about 0.10 ppm of super oxygen, and the virus infectivity drops to 5.7% after 10 hours; (3) on the basis of the positive and negative pressure humidification and disinfection machine, the positive and negative pressure automatic humidification and disinfection machine is additionally provided with an air-to-water production apparatus to provide uninterrupted self-made water for the disinfection machine, thus reducing the disadvantages of frequent manual water addition to a humidifier; (4) on the basis of the positive and negative pressure automatic humidification and disinfection machine, the positive and negative pressure multifunctional disinfection machine is additionally provided with an instantaneous disinfector or a filtering disinfection apparatus, thus providing an instantaneous disinfection function for the disinfection machine other than super oxygen disinfection to make up for the shortage that super oxygen cannot achieve instantaneous disinfection; and (5) on the basis of the positive and negative pressure multifunctional disinfection machine, the positive and negative pressure full-functional disinfection machine is additionally provided with a negative ion generator air improvement apparatus, so as to additionally provide an air improvement function for the disinfection machine.

Specific embodiment 18: an intelligent positive and negative pressure module cabinet includes a cabinet body (7). The cabinet body (7) is internally provided with an intelligent positive and negative pressure system, and a refrigeration and heating system (8.3). The positive and negative pressure system includes a positive and negative pressure cabin (1), an air evacuation pump (2), an air inflation pump (3), a positive and negative pressure intelligent regulation and control apparatus (5), a positive and negative pressure airflow carried substance generation processor (6), and a touch screen and mobile phone monitoring and identification system (12). The intelligent positive and negative pressure system also adopts a nonstandard structure other than the above standard structures in specific applications; the nonstandard structure of the intelligent positive and negative pressure system includes the air evacuation pump (2) or air inflation pump (3), and at least one or more of the positive and negative pressure cabin (1), the air evacuation pump (2), the air inflation pump (3), the intelligent positive and negative pressure regulation and control apparatus (5), the positive and negative pressure fluid carried substance generation processor (6), and the touch screen and mobile phone monitoring and identification system (12). The positive and negative pressure cabin (1) is designed as a module type positive and negative pressure cabin (1T) according to the requirements of the intelligent positive and negative pressure module cabinet, and the module type positive and negative pressure cabin (1T) is a vacuum high-pressure cabin (1-1) structure. A positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the module cabinet; the module type positive and negative pressure cabin (1T) includes a cabin body (1.1), a cabin door (1.2), a mechanical airtight mechanism (1.3), and an internal and external communicating sealer (1.4). The cabin door (1.2) is a cabinet door of the positive and negative pressure module cabinet; the airtight mechanism (1.3) is arranged between the cabin door (1.2) and the cabin body (1.1) of the module type positive and negative pressure cabin (1T). The internal and external communicating sealer (1.4) is fixedly arranged at a rear part of the module type positive and negative pressure cabin (1T), all pipelines and circuits getting in and out the cabin are connected to and pass through the internal and external communicating sealer (1.4), so as to keep the sealing property of the module type positive and negative pressure cabin (1T). The intelligent positive and negative pressure regulation and control apparatus (5) includes an integrated circuit, a chip, a regulation and control system for a fluid and carried substance thereof, and is connected to the Internet in wired and wireless modes. The positive and negative pressure fluid carried substance generation processor (6) includes an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), an airflow carried substance generator integration (6.02) or (6.021), or an integrated pipeline type positive and negative fluid carried substance generator (6.05). The air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same. The airflow carried substance generator integration (6.02) or (6.021) or the integrated pipeline type positive and negative pressure fluid carried substance generator (6.05) includes any one or more in the individual fluid carried substance generators (6.1) to (6.16) as required. The module type positive and negative pressure cabin (1T) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration and heating system (8.3), an air-to-water production apparatus (11), and the touch screen and mobile phone monitoring and identification system (12). An air evacuation pipeline (A1) is installed at an air inlet port (a) of the air evacuation pump (2), a solenoid valve (B1) is installed at a middle part of the air evacuation pipeline (A1), and the other end of the air evacuation pipeline (A1) extends into the module type positive and negative pressure cabin (1T) to form an air evacuation and air return port (c) thereof. Carrying waste gas carrying waste gas evacuation and exhaust pipelines (A8) and (A8.1) are respectively connected to the solenoid valve (B1), the other end of the carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the carrying waste gas exhaust and evacuation pipeline (A8.1) is connected to a carrying waste gas evacuation and exhaust port (s2) of the air conditioning apparatus (6.1) integrated in the positive and negative pressure airflow carried substance generator integration (6.021). An air exhaust pipeline (A2) is installed at an air outlet port (b) of the air evacuation pump (2), the carried substance decomposition processor (6.7) and a solenoid valve (B2) are installed at a middle part of the air exhaust pipeline (A2), the other end of the air exhaust pipeline (A2) communicates with an air inlet port (h) of the air-to-water production apparatus (11), passes through the air-to-water production apparatus (11), and then is connected to the atmosphere from an air outlet port (n) of the air-to-water production apparatus (11). An air inflation pipeline (A3) is installed at an air outlet port (d) of the air inflation pump (3), a solenoid valve (B3) is installed at a middle part of the air inflation pipeline (A3), and the other end of the air inflation pipeline (A3) extends into the module type positive and negative pressure cabin (1T) to form an air inflation and inlet port (f) thereof. An air intake pipeline (A4) is installed at an air inlet port (e) of the air inflation pump (3), a solenoid valve (B4) and the air filter (6.8) are installed at a middle part of the air intake pipeline (A4), and the other end of the air intake pipeline (A4) is connected to the atmosphere. A circulation pipeline (A5) is further connected to the solenoid valve (B4), and the other end of the circulation pipeline (A5) communicates with the solenoid valve (B3), and extends into the module type positive and negative pressure cabin (1T) through the air inflation pipeline (A3) to form a circulating air return port (j) thereof; carrying air introduction pipelines (A6), (A6.1) and (A6.2) are further connected to the air inflation pipeline (A3), a solenoid valve (B6) is installed at a middle part of the carrying air introduction pipeline (A6), the other end of the carrying air introduction pipeline (A6) is connected to an air inlet port (t) of the positive and negative pressure airflow carried substance generator integration (6.02). A carrying air introduction pipeline (A7) is installed at an air outlet port (s) of the positive and negative pressure airflow carried substance generator integration (6.02), a solenoid valve (B7) is installed at a middle part of the carrying air introduction pipeline (A7), and the other end of the carrying air introduction pipeline (A7) extends into the positive and negative pressure cabin (1T) to form a carrying air inlet port (i) thereof. Solenoid valves (B6.1) and (B6.2) are respectively installed at middle parts of the carrying air introduction pipelines (A6.1) and (A6.2), the other ends of the carrying air introduction pipelines (A6.1) and (A6.2) extend into the positive and negative pressure cabin (1) to be connected to air inlet ports of the positive and negative pressure airflow carried substance generator integration (6.021) and the integrated pipeline type positive and negative pressure fluid carried substance generator (6.05). An air-to-water production inflation pipeline (A9) is further connected to the air inflation pipeline (A3), the air-to-water production inflation pipeline (A9) is connected to the solenoid valve (B2) and is then connected to the air exhaust pipeline (A2). The air-to-water production apparatus (11) communicates with the middle part of the air exhaust pipeline (A2), and the other end of the air exhaust pipeline (A2) is connected to the atmosphere; the touch screen and mobile phone monitoring and identification system (12) includes a cabinet door touch screen (12.1), a mobile phone APP (12.2), a high-definition anti-fog camera device (12.3), and a radar scanning and identification apparatus (12.5). The high-definition anti-fog camera device (12.3) and the radar scanning and identification apparatus (12.5) are installed at positions inside/outside the cabinet body (7) needing to be monitored. The module type positive and negative pressure cabin (1T) is internally provided with the airflow carried substance generator integration (6.021) or the integrated pipeline type fluid carried substance generator (6.05) and sensors (C). The sensors include a sensor integration (CA), the sensor integration (CA) includes at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12). The carried substance sensor item addition and upgrading module (C12) is a combined module capable of providing and installing any individual sensor additional item into the sensor integration and upgrading and updating the same. All fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E). An operation method for the intelligent positive and negative pressure module cabinet is provided. Due to the use of an intelligent positive and negative pressure system, the intelligent positive and negative pressure module cabinet has a vacuum, high-pressure, constant-temperature, constant-humidity, constant-pressure, sterile and dust-free environment, various objects and equipment, such as tableware, kitchenware and electric appliances, can be placed and installed therein. The operation method includes: (1) vacuum abatement cleaning procedure: after placing the objects into the module type positive and negative pressure cabin (1T), closing the chamber door 1.2, and sending an instruction by the positive and negative pressure intelligent regulation and apparatus (5) immediately, enabling solenoid valves (B1) and (B2) to open air evacuation pipelines (A1) and (A2): air evacuation and air return port (c) of vacuum high-pressure cabin→(A1)→(B1)→(A1)→air inlet port (a) of air evacuation pump→air outlet port (b)→(A2)→air inlet port (g) of super oxygen decomposer→air outlet port (m)→(A2)→(B2)→(A2)→air inlet port (h) of air-to-water production apparatus→air outlet port (n)→exhaust to the atmosphere, and turning on the air evacuation pump (2) to vacuumize the module type vacuum high-pressure cabin (1T) to a moderate vacuum from −0.01 KPa to −0.1 MPa (with the local instant atmospheric pressure as the zero standard), pumping out part of bacteria and microorganisms and polluted air in the cabin to clean the air and space; (2) carried substance disinfection and sterilization procedure: sending an instruction by the positive and negative pressure intelligent regulation and control apparatus (5) to turn on a corresponding carried substance generation processor (6), which can be selected according to different disinfection requirements to disinfect and sterilize the environment and objects in the cabin; (3) sterile and undamaged storage procedure under particular conditions: providing vacuum, high-pressure, constant-temperature, constant-humidity, constant-pressure, sterile, dust-free and other particular storage environments by different types and configurations of module cabinets, thus satisfying various different storage demands, for example, the tableware, kitchenware, small electric appliances and small objects can be stored in vacuum after being disinfected by super oxygen, paintings, documents, antiques and high-end clothing may be stored in vacuum, low oxygen and dust prevention, or stored by using a particular modified atmosphere gas, or stored at constant temperature, constant humidity and constant pressure; (4) multi-purpose display refrigeration of water and wine bar: refrigerating and storing the packaged food, beverages, alcoholic beverages, fruits and dried fruits, candy and cakes and other food and objects with transparent cabinet doors in a display manner; (5) remote and short-range control monitoring and identification function: controlling, monitoring and identifying, by the touch screen and mobile phone monitoring and identification system (12), the positive and negative pressure module cabinet in real time by the WIFI and mobile phone APP at remote and short-range, thus making the module cabinet to intelligently achieve various functions at high efficiency and low consumption according to the set procedures and instant instructions; shooting, by the high-definition anti-fog camera and identification device (12.3), the statuses of objects in the module cabinet in real time, and dynamically identifying the objects, and uploading the data; displaying and reminding, by a cabinet door touch screen (12.1) or a mobile phone APP (12.2), commodity information such as production date, price, expiration date and manufacturers and the information of purchase shopping malls and online stores, automatically recording, analyzing and handling the big data such as the time, quantity, frequency and preference of storing the objects in, and taking the objects out, the module cabinet, and performing intelligent analysis, and timely reminding on the mobile phone APP 12.2, where, in addition to real-time monitoring and recording, the high-definition anti-fog camera device (12.3) is especially required to automatically and continuously shoot several panoramic photos of the objects in the cabinet with the closing movement of the cabinet door (1.2) by starting from the position remote away from the cabinet body in the process of closing the cabinet door, or automatically record small videos at the same time for the users to view in the mobile phone APP, or view the panoramic photos or videos of the objects in the cabinet with good light and wide field of vision before and during the last closing of the door in the cabinet door touch screen at any time without opening the module cabinet door, releasing the vacuum and lowering the temperature during short range, and dynamically identifying the objects, and uploading the data; (6) positive and negative pressure module combined intelligent smart home mode: designing and manufacturing the intelligent positive and negative pressure module cabinet according to the module functions, wherein the intelligent positive and negative pressure module cabinet can be independently produced and used, and also can be organically combined with other positive and negative pressure electric appliances and the module cabinets under the control of the touch screen and mobile phone APP monitoring and identification system (12) to form a fully functional intelligent positive and negative pressure module combined home; and the module cabinet not only has high energy efficiency and low consumption, but also can share the intelligent positive and negative pressure system, the refrigeration system and the air-to-water production apparatus of other positive and negative pressure electric appliances and module cabinets, and the fully functional intelligent positive and negative pressure module combined home is basically uniform, neat and harmonious in appearance, shape and color which are in line with the popular trend.

1. An intelligent positive and negative pressure system, comprising:
a positive and negative pressure cabin (1);
positive and negative pressure fluid carried substance generation processors (6);
a positive and negative pressure intelligent regulation and control apparatus (5); and
an air evacuation pump (2) and/or an air inflation pump (3),
wherein the positive and negative pressure intelligent regulation and control apparatus (5) and the air evacuation pump (2) and/or air inflation pump (3) are arranged outside the positive and negative pressure cabin (1), and
wherein the positive and negative pressure fluid carried substance generation processors (6), the air evacuation pump (2) and/or the air inflation pump (3) are connected to the positive and negative pressure intelligent regulation and control apparatus (5) by lines (D) or bundled lines (E).

2. An operation method for the intelligent positive and negative pressure system according to claim 9, comprising forming various targeted influence methods or targeted control modes with different characteristics for various affected objects by a positive and negative pressure intelligent regulation and control apparatus (5) through built-in program and based on pre-stored data and cloud data, and real-time feedback information of a touch screen and mobile phone monitoring and identification system (12) and various sensors (C11) to (C12), and sending, by the positive and negative pressure intelligent regulation and control apparatus (5), instructions to an air evacuation pump (2), an air inflation pump (3), a water suction pump (16), a water input pump (17), related positive and negative pressure fluid carried substance generators (6) and related solenoid valves (c) in real time, so as to control and timely adjust open, close and switching of various air evacuation, inflation and circulation pipelines or water suction, intake and circulation pipelines and intelligently regulate and control the level of positive and negative pressure in the positive and negative pressure cabin (1) and flowing-in, staying and flowing-out of an airflow and a carried substance thereof or a waterflow and a carried substance thereof, wherein the operation method is as follows:
operation method (I): an operation method for regulating an interior of the positive and negative pressure cabin (1) into negative pressure, namely, vacuum, is as follows: sending an instruction by the positive and negative pressure intelligent regulation and control apparatus (5) to enable a first solenoid valve (B1) and a second solenoid valve (B2) to open an air evacuation pipeline (A1) and an air exhaust pipeline (A2), exhausting air to atmosphere through an air evacuation and air return port (c) of the positive and negative pressure cabin, the air evacuation pipeline (A1), a first solenoid valve (B1), the air evacuation pipeline (A1), an air inlet port (a) of the air evacuation pump, an air outlet port (b) of the air evacuation pump, the air exhaust pipeline (A2), a second solenoid valve (B2), and the air exhaust pipeline (A2), and turning on the air evacuation pump (2) to vacuumize the interior of the positive and negative pressure cabin (1) to set negative pressure vacuum, where when any pipeline is opened, all other unrelated solenoid valves are closed;
operation method (II): an operation method for regulating and controlling the interior of the positive and negative pressure cabin 1 to positive pressure, namely, high pressure, is as follows: sending an instruction by the positive and negative pressure intelligent regulation and control apparatus (5) to enable a third solenoid valve (B3) of an air inflation pipeline (A3) and a fourth solenoid valve (B4) of an air intake pipeline (A4) to open the air inflation pipeline (A3) and the air intake pipeline (A4), intaking air from atmosphere, and then enabling the air to pass through the air intake pipeline (A4), the fourth solenoid valve (B4), the air intake pipeline (A4), an air inlet port (e) of the air inflation pump, an air outlet port (d) of the air inflation pump, the air inflation pipeline (A3), the third solenoid valve (B3), the air inflation pipeline (A3), and an air inflation and intake (f) of the positive and negative pressure cabin, and turning on the air inflation pump (3) to inflate the interior of the positive and negative pressure cabin (1) to the set positive pressure, namely, high pressure; the above operation methods (1) and (2) are conducted in a vacuum or high-pressure space;
operation method (III): an operation method for regulating airflow and a carried substance thereof to flow in, stay in or flow out of the positive and negative pressure cabin (1) is as follows:
(i) outflow, when the cabin is under the negative pressure or atmospheric, evacuating the airflow and the carried substance thereof out of the positive and negative pressure cabin (1) from air evacuation pipelines using the air evacuation pump (2) according to the above operation method (I), and when the cabin is under the high pressure, opening the corresponding solenoid valves for the airflow and the carried substance to flow out from the air evacuation pipelines;
(ii) inflow, inflating the airflow and the carried substance thereof into the positive and negative pressure cabin from air inflation pipelines or carrying pipelines using the air inflation pump (3), with a method as follows: A: inflow of the airflow: when the cabin is under the high pressure or atmospheric, inflating the airflow into the positive and negative pressure cabin from the air inflation pipeline according to the operation method (II) using the air inflation pump (3), and when the cabin is under the negative pressure, opening related solenoid valves to enable the airflow to automatically flow into the cabin from the air inflation pipeline; B: inflow of carrying airflow generated by an airflow carried substance generator integration (6.02): sending an instruction by the positive and negative pressure intelligent regulation and control apparatus (5) to enable a sixth solenoid valve (B6) and a seventh solenoid valve (B7) to open the air inflation pipelines (A3), a carrying air intake pipeline (A6.2), a first carrying air introduction pipeline (A6) and a second carrying air introduction pipeline (A7), intaking air from atmosphere, and enabling the air to pass through the air intake pipeline (A4), the fourth solenoid valve (B4), the air intake pipeline (A4), an air inlet port (e) of the air inflation pump, an air outlet port (d) of the air inflation pump, the air inflation pipeline (A3), the carrying air intake pipeline (A6.2), the first carrying air introduction pipeline (A6), the sixth solenoid valve (B6), the first carrying air introduction pipeline (A6), a related airflow carried substance generator in an airflow carried substance generator integration (6.02), the second carrying air introduction pipeline (A7), the seventh solenoid valve (B7), the second carrying air introduction pipeline (A7), and a carrying air inlet port (i) of the positive and negative pressure cabin, and turning on the air inflation pump (3) and the related airflow carried substance generator to make the pressure in the positive and negative pressure cabin (1) and inflow of the airflow carried substance reach a set standard; C: inflow of a carrying airflow generated by an integrated pipeline type fluid carried substance generator (6.05): sending an instruction by the positive and negative pressure intelligent regulation and control apparatus (5) to enable a solenoid valve (B6.2) to open the air inflation pipeline (A3) and the carrying air intake pipeline (A6.2), intaking air from atmosphere, and enabling the air to pass through the air intake pipeline (A4), the solenoid valve (B4), the air intake pipeline (A4), an air inlet port (c) of the air inflation pump, and air outlet port (d) of the air inflation pump, the air inflation pipeline (A3), the carrying air intake pipeline (A6.2), the sixth solenoid valve (B6.2), the carrying air intake pipeline (A6.2), a related airflow carried substance generator in an integrated pipeline type fluid carried substance generator (6.05) to enter the positive and negative pressure cabin, and turning on the air inflation pump (3) and the related airflow carried substance generator in the integrated pipeline type fluid carried substance generator (6.05) to make the pressure in the positive and negative pressure cabin and inflow of the airflow carried substance reach a set standard;
(iii) staying, when the airflow and the carried substance thereof need to stay in the positive and negative pressure cabin, sending an instruction by the positive and negative pressure intelligent regulation and control apparatus (5) to make the airflow and pressure and carried substance in the cabin reach the standard, and then closing the corresponding solenoid valves; and
(iv) circulation: A: air inflation circulation: when the airflow and the carried substance thereof need to circulate to flow through the positive and negative pressure fluid carried substance generator to make the pressure, concentration and composition of the air flow and the carried substance thereof in the positive and negative pressure cabin reach the standard, sending an instruction by the positive and negative pressure intelligent regulation and control apparatus (5) to open the air inflation pipeline (A3), a first carrying circulation pipeline (A5), the first carrying air introduction pipeline (A6), the carrying air intake pipeline (A6.2) and the second carrying air introduction pipeline (A7) and close the solenoid valve (B4) and corresponding solenoid valves, and turning on the air inflation pump (3) and related airflow carried substance generators to make carried substance gases circulate and flow, during the operation of an air conditioning apparatus (6.1), moderately increasing partial pressure difference between both sides of a membrane to improve air separation efficiency, and evacuating and exhausting oxygen-enriched waste gas or carried waste gas; and opening a carrying waste gas evacuation and exhaust pipeline (A8), the air evacuation pipeline (A1) and the air exhaust pipeline (A2) by the first solenoid valve (B1) and the second solenoid valve (B2), exhausting the waste gas to atmosphere through a carrying waste gas evacuation and exhaust port (s2) of the air conditioning apparatus (6.1), the first solenoid valve (B1), the air evacuation pipeline (A1), an air inlet port (a) of the air evacuation pump, an air outlet port (b) of the air evacuation pump, the air exhaust pipeline (A2), the second solenoid valve (B2), and the air exhaust pipeline (A2), and turning on the air evacuation pump (2), operating circularly until corresponding sensors in the positive and negative pressure cabin feed back that the concentration, composition or pressure of the air-conditioned or carrying gas reach the standard, and sending a stop instruction by the positive and negative pressure intelligent regulation and control apparatus (5); B: air evacuation circulation: sending an instruction by the positive and negative pressure intelligent regulation and control apparatus (5) to open the air evacuation pipeline (A1), a second air carrying circulation pipeline (A5.1), a carrying air evacuation pipeline (A6.1), the first carrying air introduction pipeline (A6), and the second carrying air introduction pipeline (A7) and close the second solenoid valve (B2) and related solenoid valves, and turning on the air evacuation pump (2) and related airflow carried substance generators to make carried substance gases circulate and flow until corresponding sensors in the positive and negative pressure cabin feed back that the concentration, composition or pressure of the air-conditioned gas reach the standard, and sending a stop instruction by the positive and negative pressure intelligent regulation and control apparatus (5);
as the operation methods for the positive and negative pressure cabin (1), namely, (i) outflow, (ii) inflow, (iii) staying and (iv) circulation, are conducted in an atmospheric space of an atmospheric circulation cabin (1-2) and an atmospheric inlet-outlet cabin (1-3);
operation method (IV): an operation method for regulating a waterflow and a carried substance thereof to flow in, stay in or flow out of the positive and negative pressure cabin (1) is as follows:
(i) outflow, pumping the waterflow and the carried substance thereof out of the positive and negative pressure cabin from water suction pipelines using a water suction pump (16), with methods as follows: sending an instruction by the positive and negative pressure intelligent regulation and control apparatus (5) to open a water suction pipeline (A20) and a water drainage pipeline (A21) by a twelfth solenoid valve (B20) and a thirteenth solenoid valve (B21), draining water to a sewer through a water suction port (L) of the positive and negative pressure cabin, the water suction pipeline (A20), the twelfth solenoid valve (B20), the water suction pipeline (A20), a water inlet port (w) of the water suction pump, a water drainage port (p) of the water suction pump, the water drainage pipeline (A21), the thirteenth solenoid valve (B21), and the water drainage pipeline (A21), and turning on the water suction pump (16) to drain the water in the positive and negative pressure cabin (1) to a set standard;
(ii) inflow, feeding the waterflow and the carried substance thereof into the positive and negative pressure cabin from air feeding and intake pipelines or carrying water intake pipelines using the water input pump (17), with methods as follows: A: water feeding and intake: sending an instruction by the positive and negative pressure intelligent regulation and control apparatus (5) to open the water intake pipeline (A14) and the water intake pipeline (A15) by an eighth solenoid valve (B14) and a ninth solenoid valve (B15), intaking water from a water source, and enabling the water to pass through the water intake pipeline (A14), the eighth solenoid valve (B14), the water intake pipeline (A14), a water inlet port (r) of the water input pump, a water output port (u) of the water input pump, the water intake pipeline (A15), the ninth solenoid valve (B15), the water intake pipeline (A15), and a water feeding and inlet port (T) of the positive and negative pressure cabin, and turning on the water input pump (17) to make the water feeding in the positive and negative pressure cabin (1) reach the standard; B: carrying water intake: sending an instruction by the positive and negative pressure intelligent regulation and control apparatus (5) to open the water intake pipeline (A14), the water intake pipeline (A15), a first carrying water introduction pipeline (A16), the carrying water intake pipeline (A16.2) and a second carrying water introduction pipeline (A17) by the eighth solenoid valve (B14), a tenth solenoid valve (B16) and an eleventh solenoid valve (B17), intaking water from the water source, and enabling the water to pass through the water intake pipeline (A14), the eighth solenoid valve (B14), the water intake pipeline (A14), a water inlet port (r) of the water input pump, a water output port (u) of the water input pump, the water intake pipeline (A15), the carrying water intake pipeline (A16.2), the tenth solenoid valve (B16), the carrying water introduction pipeline (A16), a corresponding waterflow carried substance generator, the second carrying water introduction pipeline (A17), the eleventh solenoid valve (B17), the second carrying water introduction pipeline (A17), and a carrying water inlet port (T1) of the positive and negative pressure cabin, and turning on the water input pump (17) and the corresponding waterflow carried substance generator to make water feeding in the positive and negative pressure cabin (1) and inflow of the waterflow carried substance to a set standard;
(iii) staying, when the waterflow and the carried substance thereof need to stay in the positive and negative pressure cabin, sending an instruction by the positive and negative pressure intelligent regulation and control apparatus (5), firstly making the waterflow pressure and carried substance in the cabin reach the standard, and then closing the corresponding solenoid valves until staying time reaches the standard, and sending a stop instruction by the positive and negative pressure intelligent regulation and control apparatus (5); and
(iv) circulation: A: water intake circulation: when the waterflow and the carried substance thereof need to circulate to flow through the positive and negative pressure fluid carried substance generator to make the pressure, concentration and composition of the waterflow and the carried substance thereof in the positive and negative pressure cabin reach the standard, sending an instruction by the positive and negative pressure intelligent regulation and control apparatus (5) to open the second water intake pipeline (A15), the first carrying water introduction pipeline (A16), the carrying water intake pipeline (A16.2), the second carrying water introduction pipeline (A17) and a third carrying water circulation pipeline (A19) and close the eighth solenoid valve (B14) and related solenoid valves, and turning on the water input pump (17) and related waterflow carried substance generators to make substance-carrying waterflow circulate and flow until corresponding sensors in the positive and negative pressure cabin feed back that the concentration, the composition or pressure of the carrying waterflow reach the standard, and sending a stop instruction by the positive and negative pressure intelligent regulation and control apparatus (5); B: water suction circulation: sending an instruction by the positive and negative pressure intelligent regulation and control apparatus (5) to open a water evacuation pipeline (A20), the first carrying water introduction pipeline (A16), the carrying water suction pipeline (A16.1), the second carrying water introduction pipeline (A17) and a fourth carrying circulation pipeline (A19.1) and close the thirteenth solenoid valve (B21) and related solenoid valves, and turning on the water suction pump (16) and related waterflow carried substance generators to make the substance-carrying waterflow circuit and flow until corresponding sensors in the positive and negative pressure cabin feed back that the concentration, the composition or pressure of the waterflow reach the standard, and sending a stop instruction by the positive and negative pressure intelligent regulation and control apparatus (5); and
one or more of the operation methods, such as, (i) outflow, (ii) inflow, (iii) staying and (iv) circulation, for regulating and controlling the waterflow and carried substance thereof to flow in, stay in or flow out the positive and negative pressure cabin (1) are conducted not only in a vacuum high pressure cabin, but also in the atmospheric circulation cabin (1-2) and the atmospheric inlet-outlet cabin (1-3).

3. A fresh-keeping refrigerator comprising the intelligent positive and negative pressure system according to claim 1, further comprising:
a refrigerator body (7); and
an air-to-water production apparatus (11),
wherein the refrigerator body (7) is internally provided with the intelligent positive and negative pressure system, a refrigeration system (8), and the air-to-water production apparatus (11).

4. An operation method for the fresh-keeping refrigerator according to claim 20, comprising: performing analysis one by one by the positive and negative pressure intelligent regulation and control apparatus (5) through built-in program and based on pre-stored data, and internet cloud data, camera identification, radar scanning and identification of the touch screen and mobile phone monitoring and identification system (12), and real-time feedback information of various sensors (C1) to (C12), correspondingly using different targeted fresh-keeping techniques for fresh-keeping factors and control targets of different storage products to form targeted control comprehensive fresh-keeping methods with different characteristics, and sending, by the positive and negative pressure intelligent regulation and control apparatus (5), instructions to an air evacuation pump (2), an air inflation pump (3), related positive and negative pressure fluid carried substance generators (6) and related solenoid valves (B) in real time to control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof; wherein the targeted control comprehensive fresh-keeping method and specific use methods and operation procedures of the fresh-keeping refrigerator are as follows:
(1) exhaust and vacuum abatement heat-removal cleaning procedure: closing a cabin door (1.2), sending an instruction by the positive and negative pressure intelligent regulation and control apparatus (5) to enable a fifth solenoid valve (B2) and a first solenoid valve (B1.1) or a second solenoid valve (B1.2) or a third solenoid valve (B1.3) or a fourth solenoid valve (B1.4) to open corresponding air exhaust pipeline (A2) and an air exhaust pipeline (A1) and a first air exhaust branch pipeline (A1.1) or a second air exhaust branch pipeline (A1.2) or a third air exhaust branch pipeline (A1.3) or a fourth air exhaust branch pipeline (A1.4) of the related positive and negative pressure cabin, and turning on the air evacuation pump (2) to exhaust or pump the related positive and negative pressure cabin to vacuum negative pressure between −0.001 KPa and −0.1 MPa, the local instant atmospheric pressure is used as the zero standard, and the vacuum degree is able to be improved according to the demands and refrigerator configuration;
(2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within the positive and negative pressure cabin, and meanwhile, turning on the a humidification and humidity control apparatus (6.5) to increase and control humidity in the cabin;
(3) atmospheric or pressurized sterilization, degradation and deodorization procedure for super-oxygen negative ion or catalyst positive and negative pressure carried substance: according to the procedure setting and feedback of the related sensors (C), sending, by the positive and negative pressure intelligent regulation and control apparatus (5), an instruction at a proper time to turn on a super oxygen generator (6.2), a negative ion generator (6.3), a catalyst release controller (6.4), a humidity control apparatus (6.5) or a disinfection and degradation apparatus (6.9) in each related airflow carried substance generator integration, or a carried substance generator installed in a carried substance generation item addition and upgrading module (6.16), thus enabling the super oxygen, catalyst, negative ion, water and related carried substances in the positive and negative pressure cabin reach required standards; meanwhile, enabling a sixth solenoid valve (B3) and an eleventh solenoid valve (B4) or a seventh solenoid valve (B3.1) or an eighth solenoid valve (B3.2) or a ninth solenoid valve (B3.3) or a tenth solenoid valve (B3.4) to open corresponding air intake pipeline (A4) and air inflation pipeline (A3) or a first air inflation branch pipeline (A3.1) or a second air inflation branch pipeline (A3.2) or a third air inflation branch pipeline (A3.3) or a fourth air inflation branch pipeline (A3.4) of the related positive and negative pressure cabin, turning on the air inflation pump (3) to properly pressurize the related positive and negative pressure cabin to 0.001 KPa to 10 MPa;
(4) atmospheric or pressurized sterilization, degradation and deodorization procedure for air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling the sixth solenoid valve (B3), a seventeenth solenoid valve (B6) and a twenty-second solenoid valve (B7.1), a twenty-third solenoid valve (B7.2), a twenty-fourth solenoid valve (B7.3) or a twenty-fifth solenoid valve (B7.4) to open the air inflation pipeline (A3), a carrying air introduction pipeline (A6) and a first carrying air introduction branch pipeline (A7.1), a second carrying air introduction branch pipeline (A7.2), a third carrying air introduction branch pipeline (A7.3) or a fourth carrying air introduction branch pipeline (A7.4) and turn on the air inflation pump (3) to drive the airflow to enter the related positive and negative pressure cabin after passing through an air conditioning apparatus (6.1) and other related positive and negative pressure fluid carried substance generators (6); and enabling the sixth solenoid valve (B3), the eleventh solenoid valve (B4), a thirty-first solenoid valve (B9) and a thirteenth solenoid valve (B5.1), a fourteenth solenoid valve (B5.2), a fifteenth solenoid valve (B5.3) or a sixteenth solenoid valve (B5.4) to open carrying circulation pipelines to circulate and operate; during the operation of an air conditioning apparatus (6.1), moderately increasing partial pressure difference between both sides of a membrane to improve air separation efficiency, and evacuating and exhausting oxygen-enriched waste gas or carried waste gas; enabling a twenty-sixth solenoid valve (B8) or a twenty-seventh solenoid valve (B8.1), a twenty-eighth solenoid valve (B8.2), a twenty-ninth solenoid valve (B8.3) or a thirtieth solenoid valve (B8.4) to open a first carrying waste gas evacuation and exhaust pipeline (A8) or a second carrying waste gas evacuation and exhaust pipeline (A8.1), a third carrying waste gas evacuation and exhaust pipeline (A8.2), a fourth carrying waste gas evacuation and exhaust pipeline (A8.3) or a fifth carrying waste gas evacuation and exhaust pipeline (A8.4), exhausting the waste gas to atmosphere through a carrying waste gas evacuation and exhaust port (s3) or a carrying waste gas evacuation and exhaust port (s4) or a carrying waste gas evacuation and exhaust port (s5) or a carrying waste gas evacuation and exhaust port (s6) or a carrying waste gas evacuation and exhaust port (s7) of the air conditioning apparatus (6.1), the first carrying waste gas evacuation and exhaust pipeline (A8) or the second carrying waste gas evacuation and exhaust pipeline (A8.1) or the third carrying waste gas evacuation and exhaust pipeline (A8.2) or the fourth carrying waste gas evacuation and exhaust pipeline (A8.3) or the fifth carrying waste gas evacuation and exhaust pipeline (A8.4), the twenty-sixth solenoid valve (B8) or the twenty-seventh solenoid valve (B8.1) or the twenty-eighth solenoid valve (B8.2) or the twenty-ninth solenoid valve (B8.3) or the thirtieth solenoid valve (B8.4), the first carrying waste gas evacuation and exhaust pipeline (A8) or the second carrying waste gas evacuation and exhaust pipeline (A8.1) or the third carrying waste gas evacuation and exhaust pipeline (A8.2) or the fourth carrying waste gas evacuation and exhaust pipeline (A8.3) or the fifth carrying waste gas evacuation and exhaust pipeline (A8.4), the air evacuation pipeline (A1), air inlet port (a) of the air evacuation pump, air outlet port (b) of the air evacuation pump, the air exhaust pipeline (A2), the carried substance decomposition processor (6.7), the second solenoid valve (B2), the air exhaust pipeline (A2) and the air-to-water production apparatus (11); and meanwhile, turning on the air evacuation pump (2) to exhaust decomposed waste gas;
(5) atmospheric, pressurized, or sterilized preservation procedure for leftovers: placing the leftovers into the positive and negative pressure cabin, turning on the air evacuation pump (2) and related air pipelines by the regulation and control apparatus (5) until the odor already released by the leftovers and the polluted air inside the cabin are pumped out of the cabin; turning on the air inflation pump (3) and related air inflation pipelines for properly pressurized preservation, which not only prevents the odor and water from excessively scattering and losing, but also inhibits the food spoilage; meanwhile, according to feedback information of the humidity sensor, turning on the humidity control apparatus (6.5) at proper time to supplement the water in the air, or turning on related carried substance generation processors at proper time to kill the bacteria and virus in the air in the cabin to prevent food spoilage;
(6) low-temperature assisted fresh-keeping procedure: after the positive and negative pressure fresh-keeping refrigerator is turned on, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects;
(7) open-to-exhaust protection function: if the refrigerator door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to a carried substance decomposition processor (6.7) at the rear part of the refrigerator to be exhausted to atmosphere after being decomposed, wherein, as the air flows from the outside of the refrigerator door to the positive and negative pressure cabin for supplementing air evacuation negative pressure, the super oxygen or high-nitrogen low-oxygen carried substance gas is unable to flow out of the door;
(8) air-to-water production procedure: enabling moist waste gas pumped from the vacuum high pressure cabin by the air evacuation pump 2 to enter the super oxygen decomposer (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling the sixth solenoid valve (B3), the thirty-first solenoid valve (B9) and the fifth solenoid valve (B2) to open air-to-water production inflation pipelines, thus driving the indoor air to enter the air-to-water production apparatus (11) for water production; and
(9) remote and short-range control monitoring and identification function: controlling and monitoring, by the touch screen and mobile phone monitoring and identification system (12), the fresh-keeping refrigerator and other positive and negative pressure electric appliances and a positive and negative pressure module cabinet in real time by the WIFI and mobile phone APP at remote and short-range, thus making the fresh-keeping refrigerator and other all positive and negative pressure electric appliances and module cabinets to intelligently achieve various functions at high efficiency and low consumption according to the set procedures and instant instructions, wherein a high-definition anti-fog camera and identification apparatus (12.3) and a radar scanning and identification apparatus (12.5) are installed at positions needing monitoring and identification inside and outside the refrigerator body (7); shooting and scanning statuses of objects in the refrigerator in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling various fresh-keeping environmental factors such as airflow pressure, gas composition, air cleanliness, environment temperature and humidity in the positive and negative pressure refrigerating-freezing cabin in real time; displaying and reminding, by a refrigerator door touch screen (12.1) or a mobile phone APP (12.2), commodity information such as production date, price, expiration date and manufacturers and information of purchase shopping malls and online stores, automatically recording, analyzing and handling the big data such as the time, quantity, frequency and preference of preserving the objects in, and taking the objects out, the refrigerator, and performing intelligent analysis by combining cloud data; timely reminding on the mobile phone APP (12.2) or directly pushing the suggested goods to the mobile phone APP (12.2) or the refrigerator door touch screen (12.1), wherein, in addition to real-time monitoring and identification at a fixed position, the camera and identification apparatus (12.3) is required to be installed in the refrigerator door (1.2) and to automatically and continuously shoot a plurality of panoramic photos of the objects in the refrigerator and automatically record small videos with the closing movement of the refrigerator door (1.2) by starting from the position away from the refrigerator body in the process of closing the refrigerator door, thus facilitating a user to view in the mobile phone APP, or view the panoramic photos or videos of the objects in the refrigerator with good light and wide field of vision before and during the last closing of the door in the refrigerator door touch screen at any time without opening the refrigerator door, releasing the vacuum and lowering the temperature during short range; and dynamically identifying the objects and uploading the data.

5. A washing machine comprising the intelligent and positive pressure system according to claim 1, further comprising:
a machine body (7); and
a washing-dehydrating-drying system (15),
wherein the machine body (7) is internally provided with the intelligent positive and negative pressure system and the washing-dehydrating-drying system (15).

6. An operation method for the washing machine according to claim 21, comprising: performing analysis one by one by a positive and negative pressure intelligent regulation and control apparatus (5) through built-in program and based on pre-stored data and internet cloud data, and real-time feedback information of a touch screen and mobile phone monitoring and identification system (12) and various sensors (C1) to (C12), and correspondingly using different accurate stain removal techniques for stain-causing factors and stain removal targets of various different cleaning scenarios and cleaning clothes, so as to form targeted stain removal comprehensive washing and drying methods with different characteristics; and sending, by the positive and negative pressure intelligent regulation and control apparatus (5), instructions to an air evacuation pump (2), an air inflation pump (3), related positive and negative pressure fluid carried substance generators (6) and related solenoid valves (B) in real time to accurately control and adjust open, close and switching of various air evacuation, inflation and circulation pipelines and intelligently regulate and control the level of the positive and negative pressure in the positive and negative pressure cabin (1) and the flowing-in, staying and flowing-out of a fluid and a carried substance thereof, so as to remove the stain-causing factors of the cleaning materials at fixed points and accurately maintain fragile elements of the cleaning materials to achieve the efficient and low-consumption cleaning effect;
wherein the operation methods for the targeted stain removal comprehensive washing and drying and the washing machine are as follows:
(1) water intake procedure: placing clothes into a drum (15.1), closing a cabin door (1.2) to seal and lock the washing machine type positive and negative pressure cabin (1F) after the washing machine is turned on; sending an instruction by the positive and negative pressure intelligent regulation and control apparatus (5) to enable a fifth solenoid valve (B14), a sixth solenoid valve (B15), a seventh solenoid valve (B16) and an eighth solenoid valve (B17) to open the corresponding water intake pipelines: (i) directly intaking tap water as follows, enabling the tap water to pass through a first water inlet pipeline (A14), the fifth solenoid valve, (B14), a fifth water intake pipeline (A18) the seventh solenoid valve (B16), a third water intake pipeline (A16), a water inlet port (z) of a detergent pull box, a water output port (1), a fourth water intake pipeline (A17), an eighth solenoid valve (B17), the fourth water intake pipeline, (A17), and a water inlet port (T) of a positive and negative pressure cabin; (ii) when washing-aid carried substance is required for washing, intaking water as follows, enabling water to pass through the first water intake pipeline (A14), the fifth solenoid valve (B14), the first water intake pipeline (A14), a water inlet port (r) of a water input pump, a water output port (u), a second water intake pipeline (A15), the sixth solenoid valve, (B15), the second water intake pipeline (A15), a water inlet port (v) of a waterflow carried substance generator integration, a water output port (o), a third water intake pipeline (A16), the seventh solenoid valve (B16), the third water intake pipeline (A16), the detergent pull box (z), the water output port (1), a fourth water intake pipeline (A17), the eighth solenoid valve (B17), the fourth water intake pipeline (A17), a water inlet port (T) of the positive and negative pressure cabin; (iii) when washing-aid carried substance is not required for cleaning, intaking water intake as follows, enabling the water to pass through the first water intake pipeline (A14), the fifth solenoid valve (B14), the water intake pipeline (A14), the water inlet port (r) of the water input pump, the water output port (u), the second water intake pipeline (A15), the sixth solenoid valve (B15), a sixth water intake pipeline (A19), a fifth water intake pipeline (A18), the seventh solenoid valve (B16), the third water intake pipeline (A16), the water inlet port (z) of the detergent pull box, the water output port (1), the fourth water intake pipeline (A17), the eighth solenoid valve (B17), the fourth water intake pipeline (A17), and the water inlet port (T) of the positive and negative pressure cabin, during the procedures (ii) and (iii), turning on the water input pump (17), directly feeding the tap water accordingly, or by the waterflow carried substance generator integration (6.04), rapidly mixing the tap water with the washing aid carried substance generated by waterflow carried substance generator integration (6.04), and then enabling the mixed water to enter the washing machine type positive and negative pressure cabin (1F), wherein the super-oxygen or carried substance mixed water is able to accelerate the decomposition of organic fouling on the clothes, thus facilitating the rapid cleaning; after the water intake is started, enabling, by the positive and negative pressure intelligent regulation and control apparatus (5), a first solenoid valve (B1) to open an air evacuation pipeline (A1), enabling the air to pass through an air inlet port (a) of the air evacuation pump, an air evacuation pipeline (A1), the first solenoid valve (B1), the air evacuation pipeline (A1) and an air evacuation and air return port (c) of the positive and negative pressure cabin, and turning on the air evacuation pump (2) to make the washing machine type positive and negative pressure cabin (1F) in a negative pressure, thus accelerating the water intake and shortening the time for water intake;
(2) vacuum washing procedure: after the water intake is finished, turning on, by the positive and negative pressure intelligent regulation and control apparatus (5), a motor assembly (15.5) to drive the drum mechanism (15.1) to rotate for washing, and meanwhile, enabling the first solenoid valve (B1) to open the air evacuation pipeline (A1), enabling the air to pass through the air inlet port (a) of the air evacuation pump, the air evacuation pipeline (A1), the first solenoid valve (B1), the air evacuation pipeline (A1), and the air evacuation and air return port (c) of the positive and negative pressure cabin, and turning on the air evacuation pump (2) to pump the washing machine type positive and negative pressure cabin (1F) to a moderate vacuum;
(3) high-pressure washing procedure: after washing at vacuum for proper time, closing the first solenoid valve (B1) and turning off the air evacuation pump (2) by the positive and negative pressure intelligent regulation and control apparatus (5), enabling a second solenoid valve (B3) to open an air inflation pipeline (A3), enabling the air to pass through an air outlet port (d) of the air inflation pump, the air inflation pipeline (A3), a second solenoid valve (B3), the air inflation pipeline (A3), and an air inflation port (f) of the positive and negative pressure cabin, and opening the air evacuation pipeline (A1), a first circulation pipeline (A5) and a first carrying air introduction pipeline (A6) at the same time: (i) atmospheric circulation: exhausting the air to atmosphere through a circulation air return port (i) of the positive and negative pressure cabin, the air evacuation pipeline (A1), the first solenoid valve (B1), the first circulation pipeline (A5), the third solenoid valve (B5), the first circulation pipeline (A5), and a circulation exhaust port (i2) for circulation; or (ii) in-machine circulation: enabling the air to pass through the circulation air return port (i) of the positive and negative pressure cabin, the air evacuation pipeline (A1), the first solenoid valve (B1), the first circulation pipeline (A5), the third solenoid valve (B5), a third circulation pipeline (A7), a fourth solenoid valve (B6), the second circulation pipeline (A6), and a circulation air return port (e2) of the air inflation pump, and turning on the air inflation pump (3), enabling high pressure airflow to enter the washing machine type positive and negative pressure cabin (1F) through the air inflation pipeline (A3), so as to form strong bubbles and rapid waterflow in the cabin to participate in clothes cleaning, wherein the high atmospheric pressure formed in the washing machine type positive and negative pressure cabin (1F) is beneficial for the detergent to penetrate into the clothes to facilitate the cleaning, and by circulating the air evacuation and air inflation repeatedly, and vacuum and high pressure are circulated and repeated, leading to fiber swell and turbulent rubbing, so as to greatly improve the cleaning degree and laundry efficiency and accelerate the washing and rinsing progress;
(4) water drainage and dehydration procedure: sending an instruction by the positive and negative pressure intelligent regulation and control apparatus (5) when the water drainage is required, enabling a ninth solenoid valve (B20), a tenth solenoid valve (B21) and an eleventh solenoid valve (B22) to open the corresponding water drainage pipelines: (i) when both the water suction pump and the decomposition of super oxygen and carried substance are not required, a directly draining water as follows, draining water to a sewer through a water drainage port (L) of a washing machine type positive and negative pressure cabin, a first water drainage pipeline (A20), the ninth solenoid valve (B20), a fourth water drainage pipeline (A23), the eleventh solenoid valve (B22) and a third water drainage pipeline (A22); (ii) when both the water suction pump for drainage and the decomposition of super oxygen are required, draining water as follows, draining water to a sewer through the water drainage port (L) of the positive and negative pressure cabin, the first water drainage pipeline (A20), the ninth solenoid valve (B20), the first water drainage pipeline (A20), a water inlet port (w) of a water suction pump, a water output port (p), a second water drainage pipeline (A21), the tenth solenoid valve (B21), the second water drainage pipeline (A21), a water inlet port (g) of a carried substance decomposition processor, a water output port (m), the third water drainage pipeline (A22), the eleventh solenoid valve (B22) and the third water drainage pipeline (A22); (iii) when the water suction pump is required for drainage and the decomposition of super oxygen and carried substance is not required, draining water as follows, draining water to a sewer through the water drainage port (L) of the washing machine type positive and negative pressure cabin, the first water drainage pipeline (A20), the ninth solenoid valve (B20), the first water drainage pipeline (A20), a water inlet port (w) of the water suction pump, a water output port (p), the second water drainage pipeline (A21), the tenth solenoid valve (B21), a fifth water drainage pipeline (A24), the fourth water drainage pipeline (A23), the eleventh solenoid valve (B22) and the third water drainage pipeline (A22), during the procedures (ii) and (iii), turning on the water suction pump (16) for water drainage, during water drainage, turning on the air inflation pump (3) to inflate and pressurize the washing machine type positive and negative pressure cabin (1F), thus accelerating the water drainage speed; after the water drainage is finished, sending an instruction by the positive and negative pressure intelligent regulation and control apparatus (5) to start dehydration; during dehydration, inflating and pressurizing to force the water to rapidly separate from the clothes; turning on the air evacuation pump (2) alternately to pump the cabin to a negative pressure to make the clothes fibers in the washing machine type positive and negative pressure cabin (1F) swell, wherein the water in the clothes escapes to a negative pressure space and is pumped out of the cabin, such that the clothes hardened on an inner wall of the drum during dehydrating and high pressure are loosened to prevent wrinkles, and by circulating the air evacuation and air inflation repeatedly, squeezing out water and evacuating the water out the cabin are carried out alternately, the efficiency of dehydrating the clothes is greatly improved, and time for water drainage and dehydration is shortened;
(5) vacuum high pressure drying procedure: when a drying procedure is started, turning on the air inflation pump (3) according to an instruction sent by the positive and negative pressure intelligent regulation and control apparatus (5), and opening the air inflation pipeline (A3) to inflate and pressurize the washing machine type positive and negative pressure cabin (1F), thus facilitating hot air to enter the clothes fibers to gasify the water, turning off the air inflation pump (3) and closing the air inflation pipeline (A3), turning on the air evacuation pump (2) and opening an air evacuation pipeline (A1) to pump the water out, and opening the first circulation pipeline (A5), a third circulation pipeline (A7), the second circulation pipeline (A6) and the air inflation pipeline (A3), enabling the water steam to pass through a circulation air inlet port (j2), the first circulation pipeline (A5), a third solenoid valve (B5), the third circulation pipeline (A7), a fourth solenoid valve (B6), the second circulation pipeline (A6), the second solenoid valve (B3), the air inflation pipeline (A3) and an circulation air inlet port (j) of the positive and negative pressure cabin, evacuating out the water and making the clothes fibers swell with the negative pressure to facilitate the water emission and quick drying of the clothes, such that, by circulating the air evacuation and inflation repeatedly, heating gasification and water pump-out are alternately conducted, the drying progress is greatly accelerated, the fluffy clothes improves the drying quality, and the drying time is effectively shortened; and
(6) vacuum self-cleaning sterile placement procedure: closing the cabin door (1.2) after the laundry is finished, controlling, by the positive and negative pressure intelligent regulation and control apparatus (5), the washing-dehydrating-drying control mechanism to start drying hot air to blow-dry the inside of the washing machine, especially the drum mechanism (15.1), and then turning on the air evacuation pump (2) and opening the air evacuation pipeline for air evacuation, evacuating out the residual super oxygen and carried substance in the washing machine type positive and negative pressure cabin (1F), and enabling the drum mechanism (15.1) and the equipment in the cabin to be in moderate vacuum.

7. A dish washing and fruit and vegetable cleaning machine comprising the intelligent positive and negative pressure system according to claim 1, further comprising:
a machine body (7);
a dish washing and drying system (19); and
a fruit and vegetable cleaning system (20),
wherein the machine body (7) is internally provided with the intelligent positive and negative pressure system, the dish washing and drying system (19), and the fruit and vegetable cleaning system (20).

8. An operation method for the dish washing and fruit and vegetable cleaning machine according to claim 22, comprising: regulating and controlling, by a positive and negative pressure intelligent regulation and control apparatus (5), various dish washing and fruit and vegetable cleaning and drying elements, such as waterflow and airflow pressure, gas composition, water temperature and air temperature in real time through built-in program and based on pre-stored data, and real-time feedback information of various sensors, wherein the operation method comprises the following steps:
(1) dish washing water intake and cleaning procedure: sending an instruction by the positive and negative pressure intelligent regulation and control apparatus (5) after the machine door is closed, enabling a second solenoid valve (B14), a third solenoid valve (B15), a fourth solenoid valve (B16) and a fifth solenoid valve (B25) to open corresponding water intake pipelines: (i) when the super oxygen or substance-carrying water is required for dish washing, intaking water by a water input pump as follows: enabling water to pass through a first water intake pipeline (A14), the second solenoid valve (B14), the first water intake pipeline (A14), a water inlet port (r) of the water input pump, a water output port (u), a second water intake pipeline (A15), the third solenoid valve (B15), the second water intake pipeline (A15), a water inlet port (k) of a waterflow carried substance generator integration, a water output port (q), a third water intake pipeline (A16), the fourth solenoid valve (B16), the third water intake pipeline (A16), the fifth solenoid valve (B25), a dish washing and drying water intake pipeline (A27), and a water inlet port (G) of a dish washing and drying system; (ii) when the super oxygen or substance-carrying water is not required for dish washing, intaking water by a water input pump as follows: enabling water to pass through the first water intake pipeline (A14), the second solenoid valve (B14), the first water intake pipeline (A14) (A14), the water inlet port (r) of the water input pump, the water output port (u), the second water intake pipeline (A15), the third solenoid valve (B15), a fourth water intake pipeline (A19), the fourth solenoid valve (B16), the third water intake pipeline (A16), the fifth solenoid valve (B25), the dish washing and drying water intake pipeline (A27), and the water inlet port (G) of the dish washing and drying system, turning on the water input pump (17), rapidly mixing, by the waterflow carried substance generator integration, the tap water with mixed super-oxygenated or substance-carrying water generated by the waterflow carried substance generator integration, and feeding the mixture into a dish washer type positive and negative pressure cabin (1G) to accelerate the decomposition of organic fouling on the tableware, thus facilitating the rapid cleaning; after the water intake is finished, turning on the dish washing and drying system (19) by the positive and negative pressure intelligent regulation and control apparatus (5) to spray water for dish washing, and enabling a solenoid valve (B1) to open an air evacuation pipeline (A1), enabling the air to pass through an air inlet port (a) of an air evacuation pump, an air evacuation pipeline (A1), a first solenoid valve (B1), the air evacuation pipeline (A1), and an air evacuation and air return port (c) of the positive and negative pressure cabin, and turning on the air evacuation pump (2) for vacuumizing, wherein, during the vacuumizing, the stain stained on the tableware expands in the negative vacuum pressure to make air escape from the stain, thus the adhesive force of the stain is weakened, or the stain fall off from the tableware, the cleaning efficiency is improved, and the washing process is shortened, moreover, the mixed super-oxygenated or substance-carrying water directly kills the bacteria and viral microorganism on the tableware, the organic matters in the stain on the tableware are decomposed and then dissolved into the water, the stain removability of the detergent is enhanced, the degree of cleaning is improved, the cleaning process is accelerated, and the function of sterilization and deodorization is achieved at the same time;
(2) dish washing drainage and drying disinfection procedure: when the water drainage is required at proper time of cleaning, sending an instruction by the positive and negative pressure intelligent regulation and control apparatus (5), enabling a sixth solenoid valve (B20), a seventh solenoid valve (B21) and an eighth solenoid valve (B22) to open the corresponding water drainage pipelines: (i) when both the water suction pump and the decomposition of super oxygen and carried substance are not required, directly draining water as follows, draining the water to a sewer through a water output port (H) of the dish washing and drying system, a dish washing and drying water drainage pipeline (A28), a first water drainage pipeline (A20), the sixth solenoid valve (B20), a fourth water drainage pipeline (A23), the eighth solenoid valve (B22) and a third water drainage pipeline (A22); (ii) when both the water suction pump for water drainage and the decomposition of super oxygen and carried substance are required, draining water as follows, draining water to the sewer through a water output port (H) of the dish washing and drying system, the dish washing and drying water drainage pipeline (A28), the first water drainage pipeline (A20), the sixth solenoid valve (B20), the first water drainage pipeline (A20), a water inlet port (w) of a water drainage pump, a water output port (p), a second water drainage pipeline (A21), the seventh solenoid valve (B21), the second water drainage pipeline (A21), a water inlet port (g) of a carried substance decomposition processor, a water output port (m), the third water drainage pipeline (A22), the eighth solenoid valve (B22) and the third water drainage pipeline (A22); and (iii) when the water suction pump is required for water drainage and the super oxygen does not need to be decomposed, draining water as follows, draining water to the sewer through the water output port (H) of the dish washing and drying system, the dish washing and drying water drainage pipeline (A28), the first water drainage pipeline (A20), the sixth solenoid valve (B20), a fourth water drainage pipeline (A23), the eighth solenoid valve (B22) and the third water drainage pipeline (A22), draining water directly or turning on the water suction pump 16, or turning on the carried substance decomposition decomposer (6.7) to decompose waste water and then drain the decomposed waste water into the sewer; meanwhile, spraying the tableware with clear water for cleaning completely; during the drying procedure, turning on, by the positive and negative pressure regulation and control apparatus (5), the air evacuation pump (2) again to rapidly pump out the water vapor so as to accelerate the drying progress and accelerate the dish washing time, wherein the super oxygen or carried substance also thoroughly sterilize, disinfect and removal order of the washed tableware;
(3) fruit and vegetable cleaning water intake and cleaning procedure: sending an instruction by the positive and negative pressure intelligent regulation and control apparatus (5) after the machine door is closed, enabling the second solenoid valve (B14), the third solenoid valve (B15), the fourth solenoid valve (B16) and the fifth solenoid valve (B25) to open corresponding water intake pipelines: (i) when the super-oxygenated water is required for fruit and vegetable cleaning, intaking water by the water input pump as follows, enabling water to pass through the first water intake pipeline (A14), the second solenoid valve (B14), the first water intake pipeline (A14), a water inlet port (r) of the water input pump, the water output port (u), the second water intake pipeline (A15), the third solenoid valve (B15), the second water intake pipeline (A15), the water inlet port (k) of the super oxygen generation water mixer, the water output port (q), the third water intake pipeline (A16), the fourth solenoid valve (B16), the third water intake pipeline (A16), the eighth solenoid valve (B25), a fruit and vegetable cleaning water intake pipeline (A25) and a water inlet port (e) of a fruit and vegetable cleaning system; (ii) when the super-oxygenated water is not required for fruit and vegetable cleaning, intaking water by the water input pump as follows, enabling water to pass through the first water intake pipeline (A14), the second solenoid valve (B14), the first water intake pipeline (A14), the water inlet port (r) of the water input pump, the water output port (u), the second water intake pipeline (A15), the third solenoid valve (B15), the fourth water intake pipeline (A19), the fourth solenoid valve (B16), the third water intake pipeline (A16), the eighth solenoid valve (B25), fruit and vegetable cleaning water intake pipeline (A25), and the water inlet port (e) of the fruit and vegetable cleaning system, and turning on the water input pump (17), rapidly mixing, by the waterflow carried substance generator integration (6.04), the tap water with mixed super-oxygenated or substance-carrying water generated by the waterflow carried substance generator integration, and feeding the mixture into the dish washer type positive and negative pressure cabin (1G); after the water intake is finished, turning on the fruit and vegetable cleaning system (19) by the positive and negative pressure intelligent regulation and control apparatus (5) to spray water for fruit and vegetable cleaning, and enabling a first solenoid valve (B1) to open an air evacuation pipeline (A1), enabling the air to pass through an air inlet port (a) of the air evacuation pump, the air evacuation pipeline (A1), the first solenoid valve (B1), the air evacuation pipeline (A1), and an air evacuation and air return port (c) of the positive and negative pressure cabin, and turning on the air evacuation pump (2) for vacuumizing; sending an instruction by the positive and negative pressure intelligent regulation and control apparatus (5) after cleaning by the mixed super-oxygenated or substance-carrying water, and spraying and cleaning the fruits and vegetables with clear water;
(4) water drainage procedure after fruit and vegetable cleaning: when the water drainage is required after cleaning for proper time, sending an instruction by the positive and negative pressure intelligent regulation and control apparatus (5), enabling the sixth solenoid valve (B20), the seventh solenoid valve (B21) and the eighth solenoid valve (B22) to open corresponding water drainage pipelines: (i) when both the water suction pump and the decomposition of super oxygen or carried substance are not required, directly draining water to the sewer through a water output port (F) of a fruit and vegetable cleaning system, a fruit and vegetable cleaning water drainage pipeline (A26), the first water drainage pipeline (A20), the sixth solenoid valve (B20), the fourth water drainage pipeline (A23), the eighth solenoid valve (B22), and the third water drainage pipeline (A22); (ii) when both the water suction pump for water drainage and the decomposition of super oxygen and carried substance are required, draining water to the sewer through the water output port (F) of the fruit and vegetable cleaning system, the fruit and vegetable cleaning water drainage pipeline (A26), the first water drainage pipeline (A20), the sixth solenoid valve (B20), the first water drainage pipeline (A20), a water inlet port (w) of the water suction pump, a water output port (p), the second water drainage pipeline (A21), the seventh solenoid valve (B21), the second water drainage pipeline (A21), a water output port (g) of the carried substance decomposition processor, a water output port (m), the third water drainage pipeline (A22), the eighth solenoid valve (B22) and the third water drainage pipeline (A22); (iii) when the water suction pump is required for water drainage and the super oxygen or carried substance does not need to be decomposed, draining water to the sewer through the water output port (F) of the fruit and vegetable cleaning system, the fruit and vegetable cleaning water drainage pipeline (A26), the first water drainage pipeline (A20), the sixth solenoid valve (B20), the fourth water drainage pipeline (A23), the eighth solenoid valve (B22) and the third water drainage pipeline (A22), directly draining water or turning on the water suction pump (16) or turning on the carried substance decomposition processor (6.7) again to decompose waste water, and then draining the decomposed waste water into the sewer;
(5) vacuum self-cleaning sterile placement sterile: closing the machine door (1.2) after use, starting drying hot air by the positive and negative pressure intelligent regulation and control apparatus (5) to blow-dry the inside of the dish washer type positive and negative pressure cabin (1G), especially the dish washing and drying system (19) and the fruit and vegetable cleaning system (20), and then turning on the air evacuation pump (2) and opening the air evacuation pipeline for air evacuation, evacuating out the residual super oxygen or carried substance in the dish washer type positive and negative pressure cabin (1G), and enabling the dish washing and drying system (19), the fruit and vegetable cleaning system (20) and the equipment in the cabin to be in a moderate vacuum.

9.-11. (canceled)

12. A fresh-keeping transport compartment container comprising the intelligent positive and negative pressure system according to claim 1, further comprising:
a compartment body (7); and
a refrigeration system (8),
wherein the compartment body (7) is internally provided with the intelligent positive and negative pressure system, and the refrigeration system (8).

13. An operation method for the fresh-keeping transportation compartment container according to claim 23, comprising: performing analysis one by one by the positive and negative pressure intelligent regulation and control apparatus (5) through built-in program and based on pre-stored data, and internet cloud data, camera identification, radar scanning and identification of the touch screen and mobile phone monitoring and identification system (12), and real-time feedback information of various sensors (C1) to (C12), correspondingly using different targeted fresh-keeping techniques for fresh-loss factors and control targets of different storage products to form targeted control comprehensive fresh-keeping methods with different characteristics, and sending, by the positive and negative pressure intelligent regulation and control apparatus (5), instructions to an air evacuation pump (2), an air inflation pump (3), related positive and negative pressure fluid carried substance generators (6) and related solenoid valves (B) in real time to control and adjust open, close and switching of various air evacuation, inflation and circulation carrying pipelines and intelligently regulate and control the level of the positive and negative pressure in the positive and negative pressure cabin (1) and the flowing-in, staying and flowing-out of a fluid and a carried substance thereof;
wherein the targeted control comprehensive fresh-keeping methods and use methods and operation procedures of the fresh-keeping compartment container are as follows:
(1) exhaust and vacuum abatement heat-removal cleaning procedure: closing a cabin door (1.2), sending an instruction by the positive and negative pressure intelligent regulation and control apparatus (5) to enable a second solenoid valve (B2) and a first solenoid valve (B1) to open an air exhaust pipeline (A2) and an air evacuation pipeline (A1), and turning on an air evacuation pump (2) to exhaust or pump the atmospheric compartment container type positive and negative pressure cabin (1M-2) or the compartment container type positive and negative pressure cabin (1M) to vacuum or negative pressure between −0.001 KPa and −0.1 MPa, where the local instant atmospheric pressure is used as the zero standard;
(2) pressure reduction and control or humidification and humidity control preservation procedure: according to a pressure standard of preset procedure and real-time feedback of a negative pressure sensor (C2), turning the air evacuation pump (2) on or off by the positive and negative pressure intelligent regulation and control apparatus (5) to maintain an appropriate negative-pressure, low-oxygen and low-temperature environment within compartment container type positive and negative pressure cabin (1M), and meanwhile, turning on the a humidification and humidity control apparatus (6.5) to increase and control humidity in the cabin;
(3) pressurized or atmospheric circulating sterilization, degradation and deodorization procedure for super oxygen, negative ion or catalyst positive and negative pressure carried substance: according to the procedure setting and feedback of sensors (C), sending, by the positive and negative pressure intelligent regulation and control apparatus (5), an instruction to turn on a super oxygen generator (6.2), a negative ion generator (6.3), a catalyst release controller (6.4), a humidity control apparatus (6.5) or a disinfection and degradation apparatus (6.9) in a first airflow carried substance generator integration (6.02) or a second airflow carried substance generator integration (6.021) or an integrated pipeline type fluid carried substance generator (6.05), or a carried substance generator installed in a carried substance generator module (6.16), thus enabling the super oxygen, catalyst, negative ion, water and related carried substances in the positive and negative pressure cabin reach required standards; meanwhile, enabling a third solenoid valve (B3), a fourth solenoid valve (B4) and a ninth solenoid valve (B3.1), a tenth solenoid valve (B3.2), an eleventh solenoid valve (B3.3) or a twelfth solenoid valve (B3.4) to open corresponding an air intake pipeline (A4) and an air inflation pipeline (A3) and a first air inflation branch pipeline (A3.1), a second air inflation branch pipeline (A3.2), a third air inflation branch pipeline (A3.3), or a fourth air inflation branch pipeline (A3.4), turning on the air inflation pump (3) to properly pressurize the related positive and negative pressure cabin to 0.001 KPa to 10 MPa;
(4) atmospheric, pressurized or humidified preservation procedure of air conditioning gas or other carried substances: according to the procedure setting, camera identification, radar scanning and identification, and feedback of sensors, opening related air evacuation pipelines and turning on the air evacuation pump (2) and a carried substance decomposition processor (6.7) at proper time to pump the decomposed, sterilized, disinfected and degraded polluted air out of the cabin; then enabling the third solenoid valve (B3) and the fourth solenoid valve (B4), a fifth solenoid valve (B6), a sixth solenoid valve (B6.1) or a seventh solenoid valve (B6.2) to open corresponding the air intake pipeline (A4) and the air inflation pipeline (A3), a first carrying air introduction pipeline (A6), a second first carrying air introduction pipeline (A6.1) or a third first carrying air introduction pipeline (A6.2), or enabling the third solenoid valve (B3), the fourth solenoid valve (B4), the fifth solenoid valve (B6) and an eighth solenoid valve (B7) to open the air inflation pipeline (A3), a first carrying circulation pipeline (A5), a second carrying circulation pipeline (A6) and a third carrying circulation pipeline (A7), and turning on the air inflation pump (3) for the inflation and pressurization or atmospheric circulation of the compartment container type positive and negative pressure cabin (1M) or the atmospheric compartment container type positive and negative pressure cabin (1M-2); during the operation of an air conditioning apparatus (6.1), moderately increasing partial pressure difference between both sides of a membrane to improve air separation efficiency, and evacuating and exhausting oxygen-enriched waste gas or carried waste gas; enabling a first solenoid valve (B1) and a second solenoid valve (B2) or a thirteenth solenoid valve (B8.1) to open a first carrying waste gas evacuation and exhaust pipeline (A8) or a second carrying waste gas evacuation and exhaust pipeline (A8.1), an air evacuation pipeline (A1) or an air exhaust pipeline (A2), exhausting the air to atmosphere through a carrying waste gas evacuation and exhaust port (s1) or (s2) of an air conditioning apparatus (6.1), the first carrying waste gas evacuation and exhaust pipeline (A8) or the second carrying waste gas evacuation and exhaust pipeline (A8.1), the first solenoid valve (B1), the air evacuation pipeline (A1), an air inlet port (a) of an air evacuation pump, an air outlet port (b) of the air evacuation pump, the air exhaust pipeline (A2), a carried substance decomposition processor (6.7), the air exhaust pipeline (A2), the second solenoid valve (B2), the air exhaust pipeline (A2), an air-to-water production apparatus (11) and the air exhaust pipeline (A2); turning on the air evacuation pump (2) to exhaust and pump decomposed waste gas;
(5) low-temperature assisted fresh-keeping procedure: after the positive and negative pressure fresh-keeping compartment container is turned on and the door is closed, turning on a refrigeration system (8) instantly by the positive and negative pressure intelligent regulation and control apparatus (5), providing corresponding low temperature for various positive and negative pressure cabins according to the feedback and control of a temperature sensing controller (C9) and intelligent regulation and control of the positive and negative pressure intelligent regulation and control apparatus (5), thus assisting the positive and negative pressure system to keep the freshness of the stored objects;
(6) open-to-exhaust protection function: if the compartment door is opened during super-oxygen sterilization and disinfection or air-condition preservation, providing feedback immediately by a cabin door switch, and stopping the super oxygen or air conditioning or related procedures immediately through the operation of the positive and negative pressure intelligent regulation and control apparatus (5); and meanwhile, opening the air evacuation pipelines and turning on the air evacuation pump (2) to rapidly pump the gas in the cabin to the carried substance decomposition processor (6.7) to be exhausted to atmosphere after being decomposed, wherein, as the air flows from the outside of the fresh-keeping compartment container door to the positive and negative pressure cabin for supplementing air evacuation negative pressure, the super oxygen or high-nitrogen low-oxygen carried substance gas is unable to flow out of the door;
(7) air-to-water production procedure: enabling moist waste gas pumped from the vacuum high pressure cabin by the air evacuation pump 2 to enter the carried substance decomposition processor (6.7), and enabling the moist waste gas after super oxygen decomposition to enter the air-to-water production apparatus (11), condensing water vapor and filtering the condensed water vapor to form purified water, thus providing uninterrupted source of water for drinking ice making or the humidification and humidity control apparatus (6.5); when the air inflation pump 3 is idle, turning on the air inflation pump (3) by the intelligent regulation and control apparatus (5), and enabling the third solenoid valve (B3) and the second solenoid valve (B2) to open air-to-water production inflation pipelines, thus driving the indoor air to enter the air-to-water production apparatus (11) for water production; and
(8) remote and short-range control monitoring function: installing a high-definition anti-fog camera and identification apparatus (12.3) and a radar scanning and identification apparatus (12.5) at positions needing monitoring and identification inside and outside the compartment body (7); shooting and scanning statuses of objects in the fresh-keeping compartment container in real time, and automatically scanning and identifying variety, category, composition, color, water content, disease degree and various fresh-keeping factors of the stored objects, and even fuzzy data of the number ratio of different objects in the same cabin, and computing a real-time numerical value or the greatest common divisor by combining the cloud data; through manual or automatic setting, accurately regulating and controlling, by the positive and negative pressure system, various fresh-keeping environmental factors such as airflow pressure, gas composition, air cleanliness, environment temperature and humidity in the positive and negative pressure cabin in real time.

14-15. (canceled)

16. A disinfection machine comprising the intelligent positive and negative pressure system according to claim 1, further comprising:
a machine (7); and
a power supply and rechargeable battery (33),
wherein the machine body (7) is internally provided with the intelligent positive and negative pressure system, an air-to-water production apparatus (11), and the power supply and rechargeable battery (33).

17. (canceled)

18. A module cabinet comprising the intelligent positive and negative pressure system according to claim 1, further comprising:
a cabinet body (7); and
a refrigeration and heating system (8.3),
wherein the cabinet body (7) is internally provided with the intelligent positive and negative pressure system, and the refrigeration and heating system (8.3).

19. The system according to claim 1, further comprising:
one or more of a sensor (C), a water suction pump (16), a water inlet pump (17), and a touch screen and mobile phone monitoring and identification system (12), wherein the water suction pump (16), the water inlet pump (17) and the touch screen and mobile phone monitoring and identification system (12) are arranged outside the positive and negative pressure cabin (1);
the positive and negative pressure cabin (1) is classified into a vacuum high pressure cabin (1-1), an atmospheric circulation cabin (1-2), and atmospheric inlet-outlet cabin (1-3) according to a structure and pressure thereof;
an internal structure of the positive and negative pressure cabin (1) comprises:
a cabin body (1.1), a cabin door (1.2), an airtight mechanism (1.3), and an internal and external communicating sealer (1.4); the airtight mechanism (1.3) is arranged between the cabin body (1.1) and the cabin door (1.2); the internal and external communicating sealer (1.4) is fixedly arranged at a rear part or side part of the positive and negative pressure cabin (1), and all pipelines and circuits getting in and out the cabin are connected to and pass through the internal and external communicating sealer (1.4); each of the positive and negative pressure refrigerating fresh-keeping cabin (1B), a positive and negative pressure freezing fresh-keeping cabin (1C) has a corresponding positive and negative pressure resisting structure, and is provided with an airtight mechanism (1.3), and an internal and external communicating sealer (1.4), which belongs to an airtight vacuum high pressure cabin (1-1) structure; a positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to demands and a configuration of the positive and negative pressure cabin;
the intelligent positive and negative pressure regulation and control apparatus (5) comprises:
an integrated circuit, a chip, a regulation and control system for a fluid and carried substance thereof, and is connected to the Internet in wired and wireless modes;
the positive and negative pressure fluid carried substance generation processor (6) comprises one or more of the following apparatuses: an air conditioning apparatus (6.1), a super oxygen generator (6.2), a negative ion generator (6.3), a catalyst release controller (6.4), a humidification, dehumidification and humidity control apparatus (6.5), a super oxygen generation water mixer (6.6), a disinfection, sterilization and degradation apparatus (6.9), a filtering and refreshing apparatus (6.10), a stain removal and oil dispelling cleaning apparatus (6.11), a refrigeration, heating and temperature control apparatus (6.13), a carried substance generator item addition and upgrading module (6.16), one or more airflow carried substance generator integrations, one or more waterflow carried substance generator integrations, and an integrated pipeline type positive and negative fluid carried substance generator (6.05); the air conditioning apparatus (6.1) is a membrane-based air separation type air conditioning apparatus; each of the air conditioning apparatus (6.1), the super oxygen generator (6.2), the negative ion generator (6.3), the catalyst release controller (6.4), the humidification, dehumidification and humidity control apparatus (6.5), the super oxygen generation water mixer (6.6), the disinfection, sterilization and degradation apparatus (6.9), the filtering and refreshing apparatus (6.10), the stain removal and oil dispelling cleaning apparatus (6.11), the refrigeration, heating and temperature control apparatus (6.13) and the carried substance generator item addition and upgrading module (6.16) belongs to an individual fluid carried substance generator; the carried substance generator item addition and upgrading module (6.16) is a combined module capable of providing or installing any individual fluid carried substance generator additional item in the carried substance generator integration and upgrading and updating the same; one or more airflow carried substance generator integrations, one or more waterflow carried substance generator integrations and the integrated pipeline type positive and negative pressure fluid carried substance generator (6.05) each comprise any one or more in the individual fluid carried substance generators; the integrated pipeline type positive and negative pressure fluid carried substance generator (6.05) is a small, intelligent and integrated fluid carried substance generator and sensor and connected pipelines and circuits;
an air evacuation pipeline (A1) is installed at an air inlet port (a) of the air evacuation pump (2), a first solenoid valve (B1) is installed at a middle part of the air evacuation pipeline (A1), and the other end of the air evacuation pipeline (A1) extends into the positive and negative pressure cabin (1) to form an air evacuation and air return port (c); an air exhaust pipeline (A2) is installed at an air outlet port (b) of the air evacuation pump (2), a second solenoid valve (B2) is installed at a middle part of the air exhaust pipeline (A2), and the other end of the air exhaust pipeline is an exhaust port; a second carrying circulation pipeline (A5.1) is further connected to the first solenoid valve (B1), the second carrying circulation pipeline (A5.1) extends into the positive and negative pressure cabin (1) through the first solenoid valve (B1) and the air evacuation pipeline (A1) to form a carrying air inlet port (j1), and the other end of the second carrying circulation pipeline (A5.1) is connected to a carrying circulation air inlet port (b2) of the air evacuation pump (2); a carrying waste gas evacuation and exhaust pipeline (A8) is also connected to the first solenoid valve (B1), and the other end of the carrying waste gas evacuation and exhaust pipeline (A8) is connected to a carrying waste gas evacuation and exhaust port (s2) of an air conditioning apparatus (6.1) integrated in the airflow carried substance generator integration (6.02); an air inflation pipeline (A3) is installed at an air outlet port (d) of the air inflation pump (3), a third solenoid valve (B3) is installed at a middle part of the air inflation pipeline (A3), and the other end of the air inflation pipeline (A3) extends into the positive and negative pressure cabin (1) to form an air inflation inlet (f) thereof; an air intake pipeline (A4) is installed at an air inlet port (3) of the air inflation pump (3), a fourth solenoid valve (B4) is installed at a middle part of the air intake pipeline (A4), and the other end of the air intake pipeline (A4) is connected to atmosphere; a first carrying circulation pipeline (A5) is further connected to the third solenoid valve (B3), the first carrying circulation pipeline (A5) extends into the positive and negative pressure cabin (1) through the third solenoid valve (B3) and the air inflation pipeline (A3) to form a carrying air return port (j) thereof; the other end of the first carrying circulation pipeline (A5) is connected to a carrying circulation air inlet port (c2); a first carrying air introduction pipeline (A6) is installed at an air inlet port (t) of the airflow carried substance generator integration (6.02), and a fifth solenoid valve (B6) is installed on the other end of the first carrying air introduction pipeline (A6); a second carrying air introduction pipeline (A7) is installed at an air outlet port (s) of the airflow carried substance generator integration (6.02), a seventh solenoid valve (B7) is installed at a middle part of the second carrying air introduction pipeline (A7), and the other end of the second carrying air introduction pipeline (A7) extends into the positive and negative pressure cabin (1) to form a carrying air inflation port (i); the fifth solenoid valve (B6) is respectively connected to a carrying air evacuation pipeline (A6.1) and a carrying air intake pipeline (A6.2), the carrying air evacuation pipeline (A6.1) is connected to the air evacuation pipeline (A1) and then is connected to an air evacuation port (a) of the air evacuation pump (2), and the carrying air intake pipeline (A6.2) is connected to the air inflation pipeline (A3) and then communicates with the air outlet port (d) of the air inflation pump (3); a sixth solenoid valve (B6.2) is installed at a middle part of the carrying air intake pipeline (A6.2), and the other end of the carrying air intake pipeline (A6.2) extends into the positive and negative pressure cabin (1) and then is connected to the integrated pipeline type positive and negative pressure fluid carried substance generator (6.05); when a carrying airflow entering the positive and negative pressure cabin (1) requires air inflation circulation, the air inflation inlet (f) serves as the carrying air return port (j) of the positive and negative pressure cabin (1); when the carrying airflow entering the positive and negative pressure cabin (1) requires air evacuation circulation, the air evacuation and air return port (c) serves as the carrying air inlet port (j1) of the positive and negative pressure cabin (1), and the carrying air inflation port (i) serves as a carrying air evacuation port (i1) of the positive and negative pressure cabin (1);
the positive and negative pressure cabin is externally provided with the water suction pump (16), the water input pump (17), the positive and negative pressure intelligent regulation and control apparatus (5), the touch screen and mobile phone monitoring and identification system (12) and the waterflow carried substance generator integration (6.04); a water suction pipeline (A20) is installed at a water suction port (w) of the water suction pump (16), a twelfth solenoid valve (B20) is installed at a middle part of the water suction pipeline (A20), and the other end of the water suction pipeline (A20) extends into the positive and negative pressure cabin (1) to form a water suction and water output port (L); a water drainage pipeline (A21) is installed at a water drainage port (p) of the water suction pump (16), a thirteenth solenoid valve (B21) is installed at a middle part of the water drainage pipeline (A21), and the other end of the water drainage pipeline (A21) is a water drainage port; a fourth carrying circulation pipeline (A19.1) is further connected to the eleventh solenoid valve (B20), the fourth carrying circulation pipeline (A19.1) extends into the positive and negative pressure cabin (1) through the twelfth solenoid valve (B20) and the water suction pipeline (A20) to form a carrying water inlet port (L1) of the positive and negative pressure cabin (1), and the other end of the fourth carrying circulation pipeline (A19.1) is connected to a carrying circulation water output port (p2) of the water suction pump (16); a water intake pipeline (A15) is installed at a water output port (u) of the water input pump (17), a ninth solenoid valve (B15) is installed at a middle part of the water intake pipeline (A15), and the other end of the water intake pipeline (A15) extends into the positive and negative pressure cabin (1) to form a water source intake (T); a first water intake pipeline (A14) is installed at a water inlet port (r) of the water input pump (17), an eighth solenoid valve (B14) is installed at a middle part of the first water intake pipeline (A14), and the other end of the first water intake pipeline (A14) is connected to a water source; a third carrying circulation pipeline (A19) is further connected to the ninth solenoid valve (B15), the third carrying circulation pipeline (A19) extends into the positive and negative pressure cabin (1) through the ninth solenoid valve (B15) and the second water intake pipeline (A15) to form a carrying water return port (L2) of the positive and negative pressure cabin (1), and the other end of the third carrying circulation pipeline (A19) is connected to a carrying circulation water inlet port (r2) of the water input pump (17); a first carrying water introduction pipeline (A16) is installed at a water inlet port v of the waterflow carried substance generator integration (6.04), and a tenth solenoid valve (B16) is installed at the other end of the first carrying water introduction pipeline (A16); a second carrying water introduction pipeline (A17) is installed at a water output port (o) of the waterflow carried substance generator integration (6.04), a solenoid valve (B17) is installed at a middle part of the second carrying water introduction pipeline (A17), and the other end of the second carrying water introduction pipeline (A17) extends into the positive and negative pressure cabin (1) to form a carrying water inlet port (T1) of the positive and negative pressure cabin (1); the tenth solenoid valve (B16) is respectively connected to a carrying water suction pipeline (A16.1) and a carrying water intake pipeline (A16.2), the carrying water suction pipeline (A16.1) is connected to the water suction pipeline (A20) and then communicates with a water suction port (w) of the water suction pump (16); the carrying water intake pipeline (A16.2) is connected to the second water intake pipeline (A15) and then communicates with the water output port (u) of the water input pump (7); when a carrying waterflow entering the positive and negative pressure cabin (1) requires water intake circulation, the water source intake (t) serves as the carrying water return port (L2) of the positive and negative pressure cabin (1); when a carrying waterflow entering the positive and negative pressure cabin (1) requires water suction circulation, the water suction output port (L) serves as the carrying water inlet port (L1) of the positive and negative pressure cabin (1); the touch screen and mobile phone monitoring and identification system (12) comprises a refrigerator door touch screen (12.1), a mobile phone APP (12.2), a high-definition anti-fog camera apparatus (12.3), and a radar scanning recognition apparatus (12.5); the high-definition anti-fog camera apparatus (12.3) and the radar scanning recognition apparatus (12.5) are installed at a position needing to be monitored inside and outside the positive and negative pressure cabin; the positive and negative pressure cabin (1) is internally provided with sensors (C), comprising one or more of the following sensors: a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), and a carried substance sensor item addition and upgrading module (C12); the carried substance sensor item addition and upgrading module (C12) is a combined module capable of providing and installing any individual sensor additional item into the sensor integration and upgrading and updating the same; all fluid carried substance generation processors (6), sensors (C) and solenoid valves (B) and the air evacuation pump (2), the air inflation pump (3), the water suction pump (16), the water input pump (17) and the touch screen and mobile phone monitoring and identification system (12) are connected to the positive and negative pressure intelligent regulation and control apparatus (5) by lines (D) or bundled lines (E).

20. The fresh-keeping refrigerator according to claim 3, wherein the intelligent positive and negative pressure system further one or more of a water suction pump (16), a water input pump (17), sensors (C), and a touch screen and mobile phone monitoring and identification system (12);
the positive and negative pressure cabin (1), according to cold preservation and freezing requirements of a fresh-keeping refrigerator, is designed as a positive and negative pressure refrigerating fresh-keeping cabin (1B), a positive and negative pressure freezing fresh-keeping cabin (1C), a general refrigerator refrigerating compartment type positive and negative pressure fresh-keeping cabin (1D), and a general refrigerator freezing compartment type positive and negative pressure fresh-keeping cabin (1E);
the positive and negative pressure refrigerating fresh-keeping cabin (1B) and the positive and negative pressure freezing fresh-keeping cabin (1C) are both vacuum high pressure cabins (1-1), a positive and negative pressure resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to demands and a configuration of the refrigerator; one or more of positive and negative pressure refrigerating fresh-keeping cabins (1B) and positive and negative pressure freezing fresh-keeping cabins (1C) are respectively arranged in the refrigerator body (7) in up-down or left-right arrangement and asymmetrical irregular arrangement modes;
the positive and negative pressure refrigerating fresh-keeping cabin (1B) and the positive and negative pressure freezing fresh-keeping cabin (1C), according to the shape and characteristic classification, comprise a drawer type positive and negative pressure refrigerating fresh-keeping cabin (1B-1), a drawer type positive and negative pressure freezing fresh-keeping cabin (1C-1), a side door cabinet type positive and negative pressure refrigerating fresh-keeping cabin (1B-2), and a side door cabinet type positive and negative pressure freezing fresh-keeping cabin (1C-2);
the drawer type positive and negative pressure refrigerating fresh-keeping cabin (1B-1) and the drawer type positive and negative pressure freezing fresh-keeping cabin (1C-1) comprise cabin bodies (1.1), a positive and negative pressure refrigerating fresh-keeping drawer (1.81), a positive and negative pressure freezing fresh-keeping drawer (1.82), and internal and external communicating sealers (1.4);
a drawer type airtight mechanism (1.9) is arranged between the cabin body of the drawer type positive and negative pressure refrigerating fresh-keeping cabin (1B-1) and the positive and negative pressure refrigerating fresh-keeping drawer (1.81) and between the cabin body of the drawer type positive and negative pressure freezing fresh-keeping cabin (1C-1) and the positive and negative pressure freezing fresh-keeping drawer (1.82); the drawer type airtight mechanism (1.9) comprises a locking ring (1.91), a lock bolt (1.92), and an airtight gasket (1.93); the locking ring (1.91) is in movable fit with the lock bolt (1.92), when the fresh-keeping drawer is closed, the locking bolt (1.92) extends into the locking ring (1.92) to lock a cabin door, and the airtight gasket (1.93) is used for keeping sealing;
the side door cabinet type positive and negative pressure refrigerating fresh-keeping cabin (1B-2) and the side door cabinet type positive and negative pressure freezing fresh-keeping cabin (1C-2) each comprise a cabin door (1.1), a cabin door (1.2), an airtight mechanism (3), and an internal and external communicating sealer (1.4); the airtight mechanism (1.3) is arranged between the cabin door (1.2) and the cabin body (1.1); the airtight mechanism (1.3) comprises a locking ring (1.31), a lock bolt (1.32), an airtight gasket (1.33), and a door rim sealing gasket; the locking ring (1.31) is in movable fit with the lock bolt (1.32), when the cabin door is closed, the locking bolt (1.32) extends into the locking ring (1.32) to lock the cabin door, and the airtight gasket (1.33) is used for keeping sealing; the internal and external communicating sealer (1.4) is arranged on a side part or a side part of each of the drawer type positive and negative pressure refrigerating fresh-keeping cabin (1B-1), the drawer type positive and negative pressure freezing fresh-keeping cabin (1C-1), the side door cabinet type positive and negative pressure refrigerating fresh-keeping cabin (1B-2), and the side door cabinet type positive and negative pressure freezing fresh-keeping cabin (1C-2), all pipelines and circuits getting in and out the cabin are connected to and pass through the internal and external communicating sealer (1.4) so as to keep the sealing property of the positive and negative pressure cabin (1);
the general refrigerator refrigerating compartment type positive and negative pressure fresh-keeping cabin (1D) and the general refrigerator freezing compartment type positive and negative pressure fresh-keeping cabin (1E) are both atmospheric circulation cabins, in which a positive and negative pressure system in each of the refrigerating compartment and the freezing compartment of the general refrigerator, but an airtight mechanism and internal and external communicating sealer are not provided;
the general refrigerator refrigerating compartment type positive and negative pressure fresh-keeping cabin (1D) comprises a cabin body (1.1) and a cabin door (1.2), and an airtight mechanism and an internal and external communicating sealer do not need to be provided between the cabin body (1.1) and the cabin door (1.2), and the cabin door (1.2) is a refrigerator door of the fresh-keeping refrigerator;
the general refrigerator freezing compartment type positive and negative pressure fresh-keeping cabin (1E) comprises a cabin door (1.1), and a non-airtight fresh-keeping drawer (1.83); an airtight mechanism and an internal and external communicating sealer do not need to be provided between the cabin body (1.1) and the non-airtight fresh-keeping drawer (1.83);
the positive and negative pressure intelligent regulation and control apparatus (5) comprises an integrated circuit, a chip, and a regulation and control system for a fluid and a carried substance thereof, and is connected to an Internet in a wired or wireless mode; and
the positive and negative pressure fluid carried substance generation processor (6) comprises one or more of the following apparatuses: an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), one or more airflow carried substance generator integrations; the air conditioning apparatus (6.1) is a membrane-based air separation type air conditioning apparatus; each of the air conditioning apparatus (6.1), the super oxygen generator (6.2), the negative ion generator (6.3), the catalyst release controller (6.4), the humidification, dehumidification and humidity control apparatus (6.5), the carried substance decomposition processor (6.7), the air filter (6.8), the disinfection, sterilization and degradation apparatus (6.9) and the carried substance generator item addition and upgrading module (6.16) belongs to an individual fluid carried substance generator; the carried substance generator item addition and upgrading module (6.16) is a combined module capable of providing or installing any individual fluid carried substance generator additional item in the carried substance generator integration and upgrading and updating the same; the one or more airflow carried substance generator integrations each comprise one or more of the individual fluid carried substance generators; each of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) is externally provided with an air evacuation pump (2), an air inflation pump (3), a positive and negative pressure intelligent regulation and control apparatus (5), a first airflow carried substance generator integration (6.021), a carried substance decomposition processor (6.7), an air filter (6.8), a refrigeration system (8), an air-to-water production apparatus (11), and a touch screen and mobile phone monitoring system (12); an air evacuation pipeline (A1) is installed at an air inlet port (a) of the air evacuation pump (2), a first air evacuation branch pipeline (A1.1), a second air evacuation branch pipeline (A1.2), a third air evacuation branch pipeline (A1.3) and a fourth air evacuation branch pipeline (A1.4) are respectively connected to the air evacuation pipeline (A1), a first solenoid valve (B1.1), a second solenoid valve (B1.2), a third solenoid valve (B1.3) and a fourth solenoid valve (B1.4) are respectively installed at middle parts of the first air evacuation branch pipeline (A1.1), a second air evacuation branch pipeline (A1.2), a third air evacuation branch pipeline (A1.3) and a fourth air evacuation branch pipeline (A1.4), and the other ends of the first air evacuation branch pipeline (A1.1), a second air evacuation branch pipeline (A1.2), a third air evacuation branch pipeline (A1.3) and a fourth air evacuation branch pipeline (A1.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form a first air evacuation and air return port (c1), a second air evacuation and air return port (c2), a third air evacuation and air return port (c3) and a fourth air evacuation and air return port (c4) thereof; a first carrying waste gas evacuation and exhaust pipeline (A8), a second carrying waste gas evacuation and exhaust pipeline (A8.1), a third carrying waste gas evacuation and exhaust pipeline (A8.2), a fourth carrying waste gas evacuation and exhaust pipeline (A8.3) and a fifth carrying waste gas evacuation and exhaust pipeline (A8.4) are respectively connected to the air evacuation pipeline (A1), a twenty-sixth solenoid valve (B8), a twenty-seventh solenoid valve (B8.1), a twenty-eighth solenoid valve (B8.2), a twenty-ninth solenoid valve (B8.3) and a thirtieth solenoid valve (B8.4) are respectively installed at middle parts of the first carrying waste gas evacuation and exhaust pipeline (A8), the second carrying waste gas evacuation and exhaust pipeline (A8.1), the third carrying waste gas evacuation and exhaust pipeline (A8.2), a fourth carrying waste gas evacuation and exhaust pipeline (A8.3) and a fifth carrying waste gas evacuation and exhaust pipeline (A8.4), and the other ends of the first carrying waste gas evacuation and exhaust pipeline (A8), the second carrying waste gas evacuation and exhaust pipeline (A8.1), the third carrying waste gas evacuation and exhaust pipeline (A8.2), a fourth carrying waste gas evacuation and exhaust pipeline (A8.3) and a fifth carrying waste gas evacuation and exhaust pipeline (A8.4) are respectively connected to a first carrying waste gas evacuation and exhaust port (s3), a second carrying waste gas evacuation and exhaust port (s4), a third carrying waste gas evacuation and exhaust port (s5), a fourth carrying waste gas evacuation and exhaust port (s6) and a fifth carrying waste gas evacuation and exhaust port (s7) of the air conditioning apparatus (6.1) integrated in a first airflow carried substance generator integration (6.021), a second airflow carried substance generator integration (6.022), a third airflow carried substance generator integration (6.023), a fourth airflow carried substance generator integration (6.024) and a fifth airflow carried substance generator integration (6.025); an air exhaust pipeline (A2) is installed at an air outlet port (b) of the air evacuation pump (2), a carried substance decomposition processor (6.7) and a fifth solenoid valve (B2) are installed at a middle part of the air exhaust pipeline (A2), and the other end of the air exhaust pipeline (A2) is connected to an air inlet port (h) of an air-to-water production apparatus (11), and then is connected to atmosphere via an air outlet port (n) of the air exhaust pipeline (A2) after passing through the air-to-water production apparatus (11); an air inflation pipeline (A3) is installed at an air outlet port (d) of the air inflation pump (3), a sixth solenoid valve (B3) is installed at a middle part of the air inflation pipeline (A3), a first air inflation branch pipeline (A3.1), a second air inflation branch pipeline (A3.2), a third air inflation branch pipeline (A3.3) and a fourth air inflation branch pipeline (A3.4) are respectively connected to the other end of air inflation pipeline (A3), a seventh solenoid valve (B3.1), an eight solenoid valve (B3.2), a ninth solenoid valve (B3.3) and a tenth solenoid valve (B3.4) are respectively installed at middle parts of the first air inflation branch pipeline (A3.1), the second air inflation branch pipeline (A3.2), the third air inflation branch pipeline (A3.3) and the fourth air inflation branch pipeline (A3.4), and the other ends of the first air inflation branch pipeline (A3.1), the second air inflation branch pipeline (A3.2), the third air inflation branch pipeline (A3.3) and the fourth air inflation branch pipeline (A3.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form a first air inflation and inlet port (f1), a second air inflation and inlet port (f2), a third air inflation and inlet port (f3) and a fourth air inflation and inlet port (f4) thereof; a carrying air introduction pipeline (A6) is further connected to the air inflation pipeline (A3), a seventeenth solenoid valve (B6) is installed at a middle part of the carrying air introduction pipeline (A6), and the other end of the carrying air introduction pipeline (A6) is connected to an air inlet port (t) of the positive and negative pressure airflow carried substance generator integration (6.021); a second carrying air introduction branch pipeline (A7.2) and a third carrying air introduction branch pipeline (A7.3) are respectively installed at a first air outlet port (s1) and a second air outlet port (s2) of the positive and negative pressure airflow carried substance generator integration (6.021), a twenty-third solenoid valve (B7.2) and a twenty-fourth solenoid valve (B7.3) are respectively installed at middle parts of the second carrying air introduction branch pipeline (A7.2) and the third carrying air introduction branch pipeline (A7.3), and the other ends of the second carrying air introduction branch pipeline (A7.2) and the third carrying air introduction branch pipeline (A7.3) respectively extend into the positive and negative pressure refrigerating fresh-keeping cabin (1B) and the positive and negative pressure freezing fresh-keeping cabin (1C) to form a second carrying air inlet port (i2) and a third carrying air inlet port (13) thereof; a first carrying air introduction branch pipeline (A7.1) and a fourth carrying air introduction branch pipeline (A7.4) are respectively connected to the twenty-third solenoid valve (B7.2) and the twenty-fourth solenoid valve (B7.3), a twenty-second solenoid valve (B7.1) and a twenty-fifth solenoid valve (B7.4) are respectively installed at middle parts of the first carrying air introduction branch pipeline (A7.1) and the fourth carrying air introduction branch pipeline (A7.4), and the other ends of the first carrying air introduction branch pipeline (A7.1) and the fourth carrying air introduction branch pipeline (A7.4) respectively extend into the general refrigerator refrigerating compartment type positive and negative pressure fresh-keeping cabin (1D) and the general refrigerator freezing compartment type positive and negative pressure fresh-keeping cabin (1E) to form a first carrying air inlet port (i1) and a fourth carrying air inlet port (14) thereof; a first carrying branch pipeline (A6.1), a second carrying branch pipeline (A6.2), a third carrying branch pipeline (A6.3) and a fourth carrying branch pipeline (A6.4) are respectively connected to the air inflation pipeline (A3), an eighteenth solenoid valve (B6.1), a nineteenth solenoid valve (B6.2), a twentieth solenoid valve (B6.3) and a twenty-first solenoid valve (B6.4) are respectively installed at middle parts of the first carrying branch pipeline (A6.1), the second carrying branch pipeline (A6.2), the third carrying branch pipeline (A6.3) and the fourth carrying branch pipeline (A6.4), and the other ends of the first carrying branch pipeline (A6.1), the second carrying branch pipeline (A6.2), the third carrying branch pipeline (A6.3) and the fourth carrying branch pipeline (A6.4) are respectively connected to a first air inlet port (t1), a second air inlet port (t2), a third air inlet port (t3) and a fourth air inlet port (t4) of the second airflow carried substance generator integration (6.022), the third airflow carried substance generator integration (6.023), the fourth airflow carried substance generator integration (6.024) and the fifth airflow carried substance generator integration (6.025); an air intake pipeline (A4) is installed at an air inlet port (e) of the air inflation pump (3), an air filter (6.8) and an eleventh solenoid valve (B4) are installed at middle parts of the air intake pipeline (A4), and the other end of the air intake pipeline (A4) is connected to atmosphere; a circulation pipeline (A5) is further connected to the eleventh solenoid valve (B4), and a first circulation branch pipeline (A5.1), a second circulation branch pipeline (A5.2), a third circulation branch pipeline (A5.3) and a fourth circulation branch pipeline (A5.4) are respectively connected to the circulation pipeline (A5), a thirteenth solenoid valve (B5.1), a fourteenth solenoid valve (B5.2), a fifteenth solenoid valve (B5.3) and a sixteenth solenoid valve (B5.4) are installed at middle parts of the first circulation branch pipeline (A5.1), the second circulation branch pipeline (A5.2), the third circulation branch pipeline (A5.3) and the fourth circulation branch pipeline (A5.4), and the other ends of the first circulation branch pipeline (A5.1), the second circulation branch pipeline (A5.2), the third circulation branch pipeline (A5.3) and the fourth circulation branch pipeline (A5.4) respectively extend into the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) to form a first circulation air return port (j1), a second circulation air return port (j2), a third circulation air return port (j3) and a fourth circulation air return port (j4) thereof; an air-to-water production inflation pipeline (A9) is further connected to the circulation pipeline (5) through a thirty-first solenoid valve (B9), the air-to-water production inflation pipeline (A9) is connected to the fifth solenoid valve (B2) and then communicates with an air inlet port (h) of the air-to-water production apparatus (11) through the air exhaust pipeline (A2), the air-to-water production inflation pipeline (A9) is connected to the circulation pipeline (A5) and communicates with the sixth solenoid valve (B3), and then communicates with an air outlet port (d) of the air inflation pump (3) through the air inflation pipeline (A3); the touch screen and mobile phone monitoring and identification system (12) comprises a refrigerator door touch screen (12.1), a mobile phone APP (12.2), a high-definition anti-fog camera apparatus (12.3), and a radar scanning recognition apparatus (12.5); the high-definition anti-fog camera apparatus (12.3) and the radar scanning recognition apparatus (12.5) are installed at a position needing to be monitored and recognized inside and outside the refrigerator body (7); the cabins of the positive and negative pressure refrigerating fresh-keeping cabin (1B), the positive and negative pressure freezing fresh-keeping cabin (1C), the general refrigerator refrigerating compartment type positive and negative pressure fresh-keeping cabin (1D) and the general refrigerator freezing compartment type positive and negative pressure fresh-keeping cabin (1E) are internally provided with the second airflow carried substance generator integration (6.022), the third airflow carried substance generator integration (6.023), the fourth airflow carried substance generator integration (6.024) and the fifth airflow carried substance generator integration (6.025) and sensor integrations (CA); the sensor (C) comprises one or more of the following individual sensors as required: a pressure sensor (C1), a negative pressure sensor (C2), a super oxygen sensor (C3), a negative ion sensor (C4), a chlorine dioxide sensor (C5), a humidity sensor (C6), an oxygen sensor (C7), a nitrogen sensor (C8), a disinfection, sterilization and degradation sensor (C9), an air particle sensor (C10), a temperature sensor (C11), a carried substance sensor item addition and upgrading module (C12); the carried substance sensor item addition and upgrading module (C12) is a combined module capable of providing and installing any individual sensor additional item into the sensor integration and upgrading and updating the same; the sensor integration (CA) comprises at least one of more of individual sensors (C1) to (C12); all airflow carried substance generation processors (6), sensors (C) and solenoid valves (B) and the air evacuation pump (2), the air inflation pump (3), the refrigeration system (8), the air-to-water production apparatus (11) and the mobile phone monitoring and identification system (12) are connected to the positive and negative pressure intelligent regulation and control apparatus (5) by lines (D) or bundled lines (E).

21. The washing machine according to claim 5, wherein the intelligent positive and negative pressure system further comprises one or more of a carried substance decomposition processor (6.7), a water suction pump (16), a water input pump (17), and a touch screen and mobile phone monitoring and identification system (12), wherein a positive and negative pressure carried substance generation processor (6) is a positive and negative pressure waterflow carried substance generator integration (6.04);
the positive and negative pressure cabin (1) is designed as a washing machine type positive and negative pressure cabin (1F) according to particular requirements for washing and drying; the washing machine type positive and negative pressure cabin (1F) is a vacuum high pressure cabin (1-1) structure; a positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to demands and a washing machine configuration;
the washing machine type positive and negative pressure cabin (1F) comprises a cabin body (1.1), a cabin door (1.2), an airtight mechanism (1.3), and an internal and external communicating sealer (1.4); the airtight mechanism (1.3) is arranged between the cabin door (1.2) and the cabin body (1.1); the airtight mechanism (1.3) comprises a lock ring (1.31), a lock bolt (1.32), an airtight gasket (1.33), and a door rim sealing gasket (1.34); the lock ring (1.31) is in movable fit with the lock bolt (1.32), the lock bolt (1.32) extends into the lock ring (1.31) to lock the cabin door during the closing of the cabin door; when the positive and negative atmospheric pressure is formed inside the washing machine type positive and negative pressure cabin (1F), the airtight gasket (1.33) is used for keeping sealing; the internal and external communicating sealer (1.4) is fixedly arranged at a rear part or side part of washing machine type positive and negative pressure cabin (1F), and all pipes and circuits getting in and out the cabin are connected to and pass through the internal and external communicating sealer (1.4) so as to keep the sealing property of the washing machine type positive and negative pressure cabin (1F);
the positive and negative pressure intelligent regulation and control apparatus (5) comprises an integrated circuit and chip, and a regulation and control system for a fluid and a carried substance thereof, and is connected to the Internet in a wired or wireless mode;
the waterflow carried substance generator integration (6.04) comprises one or more of the following apparatuses: an air conditioning apparatus (6.1), a super oxygen generator (6.2), a negative ion generator (6.3), a catalyst release controller (6.4), a humidification, dehumidification and humidity control apparatus (6.5), a super oxygen generation water mixer (6.6), a carried substance decomposition processor (6.7), an air filter (6.8), a disinfection, sterilization and degradation apparatus (6.9), a stain removal and oil dispelling cleaning apparatus (6.11), a refrigeration, heating and temperature control apparatus (6.13), a carried substance generation item addition and upgrading module (6.16); the carried substance generation item addition and upgrading module (6.16) is a combined module capable of providing or installing any individual fluid carried substance generator additional item in the carried substance generator integration and upgrading and updating the same;
the washing-dehydrating-drying system (15) comprises a washing machine drum mechanism (15.1), a washing-dehydrating-drying control mechanism (15.2), a drum sealing bearing housing (15.3), a drum big belt pulley (15.4), and a motor assembly (15.5); and
the washing machine type positive and negative pressure cabin (1F) is internally provided with the washing machine drum mechanism (15.1), an anti-shower air evacuation port (2.1), the drum sealing bearing housing (15.3), the sensors (C) comprise one or more of the following: a pressure sensor (C1), a negative pressure sensor (C2), a super oxygen sensor (C3), and a carried substance sensor item addition and upgrading module (C12); the carried substance sensor item addition and upgrading module (C12) is a combined module capable of providing or installing any individual sensor additional item in the sensor integration and upgrading and updating the same; the washing machine type positive and negative pressure cabin (1F) is externally provided with the air evacuation pump (2), the air inflation pump (3), the intelligent positive and negative pressure regulation and control apparatus (5), the positive and negative pressure waterflow carried substance generator integration (6.04), the carried substance decomposition processor (6.7), the washing-dehydrating-drying control mechanism (15.2), the drum big belt pulley (15.4), the motor assembly (15.5), the water suction pump (16), the water input pump (17), a detergent pull box (18), and the touch screen and mobile phone monitoring and identification system (12); an air evacuation pipeline (A1) is installed at an air inlet port (a) of the air evacuation pump (2), a first solenoid valve (B1) is installed at a middle part of the air evacuation pipeline (A1), and the other end of the air evacuation pipeline (A1) extends into the washing machine type positive and negative pressure cabin (1F) and then is connected with the anti-shower air evacuation port (2.1) to form an air evacuation and air return port (c) or a circulation air return port (i), and an air outlet port (b) of the air evacuation pump (2) is connected to the atmosphere; a first circulation pipeline (A5) is further connected to the first solenoid valve (B1), a third solenoid valve (B5) is installed at a middle part of the first circulation pipeline (A5), and the other end of the first circulation pipeline (A5) is a circulation exhaust port (i2) or a circulation air inlet port (j2); an air inflation pipeline (A3) is installed at an air outlet port (d) of the air inflation pump (3), a second solenoid valve (B3) is installed at a middle part of the air inflation pipeline (A3), the other end of the air inflation pipeline (A3) extends into washing machine type positive and negative pressure cabin (1F) to form an air inflatable inlet port (f) or a circulation air inlet port (j), and an air inlet port (e) of the air inflation pump (3) is connected to the atmosphere; a second circulation pipeline (A6) is further connected to the second solenoid valve (B3), a fourth solenoid valve (B6) is installed at a middle part of the second circulation pipeline (A6), and the other end of the second circulation pipeline (A6) is connected to a circulation air inlet port (e2) of the air inflation pump (3); a third circulation pipeline (A7) is further connected to the fourth solenoid valve (B6), and the other end of the third circulation pipeline (A7) is connected to the third solenoid valve (B5); a first water intake pipeline (A14) is installed at a water inlet port (r) of the water input pump (17), a fifth solenoid valve (B14) is installed at a middle part of the first water intake pipeline (A14), and the other end of the water intake pipeline (A14) communicates with a tap water source; a second water intake pipeline (A15) of the washing machine is installed at a water output port (u) of the water input pump (17), a sixth solenoid valve (B15) is installed at a middle part of the second water intake pipeline (A15), and the other end of the second water intake pipeline (A15) communicates with a water inlet port (v) of the waterflow carried substance generator integration (6.04); a third water intake pipeline (A16) of the washing machine is installed at a water output port (o) of the water input pump (17), a seventh solenoid valve (B16) is installed at a middle part of the third water intake pipeline (A16), and the other end of the third water intake pipeline (A16) communicates with a water inlet port (2) of the detergent pull box (18); a fourth water intake pipeline (A17) of the washing machine is installed at a water output port (1) of the detergent pull box (18), an eighth solenoid valve (B17) is installed at a middle part of the fourth water intake pipeline (A17), and the other end of the fourth water intake pipeline (A17) extends into the washing machine type positive and negative pressure cabin (1F) to form a water inlet port (T) thereof; a fifth water intake pipeline (A18) is further installed on the fifth solenoid valve (B14), and the solenoid valve (B14) communicates with the seventh solenoid valve (B16) so as to directly feed water when carrying water is not needed and the tap water pressure reaches the standard; a sixth water intake pipeline (A19) is further installed on the sixth solenoid valve (B15), and the sixth solenoid valve (B15) is connected to the fifth water intake pipeline (A18) to communicate with the seventh solenoid valve (B16), such that the water is directly fed by the water input pump (17) when the carrying water is not needed; a first water drainage pipeline (A20) is installed at a water output port (w) of the water suction pump (16), a ninth solenoid valve (B20) is installed at a middle part of the first water drainage pipeline (A20), and the other end of the first water drainage pipeline (A20) extends into the washing machine type positive and negative pressure cabin (1F) to form a water drainage port (L) thereof; a second water drainage pipeline (A21) is installed at a water output port (p) of the water suction pump (16), a tenth solenoid valve (B21) is installed at a middle part of the second water drainage pipeline (A21), and the other end of the second water drainage pipeline (A21) communicates with a water inlet port (g) of the carried substance decomposition processor (6.7); a third water drainage pipeline (A22) is installed at a water output port (m) of the carried substance decomposition processor (6.7), an eleventh solenoid valve (B22) is installed at a middle part of the third water drainage pipeline (A22), and the other end of the third water drainage pipeline (A22) is connected to a sewer; a fourth water drainage pipeline (A23) is installed at the ninth solenoid valve (B20), and the ninth solenoid valve (B20) communicates with the eleventh solenoid valve (B22), such that the water is directly drained when the carried substance does not need to be decomposed and free drainage is achieved; a fifth water drainage pipeline (A24) is further installed on the tenth solenoid valve (B21), and the tenth solenoid valve (B21) is connected to the fourth water drainage pipeline (A23) and then communicates with the eleventh solenoid valve (B22), such that the water is directly drained by the water suction pump (16) when the carried substance does not need to be decomposed; the big belt pulley (15.4) is driven by the motor assembly (15.5), and the big belt pulley (15.4) drives the drum mechanism (15.1) connected thereto to operate; all fluid carried substance generation processors (6), the sensors (C), the solenoid valves (B), and the air evacuation pump (2), the air inflation pump (3), the washing-dehydrating-drying control mechanism (15.2), the water suction pump (16), the water input pump (17), and the touch screen and mobile phone monitoring and identification system (12) are all connected to the positive and negative pressure intelligent regulation and control apparatus (5).

22. The dish washing and fruit and vegetable cleaning machine according to claim 7, wherein the intelligent positive and negative pressure system further comprises one or more of a carried substance decomposition processor (6.7), a water suction pump (16), a water input pump (17), and a touch screen and mobile phone monitoring and identification system (12); wherein the positive and negative pressure fluid carried substance generation processor (*6) is a waterflow carried substance generator integration (6.04);
the positive and negative pressure cabin (1) is designed as a dish washer type positive and negative pressure cabin (1G) according to intelligent dish washing and drying and fruit and vegetable cleaning requirements, and the dish washer type positive and negative pressure cabin (1G) is a vacuum high pressure cabin (1-1) structure; a positive and negative resistance standard of the vacuum high pressure cabin (1-1) takes the local instantaneous atmospheric pressure as zero standard, a positive pressure resistance standard is 0.01 KPa higher than the zero standard until higher, and a negative pressure resistance standard is 0.01 KPa lower than the zero standard until lower, and positive and negative pressure resistance strength is improved according to demands and a configuration of the dish washing and fruit and vegetable cleaning machine;
the dish washer type positive and negative pressure cabin (1G) comprises a cabin body (1.1), a cabin door (1.2), an airtight mechanism (1.3), and an internal and external communicating sealer (1.4), and the airtight mechanism (1.3) is arranged between the cabin door (1.2) and the cabin body (1.1) of the dish washer type positive and negative pressure cabin (1G); the airtight mechanism (1.3) comprises a lock ring (1.31), a lock bolt (1.32), and an airtight gasket (1.33); the lock ring (1.31) is in movable fit with the lock bolt (1.32), the lock bolt (1.32) extends into the lock ring (1.31) to lock the cabin door during the closing of the cabin door; when positive and negative atmospheric pressure is formed inside the dish washer type positive and negative pressure cabin (1G), the airtight gasket (1.33) is used for keeping sealing; the internal and external communicating sealer (1.4) is fixedly arranged at a rear part or side part of the dish washer type positive and negative pressure cabin (1G), and all pipes and circuits getting in and out the cabin are connected to and pass through the internal and external communicating sealer (1.4), so as to keep the sealing property of the dish washer type positive and negative pressure cabin (1G);
the positive and negative pressure intelligent regulation and control apparatus (5) comprises an integrated circuit, a chip, and a regulation and control system for a fluid and a carried substance thereof, and is connected to an Internet in a wired or wireless mode;
the waterflow carried substance generator integration (6.04) comprises one or more of the following apparatuses: a super oxygen generation water mixer (6.6), a disinfection, sterilization and degradation apparatus (6.9), a stain removal and oil dispelling cleaning apparatus (6.11), and a carried substance generator item addition and upgrading module (6.16), the carried substance generator item addition and upgrading module (6.16) is a combined module capable of providing or installing any individual fluid carried substance generator additional item in the carried substance generator integration and upgrading and updating the same; and
the dish washer type positive and negative pressure cabin (1G) is internally provided with the dish washing and drying system (19), the fruit and vegetable cleaning system (20), an anti-shower air evacuation port (2.1), and sensors (C), wherein the sensors comprise one or more of the following apparatuses: a negative pressure sensor (C2), a super oxygen sensor (C3), a temperature sensor (C9), and a carried substance sensor item addition and upgrading module (C12); the carried substance sensor item addition and upgrading module (C12) is a combined module capable of providing or installing any individual sensor additional item in the sensor integration and upgrading and updating the same; the dish washer type positive and negative pressure cabin (1G) is externally provided with an air evacuation pump (2), a positive and negative pressure intelligent regulation and control apparatus (5), a waterflow carried substance generator integration (6.04), a carried substance decomposition processor (6.7), a water suction pump (16), a water input pump (17), and a touch screen and mobile phone monitoring and identification system (12); an air evacuation pipeline (A1) is installed at an air inlet port (a) of the air evacuation pump (2), a first solenoid valve (B1) is installed at a middle part of the air evacuation pipeline (A1), the other end of the air evacuation pipeline (A1) extends into the dish washer type positive and negative pressure cabin (1G) and then is connected with the anti-shower air evacuation port (2.1), so as to form an air evacuation and air return port (c), and an air outlet port (b) of the air evacuation pump (2) is connected to the atmosphere; a first water intake pipeline (A14) is installed at a water inlet port (r) of the water input pump (17), a second solenoid valve (B14) is installed at a middle part of the first water intake pipeline (A14), and the other end of the first water intake pipeline (A14) communicates with a tap water source; a second water intake pipeline (A15) is installed at a water output port (u) of the water input pump (17), a third solenoid valve (B15) is installed at a middle part of the second water intake pipeline (A15), and the other end of the second water intake pipeline (A15) communicates with a water inlet port (k) of the waterflow carried substance generator integration (6.04); a third water intake pipeline (A16) is installed at a water output port (q) of the waterflow carried substance generator integration (6.04), a fourth solenoid valve (B16) is installed at a middle part of the third water intake pipeline (A16), and the other end of the third water intake pipeline (A16) extends into the dish washer type positive and negative pressure cabin (1G) to be connected to a fifth solenoid valve (B25); the fifth solenoid valve (B25) is respectively connected to a fruit and vegetable cleaning water intake pipeline (A25) and a dish washing and drying water intake pipeline (A27), and the fruit and vegetable cleaning water intake pipeline (A25) is connected to a water inlet port (e) of the fruit and vegetable cleaning system (20); the dish washing and drying water intake pipeline (A27) is connected to a water inlet port (G) of the dish washing and drying system (19); a fourth water intake pipeline (A19) is further installed at the third solenoid valve (B15), and the third solenoid valve (B15) is connected to the fourth solenoid valve (B16), such that the water is directly fed by the water input pump (17) when super-oxygenated water is not required; a first water drainage pipeline (A20) is installed at a water inlet port (w) of the water suction pump (16), a sixth solenoid valve (B20) is installed at a middle part of the first water drainage pipeline (A20), and the other end of the first water drainage pipeline (A20) extends into the dish washer type positive and negative pressure cabin (1G) and is respectively connected to a fruit and vegetable cleaning water drainage pipeline (A26) and a dish washing and drying water drainage pipeline (A28), and the fruit and vegetable cleaning water drainage pipeline (A26) is connected to a water output port (F) of the fruit and vegetable cleaning system (20); the dish washing and drying water drainage pipeline (A28) is connected to a water drainage port (H) of the dish washing and drying system (19); a second water drainage pipeline (A21) is installed at a water output port (p) of the water suction pump (16), a seventh solenoid valve (B21) is installed at a middle part of the second water drainage pipeline (A21), and the other end of the second water drainage pipeline (A21) communicates with a water inlet port (g) of the carried substance decomposition processor (6.7); a third water drainage pipeline (A22) is installed at a water output port (m) of the carried substance decomposition processor (6.7), an eighth solenoid valve (B22) is installed at a middle part of the third water drainage pipeline (A22), and the other end of the third water drainage pipeline (A22) communicates with a sewer; a fourth water drainage pipeline (A24) is further installed at the seventh solenoid valve (B21), and the seventh solenoid valve (B21) is connected to fourth water drainage pipeline (A23) and then communicates with the eighth solenoid valve (B22), such that the water is directly drained by the water suction pump when the super oxygen and the carried substance do not need to be decomposed; the positive and negative pressure waterflow carried substance generator integration (6), sensors (C) and solenoid valves (B) and the air evacuation pump (2), the air inflation pump (3), the water suction pump (16), the water input pump (17), the dish washing and drying system (19), the fruit and vegetable cleaning system (20), and the touch screen and mobile phone monitoring and identification system (12) are all connected to the positive and negative pressure intelligent regulation and control apparatus (5).

23. The fresh-keeping transport compartment container according to claim 12, wherein the intelligent positive and negative pressure system further comprises sensors (C), and/or a touch screen and mobile phone monitoring and identification system (12);
the positive and negative pressure cabin (1) is designed as a compartment container type positive and negative pressure cabin (1M) or an atmospheric compartment container type positive and negative pressure cabin (1M-2) according to the requirements of the intelligent positive and negative pressure fresh-keeping transport compartment container; an internal structure of the compartment container type positive and negative pressure cabin (1M) comprises a cabin body (1.1), a cabin door (1.2), an airtight mechanism (1.3), and an internal and external communicating sealer (1.4); the cabin door (1.2) is a compartment door of the positive and negative pressure fresh-keeping transport compartment container; the airtight mechanism (1.3) is arranged between the cabin door (1.2) and the cabin body (1.1) of the compartment container type positive and negative pressure cabin (1M); the airtight mechanism (1.3) comprises a lock ring (1.31), a lock bolt (1.32), and an airtight gasket (1.33); the lock ring (1.31) is in movable fit with the lock bolt (1.32), the lock bolt (1.32) extends into the lock ring (1.31) to lock the cabin door during the closing of the cabin door, and when the positive and negative pressure is formed inside the compartment container type positive and negative pressure cabin (1M), the airtight gasket (1.33) is used for keeping sealing; the internal and external communicating sealer 1.4 is fixedly arranged at a rear part of the compartment container type positive and negative pressure cabin (1M), and all pipes and circuits getting in and out the cabin are connected to and pass through the internal and external communicating sealer (1.4); the compartment container type positive and negative pressure cabin (1M), due to the installation of the airtight mechanism (1.3) and the internal and external communicating sealer (1.4) and the possess of a corresponding vacuum high pressure resistance structure, belongs to an airtight vacuum high pressure cabin (1-1) structure; a positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower, and positive and negative pressure resistance strength is improved according to specific demands and a configuration of the compartment container; the atmospheric compartment container type positive and negative pressure cabin (1M-2) is a general compartment container with the positive and negative pressure system and without the airtight mechanism (1.3) and the internal and external communicating sealer (1.4); a general compartment container space is an atmospheric positive and negative pressure cabin without a vacuum high pressure resistance structure, belonging to a general sealed atmospheric circulation cabin (1-2) or atmospheric inlet-outlet cabin (3) structure;
the intelligent positive and negative pressure regulation and control apparatus (5) comprises an integrated circuit, a chip, a regulation and control system for a fluid and carried substance thereof, and is connected to the Internet in wired and wireless modes;
the positive and negative pressure fluid carried substance generation processor (6) comprises one or more of the following apparatuses: an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), one or more airflow carried substance generator integrations, and an integrated pipeline type positive and negative fluid carried substance generator (6.05); the air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same; of the one or more airflow carried substance generator integrations (6.02) and the integrated pipeline type positive and negative pressure fluid carried substance generator (6.05) each comprise one or more of the following apparatuses: the air conditioning apparatus (6.1), the super oxygen generator (6.2), the negative ion generator (6.3), the catalyst release controller (6.4), the humidification, dehumidification and humidity control apparatus (6.5), the carried substance decomposition processor (6.7), the air filter (6.8), the disinfection, sterilization and degradation apparatus (6.9), and the carried substance generation item addition and upgrading module (6.16); and
the compartment container type positive and negative pressure cabin (1M) or the atmospheric compartment container type positive and negative pressure cabin (1M-2) is externally provided with the air evacuation pump (2), the air inflation pump (3), the positive and negative pressure intelligent regulation and control apparatus (5), a first airflow carried substance generator integration (6.02), the carried substance decomposition processor (6.7), the air filter (6.8), the refrigeration system (8), an air-to-water production apparatus (11), and a touch screen and mobile phone monitoring and identification system (12); an air evacuation pipeline (A1) is installed at an air inlet port (a) of the air evacuation pump (2), a first solenoid valve (B1) is installed at a middle part of the air evacuation pipeline (A1), and the other end of the air evacuation pipeline (A1) extends into the compartment container type positive and negative pressure cabin (1M) or the atmospheric compartment container type positive and negative pressure cabin (1M-2) to form an air evacuation and air return port (c) thereof; a first carrying waste gas evacuation and exhaust pipeline (A8) and a second carrying waste gas evacuation and exhaust pipeline (A8.1) are respectively connected to the first solenoid valve (B1), the other end of the first carrying waste gas exhaust and evacuation pipeline (A8) is connected to a carrying waste gas evacuation and exhaust port (s1) of the air conditioning apparatus (6.1) integrated in the first airflow carried substance generator integration (6.02); the other end of the second carrying waste gas exhaust and evacuation pipeline (A8.1) is connected to a carrying waste gas evacuation and exhaust port (s2) of the air conditioning apparatus (6.1) integrated in a second airflow carried substance generator integration (6.021); an air exhaust pipeline (A2) is installed at an air outlet port (b) of the air evacuation pump (2), the carried substance decomposition processor (6.7) and a second solenoid valve (B2) are installed at a middle part of the air exhaust pipeline (A2), the other end of the air exhaust pipeline (A2) communicates with an air inlet port (h) of the air-to-water production apparatus (11), and passes through the air-to-water production apparatus (11) and then is connected to the atmosphere through an air outlet port (n) of the air-to-water production apparatus (11); an air inflation pipeline (A3) is installed at an air outlet port (d) of the air inflation pump (3), a third solenoid valve (B3) is installed at a middle part of the air inflation pipeline (A3), and the other end of the air inflation pipeline (A3) extends into the compartment container type positive and negative pressure cabin (1M) or the atmospheric compartment container type positive and negative pressure cabin (1M-2) to form an air inflation and inlet port (f) thereof; an air intake pipeline (A4) is installed at an air inlet port (c) of the air inflation pump (3), a fourth solenoid valve (B4) and the air filter (6.8) are installed at a middle part of the air intake pipeline (A4), and the other end of the air intake pipeline (A4) is connected to atmosphere; a first carrying circulation pipeline (A5) is installed at an air inlet port (e2) of the air inflation pump (3), the other end of the first carrying circulation pipeline (A5) communicates with the third solenoid valve (B3), and then extends into the compartment container type positive and negative pressure cabin (1M) or the atmospheric compartment container type positive and negative pressure cabin (1M-2) through the air inflation pipeline (A3) to form a circulating air return port (j) thereof; a first carrying air introduction pipeline (A6), a second carrying air introduction pipeline (A6.1) and a third carrying air introduction pipeline (A6.2) are further connected to the air inflation pipeline (A3), a fifth solenoid valve (B6) is installed at a middle part of the first carrying air introduction pipeline (A6), and the other end of the first carrying air introduction pipeline (A6) is connected to an air inlet port (t) of the first airflow carried substance generator integration (6.02); a fourth carrying air introduction pipeline (A7) is installed at an air outlet port (s) of the first airflow carried substance generator integration (6.02), an eighth solenoid valve (B7) is installed at a middle part of the fourth carrying air introduction pipeline (A7), and the other end of the fourth carrying air introduction pipeline (A7) extends into the compartment container type positive and negative pressure cabin (1M) or the atmospheric compartment container type positive and negative pressure cabin (1M-2) to form a carrying air inlet port (i) thereof; a sixth solenoid valve (B6.1) and a seventh solenoid valve (B6.2) are respectively installed at middle parts of the air introduction pipelines (A6.1) and (A6.2), and the other ends of the air introduction pipelines (A6.1) and (A6.2) extend into the compartment container type positive and negative pressure cabin (1M) or the atmospheric compartment container type positive and negative pressure cabin (1M-2) to be connected to air inlet ports of the second airflow carried substance generator integration (6.021) and the integrated pipeline type positive and negative pressure fluid carried substance generator (6.05); an air-to-water production air inflation pipeline (A9) is further connected to the air inflation pipeline (A3), the air-to-water production air inflation pipeline (A9) is connected to the second solenoid valve (B2) and then is connected to the air exhaust pipeline (A2); the middle part of the air exhaust pipeline (A2) communicates with the air-to-water production apparatus (11), and the other end of the air exhaust pipeline (A2) is connected to the atmosphere; the touch screen and mobile phone monitoring and identification system (12) comprises a door touch screen (12.1), a mobile phone APP (12.2), a wireless anti-fog camera identification apparatus (12.4), and a wireless radar scanning and identification apparatus (12.6); the wireless anti-fog camera identification apparatus (12.4) and the wireless radar scanning and identification apparatus (12.6) are installed at positions inside/outside the compartment body (7) needing to be monitored and identified; the compartment container type positive and negative pressure cabin (1M) or the atmospheric compartment container type positive and negative pressure cabin (1M-2) is internally provided with the second airflow carried substance generator integration (6.021), an integrated pipeline type positive and negative pressure fluid carried substance generator (6.05), and corresponding sensors thereof; the sensors (C) comprise a sensor integration (CA), the sensor integration (CA) comprises at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12); the carried substance sensor item addition and upgrading module (C12) is a combined module capable of providing and installing any individual sensor additional item into the sensor integration and upgrading and updating the same; all fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).

24. The disinfection machine according to claim 16, wherein the intelligent positive and negative pressure system further comprises a touch screen and mobile phone monitoring system (12), and/or sensors (C);
the intelligent positive and negative pressure regulation and control apparatus (5) comprises an integrated circuit, a chip, a regulation and control system for a fluid and carried substance thereof, and is connected to the Internet in wired and wireless modes;
the positive and negative pressure fluid carried substance generation processor (6) comprises one or more of the following apparatuses: a super oxygen generator air disinfection apparatus (6.2), a negative ion generator air improvement apparatus (6.3), a humidification and humidity controller air regulation apparatus (6.5), an instantaneous disinfector filtering and disinfection apparatus (6.11), and a carried substance generator item addition and upgrading module (6.16); the carried substance generator item addition and upgrading module (6.16) is a combined module capable of providing or installing any individual fluid carried substance generator additional item in the carried substance generator integration and upgrading and updating the same;
the touch screen and mobile phone monitoring system (12) comprises a touch screen (12.1), a mobile phone monitoring system (12.2), a location positioning system (12.5);
the sensors (C) comprise one or more of the following sensors: a super oxygen sensor (C3), a negative ion sensor (C4), a humidity sensor (C6), a disinfection and degradation sensor (C9), and a water level sensor (13); and
an air inflation pipeline (A3) is installed at an air outlet port (d) of the air inflation pump (3), and a first air inflation branch pipeline (A3.1), a second air inflation branch pipeline (A3.2), a third air inflation branch pipeline (A3.3), a fourth air inflation branch pipeline (A3.4) and a fifth air inflation branch pipeline (A3.5) are respectively connected to the other end of the air inflation pipeline (A3); a first solenoid valve (B3.1) is installed at a middle part of the first air inflation branch pipeline (A3.1), and the other end of the first air inflation branch pipeline (A3.1) is connected to an air inflation and outlet port of the machine body (7); a second solenoid valve (B3.2) and the negative ion generator air improvement apparatus (6.3) are installed at a middle part of the second air inflation branch pipeline (A3.2), and the other end of the second air inflation branch pipeline (A3.2) is connected to a negative ion outlet port of the machine body (7); a third solenoid valve (B3.3) and the super oxygen generator air infection apparatus (6.2) are installed at a middle part of the third air inflation branch pipeline (A3.3), and the other end of the third air inflation branch pipeline (A3.3) is connected to a super oxygen outlet port of the machine body (7); a fourth solenoid valve (B3.4) and the humidification and humidity controller air regulation apparatus (6.5) are installed at a middle part of the fourth air inflation branch pipeline (A3.4), and the other end of the fourth air inflation branch pipeline (A3.4) is connected to a humidification and air outlet port of the machine body (7); a fifth solenoid valve (B3.5) and the air-to-water production apparatus (11) are installed at a middle part of the fifth air inflation branch pipeline (A3.5), and the other end of the fifth air inflation branch pipeline (A3.5) is connected to a water production and air exhaust port of the machine body (7); an air intake pipeline (A4) is installed at an air inlet port (e) of the air inflation pump (3), a sixth solenoid valve (B4) is installed at a middle part of the air intake pipeline (A4), and the other end of the air intake pipeline (A4) is connected to a direct air inlet port of the machine body (7); a filtering and air intake pipeline (A4.1) is further connected to the sixth solenoid valve (B4), the air filter (6.8) and a seventh solenoid valve (B4.1) are installed at a middle part of the filtering and air intake pipeline (A4.1), and the other end of the filtering and air intake pipeline (A4.1) is connected to a filtering and air inlet port of the machine body (7); a circulating air return pipeline (A5) is further connected to the seventh solenoid valve (B4.1), the other end of the circulating air return pipeline (A5) is connected to a circulating air return port of the machine body (7); an air-to-water production pipeline (A10) is installed at a water output port (x) of the air-to-water production apparatus (11), the air-to-water production pipeline (A10) communicates with a water inlet port (y) of a filtering water tank (11.1); a filtering water pipeline (A12) is installed at a water output port (v) of the filtering water tank (11.1), the filtering water pipeline (A12) communicates with a water inlet port of the humidification and humidity controller air regulation apparatus (6.5), a water adding pipeline (A11) is installed at a spare water adding port (u) of the filtering water tank (11.1), an eighth solenoid valve (B11) is installed at a middle part of the water adding pipeline (A11), and the other end of the water adding pipeline (A11) is connected to a spare water adding port of the machine body (7); an air exhaust pipeline (A1) is installed at an air inlet port (a) of the air evacuation pump (2), the instantaneous disinfector filtering and disinfection apparatus (6.11) is installed at a middle part of the air exhaust pipeline (A1), and the other end of the air exhaust pipeline (A1) is connected to an air evacuation and air return port of the machine body (7); an air exhaust pipeline (A2) is installed at an air outlet port (b) of the air evacuation pump (2), a ninth solenoid valve (B2) is installed at a middle part of the air exhaust pipeline (A2), and the other end of the air exhaust pipeline (A2) is connected to the atmosphere; the ninth solenoid valve (B2) is connected to a tenth solenoid valve (B3.5) through the pipeline (A2.1), communicates with the air-to-water production pipeline (A3.5) and an air inlet port (h) of the air-to-water production apparatus (11) in sequence, and then communicates with a water production and air exhaust port via the air outlet port (n) of the air-to-water production apparatus (11) after passing through the air-to-water production apparatus (11); the positive and negative pressure multifunctional disinfection machine has the same structure as the positive and negative pressure full-functional disinfection machine, but does not comprise the negative ion generator air improvement apparatus (6.3); the positive and negative pressure multifunctional disinfection machine does not comprise the negative ion generator air improvement apparatus (6.3), other structures of which are the same as those of the positive and negative pressure full-functional disinfection machine; the positive and negative pressure automatic humidification and disinfection machine does not comprise the instantaneous disinfector filtering and disinfection apparatus (6.11), other structures of which are the same as those of the positive and negative pressure full-functional disinfection machine; the positive and negative pressure humidification and disinfection machine does not comprise the air-to-water production apparatus (11), other structures of which are the same as those of the positive and negative pressure automatic humidification and disinfection machine; the positive and negative pressure disinfection machine does not comprise a humidification and humidity control air regulation apparatus (6.5), other structures of which are the same as those of the positive and negative pressure humidification and disinfection machine; the fluid carried substance generation processor (6), the sensor (C), the solenoid valve (B), the air evacuation pump (2), the air inflation pump (3), the air-to-water production apparatus (11) and the touch screen and mobile phone monitoring and identification system (12) of each machine model are connected to the positive and negative pressure regulation and control apparatus (5).

25. The module cabinet according to claim 18, wherein the positive and negative pressure system further comprises sensors (C), and/or a touch screen and mobile phone monitoring and identification system (12);
the positive and negative pressure cabin (1) is designed as a module type positive and negative pressure cabin (1T) according to the requirements of the intelligent positive and negative pressure module cabinet, and the module type positive and negative pressure cabin (1T) is a vacuum high-pressure cabin (1-1) structure; a positive and negative resistance standard of the vacuum high pressure cabin (1-1) takes the local instantaneous atmospheric pressure as a zero standard, a positive pressure resistance standard is 0.01 KPa higher than the zero standard until higher, and a negative pressure resistance standard is 0.01 KPa lower than the zero standard until lower;
the module type positive and negative pressure cabin (1T) comprises a cabin body (1.1), a cabin door (1.2), a mechanical airtight mechanism (1.3), and an internal and external communicating sealer (1.4); the cabin door (1.2) is a cabinet door of the positive and negative pressure module cabinet; the airtight mechanism (1.3) is arranged between the cabin door (1.2) and the cabin body (1.1) of the module type positive and negative pressure cabin (1T); the internal and external communicating sealer (1.4) is fixedly arranged at a rear part of the module type positive and negative pressure cabin (1T), all pipelines and circuits getting in and out the cabin are connected to and pass through the internal and external communicating sealer (1.4), so as to keep the sealing property of the module type positive and negative pressure cabin (1T);
the intelligent positive and negative pressure regulation and control apparatus (5) comprises an integrated circuit, a chip, a regulation and control system for a fluid and carried substance thereof, and is connected to the Internet in wired and wireless modes;
the positive and negative pressure fluid carried substance generation processor (6) comprises one or more of the following apparatuses: an air conditioning apparatus (6.1), a super oxygen generator (6.2), a negative ion generator (6.3), a catalyst release controller (6.4), a humidification, dehumidification and humidity control apparatus (6.5), a carried substance decomposition processor (6.7), an air filter (6.8), a disinfection, sterilization and degradation apparatus (6.9), a carried substance generator item addition and upgrading module (6.16), one or more airflow carried substance generator integrations, and an integrated pipeline type positive and negative fluid carried substance generator (6.05); the air conditioning apparatus (6.1) is a membrane-based air separation type air conditioning apparatus; the carried substance generator item addition and upgrading module (6.16) is a combined module capable of providing or installing any individual fluid carried substance generator additional item in the carried substance generator integration and upgrading and updating the same; the one or more airflow carried substance generator integrations and the integrated pipeline type positive and negative pressure fluid carried substance generator (6.05) each comprise one or more of the air conditioning apparatus (6.1), the super oxygen generator (6.2), the negative ion generator (6.3), the catalyst release controller (6.4), the humidification, dehumidification and humidity control apparatus (6.5), the carried substance decomposition processor (6.7), the air filter (6.8), the disinfection, sterilization and degradation apparatus (6.9), and the carried substance generator item addition and upgrading module (6.16); and
the module type positive and negative pressure cabin (1T) is externally provided with the air evacuation pump (2), the air inflation pump (3), the positive and negative pressure intelligent regulation and control apparatus (5), the airflow carried substance generator integration (6.02), the carried substance decomposition processor (6.7), the air filter (6.8), the refrigeration and heating system (8.3), an air-to-water production apparatus (11), and the touch screen and mobile phone monitoring and identification system (12); an air evacuation pipeline (A1) is installed at an air inlet port (a) of the air evacuation pump (2), a first solenoid valve (B1) is installed at a middle part of the air evacuation pipeline (A1), and the other end of the air evacuation pipeline (A1) extends into the module type positive and negative pressure cabin (1T) to form an air evacuation and air return port (c) thereof; a first carrying waste gas evacuation and exhaust pipeline (A8) and a second carrying waste gas evacuation and exhaust pipeline (A8.1) are respectively connected to the first solenoid valve (B1), the other end of the first carrying waste gas exhaust and evacuation pipeline (A8) is connected to a carrying waste gas evacuation and exhaust port (s1) of the air conditioning apparatus (6.1) integrated in the positive and negative pressure airflow carried substance generator integration (6.02), and the other end of the second carrying waste gas exhaust and evacuation pipeline (A8.1) is connected to a carrying waste gas evacuation and exhaust port (s2) of the air conditioning apparatus (6.1) integrated in a second airflow carried substance generator integration (6.021); an air exhaust pipeline (A2) is installed at an air outlet port (b) of the air evacuation pump (2), the carried substance decomposition processor (6.7) and a second solenoid valve (B2) are installed at a middle part of the air exhaust pipeline (A2), the other end of the air exhaust pipeline (A2) communicates with an air inlet port (h) of the air-to-water production apparatus (11), passes through the air-to-water production apparatus (11), and then is connected to the atmosphere from an air outlet port (n) of the air-to-water production apparatus (11); an air inflation pipeline (A3) is installed at an air outlet port (d) of the air inflation pump (3), a third solenoid valve (B3) is installed at a middle part of the air inflation pipeline (A3), and the other end of the air inflation pipeline (A3) extends into the module type positive and negative pressure cabin (1T) to form an air inflation and inlet port (f) thereof; an air intake pipeline (A4) is installed at an air inlet port (e) of the air inflation pump (3), a fourth solenoid valve (B4) and the air filter (6.8) are installed at a middle part of the air intake pipeline (A4), and the other end of the air intake pipeline (A4) is connected to the atmosphere; a circulation pipeline (A5) is further connected to the fourth solenoid valve (B4), and the other end of the circulation pipeline (A5) communicates with the third solenoid valve (B3), and extends into the module type positive and negative pressure cabin (1T) through the air inflation pipeline (A3) to form a circulating air return port (j) thereof; a first carrying air introduction pipeline (A6), a second carrying air introduction pipeline (A6.1) and a third carrying air introduction pipeline (A6.2) are further connected to the air inflation pipeline (A3), a fifth solenoid valve (B6) is installed at a middle part of the first carrying air introduction pipeline (A6), the other end of the first carrying air introduction pipeline (A6) is connected to an air inlet port (t) of the airflow carried substance generator integration (6.02); a fourth carrying air introduction pipeline (A7) is installed at an air outlet port (s) of the first airflow carried substance generator integration (6.02), a sixth solenoid valve (B7) is installed at a middle part of the fourth carrying air introduction pipeline (A7), and the other end of the fourth carrying air introduction pipeline (A7) extends into the positive and negative pressure cabin (1T) to form a carrying air inlet port (i) thereof; a seventh solenoid valve (B6.1) and an eighth solenoid valve (B6.2) are respectively installed at middle parts of the second carrying air introduction pipeline (A6.1) and the third carrying air introduction pipeline (A6.2), the other ends of the second carrying air introduction pipeline (A6.1) and the third carrying air introduction pipeline (A6.2) extend into the positive and negative pressure cabin (1) to be connected to air inlet ports of the second airflow carried substance generator integration (6.021) and the integrated pipeline type positive and negative pressure fluid carried substance generator (6.05); an air-to-water production inflation pipeline (A9) is further connected to the air inflation pipeline (A3), the air-to-water production inflation pipeline (A9) is connected to the second solenoid valve (B2) and is then connected to the air exhaust pipeline (A2); the air-to-water production apparatus (11) communicates with the middle part of the air exhaust pipeline (A2), and the other end of the air exhaust pipeline (A2) is connected to the atmosphere; the touch screen and mobile phone monitoring and identification system (12) comprises a cabinet door touch screen (12.1), a mobile phone APP (12.2), a high-definition anti-fog camera device (12.3), and a radar scanning and identification apparatus (12.5); the high-definition anti-fog camera device (12.3) and the radar scanning and identification apparatus (12.5) are installed at positions inside/outside the cabinet body (7) needing to be monitored; the module type positive and negative pressure cabin (1T) is internally provided with the second airflow carried substance generator integration (6.021) or the integrated pipeline type fluid carried substance generator (6.05) and sensors (C); the sensors comprise a sensor integration (CA), the sensor integration (CA) comprises at least one or more of the following individual sensors (C1) to (C12) as required: a pressure sensor (C1), a negative pressure sensor (C2), a super oxygen sensor (C3), a negative ion sensor (C4), a catalyst sensor (C5), a humidity sensor (C6), an oxygen sensor (C7), a nitrogen sensor (C8), a disinfection and degradation sensor (C9), an air particle sensor (C10), a temperature sensor (C11), and a carried substance sensor item addition and upgrading module (C12); the carried substance sensor item addition and upgrading module (C12) is a combined module capable of providing and installing any individual sensor additional item into the sensor integration and upgrading and updating the same; all fluid carried substance generation processors (6), sensors (C) and solenoid valves (B) and the air evacuation pump (2), the air inflation pump (3), the refrigeration system (8), the air-to-water production apparatus (11) and the touch screen and mobile phone monitoring and identification system (12) are connected to the positive and negative pressure intelligent regulation and control apparatus (5) by lines (D) or bundled lines (E).
